(19) United States
(12) Patent Application Publication
(10) Pub. No.: US 2026/0109711 A1
(43) Pub. Date: Apr. 23, 2026

(54) MACROCYCLIC SULFONAMIDE MODULATORS

(71) Applicant: Vertex Pharmaceuticals Incorporated, Boston, MA (US)

(72) Inventors: Jun Myun AHN, Waltham, MA (US); Emily Elizabeth ALLEN, Boston, MA (US); David John CHARBONEAU, Watertown, MA (US); Ming CHEN, Medford, MA (US); Leslie A. DAKIN, Lakeville, MA (US); Christina Anne DE BRUYN KOPS, Boston, MA (US); Travis Jon DELANO, Boston, MA (US); Pedro Manuel GARCIA BARRANTES, Melrose, MA (US); Ronald Lee GREY, JR., Mansfield, MA (US); Megan Elizabeth HOERRNER, Somerville, MA (US); Sheila NAMIREMBE, Northborough, MA (US); Arjun Venkat NARAYANAN, Cambridge, MA (US); Jessica Howard OLSEN, Jamaica Plain, MA (US); Madison Henry POWERS, Cambridge, MA (US); Tony Z. SCOTT, Cambridge, MA (US); Steven David STONE, Quincy, MA (US); Emil Francois THOMAS, Boston, MA (US); Xiaoxu WANG, Arlington, MA (US); Ashley K. Z. ZACHMANN, Wayland, MA (US); Mariam ZAKY, Boston, MA (US); Chenlong ZHANG, Wayland, MA (US)

(73) Assignee: Vertex Pharmaceuticals Incorporated, Boston, MA (US)

(21) Appl. No.: 19/331,395

(22) Filed: Sep. 17, 2025

Related U.S. Application Data
(60) Provisional application No. 63/695,953, filed on Sep. 18, 2024.
(60) Provisional application No. 63/757,604, filed on Feb. 12, 2025.

(51) Int. Cl. C07D 498/08; A61K 31/407; A61K 31/439; C07D 498/18; C07D 498/20

(52) U.S. Cl. CPC C07D 498/08; A61K 31/407; A61K 31/439; C07D 498/18; C07D 498/20

(57) ABSTRACT

Disclosed are compounds, compositions, and methods useful for preventing or treating a disease which is at least partially mediated by orexin receptor 2.

### CROSS-REFERENCE TO RELATED APPLICATIONS

This application claims the benefit of U.S. Provisional Application No. 63/695,953, filed Sep. 18, 2024, and U.S. Provisional Application No. 63/757,604, filed Feb. 12, 2025, each of which is incorporated by reference herein in its entirety.

### FIELD OF THE DISCLOSURE

The present disclosure generally relates to modulating (and more specifically, agonist or antagonist) compounds of orexin receptor 2 (OX2R) and methods of their use in treating conditions at least partially mediated by OX2R.

### BACKGROUND

Orexins are hypothalamic neuropeptides that are implicated in, for example, sleep/wake control and body weight homeostasis. The peptides act on two G protein-coupled receptors termed Orexin receptor type 1 (OX1R) and Orexin receptor type 2 (OX2R). Orexin producing neurons are exclusively localized in the lateral hypothalamic area, but their receptors are expressed in many areas of the brain.

Narcolepsy is a socially debilitating disorder characterized by an inability to properly maintain wakefulness (excessive daytime sleepiness, sleep attacks), and a pathological intrusion of signs of REM sleep into wakefulness (cataplexy, hypnagogic hallucination, sleep paralysis, etc.). Narcolepsy affects an estimated 1 in every 2,000 individuals, and is a non-progressive, life-long condition. A deficiency in orexin/OX2R signaling causes the sleep disorder narcolepsy in humans, mice and dogs. A vast majority (>90%) of human narcoleptics lack detectable levels of orexin peptides in the cerebrospinal fluid due to a highly specific (probably autoimmune) degeneration of orexin neurons, indicating that human narcolepsy is an “orexin deficiency syndrome.”

Compounds having OX2R agonism activity have previously been described (see, e.g., Nagahara, et al., J. Med. Chem., 2015, 58, 7931-37), however those compounds may possess some undesirable properties (e.g., poor pharmacokinetic properties and/or central nervous system penetration, poor selectivity for OX2R, etc.) Thus, there remains a need for OX2R agonist compounds having improved properties.

### SUMMARY

One aspect of the invention provides compounds, compositions, and methods useful for preventing or treating a disease that is at least partially mediated by orexin receptor 2. It was surprisingly discovered that the compounds disclosed and claimed herein demonstrate, among other things, high potency and selectivity for OX2R, and improved drug-like properties, such as central nervous system penetration (e.g., as determined by a MDR1-MDCK permeability assay) and a favorable pharmacokinetic profile.

In one aspect, the invention relates to a compound described herein (e.g., a compound of Formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a compound of Table A, Table B, Table C, Table D, or Table E), or a pharmaceutically acceptable salt thereof.

In another aspect, the invention relates to a pharmaceutical composition comprising the compound, or a pharmaceutically acceptable salt thereof, and at least one pharmaceutically acceptable excipient.

In still another aspect, the invention relates to a method of preventing or treating a disease associated with a deficiency in orexin 2 receptor signaling by administering the compound, pharmaceutically acceptable salt, or pharmaceutical composition.

In yet another aspect the invention relates to a method of preventing or treating a disease selected from narcolepsy, idiopathic hypersomnia, hypersomnia, sleep apnea syndrome, narcolepsy syndrome involving narcolepsy-like symptom, hypersomnia associated with Parkinson's disease, hypersomnia associated with dementia with Lewy body, hypersomnia syndrome involving daytime hypersomnia (e.g., Kleine-Levin syndrome, major depression accompanied by hypersomnia, dementia with Lewy body, Parkinson's disease, progressive supranuclear palsy, Prader-Willi syndrome, Moebius syndrome, hypoventilation syndrome, Niemann-Pick disease type C, brain contusion, cerebral infarction, brain tumor, muscular dystrophy, multiple sclerosis, acute disseminated encephalomyelitis, Guillain-Barre syndrome, Rasmussen's encephalitis, Wernicke's encephalopathy, limbic encephalitis, Hashimoto encephalopathy), coma, loss of consciousness, obesity (e.g., malignant mast cell, extrinsic obesity, hyperinsulinar obesity, hyperplasmic obesity, hypophysial obesity, hypoplasmic obesity, hypothyroid obesity, hypothalamic obesity, symptomatic obesity, childhood obesity, upper body obesity, alimentary obesity, gonadal obesity, systemic mastocytosis, primary obesity, central obesity), insulin resistance syndrome, Alzheimer, impaired consciousness such as coma, side effect or complication caused by anesthesia, sleep disturbance, sleep problem, insomnia, intermittent sleep, night myoclonus, REM sleep interruption, jet lag, jet lag syndrome, sleep disorder of shift workers, dyssomnia, sleep terror, depression, major depression, sleepwalking, enuresis, sleep disorder, Alzheimer's sundown syndrome, disease associated with circadian rhythm, fibromyalgia, condition resulting from decrease in sleeping quality, bulimia, obsessive eating disorder, obesity-related diseases, hypertension, diabetes, elevated plasma insulin level/insulin resistance, hyperlipemia, hyperlipidaemia, endometrial cancer, breast cancer, prostate cancer, colon cancer, cancer, osteoarthritis, obstructive sleep apnea, cholelithiasis, gallstone, heart disease, abnormal heartbeat, arrhythmia, myocardial infarction, congestive heart failure, heart failure, coronary heart disease, cardiovascular disease, sudden death, polycystic ovary, craniopharyngioma, Prader Willi syndrome, Froehlich syndrome, growth hormone deficiency, normal variant short stature, Turner syndrome, children suffering from acute lymphoblastic leukemia, syndrome X, reproductive hormone abnormality, decrease of fecundability, infertility, hypogonadism in men, sexual/reproductive-function dysfunction such as hirsutism in women, fetal defect associated with maternity obesity, gastrointestinal motility disorder such as obesity-related gastroesophageal reflux, obesity hypoventilation syndrome (Pickwickian syndrome), respiratory disease such as respiratory distress, inflammation such as vascular systemic inflammation, arteriosclerosis, hypercholesterolemia, hyperuricemia, low back pain, gallbladder disease, gout, renal cancer, secondary risk of obesity such as risk of left ventricle hypertrophy, migraine, headache, neuropathic pain, Parkinson's disease, psychosis, schizophrenia, facial flushing, night sweat, disease in genitalium/urinary system, disease associated with sexual function or fecundability, dysthymic disorder, bipolar disorder, bipolar I disorder, bipolar II disorder, cyclothymic disorder, acute stress disorder, agoraphobia, generalized anxiety disorder, obsessive-compulsive disorder, panic attack, panic disorder, posttraumatic stress disorder, separation anxiety disorder, social phobia, anxiety disorder, acute neurological and psychiatric disorder such as cerebral deficiency developed after heart bypass surgery or heart transplant, stroke, ischemic stroke, cerebral ischemia, spinal cord trauma, head injury, periparturient hypoxia, cardiac arrest, hypoglycemic nerve injury, Huntington's disease, amyotrophic lateral sclerosis, multiple sclerosis, eye damage, retinopathy, cognitive impairment, muscle spasm, tremor, epilepsy, disorder associated with muscle spasm, delirium, amnestic disorder, age-associated cognitive decline, schizoaffective disorder, paranoia, drug addiction, movement disorder, chronic fatigue syndrome, fatigue, medication-induced parkinsonian syndrome, Gilles de la Tourette syndrome, chorea, myoclonus, tic, restless legs syndrome, dystonia, dyskinesia, attention deficit hyperactivity disorder (ADHD), conduct disorder, urinary incontinence, withdrawal symptom, trigeminal neuralgia, hearing loss, tinnitus, nerve injury, retinopathy, macular degeneration, vomiting, cerebral edema, pain, bone pain, arthralgia, toothache, cataplexy, and traumatic brain injury by administering the compound, pharmaceutically acceptable salt, or pharmaceutical composition.

Other features, objects, and advantages of the invention will be apparent from the detailed description, and from the claims.

### DETAILED DESCRIPTION

### Definitions

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

For convenience, before further description of the present invention, certain terms employed in the specification, examples and appended claims are defined here. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein, the term “compounds of the invention” refers to the compounds of formula (I) and all of the embodiments thereof (e.g., Formulas (I-1), (1-2), (Ia), (Ia-1), (Ia-2) (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2) and (I-D-2a)), as described herein, and to the compounds identified in Table A, Table B, Table C, Table D, and Table E.

As described herein, the compounds of the invention comprise multiple variable groups (e.g., X, T, W, R¹, R⁵, etc.). As one of ordinary skill in the art will recognize, combinations of groups envisioned by this invention are those combinations that result in the formation of stable or chemically feasible compounds. The term “stable,” in this context, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

Certain compounds contained in compositions of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Unless otherwise specified, the compounds of the invention, whether identified by chemical name or chemical structure, include all stereoisomers (e.g., enantiomers and diastereomers), double bond isomers (e.g., (Z) and (E)), conformational isomers, atropisomers, and tautomers of the compounds identified by the chemical names and chemical structures provided herein. In addition, single stereoisomers, double bond isomers, conformational isomers, and tautomers as well as mixtures of stereoisomers, double bond isomers, conformational isomers, and tautomers are within the scope of the invention.

As used herein, in any chemical structure or formula, a non-bold, straight bond attached to a stereocenter of a compound, such as in

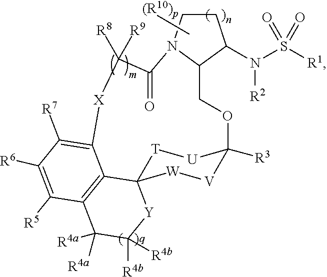

denotes that the configuration of the stereocenter is unspecified. The compound may have any configuration, or a mixture of configurations, at the stereocenter.

As used herein, in any chemical structure or formula, a wiggly bond attached to a stereocenter of a compound, such as in

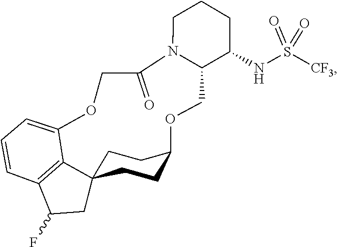

denotes that the compound has a mixture of configurations at the stereocenter.

As used herein, in any chemical structure or formula, a bold or hashed straight bond attached to a stereocenter of a compound, such as in

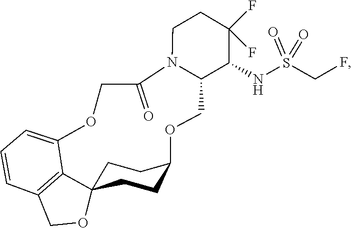

denotes the relative stereochemistry of the stereocenter, relative to other stereocenter(s) to which bold or hashed straight bonds are attached.

As used herein, in any chemical structure or formula, a bold or hashed wedge bond attached to a stereocenter of a compound, such as in

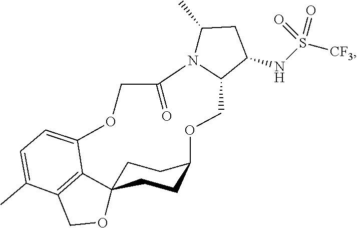

denotes the absolute stereochemistry of the stereocenter, as well as the relative stereochemistry of the stereocenter, relative to other stereocenter(s) to which bold or hashed wedge bonds are attached.

As used herein, the prefix “rac-,” when used in connection with a chiral compound, refers to a racemic mixture of the compound. In a compound bearing the “rac-” prefix, the (R)- and (S)-designators in the chemical name reflect the relative stereochemistry of the compound.

As used herein, the prefix “dia-,” when used in connection with a chiral compound, refers to a mixture of two or more diastereomers of the compound. In a compound bearing the “dia-” prefix, the (R)- and (S)-designators are assigned arbitrarily and do not necessarily reflect the absolute or relative configuration of the corresponding stereocenters in any component of the mixture. In some instances, all of the diastereomers in a mixture have a single absolute configuration at one or more stereocenters. In these instances, the stereochemical designators associated with the stereocenters having a single, known absolute configuration are marked with an asterisk (*), e.g., (R*)- and (S*)-, and the stereochemical designators associated with stereocenters having a single, unknown absolute configuration are marked with a superscript dagger (†). The stereochemical designators marked with an asterisk (*) do not necessarily reflect the relative stereochemistry of the corresponding stereocenters with respect to stereocenters associated with stereochemical designators marked with a superscript dagger (†) (and vice versa) or with respect to stereocenters associated with unmarked stereochemical designators.

As used herein, the prefix “rel-,” when used in connection with a chiral compound, refers to a single enantiomer of unknown absolute configuration. In a compound bearing the “rel-” prefix, the (R)- and (S)-designators in the chemical name reflect the relative stereochemistry of the compound, but do not necessarily reflect the absolute stereochemistry of the compound. In some instances, the absolute configuration of some stereocenters is known, while only the relative configuration of the other stereocenters is known. In these instances, the stereochemical designators associated with the stereocenters of known absolute configuration are marked with an asterisk (*), e.g., (R*)- and (S*)-, while the stereochemical designators associated with stereocenters of unknown absolute configuration are not so marked. The unmarked stereochemical designators associated with the stereocenters of unknown absolute configuration do not necessarily reflect the relative stereochemistry with respect to the stereocenters of known absolute configuration.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration.

Some of the compounds described herein include cyclic moieties having substituents at two or more positions on the cyclic moiety. In such compounds, the relative configuration at two positions on the cyclic moiety may be described as “cis” or “trans,” based on the Cahn-Ingold-Prelog (CIP) sequence rules. Where the substituents having the highest CIP priority at the two positions are on the same face of the cyclic moiety, the positions are described as “cis.” For example, the following compound may be named as 1,1,1-trifluoro-N-((5a,8-cis)-(10aR,11S)-16-oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)methanesulfonamide because the substituents having the highest CIP priority at the 5a- and 8-positions are on the same face of the cyclohexane ring:

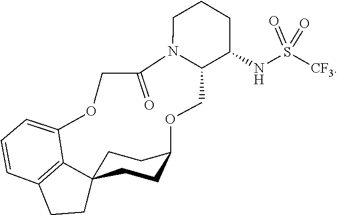

Where the substituents having the highest CIP priority at the two positions are on opposite faces of the cyclic moiety, the positions are described as “trans.” For example, the following compound may be named as 1,1,1-Trifluoro-N-((5a,8-trans)-(10aR,11S)-1-fluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)methanesulfonamide because the substituents having the highest CIP priority at the 5a- and 8-positions are on opposite faces of the cyclohexane ring:

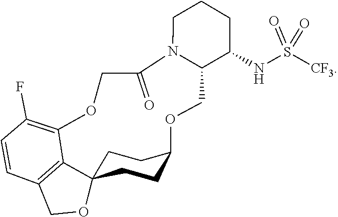

Certain of the disclosed compounds may exist in “atropisomeric” forms or as “atropisomers.” Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from a mixture of isomers. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Percent purity by mole fraction is the ratio of the moles of the enantiomer (or diastereomer) over the moles of the enantiomer (or diastereomer) plus the moles of its optical isomer (or diastereomer) in the mixture. When the stereochemistry of a disclosed compound is named or depicted by structure, the named or depicted stereoisomer is at least about 60%, about 70%, about 80%, about 90%, about 99% or about 99.9% by mole fraction pure relative to the other stereoisomers. When a single enantiomer is named or depicted by structure, the depicted or named enantiomer is at least about 60%, about 70%, about 80%, about 90%, about 99% or about 99.9% by mole fraction pure. When a single diastereomer is named or depicted by structure, the depicted or named diastereomer is at least about 60%, about 70%, about 80%, about 90%, about 99% or about 99.9% by mole fraction pure.

When a disclosed compound is named or depicted by structure without indicating the stereochemistry, and the compound has one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

In the specification and claims, unless otherwise specified, any atom not specifically designated as a particular isotope in any compound of the invention is meant to represent any stable isotope of the specified element. In the Examples, where an atom is not specifically designated as a particular isotope in any compound of the invention, no effort was made to enrich that atom in a particular isotope, and therefore a person of ordinary skill in the art would understand that such atom likely was present at approximately the natural abundance isotopic composition of the specified element.

As used herein, the term “stable,” when referring to an isotope, means that the isotope is not known to undergo spontaneous radioactive decay. Stable isotopes include, but are not limited to, the isotopes for which no decay mode is identified in V. S. Shirley & C. M. Lederer, Isotopes Project, Nuclear Science Division, Lawrence Berkeley Laboratory, Table of Nuclides (January 1980).

As used herein in the specification and claims, “H” refers to hydrogen and includes any stable isotope of hydrogen, namely ¹H and D. In the Examples, where an atom is designated as “H,” no effort was made to enrich that atom in a particular isotope of hydrogen, and therefore a person of ordinary skill in the art would understand that such hydrogen atom likely was present at approximately the natural abundance isotopic composition of hydrogen.

As used herein, “¹H” refers to protium. Where an atom in a compound of the invention, or a pharmaceutically acceptable salt thereof, is designated as protium, protium is present at the specified position with at least the natural abundance concentration of protium.

As used herein, “D,” “d,” and “²H” refer to deuterium.

In some embodiments, the compounds of the invention, and pharmaceutically acceptable salts thereof, include each constituent atom at approximately the natural abundance isotopic composition of the specified element.

In some embodiments, the compounds of the invention, and pharmaceutically acceptable salts thereof, include one or more atoms having an atomic mass or mass number which differs from the atomic mass or mass number of the most abundant isotope of the specified element (“isotope-labeled” compounds and salts). Examples of stable isotopes which are commercially available and suitable for the invention include without limitation isotopes of hydrogen, carbon, nitrogen, oxygen, and phosphorus, for example ²H, ¹³C, ¹⁵N, ¹⁸O, ¹⁷O, and ³¹P, respectively.

The isotope-labeled compounds and salts can be used in a number of beneficial ways, including as medicaments. In some embodiments, the isotope-labeled compounds and salts are deuterium (²H)-labeled. Deuterium (²H)-labeled compounds and salts are therapeutically useful with potential therapeutic advantages over the non-²H-labeled compounds. In general, deuterium (²H)-labeled compounds and salts can have higher metabolic stability as compared to those that are not isotope-labeled owing to the kinetic isotope effect described below. Higher metabolic stability translates directly into an increased in vivo half-life or lower dosages, which under most circumstances would represent a preferred embodiment of the present invention. The isotope-labeled compounds and salts can usually be prepared by carrying out the procedures disclosed in the synthesis schemes, the Examples and the related description, replacing a non-isotope-labeled reactant by a readily available isotope-labeled reactant.

The deuterium (²H)-labeled compounds and salts can manipulate the rate of oxidative metabolism of the compound by way of the primary kinetic isotope effect. The primary kinetic isotope effect is a change of the rate for a chemical reaction that results from exchange of isotopic nuclei, which in turn is caused by the change in ground state energies of the covalent bonds involved in the reaction. Exchange of a heavier isotope usually results in a lowering of the ground state energy for a chemical bond and thus causes a reduction in the rate-limiting bond breakage. If the bond breakage occurs in or in the vicinity of a saddle-point region along the coordinate of a multi-product reaction, the product distribution ratios can be altered substantially. For example, if deuterium is bonded to a carbon atom at a non-exchangeable position, rate differences of k_{H}/k_{D}=2-7 are typical. For a further discussion, see S. L. Harbeson and R. D. Tung, Deuterium In Drug Discovery and Development, Ann. Rep. Med. Chem. 2011, 46, 403-417, incorporated in its entirety herein by reference.

The concentration of an isotope (e.g., deuterium) incorporated at a given position of an isotope-labeled compound of the invention, or a pharmaceutically acceptable salt thereof, may be defined by the isotopic enrichment factor. The term “isotopic enrichment factor,” as used herein, means the ratio between the abundance of an isotope at a given position in an isotope-labeled compound (or salt) and the natural abundance of the isotope.

Where an atom in a compound of the invention, or a pharmaceutically acceptable salt thereof, is designated as deuterium, such compound (or salt) has an isotopic enrichment factor for such atom of at least 3000 (˜45% deuterium incorporation). In some embodiments, the isotopic enrichment factor is at least 3500 (˜52.5% deuterium incorporation), at least 4000 (˜60% deuterium incorporation), at least 4500 (˜67.5% deuterium incorporation), at least 5000 (˜75% deuterium incorporation), at least 5500 (˜82.5% deuterium incorporation), at least 6000 (˜90% deuterium incorporation), at least 6333.3 (˜95% deuterium incorporation), at least 6466.7 (˜97% deuterium incorporation), at least 6600 (˜99% deuterium incorporation), or at least 6633.3 (˜99.5% deuterium incorporation).

In another aspect, the invention relates to radiolabeled analogs of the compounds of the invention. As used herein, the term “radiolabeled analogs of the compounds of the invention” refers to compounds that are identical to the compounds of the invention, as described herein, including all embodiments thereof, except that one or more atoms has been replaced with a radioisotope of the atom present in the compounds of the invention.

As used herein, the term “radioisotope” refers to an isotope of an element that is known to undergo spontaneous radioactive decay. Examples of radioisotopes include ³H, ¹⁴C, ³²P ³⁵S, ¹⁸F, ³⁶Cl, and the like, as well as the isotopes for which a decay mode is identified in V. S. Shirley & C. M. Lederer, Isotopes Project, Nuclear Science Division, Lawrence Berkeley Laboratory, Table of Nuclides (January 1980).

The radiolabeled analogs can be used in a number of beneficial ways, including in various types of assays, such as substrate tissue distribution assays. For example, tritium (³H)- and/or carbon-14 (¹⁴C)-labeled compounds may be useful for various types of assays, such as substrate tissue distribution assays, due to relatively simple preparation and excellent detectability.

In another aspect, the invention relates to pharmaceutically acceptable salts of the radiolabeled analogs, in accordance with any of the embodiments described herein in connection with the compounds of the invention.

In another aspect, the invention relates to pharmaceutical compositions comprising the radiolabeled analogs, or pharmaceutically acceptable salts thereof, and a pharmaceutically acceptable carrier, adjuvant or vehicle, in accordance with any of the embodiments described herein in connection with the compounds of the invention.

In another aspect, the invention relates to a method of preventing or treating a disease associated with a deficiency in orexin 2 receptor signaling in a subject comprising administering an effective amount of the radiolabeled analogs, pharmaceutically acceptable salts thereof, and pharmaceutical compositions thereof, in accordance with any of the embodiments described herein in connection with the compounds of the invention.

In another aspect, the invention relates to a method of preventing or treating a disease selected from narcolepsy, idiopathic hypersomnia, hypersomnia, sleep apnea syndrome, narcolepsy syndrome involving narcolepsy-like symptom, hypersomnia associated with Parkinson's disease, hypersomnia associated with dementia with Lewy body, hypersomnia syndrome involving daytime hypersomnia (e.g., Kleine-Levin syndrome, major depression accompanied by hypersomnia, dementia with Lewy body, Parkinson's disease, progressive supranuclear palsy, Prader-Willi syndrome, Moebius syndrome, hypoventilation syndrome, Niemann-Pick disease type C, brain contusion, cerebral infarction, brain tumor, muscular dystrophy, multiple sclerosis, acute disseminated encephalomyelitis, Guillain-Barre syndrome, Rasmussen's encephalitis, Wernicke's encephalopathy, limbic encephalitis, Hashimoto encephalopathy), coma, loss of consciousness, obesity (e.g., malignant mast cell, extrinsic obesity, hyperinsulinar obesity, hyperplasmic obesity, hypophysial obesity, hypoplasmic obesity, hypothyroid obesity, hypothalamic obesity, symptomatic obesity, childhood obesity, upper body obesity, alimentary obesity, gonadal obesity, systemic mastocytosis, primary obesity, central obesity), insulin resistance syndrome, Alzheimer, impaired consciousness such as coma, side effect or complication caused by anesthesia, sleep disturbance, sleep problem, insomnia, intermittent sleep, night myoclonus, REM sleep interruption, jet lag, jet lag syndrome, sleep disorder of shift workers, dyssomnia, sleep terror, depression, major depression, sleepwalking, enuresis, sleep disorder, Alzheimer's sundown syndrome, disease associated with circadian rhythm, fibromyalgia, condition resulting from decrease in sleeping quality, bulimia, obsessive eating disorder, obesity-related diseases, hypertension, diabetes, elevated plasma insulin level/insulin resistance, hyperlipemia, hyperlipidaemia, endometrial cancer, breast cancer, prostate cancer, colon cancer, cancer, osteoarthritis, obstructive sleep apnea, cholelithiasis, gallstone, heart disease, abnormal heartbeat, arrhythmia, myocardial infarction, congestive heart failure, heart failure, coronary heart disease, cardiovascular disease, sudden death, polycystic ovary, craniopharyngioma, Prader Willi syndrome, Froehlich syndrome, growth hormone deficiency, normal variant short stature, Turner syndrome, children suffering from acute lymphoblastic leukemia, syndrome X, reproductive hormone abnormality, decrease of fecundability, infertility, hypogonadism in men, sexual/reproductive-function dysfunction such as hirsutism in women, fetal defect associated with maternity obesity, gastrointestinal motility disorder such as obesity-related gastroesophageal reflux, obesity hypoventilation syndrome (Pickwickian syndrome), respiratory disease such as respiratory distress, inflammation such as vascular systemic inflammation, arteriosclerosis, hypercholesterolemia, hyperuricemia, low back pain, gallbladder disease, gout, renal cancer, secondary risk of obesity such as risk of left ventricle hypertrophy, migraine, headache, neuropathic pain, Parkinson's disease, psychosis, schizophrenia, facial flushing, night sweat, disease in genitalium/urinary system, disease associated with sexual function or fecundability, dysthymic disorder, bipolar disorder, bipolar I disorder, bipolar II disorder, cyclothymic disorder, acute stress disorder, agoraphobia, generalized anxiety disorder, obsessive-compulsive disorder, panic attack, panic disorder, posttraumatic stress disorder, separation anxiety disorder, social phobia, anxiety disorder, acute neurological and psychiatric disorder such as cerebral deficiency developed after heart bypass surgery or heart transplant, stroke, ischemic stroke, cerebral ischemia, spinal cord trauma, head injury, periparturient hypoxia, cardiac arrest, hypoglycemic nerve injury, Huntington's disease, amyotrophic lateral sclerosis, multiple sclerosis, eye damage, retinopathy, cognitive impairment, muscle spasm, tremor, epilepsy, disorder associated with muscle spasm, delirium, amnestic disorder, age-associated cognitive decline, schizoaffective disorder, paranoia, drug addiction, movement disorder, chronic fatigue syndrome, fatigue, medication-induced parkinsonian syndrome, Gilles de la Tourette syndrome, chorea, myoclonus, tic, restless legs syndrome, dystonia, dyskinesia, attention deficit hyperactivity disorder (ADHD), conduct disorder, urinary incontinence, withdrawal symptom, trigeminal neuralgia, hearing loss, tinnitus, nerve injury, retinopathy, macular degeneration, vomiting, cerebral edema, pain, bone pain, arthralgia, toothache, cataplexy, and traumatic brain injury in a subject comprising administering an effective amount of the radiolabeled analogs, pharmaceutically acceptable salts thereof, and pharmaceutical compositions thereof, in accordance with any of the embodiments described herein in connection with the compounds of the invention.

In another aspect, the invention relates to radiolabeled analogs, pharmaceutically acceptable salts thereof, and pharmaceutical compositions thereof, for use, in accordance with any of the embodiments described herein in connection with the compounds of the invention.

In another aspect, the invention relates to the use of the radiolabeled analogs, or pharmaceutically acceptable salts thereof, and pharmaceutical compositions thereof, for the manufacture of medicaments, in accordance with any of the embodiments described herein in connection with the compounds of the invention.

In another aspect, the radiolabeled analogs, pharmaceutically acceptable salts thereof, and pharmaceutical compositions thereof, can be employed in combination therapies, in accordance with any of the embodiments described herein in connection with the compounds of the invention.

The term “prodrug” refers to compounds which are drug precursors which, following administration and absorption, release the drug in vivo via some metabolic process. In general, a prodrug possesses less biological activity than its parent drug. A prodrug may also improve the physical properties of the parent drug and/or it may also improve overall drug efficacy, for example through the reduction of toxicity and unwanted effects of a drug by controlling its absorption, blood levels, metabolic distribution and cellular uptake.

The term “parent compound” or “parent drug” refers to the biologically active entity that is released via enzymatic action of a metabolic or a catabolic process, or via a chemical process following administration of the prodrug. The parent compound may also be the starting material for the preparation of its corresponding prodrug.

A common method for making a prodrug is to include selected moieties that are hydrolyzed under physiological conditions to reveal the desired molecule. Prodrugs of the compounds of the invention may be, for example, amides. Amides that may be utilized as prodrugs in the present invention are phenyl amides, aliphatic (C₁-C₂₄) amides, acyloxymethyl amides, ureas, carbamates, and amino acid amides. For example, a compound of the invention that contains an NH group may be acylated at this position in its prodrug form. Other prodrug forms include esters, such as, for example phenyl esters, aliphatic (C₁-C₂₄) esters, acyloxymethyl esters, carbonates, carbamates, and amino acid esters. A thorough discussion of prodrugs is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins et al., Synthetic Communications 26(23):4351-4367, 1996, each of which is incorporated in its entirety herein by reference. In some embodiments, the present invention features a prodrug of any one of the formulas or compounds listed herein.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid or base addition salts of the compound(s). These salts can be prepared in situ during the final isolation and purification of the compound(s), or by separately reacting a purified compound(s) in its free base or acid form with a suitable organic or inorganic acid or base, and isolating the salt thus formed. Representative acid addition salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.) Representative inorganic base addition salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

The term “pharmaceutically acceptable cocrystals” refers to solid coformers that do not form formal ionic interactions with the compound.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” or “subject” refers to a mammal in need of a particular treatment or prevention of a particular disease or condition. In certain embodiments, a patient is a primate, canine, feline, or equine. In certain embodiments, a patient is a human.

“Alkyl” refers to a fully saturated, acyclic, branched or unbranched (linear) carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain embodiments, a straight chain or branched chain alkyl has 30 or fewer carbon atoms in its backbone (e.g., C₁-C₃₀ for straight chains, C₃-C₃₀ for branched chains), and more preferably 20 or fewer. Alkyl groups may be substituted or unsubstituted.

As used herein, the term “alkoxy” refers to a radical of the formula —OR where R is an alkyl group having the specified number of carbon atoms. For example, a “C₁-C₆ alkoxy” group is a radical of the formula —OR where R is an alkyl group having the between one and six carbon atoms.

As used herein, the term “haloalkyl” refers to an alkyl group as hereinbefore defined substituted with at least one halogen.

As used herein, the term “alkylene” refers to a fully saturated, acyclic, branched or unbranched divalent hydrocarbon group having the specified number of carbon atoms, for example from 2 to 12 carbon atoms. Non-limiting examples of alkylene groups include methylene —(CH₂)—, ethylene —(CH₂CH₂)—, n-propylene —(CH₂CH₂CH₂)—, isopropylene —(CH₂CH(CH₃))—, and the like. Alkylene groups may be substituted or unsubstituted.

“Cycloalkyl” means a stable, non-aromatic, mono-, bi-, or polycyclic (fused, bridged, or spiro) saturated hydrocarbon radical, having the specified number of carbon ring atoms, and which is attached to the rest of the molecule by a single bond. Preferred cycloalkyls have from 3-10 carbon atoms in their ring structure, and more preferably have 3-6 carbons in the ring structure. Cycloalkyl groups may be substituted or unsubstituted. Some examples include cyclopropyl, cyclobutyl, cyclopentyl, cyclohexyl, cycloheptyl, and cyclooctyl groups.

“Alkenyl” refers to an acyclic, branched or unbranched (linear) unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s). Alkenyl groups may be substituted or unsubstituted.

“Alkynyl” refers to an acyclic, branched or unbranched (linear) unsaturated carbon chain moiety having the number of carbon atoms specified, or up to 26 carbon atoms if no limitation on the number of carbon atoms is specified; and having one or more triple bonds in the moiety. Alkynyl groups may be substituted or unsubstituted.

The term “aryl” as used herein includes 3- to 12-membered substituted or unsubstituted mono-, bi-, and polycyclic aromatic groups in which each atom of the ring(s) is carbon. Preferably, aryl groups include 5- to 12-membered rings, more preferably 6- to 10-membered rings. The term “aryl” also includes polycyclic ring systems having two or more cyclic rings in which two or more carbons are common to two adjoining rings wherein at least one of the rings is aromatic. Aryl groups include benzene, naphthalene, phenanthrene, and the like.

The term “heteroaryl” as used herein includes 3- to 12-membered substituted or unsubstituted mono-, bi-, and polycyclic aromatic groups in which at least one atom of the ring(s) is heteroatom. Heteroaryl groups include substituted or unsubstituted aromatic 3- to 12-membered ring structures, more preferably 5- to 12-membered rings, more preferably 5- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine groups, and the like.

The term “halo”, “halide”, or “halogen” as used herein means halogen and includes, for example, and without being limited thereto, fluoro, chloro, bromo, iodo and the like, in both radioactive and non-radioactive forms. In a preferred embodiment, halo is selected from the group consisting of fluoro, chloro and bromo.

The terms “heterocyclyl” or “heterocyclic group” or “heterocycloalkyl” refer to stable, non-aromatic 3- to 12-membered ring structures, more preferably 5- to 12-membered rings, more preferably 5- to 10-membered rings, whose ring structures include one to four heteroatoms. Heterocycles can be monocyclic, bicyclic, spirocyclic, or polycyclic, and may be saturated or unsaturated. Heterocyclyl groups include, for example, aziridine, oxirane, azetidine, oxetane, pyrrolidine, oxolane, thiolane, piperidine, piperazine, morpholine, and the like. The heterocyclic ring may be substituted or unsubstituted.

The term “substituted” refers to moieties having substituents replacing a hydrogen on one or more carbons or heteroatoms of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the definition of each expression, e.g., alkyl, m, n, etc., when it occurs more than once in any structure, is intended to be independent of its definition elsewhere in the same structure.

An “effective amount” is an amount sufficient to effect beneficial or desired results. For example, a “therapeutically effective amount” is one that achieves a desired therapeutic effect, e.g., alleviating a symptom, ameliorating a condition, or slowing the onset of disease conditions according to clinically acceptable standards for the disorder or condition to be treated, e.g., at a reasonable benefit/risk ratio applicable to any medical treatment. This amount can be the same or different from a “prophylactically effective amount,” which is an amount necessary to prevent onset of disease or disease symptoms. An effective amount can be administered in one or more administrations, applications or dosages. An effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” includes up-regulation (e.g., activating or enhancing a response) and down-regulation (e.g., inhibiting or deactivating a response).

A “radiopharmaceutical agent,” as defined herein, refers to a pharmaceutical agent which contains at least one radiation-emitting radioisotope. Radiopharmaceutical agents are routinely used in nuclear medicine for the diagnosis and/or therapy of various diseases. The radiolabeled pharmaceutical agent, for example, a radiolabeled antibody, contains a radioisotope (RI) which serves as the radiation source. As contemplated herein, the term “radioisotope” includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule.

For purposes of this invention, the chemical elements are identified in accordance with the Periodic Table of the Elements, CAS version, Handbook of Chemistry and Physics, 67th Ed., 1986-87, inside cover.

### Compounds of the Invention

In one aspect, the invention relates to a compound of formula (I):

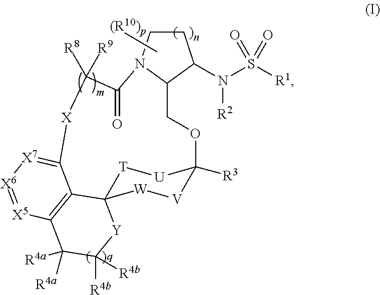

or a pharmaceutically acceptable salt thereof, wherein:
T is O, CR¹¹R¹², or absent;
U is CR¹³R¹⁴;
V is CR¹⁵R¹⁶;
W is O, CR¹⁷R¹⁸, or absent;
X is O or absent;
X⁵ is CR⁵ or N;
X⁶ is CR⁶ or N;
X⁷ is CR⁷ or N;
Y is O or C(R¹⁹)₂;
R¹ is selected from —NR^{a}R^{b}, —C₁-C₃ alkylene-NR^{a}R^{b}, —C₁-C₃ alkylene-OH, —C₁-C₆ alkyl, —C₁-C₆ haloalkyl, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, and —C₁-C₃ alkylene-(5- to 10-membered heteroaryl), wherein said —C₁-C₃ alkylene-NR^{a}R^{b}, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, or —C₁-C₃ alkylene-(5- to 10-membered heteroaryl) is optionally substituted with 1-3 substituents independently selected from halo, —OH, —C₁-C₃ alkyl, and —C₁-C₃ alkoxy;
R² is —H or —C₁-C₃ alkyl;
R³ is —H, halo, —C₁-C₆ alkyl, —C₁-C₆ haloalkyl, or —C₁-C₆ alkoxy;
each R^{4a} is independently selected from —H, halo, —OH, —CN, —C₁-C₆ alkyl, —C₁-C₆ haloalkyl, and —C₁-C₆ alkoxy; or
two R^{4a} together form an oxo;
each R^{4b} is independently selected from —H, halo, —OH, —CN, —C₁-C₆ alkyl, —C₁-C₆haloalkyl, and —C₁-C₆ alkoxy;
R⁵, R⁶, and R⁷ are each independently selected from —H, halo, —OH, —CN, —C₁-C₆ alkyl, —C₁-C₆haloalkyl, and —C₁-C₆ alkoxy;
each R⁸ and each R⁹ is independently selected from —H, halo, —C₁-C₆ alkyl, —C₁-C₆ haloalkyl, and —C₁-C₆ alkoxy; or
R⁸ and R⁹, together with together with the carbon atom to which they are attached, form a C₃-C₆ cycloalkyl optionally substituted with halo, —C₁-C₃ alkyl, —C₁-C₃ haloalkyl, or —C₁-C₃ alkoxy;
each R¹⁰ is independently selected from halo, —C₁-C₆ alkyl, —C₁-C₆haloalkyl, and —C₁-C₆ alkoxy;
R¹¹, R¹², R¹³, R¹⁴, R¹⁵, R¹⁶, R¹⁷, and R¹⁸ are each independently selected from —H, halo, —C₁-C₆ alkyl, —C₁-C₆haloalkyl, and —C₁-C₆ alkoxy;
each R¹⁹ is independently selected from —H, halo, and —C₁-C₃ alkyl;
R^{a} and R^{b} are each independently selected from —H and —C₁-C₃ alkyl;
m is 1, 2, 3, or 4;
n is 0, 1, 2, or 3;
p is 0, 1, 2, 3, or 4; and
q is 0 or 1;
   provided that:
no more than one of T and W is absent; and
no more than one of X⁵, X⁶, and X⁷ is N.

In some embodiments, the invention relates to a compound of formula (I), or a pharmaceutically acceptable salt thereof, wherein:
X⁵ is CR⁵;
X⁶ is CR⁶;
X⁷ is CR⁷; and
R¹ is selected from —NR^{a}R^{b}, —C₁-C₃ alkylene-NR^{a}R^{b}, —C₁-C₆ alkyl, —C₁-C₆ haloalkyl, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, and —C₁-C₃ alkylene-(5- to 10-membered heteroaryl), wherein said —C₁-C₃ alkylene-NR^{a}R^{b}, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, or —C₁-C₃ alkylene-(5- to 10-membered heteroaryl) is optionally substituted with 1-3 substituents independently selected from halo, —OH, —C₁-C₃ alkyl, and —C₁-C₃ alkoxy.

In some embodiments, the invention relates to a compound of formula (I), or a pharmaceutically acceptable salt thereof, wherein:
q is 0; and
each R^{4a} is independently selected from —H, halo, —OH, —CN, —C₁-C₆ alkyl, —C₁-C₆haloalkyl, and —C₁-C₆ alkoxy.

In some embodiments, the invention relates to a compound of formula (I-1):

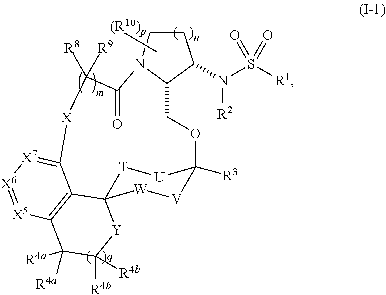

or a pharmaceutically acceptable salt thereof.

In some embodiments, the invention relates to a compound of formula (I-2):

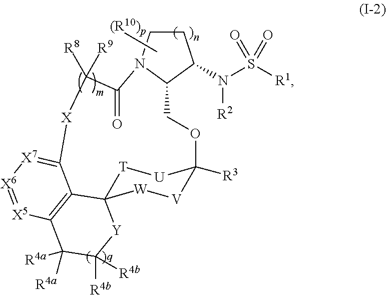

or a pharmaceutically acceptable salt thereof.

In some embodiments, the invention relates to a compound of formula (I), (I-1), or (1-2), wherein X⁵ is CR⁵. In some embodiments, the invention relates to a compound of formula (I), (I-1), or (I-2) wherein X⁵ is N.

In some embodiments, the invention relates to a compound of formula (I), (I-1), or (I-2) wherein X⁶ is CR⁶. In some embodiments, the invention relates to a compound of formula (I), (I-1), or (I-2) wherein X⁶ is N.

In some embodiments, the invention relates to a compound of formula (I), (I-1), or (I-2) wherein X⁷ is CR⁷. In some embodiments, the invention relates to a compound of formula (I), (I-1), or (I-2) wherein X⁷ is N.

In some embodiments, the invention relates to a compound of formula (I), (I-1), or (I-2), wherein X⁵ is CR⁵, X⁶ is CR⁶, and X⁷ is CR⁷. In some embodiments, the invention relates to a compound of formula (I), (I-1), or (I-2), wherein X⁵ is N, X⁶ is CR⁶, and X⁷ is CR⁷. In some embodiments, the invention relates to a compound of formula (I), (I-1), or (I-2), wherein X⁵ is CR⁵, X⁶ is N, and X⁷ is CR⁷. In some embodiments, the invention relates to a compound of formula (I), (I-1), or (I-2), wherein X⁵ is CR⁵, X⁶ is CR⁶, and X⁷ is N.

In some embodiments, the invention relates to a compound of formula (Ia):

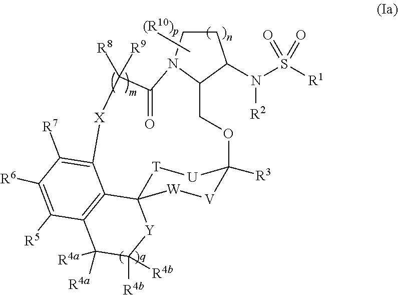

or a pharmaceutically acceptable salt thereof.

In some embodiments, the invention relates to a compound of formula (Ia-1):

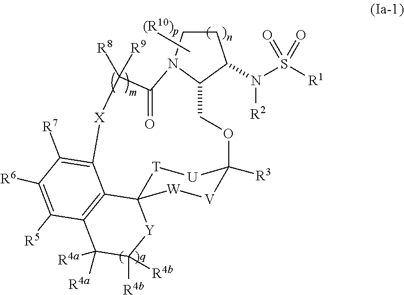

or a pharmaceutically acceptable salt thereof.

In some embodiments, the invention relates to a compound of formula (Ia-2):

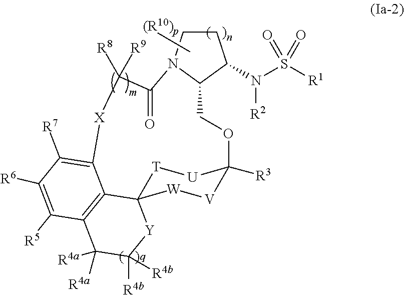

or a pharmaceutically acceptable salt thereof.

In some embodiments, the invention relates to a compound of formula (I), (I-1), (I-2), (Ia), (Ia-1), or (Ia-2), or a pharmaceutically acceptable salt thereof, wherein T is CR¹¹R¹².

In some embodiments, the invention relates to a compound of formula (I), (I-1), (I-2), (Ia), (Ia-1), or (Ia-2), or a pharmaceutically acceptable salt thereof, wherein W is CR¹⁷R¹⁸.

In some embodiments, the invention relates to a compound of formula (I), (I-1), (I-2), (Ia), (Ia-1), or (Ia-2), or a pharmaceutically acceptable salt thereof, wherein X is O.

In some embodiments, the invention relates to a compound of formula (I), (I-1), (I-2), (Ia), (Ia-1), or (Ia-2), or a pharmaceutically acceptable salt thereof, wherein Y is O. In some embodiments, the invention relates to a compound of formula (I), (I-1), (I-2), (Ia), (Ia-1), or (Ia-2), or a pharmaceutically acceptable salt thereof, wherein Y is C(R¹⁹)₂. In some embodiments, the invention relates to a compound of formula (I), (I-1), (I-2), (Ia), (Ia-1), or (Ia-2), or a pharmaceutically acceptable salt thereof, wherein Y is C(R¹⁹)₂ and each R¹⁹ is H.

In some embodiments, the invention relates to a compound of formula (I), (I-1), (I-2), (Ia), (Ia-1), or (Ia-2), or a pharmaceutically acceptable salt thereof, wherein R¹¹, R¹², R¹³, R¹⁴, R¹⁵, R¹⁶, R¹⁷, and R¹⁸ are each —H. In some embodiments, the invention relates to a compound of formula (I), (I-1), (I-2), (Ia), (Ia-1), or (Ia-2), or a pharmaceutically acceptable salt thereof, wherein R¹¹, R¹², R¹⁷, and R¹⁸ are each -D. In some embodiments, the invention relates to a compound of formula (I), (I-1), (I-2), (Ia), (Ia-1), or (Ia-2), or a pharmaceutically acceptable salt thereof, wherein R¹³, R¹⁴, R¹⁵, and R¹⁶ are each -D. In some embodiments, the invention relates to a compound of formula (I), (I-1), (I-2), (Ia), (Ia-1), or (Ia-2), or a pharmaceutically acceptable salt thereof, wherein R¹¹ is -D. In some embodiments, the invention relates to a compound of formula (I), (I-1), (I-2), (Ia), (Ia-1), or (Ia-2), or a pharmaceutically acceptable salt thereof, wherein R¹² is -D. In some embodiments, the invention relates to a compound of formula (I), (I-1), (I-2), (Ia), (Ia-1), or (Ia-2), or a pharmaceutically acceptable salt thereof, wherein R¹³ is -D. In some embodiments, the invention relates to a compound of formula (I), (I-1), (I-2), (Ia), (Ia-1), or (Ia-2), or a pharmaceutically acceptable salt thereof, wherein R¹⁴ is -D. In some embodiments, the invention relates to a compound of formula (I), (I-1), (I-2), (Ia), (Ia-1), or (Ia-2), or a pharmaceutically acceptable salt thereof, wherein R¹⁵ is -D. In some embodiments, the invention relates to a compound of formula (I), (I-1), (I-2), (Ia), (Ia-1), or (Ia-2), or a pharmaceutically acceptable salt thereof, wherein R¹⁶ is -D. In some embodiments, the invention relates to a compound of formula (I), (I-1), (I-2), (Ia), (Ia-1), or (Ia-2), or a pharmaceutically acceptable salt thereof, wherein R¹⁷ is -D. In some embodiments, the invention relates to a compound of formula (I), (I-1), (I-2), (Ia), (Ia-1), or (Ia-2), or a pharmaceutically acceptable salt thereof, wherein R¹⁸ is -D.

In some embodiments, the invention relates to a compound of formula (I), (I-1), (I-2), (Ia), (Ia-1), or (Ia-2), or a pharmaceutically acceptable salt thereof, wherein R² is —H.

In some embodiments, the invention relates to a compound of formula (I), (I-1), (I-2), (Ia), (Ia-1), or (Ia-2), or a pharmaceutically acceptable salt thereof, wherein R³ is —H. In some embodiments, the invention relates to a compound of formula (I), (I-1), (I-2), (Ia), (Ia-1), or (Ia-2), or a pharmaceutically acceptable salt thereof, wherein R³ is -D.

In some embodiments, the invention relates to a compound of formula (I), (I-1), (I-2), (Ia), (Ia-1), or (Ia-2), or a pharmaceutically acceptable salt thereof, wherein m is 1 or 2. In some embodiments, the invention relates to a compound of formula (I), (I-1), (I-2), (Ia), (Ia-1), or (Ia-2), or a pharmaceutically acceptable salt thereof, wherein m is 1. In some embodiments, the invention relates to a compound of formula (I), (I-1), (I-2), (Ia), (Ia-1), or (Ia-2), or a pharmaceutically acceptable salt thereof, wherein m is 2.

In some embodiments, the invention relates to a compound of formula (I), (I-1), (I-2), (Ia), (Ia-1), or (Ia-2), or a pharmaceutically acceptable salt thereof, wherein n is 1 or 2. In some embodiments, the invention relates to a compound of formula (I), (I-1), (I-2), (Ia), (Ia-1), or (Ia-2), or a pharmaceutically acceptable salt thereof, wherein n is 1. In some embodiments, the invention relates to a compound of formula (I), (I-1), (I-2), (Ia), (Ia-1), or (Ia-2), or a pharmaceutically acceptable salt thereof, wherein n is 2.

In some embodiments, the invention relates to a compound of formula (I), (I-1), (I-2), (Ia), (Ia-1), or (Ia-2), or a pharmaceutically acceptable salt thereof, wherein at least one R^{4a} is —H, halo, —OH, or —C₁-C₆ alkyl. In some embodiments, the invention relates to a compound of formula (I), (I-1), (I-2), (Ia), (Ia-1), or (Ia-2), or a pharmaceutically acceptable salt thereof, wherein at least one R^{4a} is —H or halo. In some embodiments, the invention relates to a compound of formula (I), (I-1), (I-2), (Ia), (Ia-1), or (Ia-2), or a pharmaceutically acceptable salt thereof, wherein each R^{4a} is independently selected from —H, halo, —OH, or —C₁-C₆ alkyl. In some embodiments, the invention relates to a compound of formula (I), (I-1), (I-2), (Ia), (Ia-1), or (Ia-2), or a pharmaceutically acceptable salt thereof, wherein each R^{4a} is independently selected from —H and halo. In some embodiments, the invention relates to a compound of formula (I), (I-1), (I-2), (Ia), (Ia-1), or (Ia-2), or a pharmaceutically acceptable salt thereof, wherein at least one R^{4a} is —H. In some embodiments, the invention relates to a compound of formula (I), (I-1), (I-2), (Ia), (Ia-1), or (Ia-2), or a pharmaceutically acceptable salt thereof, wherein at least one R^{4a} is -D. In some embodiments, the invention relates to a compound of formula (I), (I-1), (I-2), (Ia), (Ia-1), or (Ia-2), or a pharmaceutically acceptable salt thereof, wherein each R^{4a} is -D. In some embodiments, the invention relates to a compound of formula (I), (I-1), (I-2), (Ia), (Ia-1), or (Ia-2), or a pharmaceutically acceptable salt thereof, wherein each R^{4a} is —H. In some embodiments, the invention relates to a compound of formula (I), (I-1), (I-2), (Ia), (Ia-1), or (Ia-2), or a pharmaceutically acceptable salt thereof, wherein at least one R^{4a} is halo. In some embodiments, the invention relates to a compound of formula (I), (I-1), (I-2), (Ia), (Ia-1), or (Ia-2), or a pharmaceutically acceptable salt thereof, wherein each R^{4a} is independently selected from halo. In some embodiments, the invention relates to a compound of formula (I), (I-1), (I-2), (Ia), (Ia-1), or (Ia-2), or a pharmaceutically acceptable salt thereof, wherein at least one R^{4a} is —OH. In some embodiments, the invention relates to a compound of formula (I), (I-1), (I-2), (Ia), (Ia-1), or (Ia-2), or a pharmaceutically acceptable salt thereof, wherein at least one R^{4a} is —C₁-C₆ alkyl. In some embodiments, the invention relates to a compound of formula (I), (I-1), (I-2), (Ia), (Ia-1), or (Ia-2), or a pharmaceutically acceptable salt thereof, wherein each R^{4a} is independently selected from —H, —F, —Br, —OH, and —CH₃. In some embodiments, the invention relates to a compound of formula (I), (I-1), (I-2), (Ia), (Ia-1), or (Ia-2), or a pharmaceutically acceptable salt thereof, wherein each R^{4a} is independently selected from —H, —F, —OH, and —CH₃. In some embodiments, the invention relates to a compound of formula (I), (I-1), (I-2), (Ia), (Ia-1), or (Ia-2), or a pharmaceutically acceptable salt thereof, wherein at least one R^{4a} is —F. In some embodiments, the invention relates to a compound of formula (I), (I-1), (I-2), (Ia), (Ia-1), or (Ia-2), or a pharmaceutically acceptable salt thereof, wherein at least one R^{4a} is —Br. In some embodiments, the invention relates to a compound of formula (I), (I-1), (I-2), (Ia), (Ia-1), or (Ia-2), or a pharmaceutically acceptable salt thereof, wherein each R^{4a} is —F. In some embodiments, the invention relates to a compound of formula (I), (I-1), (I-2), (Ia), (Ia-1), or (Ia-2), or a pharmaceutically acceptable salt thereof, wherein at least one R^{4a} is —CH₃.

In some embodiments, the invention relates to a compound of formula (I), (I-1), (I-2), (Ia), (Ia-1), or (Ia-2), or a pharmaceutically acceptable salt thereof, wherein two R^{4a} together form an oxo.

In some embodiments, the invention relates to a compound of formula (I), (I-1), (I-2), (Ia), (Ia-1), or (Ia-2), or a pharmaceutically acceptable salt thereof, wherein q is 0.

In some embodiments, the invention relates to a compound of formula (I), (I-1), (I-2), (Ia), (Ia-1), or (Ia-2), or a pharmaceutically acceptable salt thereof, wherein T is CR¹¹R¹² and W is CR¹¹R¹⁸.

In some embodiments, the invention relates to a compound of formula (I), (I-1), (I-2), (Ia), (Ia-1), or (Ia-2), or a pharmaceutically acceptable salt thereof, wherein T is CR¹¹R¹²; W is CR¹⁷R¹⁸; and R³, R¹¹, R¹², R¹³, R¹⁴, R¹⁵, R¹⁶, R¹⁷, and R¹⁸ are each —H.

In some embodiments, the invention relates to a compound of formula (I), (I-1), (I-2), (Ia), (Ia-1), or (Ia-2), or a pharmaceutically acceptable salt thereof, wherein T is CR¹¹R¹²; W is CR¹⁷R¹⁸; R² is —H; and R³, R¹¹, R¹², R¹³, R¹⁴, R¹⁵, R¹⁶, R¹⁷, and R¹⁸ are each —H.

In some embodiments, the invention relates to a compound of formula (I), (I-1), (I-2), (Ia), (Ia-1), or (Ia-2), or a pharmaceutically acceptable salt thereof, wherein X is O, T is CR¹¹R¹², and W is CR¹⁷R¹⁸.

In some embodiments, the invention relates to a compound of formula (I), (I-1), (I-2), (Ia), (Ia-1), or (Ia-2), or a pharmaceutically acceptable salt thereof, wherein X is O; T is CR¹¹R¹²; W is CR¹⁷R¹⁸; and R² is —H.

In some embodiments, the invention relates to a compound of formula (I), (I-1), (1-2), (Ia), (Ia-1), or (Ia-2), or a pharmaceutically acceptable salt thereof, wherein X is O; T is CR¹¹R¹²; W is CR¹⁷R¹⁸; and R³, R¹¹, R¹², R¹³, R¹⁴, R¹, R¹⁶, R¹⁷, and R¹⁸ are each —H.

In some embodiments, the invention relates to a compound of formula (I), (I-1), (I-2), (Ia), (Ia-1), or (Ia-2), or a pharmaceutically acceptable salt thereof, wherein X is O; T is CR¹¹R¹²; W is CR¹⁷R¹⁸; R² is —H; and R³, R¹¹, R¹², R¹³, R¹⁴, R¹, R¹⁶, R¹⁷, and R¹⁸ are each —H.

In some embodiments, the invention relates to a compound of formula (I), (I-1), (I-2), (Ia), (Ia-1), or (Ia-2), or a pharmaceutically acceptable salt thereof, wherein X is O; T is CR¹¹R¹²; W is CR¹⁷R¹⁸; m is 1; R² is —H; and R³, R¹¹, R¹², R¹³, R¹⁴, R¹⁵, R¹⁶, R¹⁷, and R¹⁸ are each —H.

In some embodiments, the invention relates to a compound of formula (I), (I-1), (I-2), (Ia), (Ia-1), or (Ia-2), or a pharmaceutically acceptable salt thereof, wherein X is O; T is CR¹¹R¹²; W is CR¹⁷R¹⁸; m is 2; R² is —H; and R³, R¹¹, R¹², R¹³, R¹⁴, R¹⁵, R¹⁶, R¹⁷, and R¹⁸ are each —H.

In some embodiments, the invention relates to a compound of formula (I), (I-1), (I-2), (Ia), (Ia-1), or (Ia-2), or a pharmaceutically acceptable salt thereof, wherein X is O; T is CR¹¹R¹²; W is CR¹⁷R¹⁸; n is 1; R² is —H; and R³, R¹¹, R¹², R¹³, R¹⁴, R¹⁵, R¹⁶, R¹⁷, and R¹⁸ are each —H.

In some embodiments, the invention relates to a compound of formula (I), (I-1), (I-2), (Ia), (Ia-1), or (Ia-2), or a pharmaceutically acceptable salt thereof, wherein X is O; T is CR¹¹R¹²; W is CR¹⁷R¹⁸; n is 2; R² is —H; and R³, R¹¹, R¹², R¹³, R¹⁴, R¹⁵, R¹⁶, R¹⁷, and R¹⁸ are each —H.

In some embodiments, the invention relates to a compound of formula (I), (I-1), (I-2), (Ia), (Ia-1), or (Ia-2), or a pharmaceutically acceptable salt thereof, wherein X is O; T is CR¹¹R¹²; W is CR¹⁷R¹⁸; m is 1; n is 1; R² is —H; and R³, R¹¹, R¹², R¹³, R¹⁴, R¹⁵, R¹⁶, R¹⁷, and R¹⁸ are each —H.

In some embodiments, the invention relates to a compound of formula (I), (I-1), (I-2), (Ia), (Ia-1), or (Ia-2), or a pharmaceutically acceptable salt thereof, wherein X is O; T is CR¹¹R¹²; W is CR¹⁷R¹⁸; m is 1; n is 2; R² is —H; and R³, R¹¹, R¹², R¹³, R¹⁴, R¹⁵, R¹⁶, R¹⁷, and R¹⁸ are each —H.

In some embodiments, the invention relates to a compound of formula (I), (I-1), (I-2), (Ia), (Ia-1), or (Ia-2), or a pharmaceutically acceptable salt thereof, wherein X is O; T is CR¹¹R¹²; W is CR¹⁷R¹⁸; m is 2; n is 1; R² is —H; and R³, R¹¹, R¹², R¹³, R¹⁴, R¹⁵, R¹⁶, R¹⁷, and R¹⁸ are each —H.

In some embodiments, the invention relates to a compound of formula (I), (I-1), (1-2), (Ia), (Ia-1), or (Ia-2), or a pharmaceutically acceptable salt thereof, wherein X is O; Y is O; T is CR¹¹R¹², and W is CR¹⁷R¹⁸.

In some embodiments, the invention relates to a compound of formula (I), (I-1), (I-2), (Ia), (Ia-1), or (Ia-2), or a pharmaceutically acceptable salt thereof, wherein X is O; Y is O; T is CR¹¹R¹²; W is CR¹⁷R¹⁸; and R² is —H.

In some embodiments, the invention relates to a compound of formula (I), (I-1), (I-2), (Ia), (Ia-1), or (Ia-2), or a pharmaceutically acceptable salt thereof, wherein X is O; Y is O; T is CR¹¹R¹²; W is CR¹⁷R¹⁸; and R³, R¹¹, R¹², R¹³, R¹⁴, R¹⁵, R¹⁶, R¹⁷, and R¹⁸ are each —H.

In some embodiments, the invention relates to a compound of formula (I), (I-1), (I-2), (Ia), (Ia-1), or (Ia-2), or a pharmaceutically acceptable salt thereof, wherein X is O; Y is O; T is CR¹¹R¹²; W is CR¹⁷R¹⁸; R² is —H; and R³, R¹¹, R¹², R¹³, R¹⁴, R¹⁵, R¹⁶, R¹⁷, and R¹⁸ are each —H.

In some embodiments, the invention relates to a compound of formula (I), (I-1), (I-2), (Ia), (Ia-1), or (Ia-2), or a pharmaceutically acceptable salt thereof, wherein X is O; Y is O; T is CR¹¹R¹²; W is CR¹⁷R¹⁸; m is 1; R² is —H; and R³, R¹¹, R¹², R¹³, R¹⁴, R¹⁵, R¹⁶, R¹⁷, and R¹⁸ are each —H.

In some embodiments, the invention relates to a compound of formula (I), (I-1), (I-2), (Ia), (Ia-1), or (Ia-2), or a pharmaceutically acceptable salt thereof, wherein X is O; Y is O; T is CR¹¹R¹²; W is CR¹⁷R¹⁸; is 2; R² is —H; and R³, R¹¹, R¹², R¹³, R¹⁴, R¹⁵, R¹⁶, R¹⁷, and R¹⁸ are each —H.

In some embodiments, the invention relates to a compound of formula (I), (I-1), (I-2), (Ia), (Ia-1), or (Ia-2), or a pharmaceutically acceptable salt thereof, wherein X is O; Y is O; T is CR¹¹R¹²; W is CR¹⁷R¹⁸; n is 1; R² is —H; and R³, R¹¹, R¹², R¹³, R¹⁴, R¹⁵, R¹⁶, R¹⁷, and R¹⁸ are each —H.

In some embodiments, the invention relates to a compound of formula (I), (I-1), (I-2), (Ia), (Ia-1), or (Ia-2), or a pharmaceutically acceptable salt thereof, wherein X is O; Y is O; T is CR¹¹R¹²; W is CR¹⁷R¹⁸; n is 2; R² is —H; and R³, R¹¹, R¹², R¹³, R¹⁴, R¹⁵, R¹⁶, R¹⁷, and R¹⁸ are each —H.

In some embodiments, the invention relates to a compound of formula (I), (I-1), (I-2), (Ia), (Ia-1), or (Ia-2), or a pharmaceutically acceptable salt thereof, wherein X is O; Y is O; T is CR¹¹R¹²; W is CR¹⁷R¹⁸; m is 1; n is 1; R² is —H; and R³, R¹¹, R¹², R¹³, R¹⁴, R¹⁵, R¹⁶, R¹⁷, and R¹⁸ are each —H.

In some embodiments, the invention relates to a compound of formula (I), (I-1), (1-2), (Ia), (Ia-1), or (Ia-2), or a pharmaceutically acceptable salt thereof, wherein X is O; Y is O; T is CR¹¹R¹²; W is CR¹⁷R¹⁸; m is 1; n is 2; R² is —H; and R³, R¹¹, R¹², R¹³, R¹⁴, R¹⁵, R¹⁶, R¹⁷, and R¹⁸ are each —H.

In some embodiments, the invention relates to a compound of formula (I), (I-1), (I-2), (Ia), (Ia-1), or (Ia-2), or a pharmaceutically acceptable salt thereof, wherein X is O; Y is O; T is CR¹¹R¹²; W is CR¹⁷R¹⁸; m is 2; n is 1; R² is —H; and R³, R¹¹, R¹², R¹³, R¹⁴, R¹⁵, R¹⁶, R¹, and R¹⁸ are each —H.

In some embodiments, the invention relates to a compound of formula (I), (I-1), (I-2), (Ia), (Ia-1), or (Ia-2), or a pharmaceutically acceptable salt thereof, wherein X is O; m is 1; and n is 1.

In some embodiments, the invention relates to a compound of formula (I), (I-1), (I-2), (Ia), (Ia-1), or (Ia-2), or a pharmaceutically acceptable salt thereof, wherein X is O; m is 2; and n is 1.

In some embodiments, the invention relates to a compound of formula (I), (I-1), (I-2), (Ia), (Ia-1), or (Ia-2), or a pharmaceutically acceptable salt thereof, wherein X is O; m is 1; and n is 2.

In some embodiments, the invention relates to a compound of formula (I), (I-1), (I-2), (Ia), (Ia-1), or (Ia-2), or a pharmaceutically acceptable salt thereof, wherein X is O; R¹ is —CF₃; m is 1; and n is 1.

In some embodiments, the invention relates to a compound of formula (I), (I-1), (I-2), (Ia), (Ia-1), or (Ia-2), or a pharmaceutically acceptable salt thereof, wherein X is O; R¹ is —CF₃; m is 2; and n is 1.

In some embodiments, the invention relates to a compound of formula (I), (I-1), (I-2), (Ia), (Ia-1), or (Ia-2), or a pharmaceutically acceptable salt thereof, wherein X is O; R¹ is —CHF₂, —CF₃ or —CF₂CH₃; m is 1; and n is 1. In some embodiments, the invention relates to a compound of formula (I), (I-1), (I-2), (Ia), (Ia-1), or (Ia-2), or a pharmaceutically acceptable salt thereof, wherein X is O; R¹ is —CHF₂; m is 1; and n is 1. In some embodiments, the invention relates to a compound of formula (I), (I-1), (I-2), (Ia), (Ia-1), or (Ia-2), or a pharmaceutically acceptable salt thereof, wherein X is O; R¹ is —CF₃; m is 1; and n is 1. In some embodiments, the invention relates to a compound of formula (I), (I-1), (I-2), (Ia), (Ia-1), or (Ia-2), or a pharmaceutically acceptable salt thereof, wherein X is O; R¹ is —CF₂CH₃; m is 1; and n is 1.

In some embodiments, the invention relates to a compound of formula (I), (I-1), (I-2), (Ia), (Ia-1), or (Ia-2), or a pharmaceutically acceptable salt thereof, wherein X is O; R¹ is —CHF₂, —CF₃ or —CF₂CH₃; m is 1; and n is 2. In some embodiments, the invention relates to a compound of formula (I), (I-1), (I-2), (Ia), (Ia-1), or (Ia-2), or a pharmaceutically acceptable salt thereof, wherein X is O; R¹ is —CF₃ or —CF₂CH₃; m is 1; and n is 2. In some embodiments, the invention relates to a compound of formula (I), (I-1), (I-2), (Ia), (Ia-1), or (Ia-2), or a pharmaceutically acceptable salt thereof, wherein X is O; R¹ is —CHF₂; m is 1; and n is 2. In some embodiments, the invention relates to a compound of formula (I), (I-1), (I-2), (Ia), (Ia-1), or (Ia-2), or a pharmaceutically acceptable salt thereof, wherein X is O; R¹ is —CF₃; m is 1; and n is 2. In some embodiments, the invention relates to a compound of formula (I), (I-1), (I-2), (Ia), (Ia-1), or (Ia-2), or a pharmaceutically acceptable salt thereof, wherein X is O; R¹ is —CF₂CH₃; m is 1; and n is 2.

In some embodiments, the invention relates to a compound of formula (I), (I-1), (I-2), (Ia), (Ia-1), or (Ia-2), or a pharmaceutically acceptable salt thereof, wherein X is O; R¹ is —CHF₂, —CF₃ or —CF₂CH₃; m is 2; and n is 1. In some embodiments, the invention relates to a compound of formula (I), (I-1), (I-2), (Ia), (Ia-1), or (Ia-2), or a pharmaceutically acceptable salt thereof, wherein X is O; R¹ is —CHF₂; m is 2; and n is 1. In some embodiments, the invention relates to a compound of formula (I), (I-1), (I-2), (Ia), (Ia-1), or (Ia-2), or a pharmaceutically acceptable salt thereof, wherein X is O; R¹ is —CF₃; m is 2; and n is 1. In some embodiments, the invention relates to a compound of formula (I), (I-1), (I-2), (Ia), (Ia-1), or (Ia-2), or a pharmaceutically acceptable salt thereof, wherein X is O; R¹ is —CF₂CH₃; m is 2; and n is 1.

In some embodiments, the invention relates to a compound of formula (I), (I-1), (I-2), (Ia), (Ia-1), or (Ia-2), or a pharmaceutically acceptable salt thereof, wherein X is O; Y is O; m is 1; and n is 1.

In some embodiments, the invention relates to a compound of formula (I), (I-1), (I-2), (Ia), (Ia-1), or (Ia-2), or a pharmaceutically acceptable salt thereof, wherein X is O; Y is O; m is 2; and n is 1.

In some embodiments, the invention relates to a compound of formula (I), (I-1), (I-2), (Ia), (Ia-1), or (Ia-2), or a pharmaceutically acceptable salt thereof, wherein X is O; Y is O; m is 1; and n is 2.

In some embodiments, the invention relates to a compound of formula (I), (I-1), (I-2), (Ia), (Ia-1), or (Ia-2), or a pharmaceutically acceptable salt thereof, wherein X is O; Y is CH₂; m is 1; and n is 1.

In some embodiments, the invention relates to a compound of formula (I), (I-1), (I-2), (Ia), (Ia-1), or (Ia-2), or a pharmaceutically acceptable salt thereof, wherein X is O; Y is CH₂; m is 2; and n is 1.

In some embodiments, the invention relates to a compound of formula (I), (I-1), (I-2), (Ia), (Ia-1), or (Ia-2), or a pharmaceutically acceptable salt thereof, wherein X is O; Y is CH₂; m is 1; and n is 2.

In some embodiments, the invention relates to a compound of formula (I), (I-1), (I-2), (Ia), (Ia-1), or (Ia-2), or a pharmaceutically acceptable salt thereof, wherein X is O; Y is O; R¹ is —CHF₂; m is 1; and n is 1.

In some embodiments, the invention relates to a compound of formula (I), (I-1), (I-2), (Ia), (Ia-1), or (Ia-2), or a pharmaceutically acceptable salt thereof, wherein X is O; Y is O; R¹ is —CHF₂; m is 2; and n is 1.

In some embodiments, the invention relates to a compound of formula (I), (I-1), (I-2), (Ia), (Ia-1), or (Ia-2), or a pharmaceutically acceptable salt thereof, wherein X is O; Y is O; R¹ is —CHF₂; m is 1; and n is 2.

In some embodiments, the invention relates to a compound of formula (I), (I-1), (I-2), (Ia), (Ia-1), or (Ia-2), or a pharmaceutically acceptable salt thereof, wherein X is O; Y is O; R¹ is —CF₃; m is 1; and n is 1.

In some embodiments, the invention relates to a compound of formula (I), (I-1), (I-2), (Ia), (Ia-1), or (Ia-2), or a pharmaceutically acceptable salt thereof, wherein X is O; Y is O; R¹ is —CF₃; m is 2; and n is 1.

In some embodiments, the invention relates to a compound of formula (I), (I-1), (I-2), (Ia), (Ia-1), or (Ia-2), or a pharmaceutically acceptable salt thereof, wherein X is O; Y is O; R¹ is —CF₃; m is 1; and n is 2.

In some embodiments, the invention relates to a compound of formula (I), (I-1), (I-2), (Ia), (Ia-1), or (Ia-2), or a pharmaceutically acceptable salt thereof, wherein X is O; Y is O; R¹ is —CF₂CH₃; m is 1; and nis 1.

In some embodiments, the invention relates to a compound of formula (I), (I-1), (I-2), (Ia), (Ia-1), or (Ia-2), or a pharmaceutically acceptable salt thereof, wherein X is O; Y is O; R¹ is —CF₂CH₃; m is 1; and n is 2.

In some embodiments, the invention relates to a compound of formula (I), (I-1), (I-2), (Ia), (Ia-1), or (Ia-2), or a pharmaceutically acceptable salt thereof, wherein X is O; Y is O; R¹ is —CF₂CH₃; m is 2; and nis 1.

In some embodiments, the invention relates to a compound of formula (I), (I-1), (I-2), (Ia), (Ia-1), or (Ia-2), or a pharmaceutically acceptable salt thereof, wherein X is O; Y is CH₂; R¹ is —CHF₂; m is 1; and nis 1.

In some embodiments, the invention relates to a compound of formula (I), (I-1), (1-2), (Ia), (Ia-1), or (Ia-2), or a pharmaceutically acceptable salt thereof, wherein X is O; Y is CH₂; R¹ is —CHF₂; m is 2; and nis 1.

In some embodiments, the invention relates to a compound of formula (I), (I-1), (I-2), (Ia), (Ia-1), or (Ia-2), or a pharmaceutically acceptable salt thereof, wherein X is O; Y is CH₂; R¹ is —CHF₂; m is 1; and n is 2.

In some embodiments, the invention relates to a compound of formula (I), (I-1), (I-2), (Ia), (Ia-1), or (Ia-2), or a pharmaceutically acceptable salt thereof, wherein X is O; Y is CH₂; R¹ is —CF₃; m is 1; and n is 1.

In some embodiments, the invention relates to a compound of formula (I), (I-1), (I-2), (Ia), (Ia-1), or (Ia-2), or a pharmaceutically acceptable salt thereof, wherein X is O; Y is CH₂; R¹ is —CF₃; m is 2; and n is 1.

In some embodiments, the invention relates to a compound of formula (I), (I-1), (I-2), (Ia), (Ia-1), or (Ia-2), or a pharmaceutically acceptable salt thereof, wherein X is O; Y is CH₂; R¹ is —CF₃; m is 1; and n is 2.

In some embodiments, the invention relates to a compound of formula (I), (I-1), (I-2), (Ia), (Ia-1), or (Ia-2), or a pharmaceutically acceptable salt thereof, wherein X is O; Y is CH₂; R¹ is —CF₂CH₃; m is 1; and nis 1.

In some embodiments, the invention relates to a compound of formula (I), (I-1), (I-2), (Ia), (Ia-1), or (Ia-2), or a pharmaceutically acceptable salt thereof, wherein X is O; Y is CH₂; R¹ is —CF₂CH₃; m is 2; and nis 1.

In some embodiments, the invention relates to a compound of formula (I), (I-1), (I-2), (Ia), (Ia-1), or (Ia-2), or a pharmaceutically acceptable salt thereof, wherein X is O; Y is CH₂; R¹ is —CF₂CH₃; m is 1; and n is 2.

In some embodiments, the invention relates to a compound of formula (I), (I-1), (I-2), (Ia), (Ia-1), or (Ia-2), or a pharmaceutically acceptable salt thereof, wherein X is O; Y is O; R¹ is —CHF₂; m is 1; n is 1; and q is 0.

In some embodiments, the invention relates to a compound of formula (I), (I-1), (I-2), (Ia), (Ia-1), or (Ia-2), or a pharmaceutically acceptable salt thereof, wherein X is O; Y is O; R¹ is —CHF₂; m is 2; n is 1; and q is 0.

In some embodiments, the invention relates to a compound of formula (I), (I-1), (I-2), (Ia), (Ia-1), or (Ia-2), or a pharmaceutically acceptable salt thereof, wherein X is O; Y is O; R¹ is —CHF₂; m is 1; n is 2; and q is 0.

In some embodiments, the invention relates to a compound of formula (I), (I-1), (I-2), (Ia), (Ia-1), or (Ia-2), or a pharmaceutically acceptable salt thereof, wherein X is O; Y is O; R¹ is —CF₃; m is 1; n is 1; and q is 0.

In some embodiments, the invention relates to a compound of formula (I), (I-1), (I-2), (Ia), (Ia-1), or (Ia-2), or a pharmaceutically acceptable salt thereof, wherein X is O; Y is O; R¹ is —CF₃; m is 2; n is 1; and q is 0.

In some embodiments, the invention relates to a compound of formula (I), (I-1), (I-2), (Ia), (Ia-1), or (Ia-2), or a pharmaceutically acceptable salt thereof, wherein X is O; Y is O; R¹ is —CF₃; m is 1; n is 2; and q is 0.

In some embodiments, the invention relates to a compound of formula (I), (I-1), (I-2), (Ia), (Ia-1), or (Ia-2), or a pharmaceutically acceptable salt thereof, wherein X is O; Y is O; R¹ is —CF₂CH₃; m is 1; n is 1; and q is 0.

In some embodiments, the invention relates to a compound of formula (I), (I-1), (I-2), (Ia), (Ia-1), or (Ia-2), or a pharmaceutically acceptable salt thereof, wherein X is O; Y is O; R¹ is —CF₂CH₃; m is 2; n is 1; and q is 0.

In some embodiments, the invention relates to a compound of formula (I), (I-1), (I-2), (Ia), (Ia-1), or (Ia-2), or a pharmaceutically acceptable salt thereof, wherein X is O; Y is O; R¹ is —CF₂CH₃; m is 1; n is 2; and q is 0.

In some embodiments, the invention relates to a compound of formula (I), (I-1), (I-2), (Ia), (Ia-1), or (Ia-2), or a pharmaceutically acceptable salt thereof, wherein X is O; Y is CH₂; R¹ is —CHF₂; m is 1; n is 1; and q is 0.

In some embodiments, the invention relates to a compound of formula (I), (I-1), (I-2), (Ia), (Ia-1), or (Ia-2), or a pharmaceutically acceptable salt thereof, wherein X is O; Y is CH₂; R¹ is —CHF₂; m is 2; n is 1; and q is 0.

In some embodiments, the invention relates to a compound of formula (I), (I-1), (I-2), (Ia), (Ia-1), or (Ia-2), or a pharmaceutically acceptable salt thereof, wherein X is O; Y is CH₂; R¹ is —CHF₂; m is 2; n is 2; and q is 0.

In some embodiments, the invention relates to a compound of formula (I), (I-1), (I-2), (Ia), (Ia-1), or (Ia-2), or a pharmaceutically acceptable salt thereof, wherein X is O; Y is CH₂; R¹ is —CF₃; m is 1; n is 1; and q is 0.

In some embodiments, the invention relates to a compound of formula (I), (I-1), (I-2), (Ia), (Ia-1), or (Ia-2), or a pharmaceutically acceptable salt thereof, wherein X is O; Y is CH₂; R¹ is —CF₃; m is 2; n is 1; and q is 0.

In some embodiments, the invention relates to a compound of formula (I), (I-1), (I-2), (Ia), (Ia-1), or (Ia-2), or a pharmaceutically acceptable salt thereof, wherein X is O; Y is CH₂; R¹ is —CF₃; m is 1; n is 2; and q is 0.

In some embodiments, the invention relates to a compound of formula (I), (I-1), (I-2), (Ia), (Ia-1), or (Ia-2), or a pharmaceutically acceptable salt thereof, wherein X is O; Y is CH₂; R¹ is —CF₂CH₃; m is 1; n is 1; and q is 0.

In some embodiments, the invention relates to a compound of formula (I), (I-1), (I-2), (Ia), (Ia-1), or (Ia-2), or a pharmaceutically acceptable salt thereof, wherein X is O; Y is CH₂; R¹ is —CF₂CH₃; m is 2; n is 1; and q is 0.

In some embodiments, the invention relates to a compound of formula (I), (I-1), (I-2), (Ia), (Ia-1), or (Ia-2), or a pharmaceutically acceptable salt thereof, wherein X is O; Y is CH₂; R¹ is —CF₂CH₃; m is 2; n is 2; and q is 0.

In some embodiments, the invention relates to a compound of formula (I), (I-1), (I-2), (Ia), (Ia-1), or (Ia-2), or a pharmaceutically acceptable salt thereof, wherein q is 0 and Y is O.

In some embodiments, the invention relates to a compound of formula (I-A):

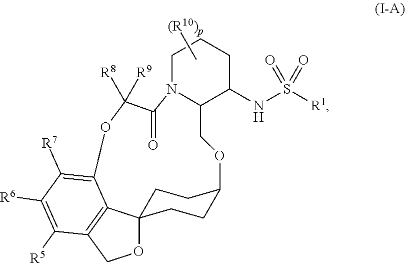

or a pharmaceutically acceptable salt thereof.

In some embodiments, the invention relates to a compound of formula (I-A-1):

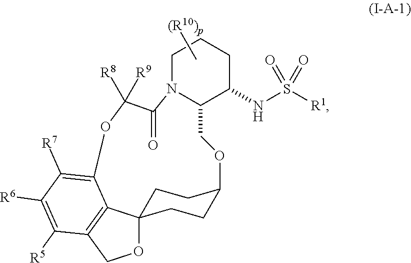

or a pharmaceutically acceptable salt thereof.

In some embodiments, the invention relates to a compound of formula (I-A-2):

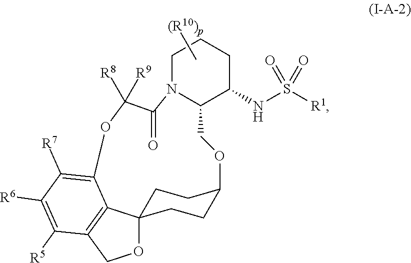

or a pharmaceutically acceptable salt thereof.

In some embodiments, the invention relates to a compound of formula (I-B):

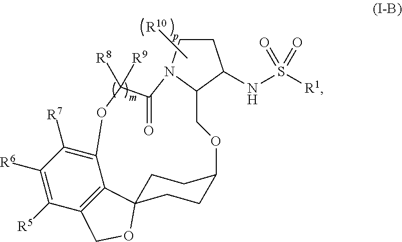

or a pharmaceutically acceptable salt thereof.

In some embodiments, the invention relates to a compound of formula (I-B-a):

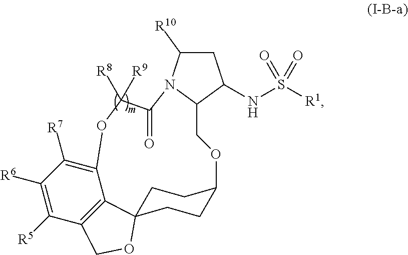

or a pharmaceutically acceptable salt thereof.

In some embodiments, the invention relates to a compound of formula (I-B-1):

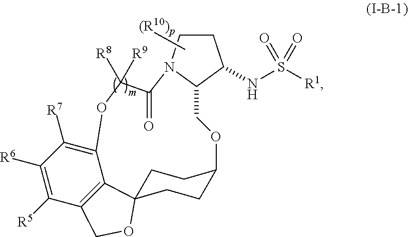

or a pharmaceutically acceptable salt thereof.

In some embodiments, the invention relates to a compound of formula (I-B-1a):

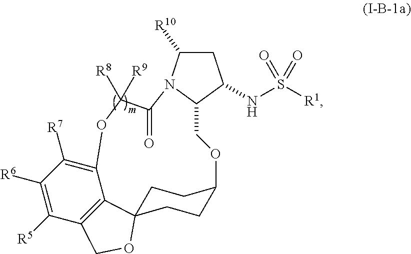

or a pharmaceutically acceptable salt thereof.

In some embodiments, the invention relates to a compound of formula (I-B-2):

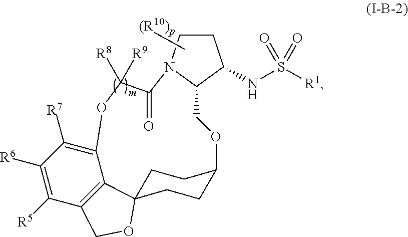

or a pharmaceutically acceptable salt thereof.

In some embodiments, the invention relates to a compound of formula (I-B-2a):

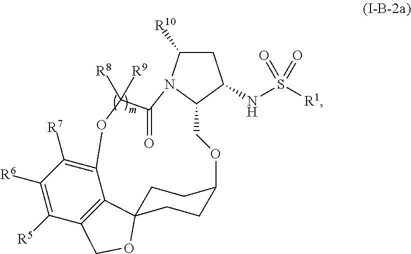

or a pharmaceutically acceptable salt thereof.

In some embodiments, the invention relates to a compound of formula (I-B), (I-B-a), (I-B-1), (I-B-1a), (I-B-2), or (I-B-2a), or a pharmaceutically acceptable salt thereof, wherein m is 1 or 2. In some embodiments, the invention relates to a compound of formula (I-B), (I-B-a), (I-B-1), (I-B-1a), (I-B-2), or (I-B-2a), or a pharmaceutically acceptable salt thereof, wherein m is 1. In some embodiments, the invention relates to a compound of formula (I-B), (I-B-a), (I-B-1), (I-B-1a), (I-B-2), or (I-B-2a), or a pharmaceutically acceptable salt thereof, wherein m is 2.

In some embodiments, the invention relates to a compound of formula (I-B), (I-B-a), (I-B-1), (I-B-1a), (I-B-2), or (I-B-2a), or a pharmaceutically acceptable salt thereof, wherein R¹⁰ is —C₁-C₆ alkyl. In some embodiments, the invention relates to a compound of formula (I-B), (I-B-a), (I-B-1), (I-B-1a), (I-B-2), or (I-B-2a), or a pharmaceutically acceptable salt thereof, wherein R¹⁰ is —CH₃.

In some embodiments, the invention relates to a compound of formula (I), (I-1), (I-2), (Ia), (Ia-1), or (Ia-2), or a pharmaceutically acceptable salt thereof, wherein q is 0 and Y is C(R¹⁹)₂, and each R¹⁹ is H.

In some embodiments, the invention relates to a compound of formula (I-C):

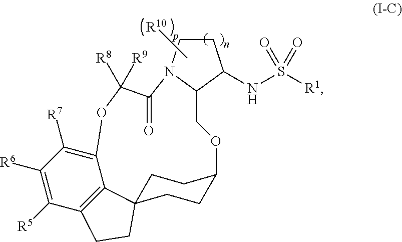

or a pharmaceutically acceptable salt thereof.

In some embodiments, the invention relates to a compound of formula (I-C-a):

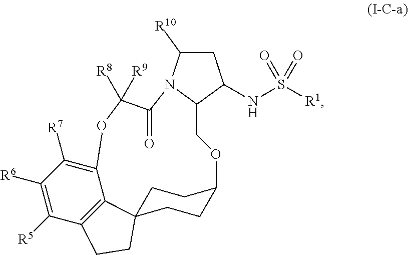

or a pharmaceutically acceptable salt thereof.

In some embodiments, the invention relates to a compound of formula (I-C-1):

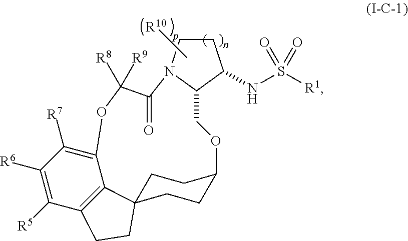

or a pharmaceutically acceptable salt thereof.

In some embodiments, the invention relates to a compound of formula (I-C-1a):

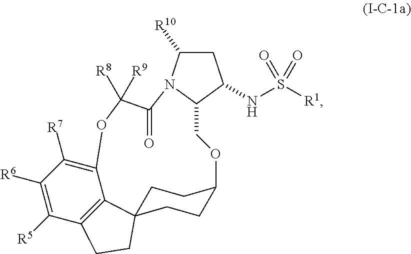

or a pharmaceutically acceptable salt thereof.

In some embodiments, the invention relates to a compound of formula (I-C-2):

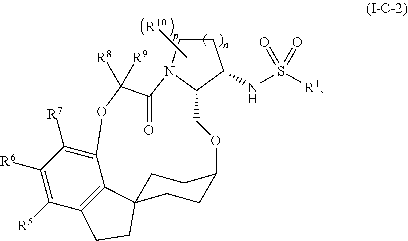

or a pharmaceutically acceptable salt thereof.

In some embodiments, the invention relates to a compound of formula (I-C-2a):

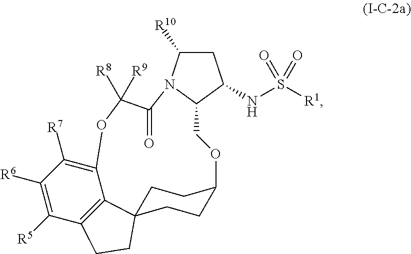

or a pharmaceutically acceptable salt thereof.

In some embodiments, the invention relates to a compound of formula (I-C), (I-C-1), or (I-C-2), or a pharmaceutically acceptable salt thereof, wherein n is 1 or 2. In some embodiments, the invention relates to a compound of formula (I-C), (I-C-1), or (I-C-2), or a pharmaceutically acceptable salt thereof, wherein n is 1. In some embodiments, the invention relates to a compound of formula (I-C), (I-C-1), or (I-C-2), or a pharmaceutically acceptable salt thereof, wherein n is 2.

In some embodiments, the invention relates to a compound of formula (I-C-a), (I-C-1a), or (I-C-2a), or a pharmaceutically acceptable salt thereof, wherein R¹⁰ is —C₁-C₆ alkyl. In some embodiments, the invention relates to a compound of formula (I-C-a), (I-C-1a), or (I-C-2a), or a pharmaceutically acceptable salt thereof, wherein R¹⁰ is —CH₃.

In some embodiments, the invention relates to a compound of formula (I), (I-1), (I-2), (Ia), (Ia-1), or (Ia-2), or a pharmaceutically acceptable salt thereof, wherein q is 1.

In some embodiments, the invention relates to a compound of formula (I), (I-1), (I-2), (Ia), (Ia-1), or (Ia-2), or a pharmaceutically acceptable salt thereof, wherein q is 1 and Y is O.

In some embodiments, the invention relates to a compound of formula (I-D):

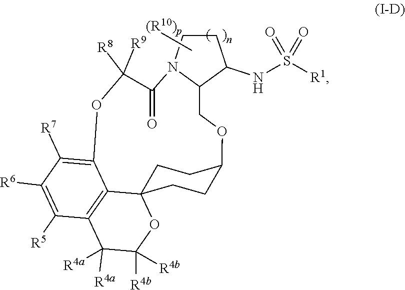

or a pharmaceutically acceptable salt thereof.

In some embodiments, the invention relates to a compound of formula (I-D-a):

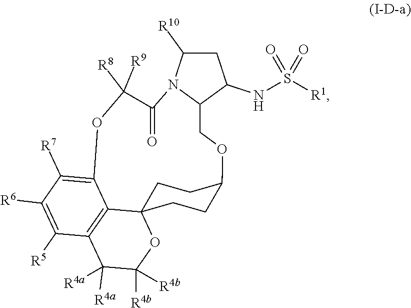

or a pharmaceutically acceptable salt thereof.

In some embodiments, the invention relates to a compound of formula (I-D-1):

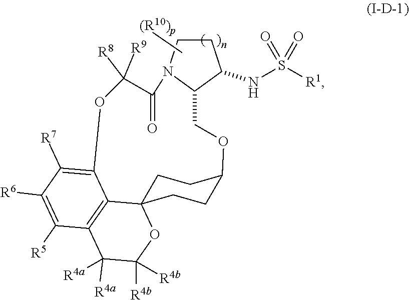

or a pharmaceutically acceptable salt thereof.

In some embodiments, the invention relates to a compound of formula (I-D-1a):

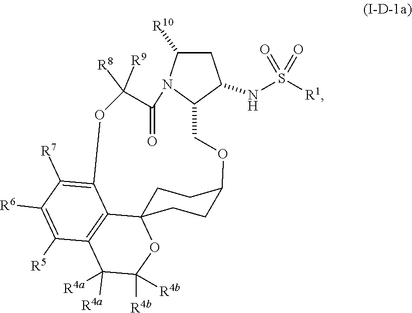

or a pharmaceutically acceptable salt thereof.

In some embodiments, the invention relates to a compound of formula (I-D-2):

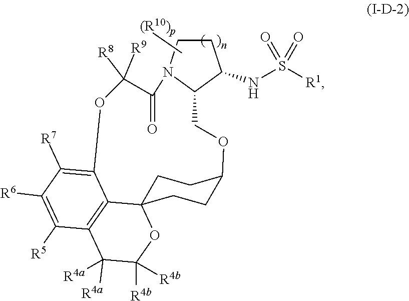

or a pharmaceutically acceptable salt thereof.

In some embodiments, the invention relates to a compound of formula (I-D-2a):

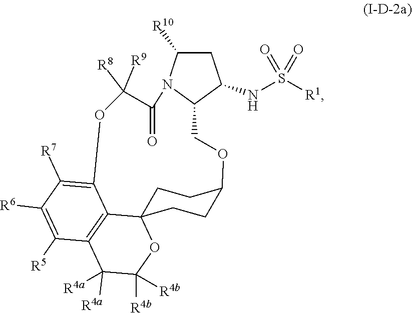

or a pharmaceutically acceptable salt thereof.

In some embodiments, the invention relates to a compound of formula (I), (I-1), (I-2), (Ia), (Ia-1), (Ia-2), (I-D), (I-D-a), (I-D-1), (I-D-1a), (I-D-2), or (I-D-2a), or a pharmaceutically acceptable salt thereof, wherein each R^{4b} is H.

In some embodiments, the invention relates to a compound of formula (I-D-a), (I-D-1a), or (I-D-2a) or a pharmaceutically acceptable salt thereof, wherein R¹⁰ is —C₁-C₆ alkyl. In some embodiments, the invention relates to a compound of formula (I-D-a), (I-D-1a), or (I-D-2a) or a pharmaceutically acceptable salt thereof, wherein R¹⁰ is —CH₃.

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is selected from —C₁-C₆ alkyl, —C₁-C₆ haloalkyl, —C₁-C₃ alkylene-OH, —C₃-C₈ cycloalkyl, 4- to 10-membered heterocyclyl, and —C₆-C₁₀ aryl, wherein said —C₃-C₈ cycloalkyl, 4- to 10-membered heterocyclyl, and —C₆-C₁₀ aryl, is optionally substituted with 1-3 substituents independently selected from halo, —OH, —C₁-C₃ alkyl, and —C₁-C₃ alkoxy. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is selected from —C₁-C₆ alkyl, —C₁-C₆ haloalkyl, —C₃-C₈cycloalkyl, 4- to 10-membered heterocyclyl, and —C₆-C₁₀ aryl, wherein said —C₃-C₈ cycloalkyl, 4- to 10-membered heterocyclyl, and —C₆-C₁₀ aryl, is optionally substituted with 1-3 substituents independently selected from halo, —OH, —C₁-C₃ alkyl, and —C₁-C₃ alkoxy. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is selected from —C₁-C₆ alkyl, —C₁-C₆ haloalkyl, —C₃-C₈ cycloalkyl, and 4- to 10-membered heterocyclyl, wherein said —C₃-C₈ cycloalkyl or 4- to 10-membered heterocyclyl is optionally substituted with 1-3 substituents independently selected from halo, —OH, —C₁-C₃ alkyl, and —C₁-C₃ alkoxy. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is selected from —C₁-C₆ alkyl, —C₁-C₆ haloalkyl, —C₃-C₈ cycloalkyl, and 4- to 10-membered heterocyclyl, wherein said —C₃-C₈ cycloalkyl is optionally substituted with 1 substituent selected from halo, —OH, —C₁-C₃ alkyl, and —C₁-C₃ alkoxy. In some embodiments, the invention relates to a compound of formula (I), (I-1), (I-2), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is —C₁-C₆ alkyl. In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is —C₁-C₆ haloalkyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is —C₁-C₃ alkylene-OH. In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is unsubstituted —C₃-C₈ cycloalkyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is unsubstituted 4- to 10-membered heterocyclyl. In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is unsubstituted —C₆-C₁₀ aryl. In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is —C₃-C₈ cycloalkyl optionally substituted with 1-3 substituents independently selected from halo, —OH, —C₁-C₃ alkyl, and —C₁-C₃ alkoxy. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is 4- to 10-membered heterocyclyl optionally substituted with 1-3 substituents independently selected from halo, —OH, —C₁-C₃ alkyl, and —C₁-C₃ alkoxy. In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is —C₆-C₁₀ aryl optionally substituted with 1-3 substituents independently selected from halo, —OH, —C₁-C₃ alkyl, and —C₁-C₃ alkoxy. In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is —C₃-C₈ cycloalkyl substituted with 1 substituent selected from halo, —OH, —C₁-C₃ alkyl, and —C₁-C₃ alkoxy. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is —C₆-C₁₀ aryl substituted with 1 substituent independently selected from halo, —OH, —C₁-C₃ alkyl, and —C₁-C₃ alkoxy. In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is —C₃-C₈ cycloalkyl substituted with 1 substituent selected from halo. In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is —C₆-C₁₀ aryl substituted with 1 substituent selected from halo.

In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is selected from —CH₃, —CH₂CH₃, —CH₂CH₂CH₃, —CH(CH₃)₂, —CH(CH₃)CH₂CH₃, —CH₂F, —CHF₂, —CF₃, —CHFCH₃, —CH₂CF₃, —CF₂CH₃, —CH₂CH₂OH, cyclopropyl, 1-fluorocyclopropyl, 1-methylcyclopropyl, cyclobutyl, 3,3-difluorocyclobutyl, azetidinyl, oxetanyl, pyrrolidinyl, tetrahydrofuranyl, and 2-fluorophenyl. In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is selected from —CH₃, —CH₂CH₃, —CH₂CH₂CH₃, —CH(CH₃)₂, —CH(CH₃)CH₂CH₃, —CH₂F, —CHF₂, —CF₃, —CHFCH₃, —CH₂CF₃, —CF₂CH₃, cyclopropyl, 1-fluorocyclopropyl, 1-methylcyclopropyl, cyclobutyl, 3,3-difluorocyclobutyl, oxetanyl, tetrahydrofuranyl, and 2-fluorophenyl. In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is selected from —CH₃, —CH₂CH₃, —CH₂F, —CHF₂, —CF₃, 1-fluorocyclopropyl, and oxetanyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is —CH₃. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is —CH₂CH₃. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is —CH₂CH₂CH₃. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is —CH(CH₃)₂. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is —CH(CH₃)CH₂CH₃. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is —CH₂F. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is —CHF₂. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is —CF₃. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is —CHFCH₃. In some embodiments, the invention relates to a compound of formula (I), (I-1), (I-2), (Ia), (Ia-1), (Ia-2), (I-A), (I-A-1), (I-A-2), (I-D-a), (I-D-1), (I-D-1a), (I-D-2), or (I-D-2a), or a pharmaceutically acceptable salt thereof, wherein R¹ is —CH₂CF₃. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is —CF₂CH₃. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is CH₂CH₂OH. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is cyclopropyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is 1-fluorocyclopropyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is 1-methylcyclopropyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is cyclobutyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is 3,3-difluorocyclobutyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is azetidinyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is unsubstituted azetidinyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is oxetanyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is unsubstituted oxetanyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is pyrrolidinyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is unsubstituted pyrrolidinyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is tetrahydrofuranyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is unsubstituted tetrahydrofuranyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is 2-fluorophenyl.

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is selected from —CH₃, —CH₂CH₃, —CH₂CH₂CH₃, —CH(CH₃)₂, —CH(CH₃)CH₂CH₃, —CH₂F, —CHF₂, —CF₃, —CHFCH₃, —CH₂CF₃, —CF₂CH₃, —CH₂CH₂OH,

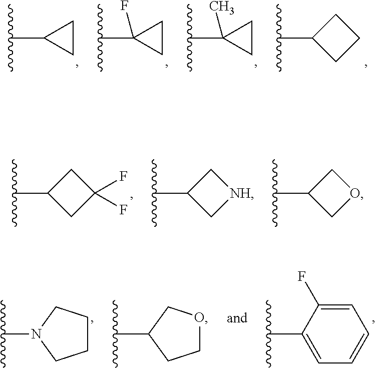

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is

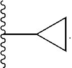 [SMILES: *C1CC1]

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is

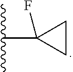 [SMILES: *C1(F)CC1]

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is

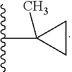 [SMILES: *C1(C)CC1]

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is

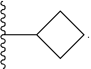 [SMILES: *C1CCC1]

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is

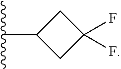 [SMILES: *C1CC(F)(F)C1]

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is

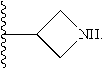 [SMILES: *C1CNC1]

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is

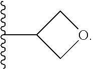 [SMILES: *C1COC1]

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is

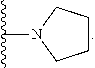 [SMILES: *N1CCCC1]

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is

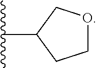 [SMILES: *C1CCOC1]

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is

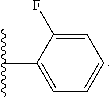 [SMILES: *c1ccccc1F]

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is selected from —CHF₂, —CF₃, and —CF₂CH₃.

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵, R⁶, and R⁷ are each independently selected from —H halo, —C₁-C₆ alkyl, and —C₁-C₆ haloalkyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵, R⁶, and R⁷ are each independently selected from —H and halo.

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is —H, halo, or —C₁-C₆ alkyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is —H or halo. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is —H or —Cl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is —H. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is halo. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is —C₁-C₆ alkyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is —H, —F, —Cl, —Br, or —CH₃. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is —F. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is —Cl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is —Br. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is —CH₃.

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁶ is —H or halo. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁶ is halo. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁶ is —H. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁶ is —H, —F, or —Br. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁶ is —H or —F. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁶ is —F. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁶ is —Br.

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁷ is —H, halo, —C₁-C₆ alkyl, and —C₁-C₆haloalkyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁷ is —H. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁷ is halo. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁷ is —C₁-C₆ alkyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁷ is —C₁-C₆ haloalkyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R is —H, —F, —Br, —CH₃, or —CF₃. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁷ is —H, —F, —CH₃, or —CF₃. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R is —F. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁷ is —Br. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁷ is —CH₃. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R¹ is —CF₃.

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁷ is —H; R⁶ is —H; and R⁵ is —H or halo. In some embodiments, the invention relates to a compound of formula (I), (I-1), (I-2), (Ia), (Ia-1), (Ia-2), (I-A), (I-A-1), (I-A-2), (I-D-a), (I-D-1), (I-D-1a), (I-D-2), or (I-D-2a), or a pharmaceutically acceptable salt thereof, wherein R is —H; R⁶ is —H; and R⁵ is —H. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁷ is —H; R⁶ is —H; and R⁵ is halo. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁷ is —H; R⁶ is —H; and R⁵ is —H, —F, or —Cl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁷ is —H; R⁶ is —H; and R⁵ is —H or —Cl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁷ is —H; R⁶ is —H; and R⁵ is —F. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁷ is —H; R⁶ is —H; and R⁵ is —Cl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁷ is —H; R⁶ is —H; and R⁵ is —Br. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is halo; R⁶ is halo; and R⁷ is —H. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is —F; R⁶ is —F; and R⁷ is —H. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is —H; R⁶ is —H; and R⁷ is halo. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is —H; R⁶ is —H; and R⁷ is —F. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is halo; R⁶ is —H; and R⁷ is halo. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is —F; R⁶ is —H; and R⁷ is —F. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is —Br; R⁶ is —H; and R⁷ is —Br. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is —Cl; R⁶ is —H; and R⁷ is —F. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is halo; R⁶ is —H; and R⁷ is —C₁-C₆ alkyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is —F; R⁶ is —H; and R⁷ is —CH₃. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is —C₁-C₆ alkyl; R⁶ is —H; and R⁷ is —H. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is —CH₃; R⁶ is —H; and R⁷ is —H. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is —H; R⁶ is —H; and R⁷ is —C₁-C₆ haloalkyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is —H; R⁶ is —H; and R⁷ is —CF₃. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁵ is halo; R⁶ is halo; and R⁷ is halo. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R is —F; R⁶ is —Br; and R⁷ is —F.

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein each R⁸ and each R⁹ is independently selected from —H, halo, and —C₁-C₆ alkyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein at least one R⁸ or R⁹ is —H. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein at least one R⁸ or R⁹ is halo. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein at least one R⁸ or R⁹ is —C₁-C₆ alkyl.

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein each R⁸ and each R⁹ is independently selected from —H, —F, —CH₃, and —CH₂CH₃. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, at least one R⁸ or R⁹ is —H. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, at least one R⁸ or R⁹ is —F. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, at least one R⁸ or R⁹ is —CH₃. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, at least one R⁸ or R⁹ is —CH₂CH₃. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, R⁸ and R⁹ are each halo. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, R⁸ and R⁹ are each —F.

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein each R⁸ is —H. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁸ is —H. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein at least one R⁸ is halo. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein at least one R⁸ is —F. In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein at least one R⁸ is —C₁-C₆ alkyl. In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein at least one R⁸ is —CH₃. In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein at least one R⁸ is —CH₂CH₃. In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein at least one R⁸ is -D.

In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein each R⁹ is —H. In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁹ is —H. In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein at least one R⁹ is halo. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein at least one R⁹ is —F. In some embodiments, the invention relates to a compound of formula (I), (I-1), (1-2), (Ia), (Ia-1), (Ia-2), (I-A), (I-C-2), (I-C-2a), (I-D), (I-D-a), (I-D-1), (I-D-1a), (I-D-2), or (I-D-2a), or a pharmaceutically acceptable salt thereof, wherein at least one R⁹ is —C₁-C₆ alkyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein at least one R⁹ is —CH₃. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein at least one R⁹ is —CH₂CH₃. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein at least one R⁹ is -D.

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein each R⁸ and R⁹ are each —H. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁸ and R⁹ are each —H.

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein p is 0, 1, or 2. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein p is 0. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein p is 0. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein p is 1. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein p is 2.

In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein each R¹⁰ is independently selected from halo and —C₁-C₆ alkyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein at least one R¹⁰ is halo or —C₁-C₆ alkyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein at least one R¹⁰ is halo. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein at least one R¹⁰ is —C₁-C₆ alkyl. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein each R¹⁰ is independently selected from —F and —CH₃. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein at least one R¹⁰ is —F or —CH₃. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein at least one R¹⁰ is —F. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein at least one R¹⁰ is —CH₃.

In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein p is 1 and R¹⁰ is —C₁-C₆ alkyl. In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein p is 1 and R¹⁰ is —CH₃.

In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein p is 2 and each R¹⁰ is independently selected from halo. In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein p is 2 and each R¹⁰ is —F.

In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁷ is —H; R⁶ is —H; R⁵ is —H or halo; R⁸ is —H; and R⁹ is —H. In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁷ is —H; R⁶ is —H; R⁵ is —H; R⁸ is —H; and R⁹ is —H. In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁷ is —H; R⁶ is —H; R⁵ is halo; R⁸ is —H; and R⁹ is —H. In some embodiments, the invention relates to a compound of formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁷ is —H; R⁶ is —H; R⁵ is —H or —Cl; R⁸ is —H; and R⁹ is —H. In some embodiments, the invention relates to a compound of formula (I), (I-1), (I-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), or (I-D-2a), or a pharmaceutically acceptable salt thereof, wherein R⁷ is —H; R⁶ is —H; R⁵ is —Cl; R⁸ is —H; and R⁹ is —H.

In some embodiments, the invention relates to a compound of formula (I), (I-1), (I-2), (Ia), (Ia-1), (Ia-2), (I-A), (I-A-1), (I-A-2), (I-B), (I-B-1), (I-B-2), (I-C), (I-C-1), (I-C-2), (I-D), (I-D-1), or (I-D-2), or a pharmaceutically acceptable salt thereof, wherein R⁷ is —H; R⁶ is —H; R⁵ is —H or halo; R⁸ is —H; R⁹ is —H; and p is 0. In some embodiments, the invention relates to a compound of formula (I), (I-1), (I-2), (Ia), (Ia-1), (Ia-2), (I-A), (I-A-1), (I-A-2), (I-B), (I-B-1), (I-B-2), (I-C), (I-C-1), (I-C-2), (I-D), (I-D-1), or (I-D-2), or a pharmaceutically acceptable salt thereof, wherein R⁷ is —H; R⁶ is —H; R⁵ is —H; R⁸ is —H; R⁹ is —H; and p is 0. In some embodiments, the invention relates to a compound of formula (I), (I-1), (I-2), (Ia), (Ia-1), (Ia-2), (I-A), (I-A-1), (I-A-2), (I-B), (I-B-1), (I-B-2), (I-C), (I-C-1), (I-C-2), (I-D), (I-D-1), or (I-D-2), or a pharmaceutically acceptable salt thereof, wherein R⁷ is —H; R⁶ is —H; R⁵ is halo; R⁸ is —H; R⁹ is —H; and p is 0. In some embodiments, the invention relates to a compound of formula (I), (I-1), (I-2), (Ia), (Ia-1), (Ia-2), (I-A), (I-A-1), (I-A-2), (I-B), (I-B-1), (I-B-2), (I-C), (I-C-1), (I-C-2), (I-D), (I-D-1), or (I-D-2), or a pharmaceutically acceptable salt thereof, wherein R⁷ is —H; R⁶ is —H; R⁵ is —H or —Cl; R⁸ is —H; R⁹ is —H; and p is 0. In some embodiments, the invention relates to a compound of formula (I), (I-1), (I-2), (Ia), (Ia-1), (Ia-2), (I-A), (I-A-1), (I-A-2), (I-B), (I-B-1), (I-B-2), (I-C), (I-C-1), (I-C-2), (I-D), (I-D-1), or (I-D-2), or a pharmaceutically acceptable salt thereof, wherein R⁷ is —H; R⁶ is —H; R⁵ is —Cl; R^{B} is —H; R⁹ is —H; and p is 0.

In some embodiments, the invention relates to a compound selected from Table A, or a pharmaceutically acceptable salt thereof. In other embodiments, the invention relates to a compound selected from Table A, i.e., the compound in non-salt form.

TABLE A
Compound Structures and Names

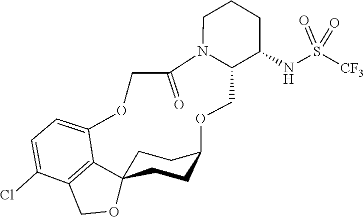

N-((5aR,8R,10aR,11S)-3-
Chloro-16-oxo-
7,8,10,10a,11,12,13,14,16,17-
decahydro-4H,6H,5a,8-
ethanoisobenzofuro[7,1-
hi]pyrido[2,1-
c][1,7]dioxa[4]azacyclotridecin-
11-yl)-1,1,1-
trifluoromethanesulfonamide

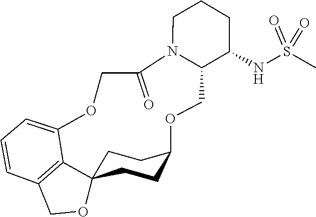

N-((5aR,8R,10aR,11S)-16-Oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)methanesulfonamide

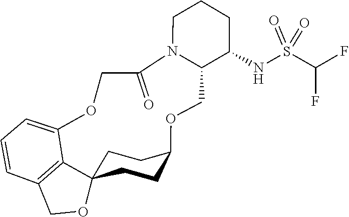

1,1-Difluoro-N-
((5aR,8R,10aR,11S)-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)methanesulfonamide

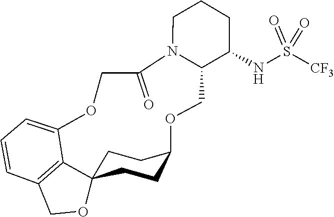

1,1,1-Trifluoro-N-
((5aR,8R,10aR,11S)-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)methanesulfonamide

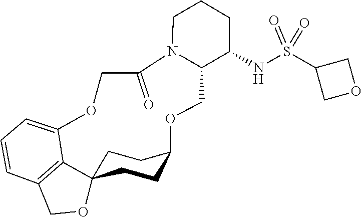

N-((5aR,8R,10aR,11S)-16-Oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)oxetane-3-sulfonamide

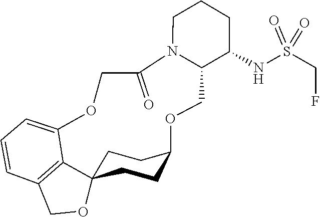

1-Fluoro-N-((5aR,8R,10aR,11S)-
16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)methanesulfonamide

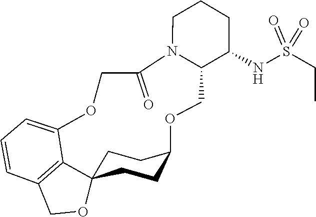

N-((5aR,8R,10aR,11S)-16-Oxo-
7,8,10,10a,11,12,13,14,16,17-decahydro-4H,6H-
5a,8-ethanoisobenzofuro[7,1-hi]pyrido[2,1-
c][1,7]dioxa[4]azacyclotridecin-11-
yl)ethanesulfonamide

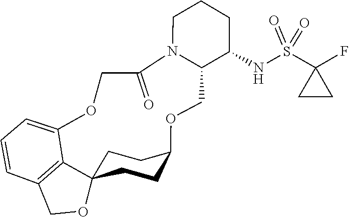

1-Fluoro-N-((5aR,8R,10aR,11S)-16-oxo-
7,8,10,10a,11,12,13,14,16,17-decahydro-4H,6H-
5a,8-ethanoisobenzofuro[7,1-hi]pyrido[2,1-
c][1,7]dioxa[4]azacyclotridecin-11-
yl)cyclopropane-1-sulfonamide

In some embodiments, the invention relates to a compound selected from Table B, or a pharmaceutically acceptable salt thereof. In other embodiments, the invention relates to a compound selected from Table B, i.e., the compound in non-salt form.

TABLE B
Compound Structures and Names

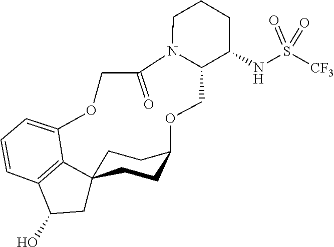

1,1,1-Trifluoro-N-((5a,8-cis)-
(4S,10aR,11S)-4-hydroxy-16-
oxo-
4,5,7,8,10,10a,11,12,13,14,16,17-
dodecahydro-6H-5a,8-
ethanoindeno[7,1-hi]pyrido[2,1-
c][1,7]dioxa[4]azacyclotridecin-
11-yl)methanesulfonamide

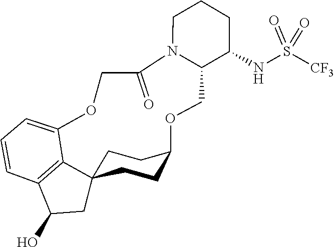

1,1,1-Trifluoro-N-((5a,8-cis)-
((4R,10aR,11S)-4-hydroxy-16-
oxo-
4,5,7,8,10,10a,11,12,13,14,16,17-
dodecahydro-6H-5a,8-
ethanoindeno[7,1-hi]pyrido[2,1-
c][1,7]dioxa[4]azacyclotridecin-
11-yl)methanesulfonamide

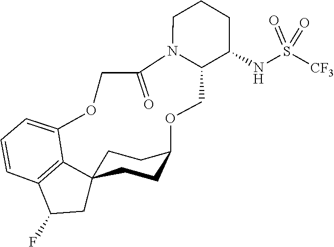

1,1,1-Trifluoro-N-((5a,8-cis)-
(4S,10aR,11S)-4-fluoro-16-oxo-
4,5,7,8,10,10a,11,12,13,14,16,17-
dodecahydro-6H-5a,8-
ethanoindeno[7,1-h]pyrido[2,1-
c][1,7-dioxa[4]azacyclotridecin-
11-yl)methanesulfonamide

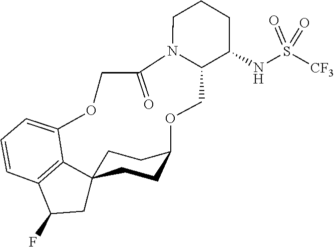

1,1,1-Trifluoro-N-((5a,8-cis)-
(4R,10aR,11S)-4-fluoro-16-oxo-
4,5,7,8,10,10a,11,12,13,14,16,17-
dodecahydro-6H-5a,8-
ethanoindeno[7,1-hi]pyrido[2,1-
c][1,7]dioxa[4]azacyclotridecin-
11-yl)methanesulfonamide

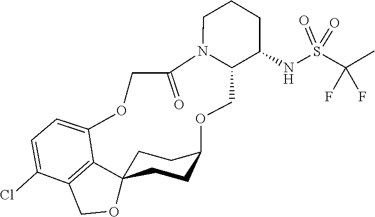

N-((5a,8-trans)-(10aR,11S)-3-
Chloro-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)-1,1-difluoroethane-1-
sulfonamide

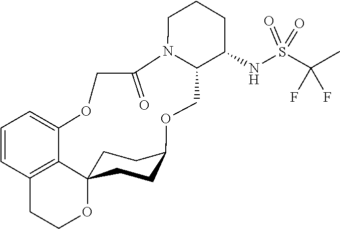

1,1-Difluoro-N-((6a,9-trans)-
(11aR,12S)-17-oxo-
4,5,8,9,11,11a,12,13,14,15,17,18-
dodecahydro-7H-6a,9-
ethanoisochromeno[8,1-
hi]pyrido[2,1-
c][1,7]dioxa[4]azacyclotridecin-
12-yl)ethane-1-sulfonamide

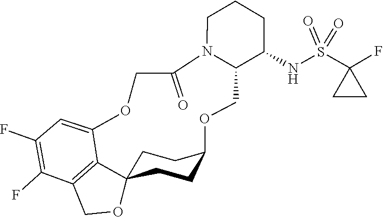

N-((5a,8-trans)-(10aR,11S)-2,3-
Difluoro-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)-1-fluorocyclopropane-1-
sulfonamide

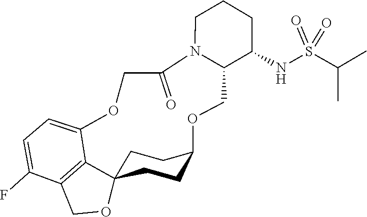

N-((5a,8-trans)-(10aR,11S)-3-
Fluoro-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)propane-2-sulfonamide

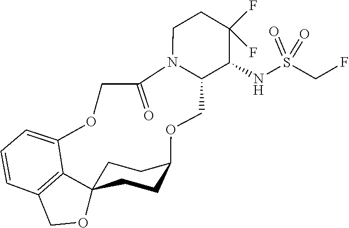

N-((5a,8-trans)-(10aR,11R)-
12,12-Difluoro-16-oxo-
7,8,10,10a,11,12,13,14,16,17-
decahydro-4H,6H,5a,8-
ethanoisobenzofuro[7,1-
hi]pyrido[2,1-
c][1,7]dioxa[4]azacyclotridecin-
11-yl)-1-
fluoromethanesulfonamide

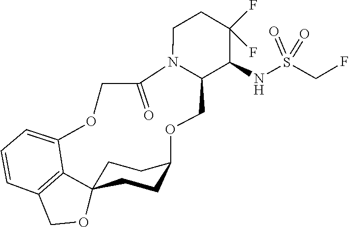

N-((5a,8-trans)-(10aS,11S)-
12,12-Difluoro-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)-1-
fluoromethanesulfonamide

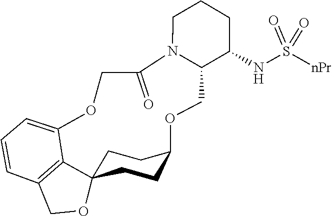

N-((5a,8-trans)-(10aR,11S)-16-
Oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)propane-1-sulfonamide

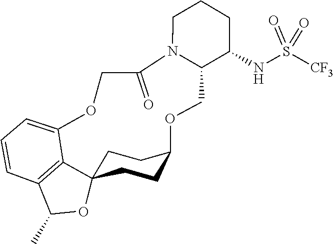

1,1,1-Trifluoro-N-((5a,8-trans)-
(4R,10aR,11S)-4-methyl-16-
oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)methanesulfonamide

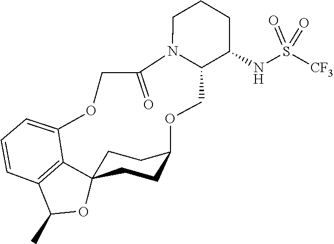

1,1,1-Trifluoro-N-((5a,8-trans)-
(4S,10aR,11S)-4-methyl-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)methanesulfonamide

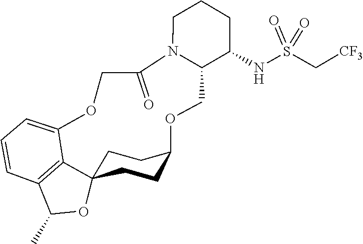

2,2,2-Trifluoro-N-((5a,8-trans)-
(4R,10aR,11S)-4-methyl-16-
oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)ethane-1-sulfonamide

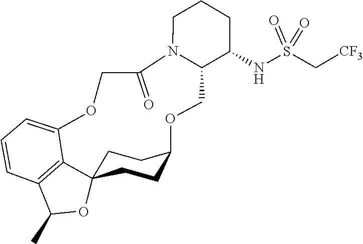

2,2,2-Trifluoro-N-((5a,8-trans)-
(4S,10aR,11S)-4-methyl-16-oxo-
7,8,10,10a,11,12,13,14,16,17-
decahydro-4H,6H-5a,8-
ethanoisobenzofuro[7,1-
c][1,7]dioxa[4]azacyclotridecin-
11-yl)ethane-1-sulfonamide

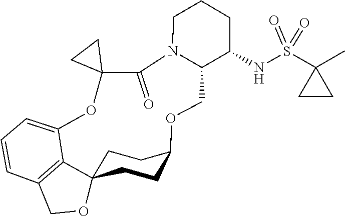

1-Methyl-N-((5a,8-trans)-
(10a′R,11′S)-16′-oxo-
7′,8′,10′,10a′,11′,12′,13′,14′-
octahydro-4′H,6′H,16′H-
spiro[cyclopropane-1,17′-
[5a,8]ethanoisobenzofuro[7,1-
hi]pyrido[2,1-
c][1,7]dioxa[4]azacyclotridecin]-
11′-yl)cyclopropane-1-
sulfonamide

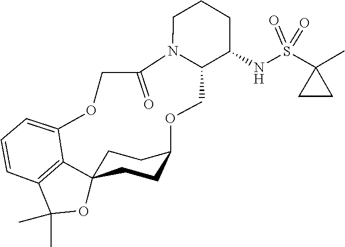

N-((5a,8-trans)-(10aR,11S)-4,4-
Dimethyl-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)-1-methylcyclopropane-1-
sulfonamide

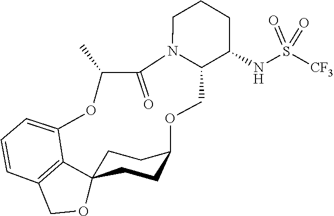

1,1,1-Trifluoro-N-((5a,8-trans)-
(10aR,11S,17R)-17-methyl-16-
oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)methanesulfonamide

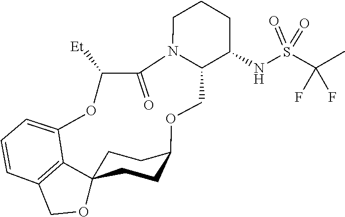

N-((5a,8-trans)-(10aR,11S,17R)-
17-Ethyl-16-oxo-7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)-1,1-difluoroethane-1-
sulfonamide

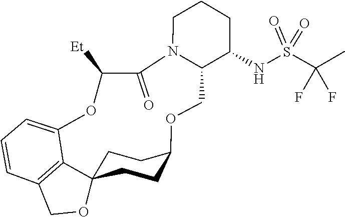

N-((5a,8-trans)-(10aR,11S,17S)-
17-Ethyl-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)-1,1-difluoroethane-1-
sulfonamide

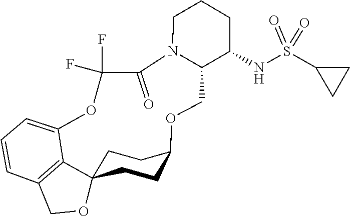

N-((5a,8-trans)-(10aR,11S)-
17,17-Difluoro-16-oxo-
7,8,10,10a,11,12,13,14,16,17-
decahydro-4H,6H-5a,8-
ethanoisobenzofuran[7,1-
hi]pyrido[2,1-
c][1,7]dioxa[4]azacyclotridecin-
11-yl)cyclopropanesulfonamide

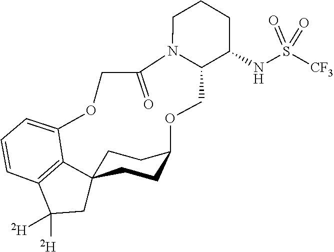

1,1,1-Trifluoro-N-((5a,8-cis)-
(10aR,11S)-16-oxo-
4,5,7,8,10,10a,11,12,13,14,16,17-
dodecahydro-6H-5a,8-
ethanoindeno[7,1-hi]pyrido[2,1-
c][1,7]dioxa[4]azacyclotridecin-
11-yl-4,4-
d₂)methanesulfonamide

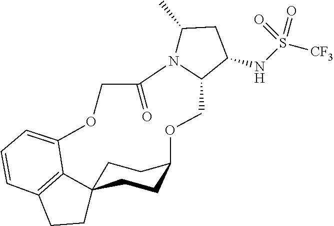

1,1,1-Trifluoro-N-((5a,8-cis)-
(10aR,11S,13R)-13-methyl-15-
oxo-4,5,7,8,10a,11,12,13,15,16-
decahydro-6H,10H-5a,8-
ethanoindeno[7,1-
hi]pyrrolo[2,1-
c][1,7]dioxa[4]azacyclotridecin-
11-yl)methanesulfonamide

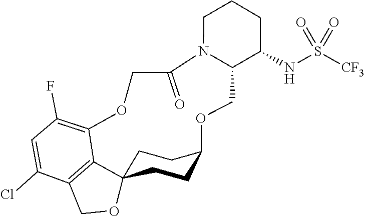

N-((5a,8-trans)-(10aR,11S)-3-
Chloro-1-fluoro-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)-1,1,1-
trifluoromethanesulfonamide

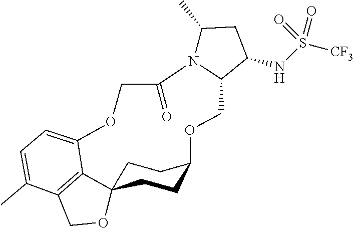

N-((5a,8-trans)-(10aR,11S,13R)-
3,13-Dimethyl-15-oxo-
7,8,10a,11,12,13,15,16-
octahydro-4H,6H,10H-5a,8-
ethanoisobenzofuro[7,1-
hi]pyrrolo[2,1-
c][1,7]dioxa[4]azacyclotridecin-
11-yl)-1,1,1-
trifluoromethanesulfonamide

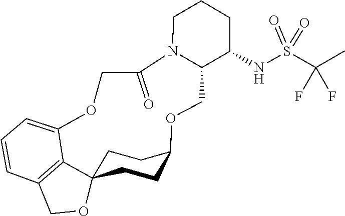

1,1-Difluoro-N-((5a,8-trans)-
(10aR,11S)-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)ethane-1-sulfonamide

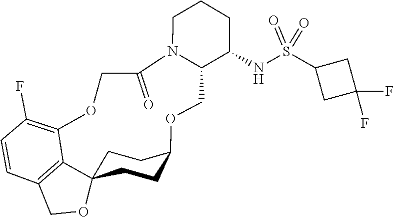

3,3-Difluoro-N-((5a,8-trans)-
(10aR,11S)-1-fluoro-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)cyclobutane-1-
sulfonamide

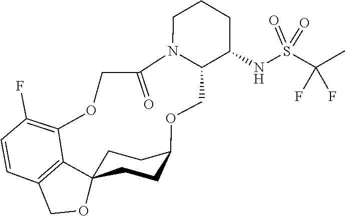

1,1-Difluoro-N-((5a,8-trans)-
(10aR,11S)-1-fluoro-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)ethane-1-sulfonamide

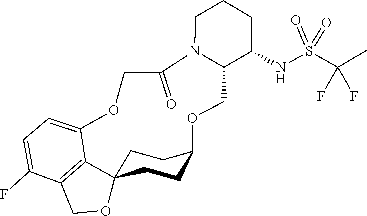

1,1-Difluoro-N-((5a,8-trans)-
(10aR,11S)-3-fluoro-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)ethane-1-sulfonamide

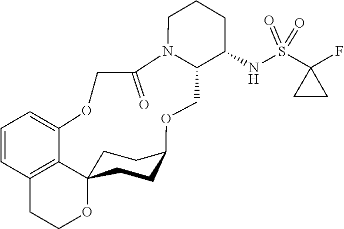

1-Fluoro-N-((6a,9-trans)-
(11aR,12S)-17-oxo-
4,5,8,9,11,11a,12,13,14,15,17,18-
dodecahydro-7H-6a,9-
ethanoisochromeno[8,1-
hi]pyrido[2,1-
c][1,7]dioxa[4]azacyclotridecin-
12-yl)cyclopropane-1-
sulfonamide

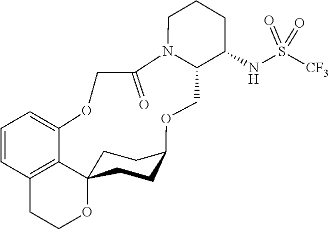

1,1,1-Trifluoro-N-((6a,9-trans)-
(11aR,12S)-17-oxo-
4,5,8,9,11,11a,12,13,14,15,17,18-
dodecahydro-7H-6a,9-
ethanoisochromeno[8,1-
hi]pyrido[2,1-
c][1,7]dioxa[4]azacyclotridecin-
12-yl)methanesulfonamide

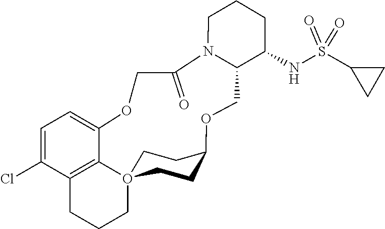

N-((6a,9-trans)-(11aR,12S)-3-
Chloro-17-oxo-
4,5,8,9,11,11a,12,13,14,15,17,18-
dodecahydro-7H-6a,9-
ethanoisochromeno[8,1-
hi]pyrido[2,1-
c][1,7]dioxa[4]azacyclotridecin-
12-yl)cyclopropanesulfonamide

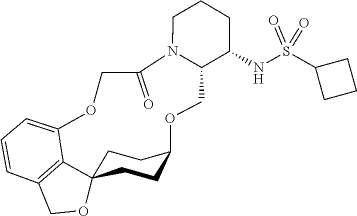

N-((5a,8-trans)-(10aR,11S)-16-
Oxo-
7,8,10,10a,11,12,13,14,16,17-
decahydro-4H,6H-5a,8-
ethanoisobenzofuro[7,1-
hi]pyrido[2,1-
c][1,7-dioxa[4]azacyclotridecin-
11-yl)cyclobutanesulfonamide

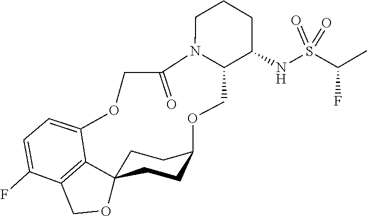

(R)-1-Fluoro-N-((5a,8-trans)-
(10aR,11S)-3-fluoro-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)ethane-1-sulfonamide

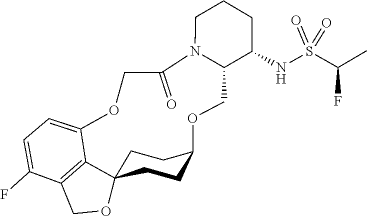

(S)-1-Fluoro-N-((5a,8-trans)-
(10aR,11S)-3-fluoro-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)ethane-1-sulfonamide

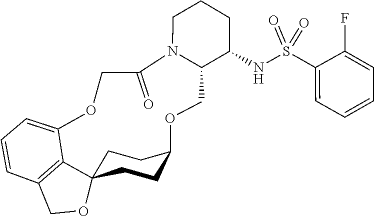

2-Fluoro-N-((5a,8-trans)-
(10aR,11S)-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)benzenesulfonamide

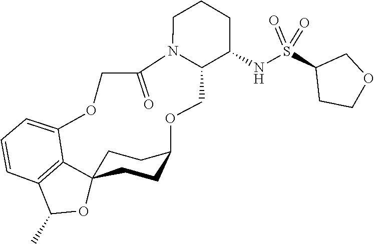

(R)-N-((5a,8-trans)-
(4R,10aR,11S)-4-Methyl-16-
oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)tetrahydrofuran-3-
sulfonamide

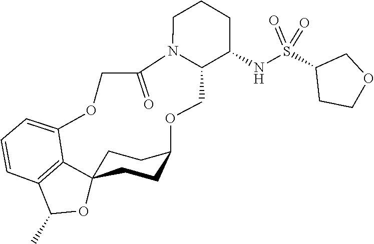

(S)-N-((5a,8-trans)-
(4R,10aR,11S)-4-Methyl-16-
oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)tetrahydrofuran-3-
sulfonamide

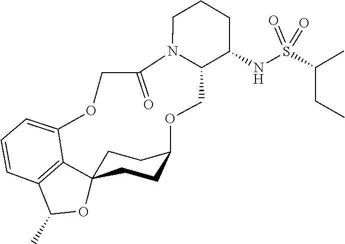

(R)-N-((5a,8-trans)-
(4R,10aR,11S)-4-Methyl-16-
oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)butane-2-sulfonamide

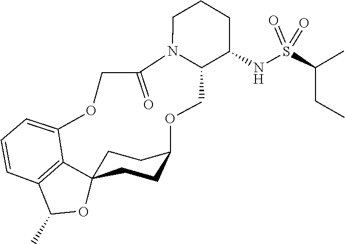

(S)-N-((5a,8-trans)-
(4R,10aR,11S)-4-Methyl-16-
oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)butane-2-sulfonamide

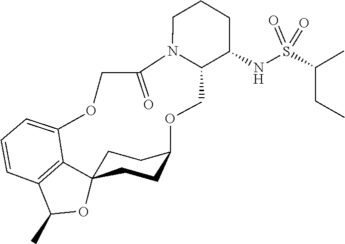

(R)-N-((5a,8-trans)-
(4S,10aR,11S)-4-Methyl-16-
oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)butane-2-sulfonamide

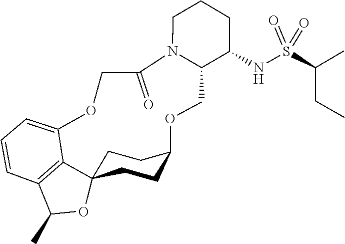

(S)-N-((5a,8-trans)-
(4S,10aR,11S)-4-Methyl-16-
oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)butane-2-sulfonamide

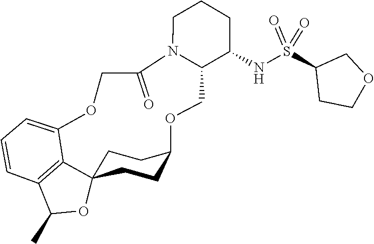

(R)-N-((5a,8-trans)-
(4S,10aR,11S)-4-Methyl-16-
oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)tetrahydrofuran-3-
sulfonamide

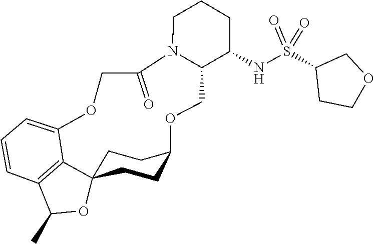

(S)-N-((5a,8-trans)-
(4S,10aR,11S)-4-Methyl-16-
oxo-
7,8,10,10a,11,12,13,14,16,17-
decahydro-4H,6H-5a,8-
ethanoisobenzofuro[7,1-
c][1,7]dioxa[4]azacyclotridecin-
11-yl)tetrahydrofuran-3-
sulfonamide

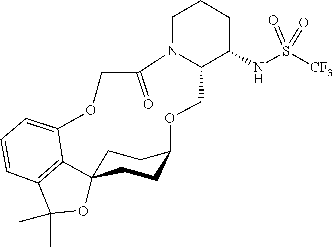

N-((5a,8-trans)-(10aR,11S)-4,4-
Dimethyl-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)-1,1,1-
trifluoromethanesulfonamide

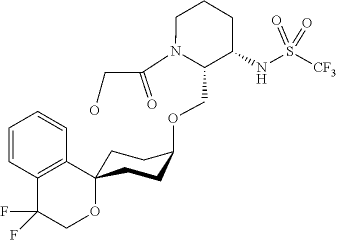

N-((6a,9-trans)-(11aR,12S)-4,4-
Difluoro-17-oxo-
4,5,8,9,11,11a,12,13,14,15,17,18-
dodecahydro-7H-6a,9-
ethanoisochromeno[8,1-
hi]pyrido[2,1-
c][1,7]dioxa[4]azacyclotridecin-
12-yl)-1,1,1-
trifluoromethanesulfonamide

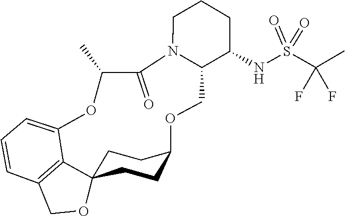

1,1-Difluoro-N-((5a,8-trans)-
(10aR,11S,17S)-17-methyl-16-
oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)ethane-1-sulfonamide

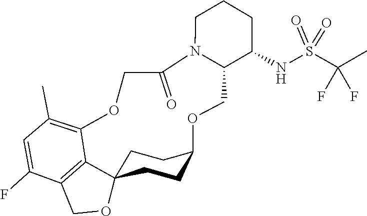

1,1-Difluoro-N-((5a,8-trans)-
(10aR,11S)-3-fluoro-1-methyl-
16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)ethane-1-sulfonamide

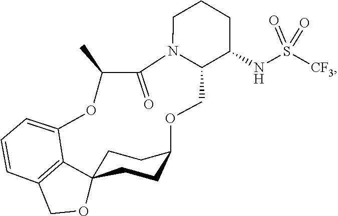

1,1,1-Trifluoro-N-((5a,8-trans)-
(10aR,11S,17S)-17-methyl-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)methanesulfonamide

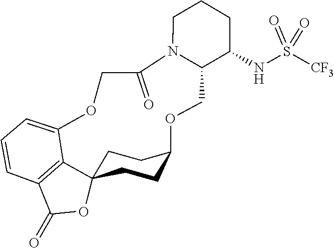

N-((5a,8-trans)-(10aR,11S)-
4,16-Dioxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)-1,1,1-
trifluoromethanesulfonamide

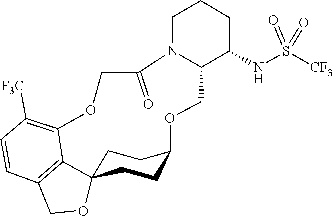

1,1,1-Trifluoro-N-((5a,8-trans)-
(10aR,11S)-16-oxo-1-
(trifluoromethyl)-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)methanesulfonamide

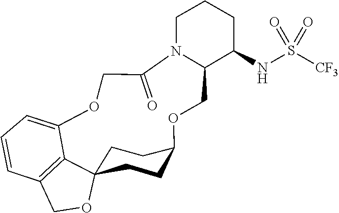

1,1,1-Trifluoro-N-((5a,8-trans)-
(10aS,11R)-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)methanesulfonamide

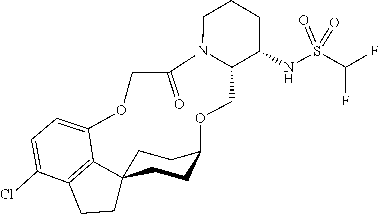

N-((5a,8-cis)-(10aR,11S)-3-
Chloro-16-oxo-
4,5,7,8,10,10a,11,12,13,14,16,17-
dodecahydro-6H-5a,8-
ethanoindeno[7,1-hi]pyrido[2,1-
c][1,7]dioxa[4]azacyclotridecin-
11-yl)-1,1-
difluoromethanesulfonamide

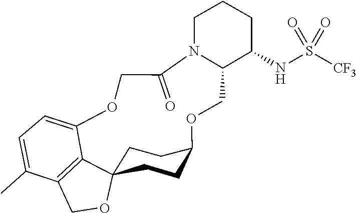

1,1,1-Trifluoro-N-((5a,8-trans)-
(10aR,11S)-3-methyl-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)methanesulfonamide

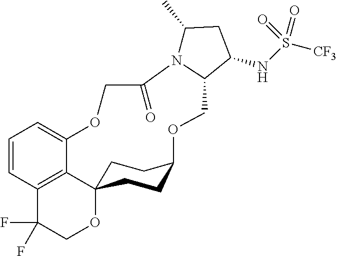

N-((6a,9-trans)-(11aR,12S,14R)-
4,4-
Difluoro-14-methyl-16-oxo-
4,5,8,9,11a,12,13,14,16,17-
decahydro-7H,11H-6a,9-
ethanoisochromeno[8,1-
hi]pyrrolo[2,1-
c][1,7]dioxa[4]azacyclotridecin-
12-yl)-1,1,1-
trifluoromethanesulfonamide

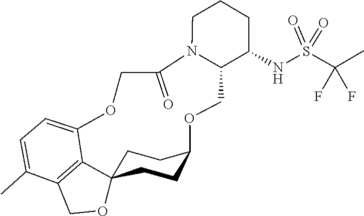

1,1-Difluoro-N-((5a,8-trans)-
(10aR,11S)-3-methyl-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)ethane-1-sulfonamide

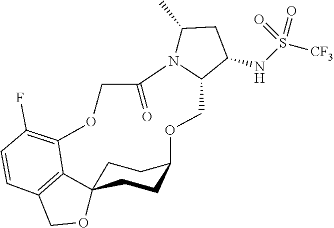

1,1,1-Trifluoro-N-((5a,8-trans)-
(10aR,11S,13R)-1-fluoro-13-
methyl-15-oxo-
7,8,10a,11,12,13,15,16-
octahydro-4H,6H,10H-5a,8-
ethanoisobenzofuro[7,1-
hi]pyrrolo[2,1-
c][1,7]dioxa[4]azacyclotridecin-
11-yl)methanesulfonamide

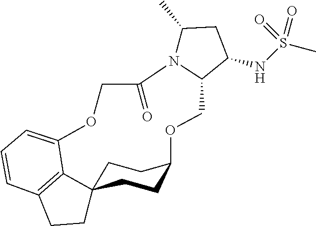

N-((5a,8-cis)-(10aR,11S,13R)-13-Methyl-15-oxo-
4,5,7,8,10a,11,12,13,15,16-decahydro-6H,10H-
5a,8-ethanoindeno[7,1-hi]pyrrolo[2,1-
c][1,7-dioxa[4]azacyclotridecin-11-
yl)methanesulfonamide

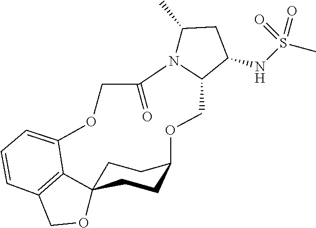

N-((5a,8-trans)-(10aR,11S,13R)-13-Methyl-15-
oxo-7,8,10a,11,12,13,15,16-octahydro-
4H,6H,10H-5a,8-ethanoisobenzofuro[7,1-
hi]pyrrolo[2,1-c][1,7]dioxa[4]azacyclotridecin-11-
yl)methanesulfonamide

In some embodiments, the invention relates to a compound selected from Table C, or a pharmaceutically acceptable salt thereof. In other embodiments, the invention relates to a compound selected from Table C, i.e., the compound in non-salt form.

TABLE C
Compound Structures and Names

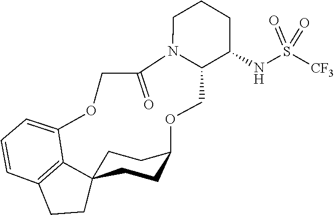

1,1,1-Trifluoro-N-((5a,8-cis)-
(10aR,11S)-16-oxo-
4,5,7,8,10,10a,11,12,13,14,16,
17-dodecahydro-6H-5a,8-
ethanoindeno[7,1-
hi]pyrido[2,1-
c][1,7]dioxa[4]azacyclotridecin-
11-yl)methanesulfonamide

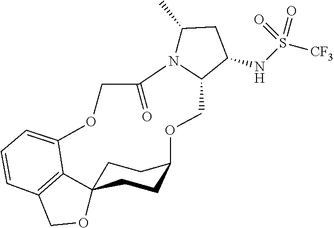

1,1,1-Trifluoro-N-((5a,8-trans)-
(10aR,11S,13R)-13-methyl-15-
oxo-7,8,10a,11,12,13,15,16-
octahydro-4H,6H,10H-5a,8-
ethanoisobenzofuro[7,1-
hi]pyrrolo[2,1-
c][1,7]dioxa[4]azacyclotridecin-
11-yl)methanesulfonamide

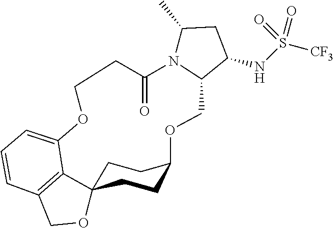

1,1,1-Trifluoro-N-((5a,8-trans)-
(10aR,11S,13R)-13-methyl-15-
oxo-7,8,10a,11,12,13,16,17-
octahydro-4H,6H,10H,15H-
5a,8-ethanoisobenzofuro[7,1-
ij]pyrrolo[2,1-
c][1,8]dioxa[4]azacyclotetradecin-
11-yl)methanesulfonamide

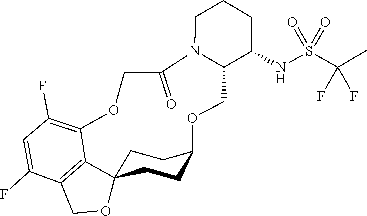

N-((5a,8-trans)-(10aR,11S)-1,3-
Difluoro-16-oxo-
7,8,10,10a,11,12,13,14,16,17-
decahydro-4H,6H-5a,8-
ethanoisobenzofuro[7,1-
hi]pyrido[2,1-c][1,7]dioxa[4]
azacyclotridecin-11-yl)-1,1-
difluoroethane-1-sulfonamide

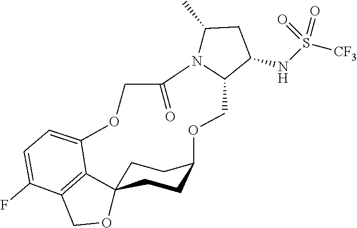

1,1,1-Trifluoro-N-((5a,8-trans)-
(10aR,11S,13R)-3-fluoro-13-
methyl-15-oxo-
7,8,10a,11,12,13,15,16-
octahydro-4H,6H,10H-5a,8-
ethanoisobenzofuro[7,1-
hi]pyrrolo[2,1-
c][1,7]dioxa[4]azacyclotridecin-
11-yl)methanesulfonamide

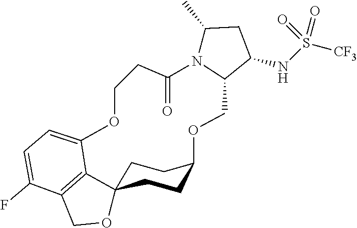

1,1,1-Trifluoro-N-((5a,8-trans)-
(10aR,11S,13R)-3-fluoro-13-
methyl-15-oxo-
7,8,10a,11,12,13,16,17-
octahydro-4H,6H,10H,15H-
5a,8-ethanoisobenzofuro[7,1-
ij]pyrrolo[2,1-
c][1,8]dioxa[4]azacyclotetradecin-
11-yl)methanesulfonamide

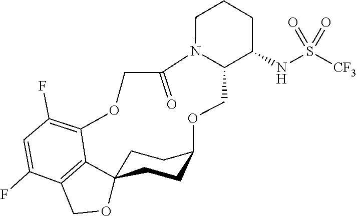

N-((5a,8-trans)-(10aR,11S)-1,3-
Difluoro-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)-1,1,1-
trifluoromethanesulfonamide

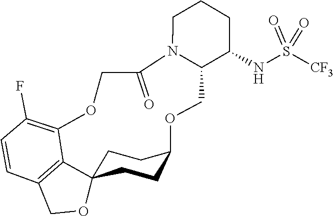

1,1,1-Trifluoro-N-((5a,8-
trans)(10aR,11S)-1-fluoro-16-
oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)methanesulfonamide

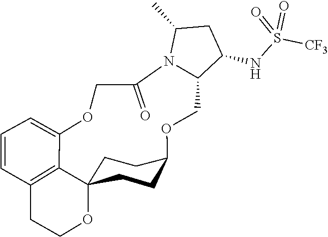

1,1,1-Trifluoro-N-(6a,9-trans)-
(11aR,12S,14R)-14-methyl-16-
oxo-4,5,8,9,11a,12,13,14,16,17-
decahydro-7H,11H-6a,9-
ethanoisochromeno[8,1-
hi]pyrrolo[2,1-
c][1,7]dioxa[4]azacyclotridecin-
12-yl)methanesulfonamide

In some embodiments, the invention relates to a compound selected from Table D, or a pharmaceutically acceptable salt thereof. In other embodiments, the invention relates to a compound selected from Table D, i.e., the compound in non-salt form.

TABLE D
Compound Structures and Names

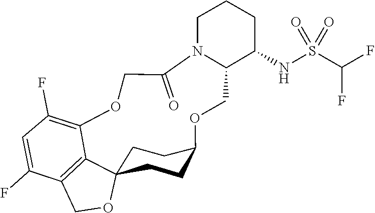

N-((5a,8-trans)-(10aR,11S)-1,3-
Difluoro-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)-1,1-
difluoromethanesulfonamide

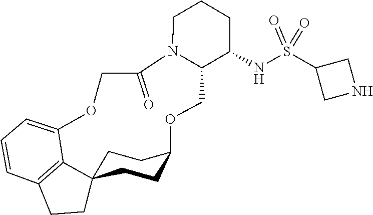

N-((5a,8-cis)-(10aR,11S)-16-Oxo-
4,5,7,8,10,10a,11,12,13,14,16,17-
dodecahydro-6H-5a,8-
ethanoindeno[7,1-hi]pyrido[2,1-
c][1,7]dioxa[4]azacyclotridecin-
11-yl)azetidine-3-sulfonamide

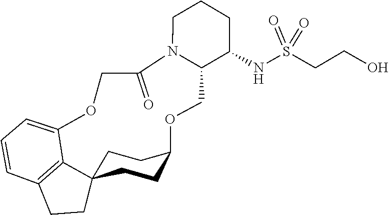

2-Hydroxy-N-((5a,8-cis)-
(10aR,11S)-16-oxo-
4,5,7,8,10,10a,11,12,13,14,16,17-
dodecahydro-6H-5a,8-
ethanoindeno[7,1-
hi]pyrido[2,1-
c][1,7]dioxa[4]azacyclotridecin-
11-yl)ethane-1-sulfonamide

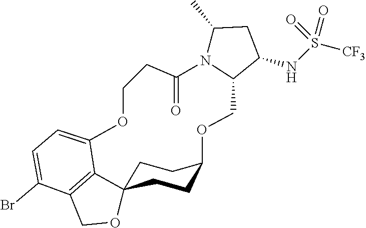

N-((5a,8-trans)-
(10aR,11S,13R)-3-Bromo-13-
methyl-15-oxo-
7,8,10a,11,12,13,16,17-
octahydro-4H,6H,10H,15H-
5a,8-ethanoisobenzofuro[7,1-
ij]pyrrolo[2,1-
c][1,8]dioxa[4]azacyclotetradecin-
11-yl)-1,1,1-
trifluoromethanesulfonamide

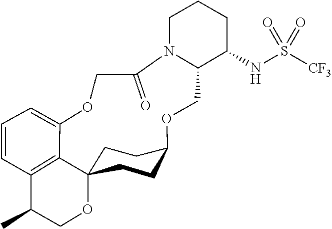

1,1,1-Trifluoro-N-((6a,9-trans)-
(4S,11aR,12S)-4-methyl-17-oxo-
4,5,8,9,11,11a,12,13,14,15,17,18-
dodecahydro-7H-6a,9-
ethanoisochromeno[8,1-
hi]pyrido[2,1-
c][1,7]dioxa[4]azacyclotridecin-
12-yl)methanesulfonamide

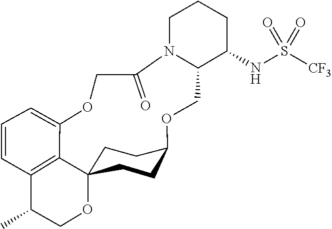

1,1,1-Trifluoro-N-((6a,9-trans)-
(4R,11aR,12S)-4-methyl-17-
oxo-
4,5,8,9,11,11a,12,13,14,15,17,18-
dodecahydro-7H-6a,9-
ethanoisochromeno[8,1-
hi]pyrido[2,1-
c][1,7]dioxa[4]azacyclotridecin-
12-yl)methanesulfonamide

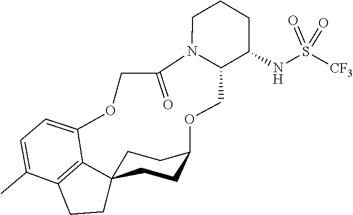

1,1,1-Trifluoro-N-((5a,8-cis)-
(10aR,11S)-3-methyl-16-oxo-
4,5,7,8,10,10a,11,12,13,14,16,17-
dodecahydro-6H-5a,8-
ethanoindeno[7,1-
hi]pyrido[2,1-
c][1,7]dioxa[4]azacyclotridecin-
11-yl)methanesulfonamide

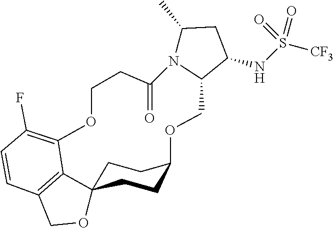

1,1,1-Trifluoro-N-((5a,8-trans)-
(10aR,11S,13R)-1-fluoro-13-
methyl-15-oxo-
7,8,10a,11,12,13,16,17-
octahydro-4H,6H,10H,15H-5a,8-
ethanoisobenzofuro[7,1-
ij]pyrrolo[2,1-
c][1,8]dioxa[4]azacyclotetradecin-
11-yl)methanesulfonamide

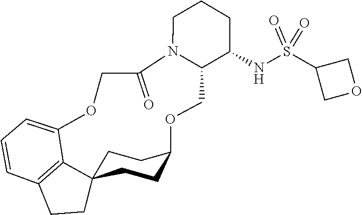

N-((5a,8cis)-(10aR,11S)-16-
Oxo-
4,5,7,8,10,10a,11,12,13,14,16,17-
dodecahydro-6H-5a,8-
ethanoindeno[7,1-
hi]pyrido[2,1-
c][1,7]dioxa[4]azacyclotridecin-
11-yl)oxetane-3-sulfonamide

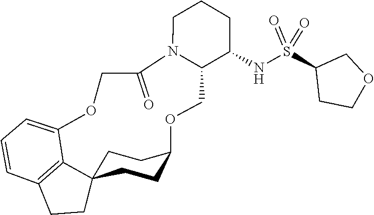

(R)-N-((5a,8-cis)-(10aR,11S)-
16-Oxo-
4,5,7,8,10,10a,11,12,13,14,16,17-
dodecahydro-6H-5a,8-
ethanoindeno[7,1-
hi]pyrido[2,1-
c][1,7]dioxa[4]azacyclotridecin-
11-yl)tetrahydrofuran-3-
sulfonamide

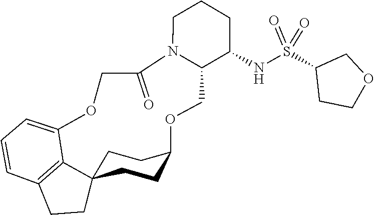

(S)-N-((5a,8-cis)-(10aR,11S)-16-
Oxo-
4,5,7,8,10,10a,11,12,13,14,16,17-
dodecahydro-6H-5a,8-
ethanoindeno[7,1-hi]pyrido[2,1-
c][1,7]dioxa[4]azacyclotridecin-
11-yl)tetrahydrofuran-3-
sulfonamide

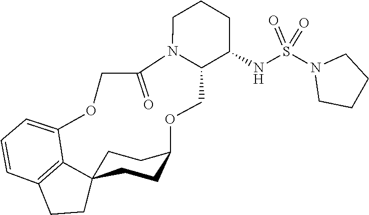

N-((5a,8-cis)-(10aR,11S)-16-
Oxo-
4,5,7,8,10,10a,11,12,13,14,16,17-
dodecahydro-6H-5a,8-
ethanoindeno[7,1-
hi]pyrido[2,1-
c][1,7]dioxa[4]azacyclotridecin-
11-yl)pyrrolidine-1-
sulfonamide

In some embodiments, the invention relates to a compound selected from Table E, or a pharmaceutically acceptable salt thereof. In other embodiments, the invention relates to a compound selected from Table E, i.e., the compound in non-salt form.

TABLE E
Compound Structures and Names

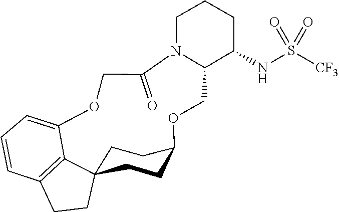

1,1,1-Trifluoro-N-((5a,8-cis)-
(10aR,11S)-16-oxo-
4,5,7,8,10,10a,11,12,13,14,16,17-
dodecahydro-6H-5a,8-
ethanoindeno[7,1-
hi]pyrido[2,1-
c][1,7]dioxa[4]azacyclotridecin-
11-yl)methanesulfonamide

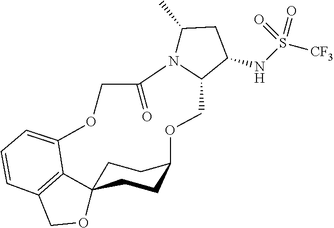

1,1,1-Trifluoro-N-((5a,8-trans)-
(10aR,11S,13R)-13-methyl-15-
oxo-7,8,10a,11,12,13,15,16-
octahydro-4H,6H,10H-5a,8-
ethanoisobenzofuro[7,1-
hi]pyrrolo[2,1-
c][1,7]dioxa[4]azacyclotridecin-
11-yl)methanesulfonamide

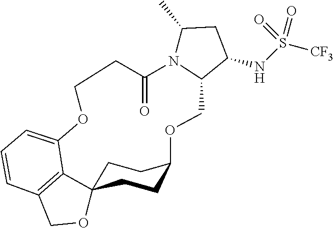

1,1,1-Trifluoro-N-((5a,8-trans)-
(10aR,11S,13R)-13-methyl-15-
oxo-7,8,10a,11,12,13,16,17-
octahydro-4H,6H,10H,15H-
5a,8-ethanoisobenzofuro[7,1-
ij]pyrrolo[2,1-
c][1,8]dioxa[4]azacyclotetradecin-
11-yl)methanesulfonamide

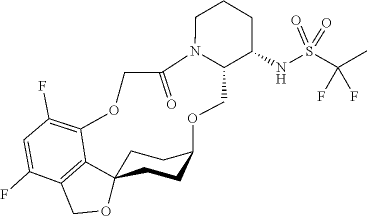

N-((5a,8-trans)-(10aR,11S)-1,3-
Difluoro-16-oxo-
7,8,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)-1,1-difluoroethane-1-
sulfonamide

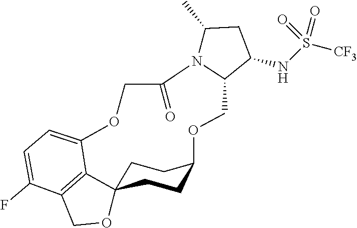

1,1,1-Trifluoro-N-((5a,8-trans)-
(10aR,11S,13R)-3-fluoro-13-
methyl-15-oxo-
7,8,10a,11,12,13,15,16-
octahydro-4H,6H,10H-5a,8-
ethanoisobenzofuro[7,1-
hi]pyrrolo[2,1-
c][1,7]dioxa[4]azacyclotridecin-
11-yl)methanesulfonamide

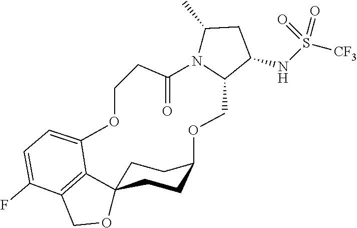

1,1,1-Trifluoro-N-((5a,8-trans)-
(10aR,11S,13R)-3-fluoro-13-
methyl-15-oxo-
7,8,10a,11,12,13,16,17-
octahydro-4H,6H,10H,15H-
5a,8-ethanoisobenzofuro[7,1-
ij]pyrrolo[2,1-
c][1,8]dioxa[4]azacyclotetradecin-
11-yl)methanesulfonamide

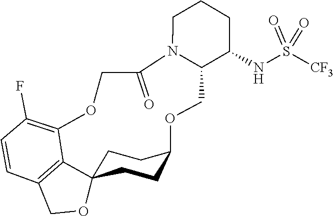

1,1,1-Trifluoro-N-((5a,8-
trans)(10aR,11S)-1-fluoro-16-
oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)methanesulfonamide

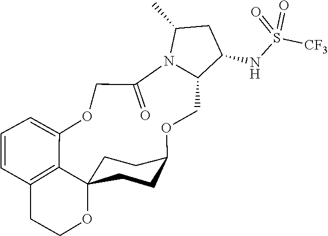

1,1,1-Trifluoro-N-(6a,9-trans)-
(11aR,12S,14R)-14-methyl-16-
oxo-4,5,8,9,11a,12,13,14,16,17-
decahydro-7H,11H-6a,9-
ethanoisochromeno[8,1-
hi]pyrrolo[2,1-
c][1,7]dioxa[4]azacyclotridecin-
12-yl)methanesulfonamide

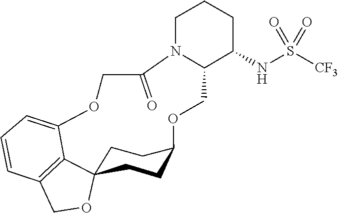

1,1,1-Trifluoro-N-
((5aR,8R,10aR,11S)-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)methanesulfonamide

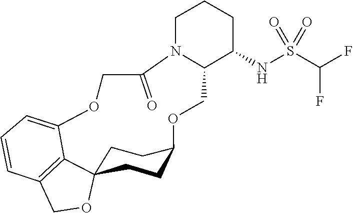

1,1-Difluoro-N-
((5aR,8R,10aR,11S)-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)methanesulfonamide

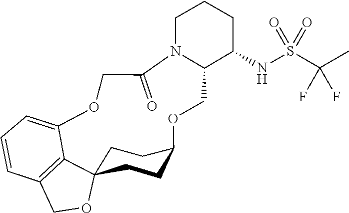

1,1-Difluoro-N-((5a,8-trans)-
(10aR,11S)-16-oxo-
7,8,10,10a,11,12,13,14,16,17-
decahydro-4H,6H-5a,8-
ethanoisobenzofuro[7,1-
hi]pyrido[2,1-
c][1,7-dioxa[4]azacyclotridecin-
11-yl)ethane-1-sulfonamide

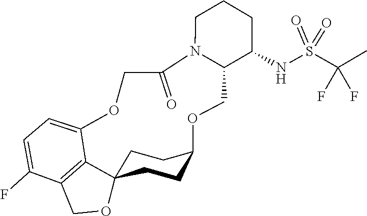

1,1-Difluoro-N-((5a,8-trans)-
(10aR,11S)-3-fluoro-16-oxo-
7,8,10,10a,11,12,13,14,16,17-
decahydro-4H,6H-5a,8-
ethanoisobenzofuro[7,1-
hi]pyrido[2,1-
c][1,7]dioxa[4]azacyclotridecin-
11-yl)ethane-1-sulfonamide

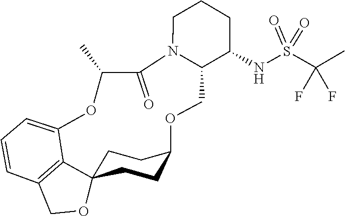

1,1-Difluoro-N-((5a,8-trans)-(10aR,11S,17S)-17-
methyl-16-oxo-7,8,10,10a,11,12,13,14,16,17-
decahydro-4H,6H-5a,8-ethanoisobenzofuro[7,1-
hi]pyrido[2,1-c][1,7]dioxa[4]azacyclotridecin-11-
yl)ethane-1-sulfonamide

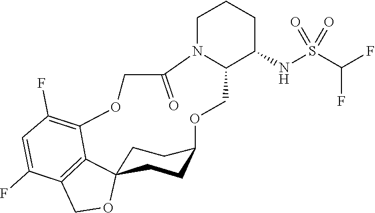

N-((5a,8-trans)-(10aR,11S)-1,3-Difluoro-16-oxo-
7,8,10,10a,11,12,13,14,16,17-decahydro-4H,6H-
5a,8-ethanoisobenzofuro[7,1-hi]pyrido[2,1-
c][1,7-dioxa[4]azacyclotridecin-11-yl)-1,1-
difluoromethanesulfonamide

In some embodiments, the invention relates to a compound of formula

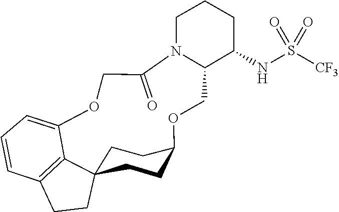

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 1,1,1-Trifluoro-N-((5a,8-cis)-(10aR,11S)-16-oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)methanesulfonamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

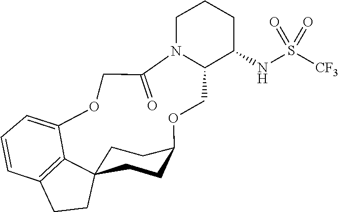

Such compound is considered to be a “compound of the invention,” as that term is used herein.

In some embodiments, the invention relates to the compound 1,1,1-Trifluoro-N-((5a,8-cis)-(10aR,11S)-16-oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)methanesulfonamide. Such compound is considered to be a “compound of the invention,” as that term is used herein.

In some embodiments, the invention relates to a compound of formula

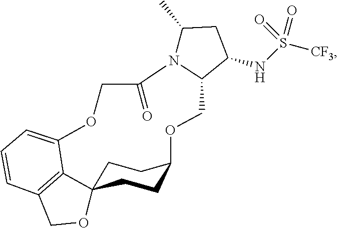

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 1,1,1-Trifluoro-N-((5a,8-trans)-(10aR,11S,13R)-13-methyl-15-oxo-7,8,10a,11,12,13,15,16-octahydro-4H,6H,10H-5a,8-ethanoisobenzofuro[7,1-hi]pyrrolo[2,1-c][1,7]dioxa[4]azacyclotridecin-11-yl)methanesulfonamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

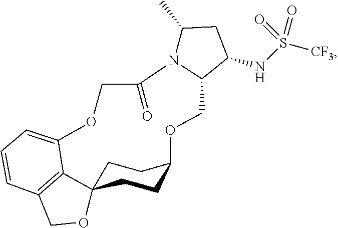

Such compound is considered to be a “compound of the invention,” as that term is used herein.

In some embodiments, the invention relates to the compound 1,1,1-Trifluoro-N-((5a,8-trans)-(10aR,11S,13R)-13-methyl-15-oxo-7,8,10a,11,12,13,15,16-octahydro-4H,6H,10H-5a,8-ethanoisobenzofuro[7,1-hi]pyrrolo[2,1-c][1,7]dioxa[4]azacyclotridecin-11-yl)methanesulfonamide. Such compound is considered to be a “compound of the invention,” as that term is used herein.

In some embodiments, the invention relates to a compound of formula

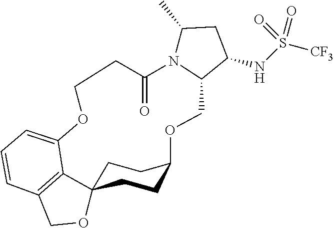

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 1,1,1-Trifluoro-N-((5a,8-trans)-(10aR,11S,13R)-13-methyl-15-oxo-7,8,10a,11,12,13,16,17-octahydro-4H,6H,10H,15H-5a,8-ethanoisobenzofuro[7,1-ij]pyrrolo[2,1-c][1,8]dioxa[4]azacyclotetradecin-11-yl)methanesulfonamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

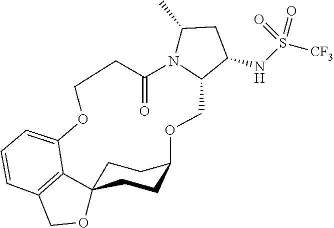

Such compound is considered to be a “compound of the invention,” as that term is used herein.

In some embodiments, the invention relates to the compound 1,1,1-Trifluoro-N-((5a,8-trans)-(10aR,11S,13R)-13-methyl-15-oxo-7,8,10a,11,12,13,16,17-octahydro-4H,6H,10H,15H-5a,8-ethanoisobenzofuro[7,1-ij]pyrrolo[2,1-c][1,8]dioxa[4]azacyclotetradecin-11-yl)methanesulfonamide. Such compound is considered to be a “compound of the invention,” as that term is used herein.

In some embodiments, the invention relates to a compound of formula

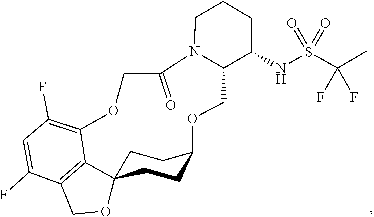

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound N-((5a,8-trans)-(10aR,11S)-1,3-Difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1,1-difluoroethane-1-sulfonamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

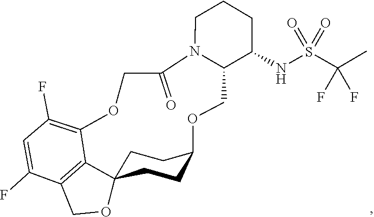

Such compound is considered to be a “compound of the invention,” as that term is used herein.

In some embodiments, the invention relates to the compound N-((5a,8-trans)-(10aR,11S)-1,3-Difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1,1-difluoroethane-1-sulfonamide. Such compound is considered to be a “compound of the invention,” as that term is used herein.

In some embodiments, the invention relates to a compound of formula

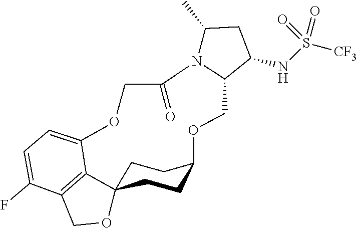

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 1,1,1-Trifluoro-N-((5a,8-trans)-(10aR,11S,13R)-3-fluoro-13-methyl-15-oxo-7,8,10a,11,12,13,15,16-octahydro-4H,6H,10H-5a,8-ethanoisobenzofuro[7,1-hi]pyrrolo[2,1-c][1,7]dioxa[4]azacyclotridecin-11-yl)methanesulfonamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

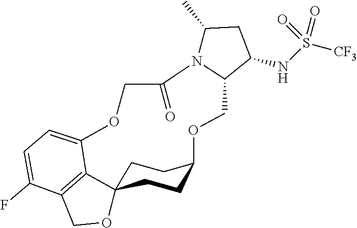

Such compound is considered to be a “compound of the invention,” as that term is used herein.

In some embodiments, the invention relates to the compound 1,1,1-Trifluoro-N-((5a,8-trans)-(10aR,11S,13R)-3-fluoro-13-methyl-15-oxo-7,8,10a,11,12,13,15,16-octahydro-4H,6H,10H-5a,8-ethanoisobenzofuro[7,1-hi]pyrrolo[2,1-c][1,7]dioxa[4]azacyclotridecin-11-yl)methanesulfonamide. Such compound is considered to be a “compound of the invention,” as that term is used herein.

In some embodiments, the invention relates to a compound of formula

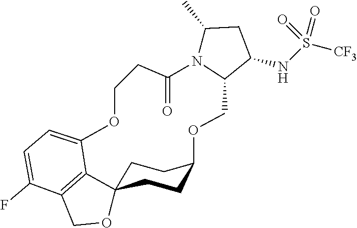

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 1,1,1-Trifluoro-N-((5a,8-trans)-(10aR,11S,13R)-3-fluoro-13-methyl-15-oxo-7,8,10a,11,12,13,16,17-octahydro-4H,6H,10H,15H-5a,8-ethanoisobenzofuro[7,1-ij]pyrrolo[2,1-c][1,8]dioxa[4]azacyclotetradecin-11-yl)methanesulfonamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

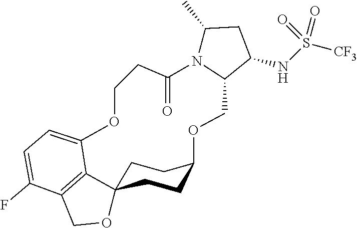

Such compound is considered to be a “compound of the invention,” as that term is used herein.

In some embodiments, the invention relates to the compound 1,1,1-Trifluoro-N-((5a,8-trans)-(10aR,11S,13R)-3-fluoro-13-methyl-15-oxo-7,8,10a,11,12,13,16,17-octahydro-4H,6H,10H,15H-5a,8-ethanoisobenzofuro[7,1-ij]pyrrolo[2,1-c][1,8]dioxa[4]azacyclotetradecin-11-yl)methanesulfonamide. Such compound is considered to be a “compound of the invention,” as that term is used herein.

In some embodiments, the invention relates to a compound of formula

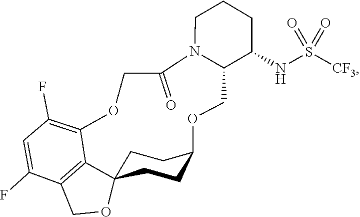

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound N-((5a,8-trans)-(10aR,11S)-1,3-Difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1,1,1-trifluoromethanesulfonamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

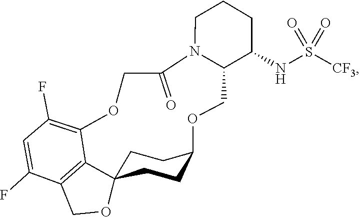

Such compound is considered to be a “compound of the invention,” as that term is used herein.

In some embodiments, the invention relates to the compound N-((5a,8-trans)-(10aR,11S)-1,3-Difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1,1,1-trifluoromethanesulfonamide. Such compound is considered to be a “compound of the invention,” as that term is used herein.

In some embodiments, the invention relates to a compound of formula

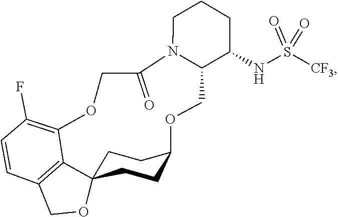

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 1,1,1-Trifluoro-N-((5a,8-trans)(10aR,11S)-1-fluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)methanesulfonamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

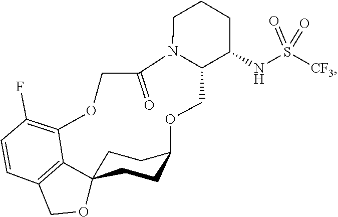

Such compound is considered to be a “compound of the invention,” as that term is used herein.

In some embodiments, the invention relates to the compound 1,1,1-Trifluoro-N-((5a,8-trans)(10aR,11S)-1-fluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)methanesulfonamide. Such compound is considered to be a “compound of the invention,” as that term is used herein.

In some embodiments, the invention relates to a compound of formula

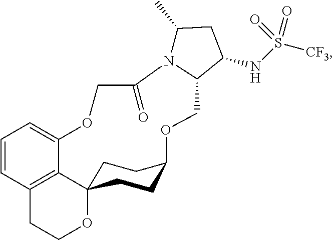

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 1,1,1-Trifluoro-N-(6a,9-trans)-(11aR,12S,14R)-14-methyl-16-oxo-4,5,8,9,11a,12,13,14,16,17-decahydro-7H,11H-6a,9-ethanoisochromeno[8,1-hi]pyrrolo[2,1-c][1,7]dioxa[4]azacyclotridecin-12-yl)methanesulfonamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

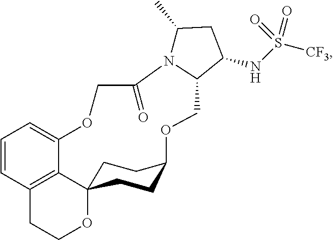

Such compound is considered to be a “compound of the invention,” as that term is used herein.

In some embodiments, the invention relates to the compound 1,1,1-Trifluoro-N-(6a,9-trans)-(11aR,12S,14R)-14-methyl-16-oxo-4,5,8,9,11a,12,13,14,16,17-decahydro-7H,11H-6a,9-ethanoisochromeno[8,1-hi]pyrrolo[2,1-c][1,7]dioxa[4]azacyclotridecin-12-yl)methanesulfonamide. Such compound is considered to be a “compound of the invention,” as that term is used herein.

In some embodiments, the invention relates to a compound of formula:

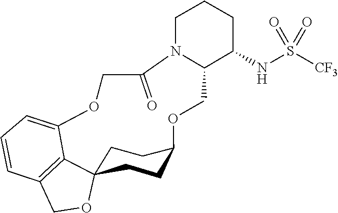

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 1,1,1-Trifluoro-N-((5aR,8R,10aR,11S)-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)methanesulfonamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

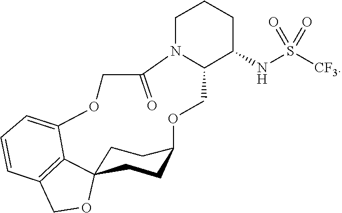

Such compound is considered to be a “compound of the invention,” as that term is used herein.

In some embodiments, the invention relates to the compound 1,1,1-Trifluoro-N-((5aR,8R,10aR,11S)-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)methanesulfonamide. Such compound is considered to be a “compound of the invention,” as that term is used herein.

In some embodiments, the invention relates to a compound of formula

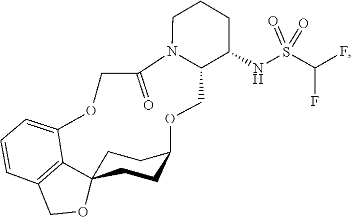

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 1,1-Difluoro-N-((5aR,8R,10aR,11S)-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)methanesulfonamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

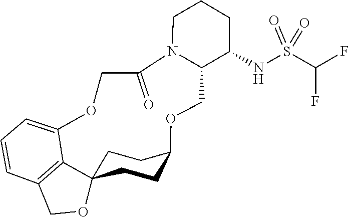

Such compound is considered to be a “compound of the invention,” as that term is used herein.

In some embodiments, the invention relates to the compound 1,1-Difluoro-N-((5aR,8R,10aR,11S)-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)methanesulfonamide. Such compound is considered to be a “compound of the invention,” as that term is used herein.

In some embodiments, the invention relates to a compound of formula

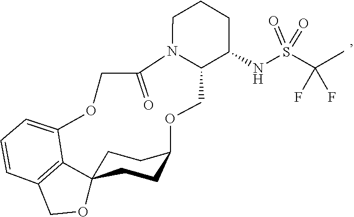

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 1,1-Difluoro-N-((5a,8-trans)-(10aR,11S)-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)ethane-1-sulfonamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

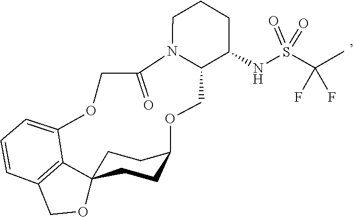

Such compound is considered to be a “compound of the invention,” as that term is used herein.

In some embodiments, the invention relates to the compound 1,1-Difluoro-N-((5a,8-trans)-(10aR,11S)-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)ethane-1-sulfonamide. Such compound is considered to be a “compound of the invention,” as that term is used herein.

In some embodiments, the invention relates to a compound of formula

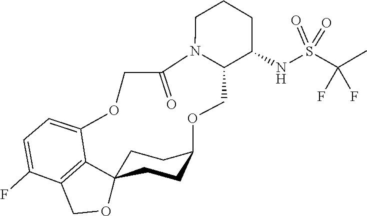

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 1,1-Difluoro-N-((5a,8-trans)-(10aR,11S)-3-fluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)ethane-1-sulfonamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

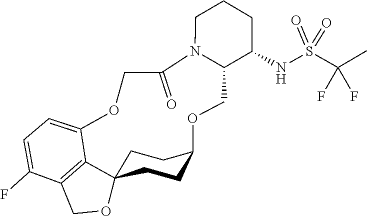

Such compound is considered to be a “compound of the invention,” as that term is used herein.

In some embodiments, the invention relates to the compound 1,1-Difluoro-N-((5a,8-trans)-(10aR,11S)-3-fluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)ethane-1-sulfonamide. Such compound is considered to be a “compound of the invention,” as that term is used herein.

In some embodiments, the invention relates to a compound of formula

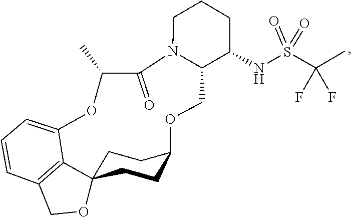

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 1,1-Difluoro-N-((5a,8-trans)-(10aR,11S,17S)-17-methyl-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)ethane-1-sulfonamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

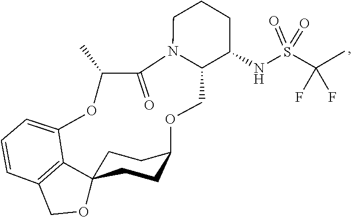

Such compound is considered to be a “compound of the invention,” as that term is used herein.

In some embodiments, the invention relates to the compound 1,1-Difluoro-N-((5a,8-trans)-(10aR,11S,17S)-17-methyl-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)ethane-1-sulfonamide. Such compound is considered to be a “compound of the invention,” as that term is used herein.

In some embodiments, the invention relates to a compound of formula

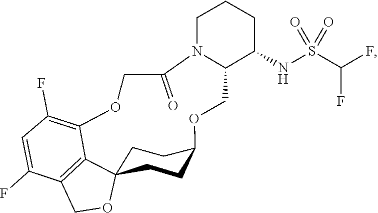

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound N-((5a,8-trans)-(10aR,11S)-1,3-Difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1,1-difluoromethanesulfonamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

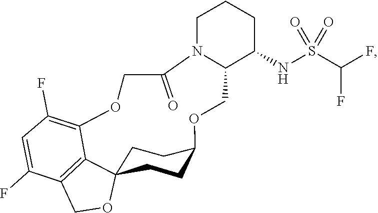

Such compound is considered to be a “compound of the invention,” as that term is used herein.

In some embodiments, the invention relates to the compound N-((5a,8-trans)-(10aR,11S)-1,3-Difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1,1-difluoromethanesulfonamide. Such compound is considered to be a “compound of the invention,” as that term is used herein.

Any compound of the invention can also be radiolabeled for the preparation of a radiopharmaceutical agent.

Any compound of the invention can also be prepared as a prodrug.

In some embodiments, the invention is directed to a compound of Formula (I), (I-1), (1-2), (Ia), (Ia-1), (Ia-2), (I-A), (I-A-1), (I-A-2), (I-B), (I-B-a), (I-B-1), (I-B-1a), (I-B-2), (I-B-2a), (I-C), (I-C-a), (I-C-1), (I-C-1a), (I-C-2), (I-C-2a), (I-D), (I-D-a), (I-D-1), (I-D-1a), (I-D-2), (I-D-2a), or a compound of Table A, Table B, Table C, Table D, or Table E or a deuterated derivative, pharmaceutically acceptable salt, or prodrug thereof.

### Methods of Treatment

One aspect of the invention provides compounds, compositions, and methods useful for preventing or treating a disease which is at least partially mediated by orexin receptor 2. In certain embodiment, the compounds act as agonists of orexin receptor 2. In other embodiments, the compounds act as antagonists of orexin receptor 2.

Another aspect of the invention relates to methods of preventing or treating a disease selected from the group consisting of narcolepsy (e.g., narcolepsy type 1 and/or narcolepsy type 2), idiopathic hypersomnia, hypersomnia, sleep apnea syndrome, narcolepsy syndrome involving narcolepsy-like symptom, hypersomnia associated with Parkinson's disease, hypersomnia associated with dementia with Lewy body, hypersomnia syndrome involving daytime hypersomnia (e.g., Kleine-Levin syndrome, major depression accompanied by hypersomnia, dementia with Lewy body, Parkinson's disease, progressive supranuclear palsy, Prader-Willi syndrome, Moebius syndrome, hypoventilation syndrome, Niemann-Pick disease type C, brain contusion, cerebral infarction, brain tumor, muscular dystrophy, multiple sclerosis, acute disseminated encephalomyelitis, Guillain-Barre syndrome, Rasmussen's encephalitis, Wernicke's encephalopathy, limbic encephalitis, Hashimoto encephalopathy), coma, loss of consciousness, obesity (e.g. malignant mast cell, extrinsic obesity, hyperinsulinar obesity, hyperplasmic obesity, hypophysial obesity, hypoplasmic obesity, hypothyroid obesity, hypothalamic obesity, symptomatic obesity, childhood obesity, upper body obesity, alimentary obesity, gonadal obesity, systemic mastocytosis, primary obesity, central obesity), insulin resistance syndrome, Alzheimer, impaired consciousness such as coma, side effect or complication caused by anesthesia, sleep disturbance, sleep problem, insomnia, intermittent sleep, night myoclonus, REM sleep interruption, jet lag, jet lag syndrome, sleep disorder of shift workers, dyssomnia, sleep terror, depression, major depression, sleepwalking, enuresis, sleep disorder, Alzheimer's sundown syndrome, disease associated with circadian rhythm, fibromyalgia, condition resulting from decrease in sleeping quality, bulimia, obsessive eating disorder, obesity-related diseases, hypertension, diabetes, elevated plasma insulin level/insulin resistance, hyperlipemia, hyperlipidaemia, endometrial cancer, breast cancer, prostate cancer, colon cancer, cancer, osteoarthritis, obstructive sleep apnea, cholelithiasis, gallstone, heart disease, abnormal heartbeat, arrhythmia, myocardial infarction, congestive heart failure, heart failure, coronary heart disease, cardiovascular disease, sudden death, polycystic ovary, craniopharyngioma, Prader Willi syndrome, Froehlich syndrome, growth hormone deficiency, normal variant short stature, Turner syndrome, children suffering from acute lymphoblastic leukemia, syndrome X, reproductive hormone abnormality, decrease of fecundability, infertility, hypogonadism in men, sexual/reproductive-function dysfunction such as hirsutism in women, fetal defect associated with maternity obesity, gastrointestinal motility disorder such as obesity-related gastroesophageal reflux, obesity hypoventilation syndrome (Pickwickian syndrome), respiratory disease such as respiratory distress, inflammation such as vascular systemic inflammation, arteriosclerosis, hypercholesterolemia, hyperuricemia, low back pain, gallbladder disease, gout, renal cancer, secondary risk of obesity such as risk of left ventricle hypertrophy, migraine, headache, neuropathic pain, Parkinson's disease, psychosis, schizophrenia, facial flushing, night sweat, disease in genitalium/urinary system, disease associated with sexual function or fecundability, dysthymic disorder, bipolar disorder, bipolar I disorder, bipolar II disorder, cyclothymic disorder, acute stress disorder, agoraphobia, generalized anxiety disorder, obsessive-compulsive disorder, panic attack, panic disorder, posttraumatic stress disorder, separation anxiety disorder, social phobia, anxiety disorder, acute neurological and psychiatric disorder such as cerebral deficiency developed after heart bypass surgery or heart transplant, stroke, ischemic stroke, cerebral ischemia, spinal cord trauma, head injury, periparturient hypoxia, cardiac arrest, hypoglycemic nerve injury, Huntington's disease, amyotrophic lateral sclerosis, multiple sclerosis, eye damage, retinopathy, cognitive impairment, muscle spasm, tremor, epilepsy, disorder associated with muscle spasm, delirium, amnestic disorder, age-associated cognitive decline, schizoaffective disorder, paranoia, drug addiction, movement disorder, chronic fatigue syndrome, fatigue, medication-induced parkinsonian syndrome, Gilles de la Tourette syndrome, chorea, myoclonus, tic, restless legs syndrome, dystonia, dyskinesia, attention deficit hyperactivity disorder (ADHD), conduct disorder, urinary incontinence, withdrawal symptom, trigeminal neuralgia, hearing loss, tinnitus, nerve injury, retinopathy, macular degeneration, vomiting, cerebral edema, pain, bone pain, arthralgia, toothache, cataplexy, and traumatic brain injury, in a subject in need thereof comprising administering to the subject an effective amount of a compound of the invention, or a pharmaceutically acceptable salt thereof. In certain embodiments, the method comprises administering to the subject an effective amount of a compound of Formula (I).

In certain embodiments, the invention relates to methods of treating or preventing a disease selected from the group consisting of narcolepsy, idiopathic hypersomnia, hypersomnia, sleep apnea syndrome, narcolepsy syndrome involving narcolepsy-like symptoms, hypersomnia associated with Parkinson's disease, and hypersomnia associated with dementia with Lewy body in a subject in need thereof comprising administering to the subject an effective amount of a compound of the invention, or a pharmaceutically acceptable salt thereof. In certain embodiments, the method comprises administering to the subject an effective amount of a compound of Formula (I).

In certain embodiments, the disease is narcolepsy. In particular embodiments, the disease is narcolepsy type 1.

In certain embodiments, the disease is hypersomnolence.

In certain embodiments, the disease is idiopathic hypersomnia.

In certain embodiments, the disease is hypersomnia.

In certain embodiments, the disease is sleep apnea syndrome.

In certain embodiments, the disease is narcolepsy syndrome involving narcolepsy-like symptoms.

In certain embodiments, the disease is hypersomnia associated with Parkinson's disease.

In certain embodiments, the disease is hypersomnia associated with dementia with Lewy body.

In some embodiments, the compound, or pharmaceutically acceptable salt thereof, is administered orally to the subject. In certain embodiments, the compound is administered orally to the subject.

In some embodiments, the compound, or pharmaceutically acceptable salt thereof, is administered parenterally to the subject. In certain embodiments, the compound is administered parenterally to the subject.

In certain embodiments, the disease is prevented. In other embodiments, the disease is treated.

In certain embodiments of any one of the disclosed methods, the compound of the invention is selected from Table A, Table B, Table C, Table D, or Table E. In certain embodiments of any one of the disclosed methods, the compound of the invention is selected from Table A, Table B, or Table C. In certain embodiments of any one of the disclosed methods, the compound of the invention is selected from Table A. In certain embodiments of any one of the disclosed methods, the compound of the invention is selected from Table B. In certain embodiments of any one of the disclosed methods, the compound of the invention is selected from Table C. In certain embodiments of any one of the disclosed methods, the compound of the invention is selected from Table D. In certain embodiments of any one of the disclosed methods, the compound of the invention is selected from Table E.

In some embodiments, the compounds of the invention, and pharmaceutically acceptable salts thereof, modulate orexin 2 receptor in the subject. In other embodiments, the compounds of the invention, and pharmaceutically acceptable salts thereof, activates orexin 2 receptor in the subject. In certain embodiments, the compound of Formula (I) activates orexin receptor 2 in the subject.

In another aspect, the invention relates to the use of a compound of the invention, or a pharmaceutically acceptable salt thereof, in the manufacture of a medicament for the treatment or prevention of a disease in accordance with any of the foregoing methods.

In another aspect, the invention relates to a compound of the invention, or pharmaceutically acceptable salt thereof, for use in the treatment or prevention of a disease in accordance with any of the foregoing methods.

### Pharmaceutical Compositions, Routes of Administration, and Dosing

In certain embodiments, the invention is directed to a pharmaceutical composition, comprising a compound of the invention, or a deuterated derivative, pharmaceutically acceptable salt, or prodrug thereof, e.g., a compound of Formula (I), and a pharmaceutically acceptable carrier.

In certain embodiments, the invention is directed to a pharmaceutical composition, comprising a compound of the invention, or a pharmaceutically acceptable salt thereof, e.g., a compound of Formula (I), and a pharmaceutically acceptable carrier.

In certain embodiments, the invention is directed to a pharmaceutical composition, comprising a compound of the invention, and a pharmaceutically acceptable carrier.

In some embodiments, the invention is directed to a pharmaceutical composition, comprising one or more of the compounds of the invention, or pharmaceutically acceptable salts thereof, and a pharmaceutically acceptable carrier. In certain embodiments, the invention is directed to a pharmaceutical composition, comprising a compound selected from Table A, Table B, Table C, Table D, or Table E, or a pharmaceutically acceptable salt thereof, and a pharmaceutically acceptable carrier. In certain embodiments, the invention is directed to a pharmaceutical composition, comprising a compound selected from Table A, Table B, or Table C, or a pharmaceutically acceptable salt thereof, and a pharmaceutically acceptable carrier. In certain embodiments, the invention is directed to a pharmaceutical composition, comprising a compound selected from Table A, or a pharmaceutically acceptable salt thereof, and a pharmaceutically acceptable carrier. In certain embodiments, the invention is directed to a pharmaceutical composition, comprising a compound selected from Table B, or a pharmaceutically acceptable salt thereof, and a pharmaceutically acceptable carrier. In certain embodiments, the invention is directed to a pharmaceutical composition, comprising a compound selected from Table C, or a pharmaceutically acceptable salt thereof, and a pharmaceutically acceptable carrier. In certain embodiments, the invention is directed to a pharmaceutical composition, comprising a compound selected from Table D, or a pharmaceutically acceptable salt thereof, and a pharmaceutically acceptable carrier. In certain embodiments, the invention is directed to a pharmaceutical composition, comprising a compound selected from Table E, or a pharmaceutically acceptable salt thereof, and a pharmaceutically acceptable carrier.

In certain embodiments, the pharmaceutical composition comprises a plurality of compounds of the invention and a pharmaceutically acceptable carrier.

In certain embodiments, a pharmaceutical composition of the invention further comprises at least one additional pharmaceutically active agent other than a compound of the invention. The at least one additional pharmaceutically active agent can be an agent useful in the treatment of ischemia-reperfusion injury.

Pharmaceutical compositions of the invention can be prepared by combining one or more compounds of the invention with a pharmaceutically acceptable carrier and, optionally, one or more additional pharmaceutically active agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

In certain embodiments, intravenous administration of a compound may typically be from 0.1 mg/kg/day to 20 mg/kg/day. In one embodiment, intravenous administration of a compound may typically be from 0.1 mg/kg/day to 2 mg/kg/day. In one embodiment, intravenous administration of a compound may typically be from 0.5 mg/kg/day to 5 mg/kg/day. In one embodiment, intravenous administration of a compound may typically be from 1 mg/kg/day to 20 mg/kg/day. In one embodiment, intravenous administration of a compound may typically be from 1 mg/kg/day to 10 mg/kg/day.

Generally, daily oral doses of a compound will be, for human subjects, from about 0.01 milligrams/kg per day to 1000 milligrams/kg per day. Oral doses in the range of 0.5 to 50 milligrams/kg, in one or more administrations per day, may yield therapeutic results. Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode that delivers the compound to the desired surface. Administering a pharmaceutical composition may be accomplished by any means known to the skilled artisan. Routes of administration include but are not limited to intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound of the invention can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol moieties are suitable.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound of the invention (or derivative) or by release of the biologically active material beyond the stomach environment, such as in the intestine.

A coating impermeable to at least pH 5.0 may ensure full gastric resistance. Examples of the more common inert ingredients that are used as enteric coatings include cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the invention (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the invention or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the compound may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of the compounds disclosed herein (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (α1-antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J=Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this invention are mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, North Carolina; and the Spinhaler powder inhaler, manufactured by Fisons Corp., Bedford, Mass.

All such devices require the use of formulations suitable for the dispensing of the compounds of the invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compound of the invention may also be prepared in different formulations depending on the type of chemical modification or the type of device employed.

Formulations suitable for use with a nebulizer, either jet or ultrasonic, will typically comprise a compound of the invention (or derivative) dissolved in water at a concentration of about 0.1 to 25 mg of biologically active compound of the invention per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound of the invention caused by atomization of the solution in forming the aerosol.

Formulations for use with a metered-dose inhaler device will generally comprise a finely divided powder containing the compound of the invention (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing a compound of the invention (or derivative) and may also include a bulking agent, such as lactose, sorbitol, sucrose, or mannitol in amounts which facilitate dispersal of the powder from the device, e.g., 50 to 90% by weight of the formulation. The compound of the invention (or derivative) should advantageously be prepared in particulate form with an average particle size of less than 10 micrometers (μm), most preferably 0.5 to 5 m, for most effective delivery to the deep lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described above, a compound may also be formulated as a depot preparation. Such long-acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990).

The compound of the invention and optionally other therapeutics may be administered per se (neat) or in the form of a pharmaceutically acceptable salt or cocrystal. When used in medicine the salts or cocrystals should be pharmaceutically acceptable, but non-pharmaceutically acceptable salts or cocrystals may conveniently be used to prepare pharmaceutically acceptable salts or cocrystals thereof. Such salts include, but are not limited to, those prepared from the following acids: hydrochloric, hydrobromic, sulfuric, nitric, phosphoric, maleic, acetic, salicylic, p-toluene sulfonic, tartaric, citric, methane sulfonic, formic, malonic, succinic, naphthalene-2-sulfonic, and benzene sulfonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound as described herein and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to a compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, non-erodible, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bio-erodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### Synthesis of Compounds of the Invention

The compounds of the invention can be prepared from known materials by the methods described in the Examples, other similar methods, and other methods known to one skilled in the art. As one skilled in the art would appreciate, the functional groups of the intermediate compounds in the methods described below may need to be protected by suitable protecting groups. Protecting groups may be added or removed in accordance with standard techniques, which are well-known to those skilled in the art. The use of protecting groups is described in detail in T.G.M. Wuts et al., Greene's Protective Groups in Organic Synthesis (4th ed. 2006).

### ENUMERATED EMBODIMENTS

Additional embodiments, features, and advantages of the disclosure will be apparent from the following detailed description and through practice of the disclosure. The compounds and methods of the present disclosure can be described as embodiments in any of the following enumerated clauses. It will be understood that any of the embodiments described herein can be used in connection with any other embodiments described herein to the extent that the embodiments do not contradict one another.
1. A compound of formula (I):

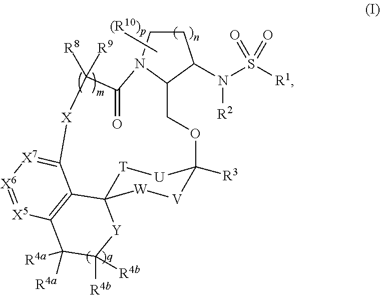

or a pharmaceutically acceptable salt thereof, wherein:
   T is O, CR¹¹R¹², or absent;
   U is CR¹³R¹⁴;
   V is CR¹⁵R¹⁶;
   W is O, CR¹⁷R¹⁸, or absent;
   X is O or absent;
   X⁵ is CR⁵ or N;
   X⁶ is CR⁶ or N;
   X⁷ is CR⁷ or N;
   Y is O or C(R¹⁹)₂;
   R¹ is selected from —NR^{a}R^{b}, —C₁-C₃ alkylene-NR^{a}R^{b}, —C₁-C₃ alkylene-OH, —C₁-C₆ alkyl, —C₁-C₆ haloalkyl, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, and —C₁-C₃ alkylene-(5- to 10-membered heteroaryl), wherein said —C₁-C₃ alkylene-NR^{a}R^{b}, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, or —C₁-C₃ alkylene-(5- to 10-membered heteroaryl) is optionally substituted with 1-3 substituents independently selected from halo, —OH, —C₁-C₃ alkyl, and —C₁-C₃ alkoxy;
   R² is —H or —C₁-C₃ alkyl;
   R³ is —H, halo, —C₁-C₆ alkyl, —C₁-C₆haloalkyl, or —C₁-C₆ alkoxy;
   each R^{4a} is independently selected from —H, halo, —OH, —CN, —C₁-C₆ alkyl, —C₁-C₆haloalkyl, and —C₁-C₆ alkoxy; or
   two R^{4a} together form an oxo;
   each R^{4b} is independently selected from —H, halo, —OH, —CN, —C₁-C₆ alkyl, —C₁-C₆haloalkyl, and —C₁-C₆ alkoxy;
   R⁵, R⁶, and R⁷ are each independently selected from —H, halo, —OH, —CN, —C₁-C₆ alkyl, —C₁-C₆haloalkyl, and —C₁-C₆ alkoxy;
   each R⁸ and each R⁹ is independently selected from —H, halo, —C₁-C₆ alkyl, —C₁-C₆ haloalkyl, and —C₁-C₆ alkoxy; or
   R⁸ and R⁹, together with together with the carbon atom to which they are attached, form a C₃-C₆ cycloalkyl optionally substituted with halo, —C₁-C₃ alkyl, —C₁-C₃ haloalkyl, or —C₁-C₃ alkoxy;
   each R¹⁰ is independently selected from halo, —C₁-C₆ alkyl, —C₁-C₆haloalkyl, and —C₁-C₆ alkoxy;
   R¹¹, R¹², R¹³, R¹⁴, R¹⁵, R¹⁶, R¹⁷, and R¹⁸ are each independently selected from —H, halo, —C₁-C₆ alkyl, —C₁-C₆haloalkyl, and —C₁-C₆ alkoxy;
   each R¹⁹ is independently selected from —H, halo, and —C₁-C₃ alkyl;
   R^{a} and R^{b} are each independently selected from —H and —C₁-C₃ alkyl;
   m is 1, 2, 3, or 4;
   n is 0, 1, 2, or 3;
   p is 0, 1, 2, 3, or 4; and
   q is 0 or 1;
provided that:
   no more than one of T and W is absent; and
   no more than one of X⁵, X⁶, and X⁷ is N.
2. The compound of clause 1, or a pharmaceutically acceptable salt thereof, wherein:
   X⁵ is CRS;
   X⁶ is CR⁶;
   X⁷ is CR⁷; and
   R¹ is selected from —NR^{a}R^{b}, —C₁-C₃ alkylene-NR^{a}R^{b}, —C₁-C₆ alkyl, —C₁-C₆ haloalkyl, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, and —C₁-C₃ alkylene-(5- to 10-membered heteroaryl), wherein said —C₁-C₃ alkylene-NR^{a}R^{b}, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, or —C₁-C₃ alkylene-(5- to 10-membered heteroaryl) is optionally substituted with 1-3 substituents independently selected from halo, —OH, —C₁-C₃ alkyl, and —C₁-C₃ alkoxy.
3. The compound of clause 1, or a pharmaceutically acceptable salt thereof, wherein:
   X⁵ is CR;
   X⁶ is CR⁶;
   X⁷ is CR;
   q is 0;
   R¹ is selected from —NR^{a}R^{b}, —C₁-C₃ alkylene-NR^{a}R^{b}, —C₁-C₆ alkyl, —C₁-C₆ haloalkyl, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, and —C₁-C₃ alkylene-(5- to 10-membered heteroaryl), wherein said —C₁-C₃ alkylene-NR^{a}R^{b}, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, or —C₁-C₃ alkylene-(5- to 10-membered heteroaryl) is optionally substituted with 1-3 substituents independently selected from halo, —OH, —C₁-C₃ alkyl, and —C₁-C₃ alkoxy; and
   each R^{4a} is independently selected from —H, halo, —OH, —CN, —C₁-C₆ alkyl, —C₁-C₆haloalkyl, and —C₁-C₆ alkoxy.
4. The compound of any one of clauses 1 to 3, wherein the compound is of formula (I-1):

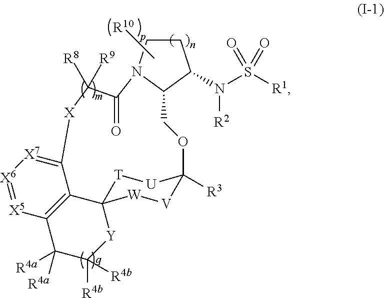

or a pharmaceutically acceptable salt thereof.
5. The compound of any one of clauses 1 to 3, wherein the compound is of formula (I-2):

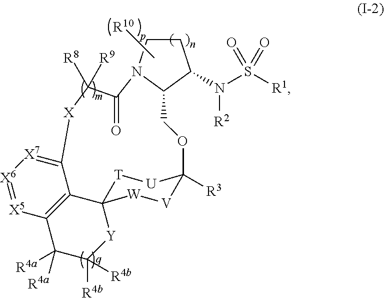

or a pharmaceutically acceptable salt thereof.
6. The compound of any one of clauses 1 to 5, or a pharmaceutically acceptable salt thereof, wherein X⁵ is CR⁵.
7. The compound of any one of clauses 1 to 6, or a pharmaceutically acceptable salt thereof, wherein X⁶ is CR⁶.
8. The compound of any one of clauses 1 to 7, or a pharmaceutically acceptable salt thereof, wherein X⁷ is CR⁷.
9. The compound of any one of clauses 1 to 8, wherein the compound is of formula (Ia-2):

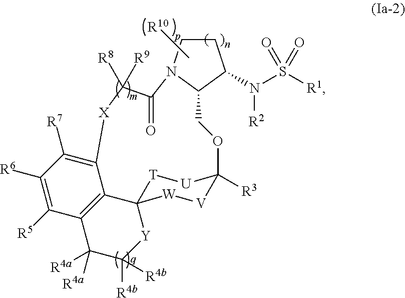

or a pharmaceutically acceptable salt thereof.
10. The compound of any one of clauses 1 to 9, or a pharmaceutically acceptable salt thereof, wherein X is O.
11. The compound of any one of clauses 1 to 10, or a pharmaceutically acceptable salt thereof, wherein T is CR¹¹R¹²
12. The compound of any one of clauses 1 to 11, or a pharmaceutically acceptable salt thereof, wherein W is CR¹⁷R¹⁸.
13. The compound of any one of clauses 1 to 12, or a pharmaceutically acceptable salt thereof, wherein R¹¹, R¹², R¹³, R¹⁴, R¹⁵, R¹⁶, R¹⁷, and R¹⁸ are each —H.
14. The compound of any one of clauses 1 to 13, or a pharmaceutically acceptable salt thereof, wherein R² is —H.
15. The compound of any one of clauses 1 to 14, or a pharmaceutically acceptable salt thereof, wherein R³ is —H.
16. The compound of any one of clauses 1 to 15, or a pharmaceutically acceptable salt thereof, wherein m is 1 or 2.
17. The compound of any one of clauses 1 to 16, or a pharmaceutically acceptable salt thereof, wherein n is 1 or 2.
18. The compound of any one of clauses 1 to 17, or a pharmaceutically acceptable salt thereof, wherein each R^{4a} is independently selected from —H, halo, —OH, and —C₁-C₆ alkyl.
19. The compound of any one of clauses 1 to 18, or a pharmaceutically acceptable salt thereof, wherein each R^{4a} is independently selected from —H, —F, —Br, —OH, and —CH₃.
20. The compound of any one of clauses 1 to 19, or a pharmaceutically acceptable salt thereof, wherein two R^{4a} together form an oxo.
21. The compound of any one of clauses 1 to 20, or a pharmaceutically acceptable salt thereof, wherein q is 0.
22. The compound of any one of clauses 1 to 21, or a pharmaceutically acceptable salt thereof, wherein Y is O.
23. The compound of any one of clauses 1 to 22, wherein the compound is of formula (I-A-2):

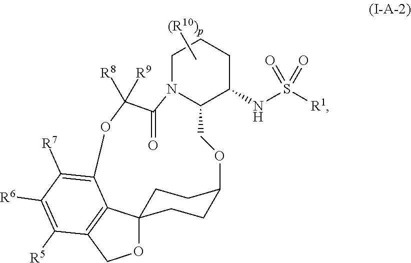

or a pharmaceutically acceptable salt thereof.
24. The compound of any one of clauses 1 to 22, wherein the compound is of formula (I-B-2):

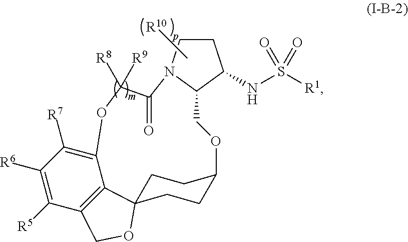

or a pharmaceutically acceptable salt thereof.
25. The compound of any one of clauses 1 to 22, wherein the compound is of formula (I-B-2a):

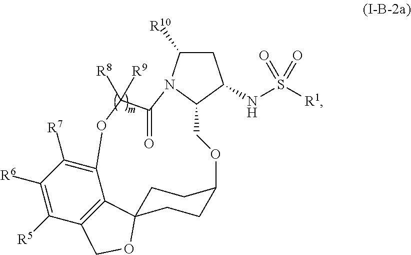

or a pharmaceutically acceptable salt thereof.
26. The compound of clause 24 or 25, wherein m is 1.
27. The compound of clause 24 or 25, wherein m is 2.
28. The compound of any one of clauses 1 to 21, or a pharmaceutically acceptable salt thereof, wherein Y is C(R¹⁹)₂ and each R¹⁹ is H.
29. The compound of any one of clauses 1 to 21 and 28, wherein the compound is of formula (I-C-2):

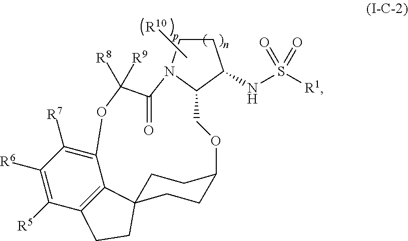

or a pharmaceutically acceptable salt thereof.
30. The compound of clause 29, or a pharmaceutically acceptable salt thereof, wherein n is 1.
31. The compound of any one of clauses 1 to 21 and 28 to 30, wherein the compound is of formula (I-C-2a):

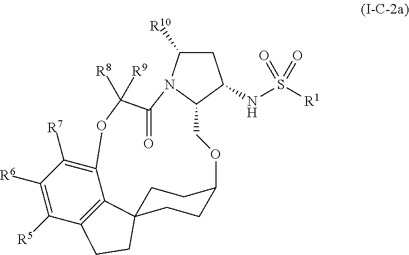

or a pharmaceutically acceptable salt thereof.
32. The compound of clause 29, or a pharmaceutically acceptable salt thereof, wherein n is 2.
33. The compound of any one of clauses 1, 2, and 4 to 20, or a pharmaceutically acceptable salt thereof, wherein q is 1.
34. The compound of any one of clauses 1, 2, 4 to 20, and 33, or a pharmaceutically acceptable salt thereof, wherein Y is O.
35. The compound of any one of clauses 1, 2, 4 to 20, 33, and 34, wherein the compound is of formula (I-D-2):

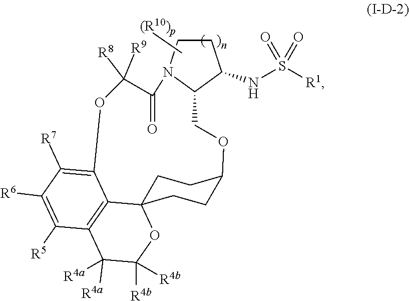

or a pharmaceutically acceptable salt thereof.
36. The compound of any one of clauses 1, 2, 4 to 20, and 33 to 35, wherein the compound is of formula (I-D-2a):

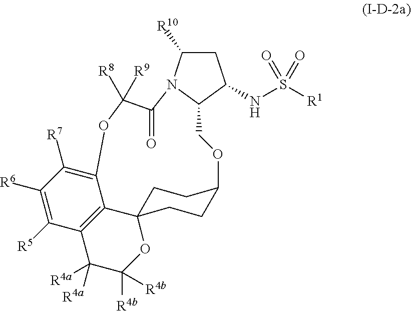

or a pharmaceutically acceptable salt thereof.
37. The compound of any one of clauses 1, 2, 4 to 20, and 33 to 36, or a pharmaceutically acceptable salt thereof, wherein each R^{4b} is H.
38. The compound of any one of clauses 1 to 37, or a pharmaceutically acceptable salt thereof, wherein R¹ is selected from —C₁-C₆ alkyl, —C₁-C₆ haloalkyl, —C₁-C₃ alkylene-OH, —C₃-C₈ cycloalkyl, 4- to 10-membered heterocyclyl, and —C₆-C₁₀ aryl, wherein said —C₃-C₈ cycloalkyl, 4- to 10-membered heterocyclyl, and —C₆-C₁₀ aryl, is optionally substituted with 1-3 substituents independently selected from halo, —OH, —C₁-C₃ alkyl, and —C₁-C₃ alkoxy.
39. The compound of any one of clauses 1 to 38, or a pharmaceutically acceptable salt thereof, wherein R¹ is selected from —CH₃, —CH₂CH₃, —CH₂CH₂CH₃, —CH(CH₃)₂, —CH(CH₃)CH₂CH₃, —CH₂F, —CHF₂, —CF₃, —CHFCH₃, —CH₂CF₃, —CF₂CH₃, —CH₂CH₂OH, cyclopropyl, 1-fluorocyclopropyl, 1-methylcyclopropyl, cyclobutyl, 3,3-difluorocyclobutyl, azetidinyl, oxetanyl, pyrrolidinyl, tetrahydrofuranyl, and 2-fluorophenyl.
40. The compound of any one of clauses 1 to 39, or a pharmaceutically acceptable salt thereof, wherein R¹ is selected from —CH₃, —CH₂CH₃, —CH₂CH₂CH₃, —CH(CH₃)₂, —CH(CH₃)CH₂CH₃, —CH₂F, —CHF₂, —CF₃, —CHFCH₃, —CH₂CF₃, —CF₂CH₃, —CH₂CH₂OH,

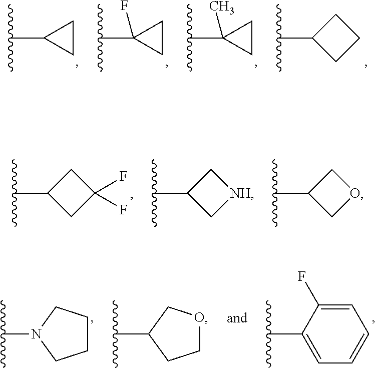

41. The compound of any one of clauses 1 to 40, or a pharmaceutically acceptable salt thereof, wherein R¹ is selected from —CHF₂, —CF₃, and —CF₂CH₃.
42. The compound of any one of clauses 1 to 41, or a pharmaceutically acceptable salt thereof, wherein R⁵, R⁶, and R⁷ are each independently selected from —H, halo, —C₁-C₆ alkyl, and —C₁-C₆ haloalkyl.
43. The compound of any one of clauses 1 to 42, or a pharmaceutically acceptable salt thereof, wherein R⁵ is —H, —F, —Cl, —Br, or —CH₃.
44. The compound of any one of clauses 1 to 43, or a pharmaceutically acceptable salt thereof, wherein R⁶ is —H —F, or —Br.
45. The compound of any one of clauses 1 to 44, or a pharmaceutically acceptable salt thereof, wherein R⁷ is —H, —F, —Br, —CH₃, or —CF₃.
46. The compound of any one of clauses 1 to 45, or a pharmaceutically acceptable salt thereof, wherein each R⁸ and each R⁹ is independently selected from —H, halo, and —C₁-C₆ alkyl.
47. The compound of any one of clauses 1 to 46, or a pharmaceutically acceptable salt thereof, wherein each R⁸ and each R⁹ is independently selected from —H, —F, —CH₃, and —CH₂CH₃.
48. The compound of any one of clauses 1 to 45, or a pharmaceutically acceptable salt thereof, wherein R⁸ and R⁹, together with together with the carbon atom to which they are attached, form a C₃-C₆ cycloalkyl optionally substituted with halo, —C₁-C₃ alkyl, —C₁-C₃ haloalkyl, or —C₁-C₃ alkoxy.
49. The compound of any one of clauses 1 to 45 and 48, or a pharmaceutically acceptable salt thereof, wherein R⁸ and R⁹, together with together with the carbon atom to which they are attached, form a cyclopropyl optionally substituted with halo, —C₁-C₃ alkyl, —C₁-C₃ haloalkyl, or —C₁-C₃ alkoxy.
50. The compound of any one of clauses 1 to 49, or a pharmaceutically acceptable salt thereof, wherein p is 0, 1, or 2.
51. The compound of any one of clauses 1 to 50, or a pharmaceutically acceptable salt thereof, wherein each R¹⁰ is independently selected from halo and —C₁-C₆ alkyl.
52. The compound of any one of clauses 1 to 51, or a pharmaceutically acceptable salt thereof, wherein each R¹⁰ is independently selected from —F and —CH₃.
53. The compound of any one of clauses 1 to 21, or a pharmaceutically acceptable salt thereof, wherein:
   X is O;
   m is 1; and
   n is 1.
54. The compound of any one of clauses 1 to 21, or a pharmaceutically acceptable salt thereof, wherein:
   X is O;
   m is 2; and
   n is 1.
55. The compound of any one of clauses 1 to 21, or a pharmaceutically acceptable salt thereof, wherein:
   X is O;
   m is 1; and
   n is 2.
56. The compound of any one of clauses 1 to 21, or a pharmaceutically acceptable salt thereof, wherein:
   X is O;
   R¹ is —CF₃;
   m is 1; and
   n is 1.
57. The compound of any one of clauses 1 to 21, or a pharmaceutically acceptable salt thereof, wherein:
   X is O;
   R¹ is —CF₃;
   m is 2; and
   n is 1.
58. The compound of any one of clauses 1 to 21, or a pharmaceutically acceptable salt thereof, wherein:
   X is O;
   R¹ is —CHF₂, —CF₃, or —CF₂CH₃;
   m is 1; and
   n is 2.
59. The compound of any one of clauses 1 to 21, or a pharmaceutically acceptable salt thereof, wherein:
   X is O;
   Y is O;
   m is 1; and
   n is 1.
60. The compound of any one of clauses 1 to 21, or a pharmaceutically acceptable salt thereof, wherein:
   X is O;
   Y is O;
   m is 2; and
   n is 1.
61. The compound of any one of clauses 1 to 21, or a pharmaceutically acceptable salt thereof, wherein:
   X is O;
   Y is O;
   m is 1; and
   n is 2.
62. The compound of any one of clauses 1 to 21, or a pharmaceutically acceptable salt thereof, wherein:
   X is O;
   Y is CH₂;
   m is 1; and
   n is 1.
63. The compound of any one of clauses 1 to 21, or a pharmaceutically acceptable salt thereof, wherein:
   X is O;
   Y is CH₂;
   m is 2; and
   n is 1.
64. The compound of any one of clauses 1 to 21, or a pharmaceutically acceptable salt thereof, wherein:
   X is O;
   Y is CH₂;
   m is 1; and
   n is 2.
65. The compound of any one of clauses 1 to 21, or a pharmaceutically acceptable salt thereof, wherein:
   X is O;
   Y is O;
   R¹ is —CF₃;
   m is 1; and
   n is 1.
66. The compound of any one of clauses 1 to 21, or a pharmaceutically acceptable salt thereof, wherein:
   X is O;
   Y is O;
   R¹ is —CF₃;
   m is 2; and
   n is 1.
67. The compound of any one of clauses 1 to 21, or a pharmaceutically acceptable salt thereof, wherein:
   X is O;
   Y is O;
   R¹ is —CHF₂, —CF₃, or —CF₂CH₃;
   m is 1; and
   n is 2.
68. The compound of any one of clauses 1 to 21, or a pharmaceutically acceptable salt thereof, wherein:
   X is O;
   Y is CH₂;
   R¹ is —CF₃;
   m is 1; and
   n is 1.
69. The compound of any one of clauses 1 to 21, or a pharmaceutically acceptable salt thereof, wherein:
   X is O;
   Y is CH₂;
   R¹ is —CF₃;
   m is 2; and
   n is 1.
70. The compound of any one of clauses 1 to 21, or a pharmaceutically acceptable salt thereof, wherein:
   X is O;
   Y is CH₂;
   R¹ is —CF₃;
   m is 1; and
   n is 2.
71. The compound of any one of clauses 1 to 21, or a pharmaceutically acceptable salt thereof, wherein:
   X is O;
   Y is O;
   R¹ is —CF₃;
   m is 1;
   n is 1; and
   q is 0.
72. The compound of any one of clauses 1 to 21, or a pharmaceutically acceptable salt thereof, wherein:
   X is O;
   Y is O;
   R¹ is —CF₃;
   mis 2;
   n is 1; and
   q is 0.
73. The compound of any one of clauses 1 to 21, or a pharmaceutically acceptable salt thereof, wherein:
   X is O;
   Y is O;
   R¹ is —CHF₂, —CF₃, or —CF₂CH₃;
   m is 1;
   n is 2; and
   q is 0.
74. The compound of any one of clauses 1 to 21, or a pharmaceutically acceptable salt thereof, wherein:
   X is O;
   Y is CH₂;
   R¹ is —CF₃;
   m is 1;
   n is 1; and
   q is 0.
75. The compound of any one of clauses 1 to 21, or a pharmaceutically acceptable salt thereof, wherein:
   X is O;
   Y is CH₂;
   R¹ is —CF₃;
   m is 2;
   n is 1; and
   q is 0.
76. The compound of any one of clauses 1 to 21, or a pharmaceutically acceptable salt thereof, wherein:
   X is O;
   Y is CH₂;
   R¹ is —CF₃;
   m is 1;
   n is 2; and
   q is 0.
77. A compound selected from Table A, or a pharmaceutically acceptable salt thereof.
78. A compound selected from Table B, or a pharmaceutically acceptable salt thereof.
79. A compound selected from Table C, or a pharmaceutically acceptable salt thereof.
80. A compound selected from Table D, or a pharmaceutically acceptable salt thereof.
81. A compound selected from Table E, or a pharmaceutically acceptable salt thereof.
82. The compound of any one of clauses 1 to 81 in non-salt form.
83. A pharmaceutical composition comprising a compound of any one of clauses 1 to 81, or a pharmaceutically acceptable salt thereof, or the compound of clause 82; and at least one pharmaceutically acceptable excipient.
84. A method of preventing or treating a disease associated with a deficiency in orexin 2 receptor signaling, comprising administering to a subject in need thereof a therapeutically effective amount of a compound of any one of clauses 1 to 81, or a pharmaceutically acceptable salt thereof, a compound of clause 82, or a pharmaceutical composition of clause 83.
85. A method of preventing or treating a disease selected from narcolepsy, idiopathic hypersomnia, hypersomnia, sleep apnea syndrome, narcolepsy syndrome involving narcolepsy-like symptom, hypersomnia associated with Parkinson's disease, hypersomnia associated with dementia with Lewy body, hypersomnia syndrome involving daytime hypersomnia (e.g., Kleine-Levin syndrome, major depression accompanied by hypersomnia, dementia with Lewy body, Parkinson's disease, progressive supranuclear palsy, Prader-Willi syndrome, Moebius syndrome, hypoventilation syndrome, Niemann-Pick disease type C, brain contusion, cerebral infarction, brain tumor, muscular dystrophy, multiple sclerosis, acute disseminated encephalomyelitis, Guillain-Barre syndrome, Rasmussen's encephalitis, Wernicke's encephalopathy, limbic encephalitis, Hashimoto encephalopathy), coma, loss of consciousness, obesity (e.g., malignant mast cell, extrinsic obesity, hyperinsulinar obesity, hyperplasmic obesity, hypophysial obesity, hypoplasmic obesity, hypothyroid obesity, hypothalamic obesity, symptomatic obesity, childhood obesity, upper body obesity, alimentary obesity, gonadal obesity, systemic mastocytosis, primary obesity, central obesity), insulin resistance syndrome, Alzheimer, impaired consciousness such as coma, side effect or complication caused by anesthesia, sleep disturbance, sleep problem, insomnia, intermittent sleep, night myoclonus, REM sleep interruption, jet lag, jet lag syndrome, sleep disorder of shift workers, dyssomnia, sleep terror, depression, major depression, sleepwalking, enuresis, sleep disorder, Alzheimer's sundown syndrome, disease associated with circadian rhythm, fibromyalgia, condition resulting from decrease in sleeping quality, bulimia, obsessive eating disorder, obesity-related diseases, hypertension, diabetes, elevated plasma insulin level/insulin resistance, hyperlipemia, hyperlipidaemia, endometrial cancer, breast cancer, prostate cancer, colon cancer, cancer, osteoarthritis, obstructive sleep apnea, cholelithiasis, gallstone, heart disease, abnormal heartbeat, arrhythmia, myocardial infarction, congestive heart failure, heart failure, coronary heart disease, cardiovascular disease, sudden death, polycystic ovary, craniopharyngioma, Prader Willi syndrome, Froehlich syndrome, growth hormone deficiency, normal variant short stature, Turner syndrome, children suffering from acute lymphoblastic leukemia, syndrome X, reproductive hormone abnormality, decrease of fecundability, infertility, hypogonadism in men, sexual/reproductive-function dysfunction such as hirsutism in women, fetal defect associated with maternity obesity, gastrointestinal motility disorder such as obesity-related gastroesophageal reflux, obesity hypoventilation syndrome (Pickwickian syndrome), respiratory disease such as respiratory distress, inflammation such as vascular systemic inflammation, arteriosclerosis, hypercholesterolemia, hyperuricemia, low back pain, gallbladder disease, gout, renal cancer, secondary risk of obesity such as risk of left ventricle hypertrophy, migraine, headache, neuropathic pain, Parkinson's disease, psychosis, schizophrenia, facial flushing, night sweat, disease in genitalium/urinary system, disease associated with sexual function or fecundability, dysthymic disorder, bipolar disorder, bipolar I disorder, bipolar II disorder, cyclothymic disorder, acute stress disorder, agoraphobia, generalized anxiety disorder, obsessive-compulsive disorder, panic attack, panic disorder, posttraumatic stress disorder, separation anxiety disorder, social phobia, anxiety disorder, acute neurological and psychiatric disorder such as cerebral deficiency developed after heart bypass surgery or heart transplant, stroke, ischemic stroke, cerebral ischemia, spinal cord trauma, head injury, periparturient hypoxia, cardiac arrest, hypoglycemic nerve injury, Huntington's disease, amyotrophic lateral sclerosis, multiple sclerosis, eye damage, retinopathy, cognitive impairment, muscle spasm, tremor, epilepsy, disorder associated with muscle spasm, delirium, amnestic disorder, age-associated cognitive decline, schizoaffective disorder, paranoia, drug addiction, movement disorder, chronic fatigue syndrome, fatigue, medication-induced parkinsonian syndrome, Gilles de la Tourette syndrome, chorea, myoclonus, tic, restless legs syndrome, dystonia, dyskinesia, attention deficit hyperactivity disorder (ADHD), conduct disorder, urinary incontinence, withdrawal symptom, trigeminal neuralgia, hearing loss, tinnitus, nerve injury, retinopathy, macular degeneration, vomiting, cerebral edema, pain, bone pain, arthralgia, toothache, cataplexy, and traumatic brain injury, comprising administering to a subject in need thereof a therapeutically effective amount of a compound of any one of clauses 1 to 81, or a pharmaceutically acceptable salt thereof, a compound of clause 82, or a pharmaceutical composition of clause 83.
86. A method of preventing or treating a disease selected from narcolepsy, idiopathic hypersomnia, hypersomnia, sleep apnea syndrome, narcolepsy syndrome involving narcolepsy-like symptoms, hypersomnia associated with Parkinson's disease, and hypersomnia associated with dementia with Lewy body, comprising administering to a subject in need thereof a therapeutically effective amount of a compound of any one of clauses 1 to 81, or a pharmaceutically acceptable salt thereof, a compound of clause 82, or a pharmaceutical composition of clause 83.
87. The method of any one of clauses 84 to 86, wherein said subject is treated with one or more additional therapeutic agents administered concurrently with, prior to, or subsequent to treatment with the compound, pharmaceutically acceptable salt, or pharmaceutical composition.
88. Use of a compound of any one of clauses 1 to 81, or a pharmaceutically acceptable salt thereof, a compound of clause 82, or a pharmaceutical composition of clause 83 in the manufacture of a medicament for the treatment or prevention of a disease selected from narcolepsy, idiopathic hypersomnia, hypersomnia, sleep apnea syndrome, narcolepsy syndrome involving narcolepsy-like symptom, hypersomnia associated with Parkinson's disease, hypersomnia associated with dementia with Lewy body, hypersomnia syndrome involving daytime hypersomnia (e.g., Kleine-Levin syndrome, major depression accompanied by hypersomnia, dementia with Lewy body, Parkinson's disease, progressive supranuclear palsy, Prader-Willi syndrome, Moebius syndrome, hypoventilation syndrome, Niemann-Pick disease type C, brain contusion, cerebral infarction, brain tumor, muscular dystrophy, multiple sclerosis, acute disseminated encephalomyelitis, Guillain-Barre syndrome, Rasmussen's encephalitis, Wernicke's encephalopathy, limbic encephalitis, Hashimoto encephalopathy), coma, loss of consciousness, obesity (e.g., malignant mast cell, extrinsic obesity, hyperinsulinar obesity, hyperplasmic obesity, hypophysial obesity, hypoplasmic obesity, hypothyroid obesity, hypothalamic obesity, symptomatic obesity, childhood obesity, upper body obesity, alimentary obesity, gonadal obesity, systemic mastocytosis, primary obesity, central obesity), insulin resistance syndrome, Alzheimer, impaired consciousness such as coma, side effect or complication caused by anesthesia, sleep disturbance, sleep problem, insomnia, intermittent sleep, night myoclonus, REM sleep interruption, jet lag, jet lag syndrome, sleep disorder of shift workers, dyssomnia, sleep terror, depression, major depression, sleepwalking, enuresis, sleep disorder, Alzheimer's sundown syndrome, disease associated with circadian rhythm, fibromyalgia, condition resulting from decrease in sleeping quality, bulimia, obsessive eating disorder, obesity-related diseases, hypertension, diabetes, elevated plasma insulin level/insulin resistance, hyperlipemia, hyperlipidaemia, endometrial cancer, breast cancer, prostate cancer, colon cancer, cancer, osteoarthritis, obstructive sleep apnea, cholelithiasis, gallstone, heart disease, abnormal heartbeat, arrhythmia, myocardial infarction, congestive heart failure, heart failure, coronary heart disease, cardiovascular disease, sudden death, polycystic ovary, craniopharyngioma, Prader Willi syndrome, Froehlich syndrome, growth hormone deficiency, normal variant short stature, Turner syndrome, children suffering from acute lymphoblastic leukemia, syndrome X, reproductive hormone abnormality, decrease of fecundability, infertility, hypogonadism in men, sexual/reproductive-function dysfunction such as hirsutism in women, fetal defect associated with maternity obesity, gastrointestinal motility disorder such as obesity-related gastroesophageal reflux, obesity hypoventilation syndrome (Pickwickian syndrome), respiratory disease such as respiratory distress, inflammation such as vascular systemic inflammation, arteriosclerosis, hypercholesterolemia, hyperuricemia, low back pain, gallbladder disease, gout, renal cancer, secondary risk of obesity such as risk of left ventricle hypertrophy, migraine, headache, neuropathic pain, Parkinson's disease, psychosis, schizophrenia, facial flushing, night sweat, disease in genitalium/urinary system, disease associated with sexual function or fecundability, dysthymic disorder, bipolar disorder, bipolar I disorder, bipolar II disorder, cyclothymic disorder, acute stress disorder, agoraphobia, generalized anxiety disorder, obsessive-compulsive disorder, panic attack, panic disorder, posttraumatic stress disorder, separation anxiety disorder, social phobia, anxiety disorder, acute neurological and psychiatric disorder such as cerebral deficiency developed after heart bypass surgery or heart transplant, stroke, ischemic stroke, cerebral ischemia, spinal cord trauma, head injury, periparturient hypoxia, cardiac arrest, hypoglycemic nerve injury, Huntington's disease, amyotrophic lateral sclerosis, multiple sclerosis, eye damage, retinopathy, cognitive impairment, muscle spasm, tremor, epilepsy, disorder associated with muscle spasm, delirium, amnestic disorder, age-associated cognitive decline, schizoaffective disorder, paranoia, drug addiction, movement disorder, chronic fatigue syndrome, fatigue, medication-induced parkinsonian syndrome, Gilles de la Tourette syndrome, chorea, myoclonus, tic, restless legs syndrome, dystonia, dyskinesia, attention deficit hyperactivity disorder (ADHD), conduct disorder, urinary incontinence, withdrawal symptom, trigeminal neuralgia, hearing loss, tinnitus, nerve injury, retinopathy, macular degeneration, vomiting, cerebral edema, pain, bone pain, arthralgia, toothache, cataplexy, and traumatic brain injury.
89. A compound of any one of clauses 1 to 81, or a pharmaceutically acceptable salt thereof, a compound of clause 82, or a pharmaceutical composition of clause 83 for use in the treatment or prevention of a disease selected from narcolepsy, idiopathic hypersomnia, hypersomnia, sleep apnea syndrome, narcolepsy syndrome involving narcolepsy-like symptom, hypersomnia associated with Parkinson's disease, hypersomnia associated with dementia with Lewy body, hypersomnia syndrome involving daytime hypersomnia (e.g., Kleine-Levin syndrome, major depression accompanied by hypersomnia, dementia with Lewy body, Parkinson's disease, progressive supranuclear palsy, Prader-Willi syndrome, Moebius syndrome, hypoventilation syndrome, Niemann-Pick disease type C, brain contusion, cerebral infarction, brain tumor, muscular dystrophy, multiple sclerosis, acute disseminated encephalomyelitis, Guillain-Barre syndrome, Rasmussen's encephalitis, Wernicke's encephalopathy, limbic encephalitis, Hashimoto encephalopathy), coma, loss of consciousness, obesity (e.g., malignant mast cell, extrinsic obesity, hyperinsulinar obesity, hyperplasmic obesity, hypophysial obesity, hypoplasmic obesity, hypothyroid obesity, hypothalamic obesity, symptomatic obesity, childhood obesity, upper body obesity, alimentary obesity, gonadal obesity, systemic mastocytosis, primary obesity, central obesity), insulin resistance syndrome, Alzheimer, impaired consciousness such as coma, side effect or complication caused by anesthesia, sleep disturbance, sleep problem, insomnia, intermittent sleep, night myoclonus, REM sleep interruption, jet lag, jet lag syndrome, sleep disorder of shift workers, dyssomnia, sleep terror, depression, major depression, sleepwalking, enuresis, sleep disorder, Alzheimer's sundown syndrome, disease associated with circadian rhythm, fibromyalgia, condition resulting from decrease in sleeping quality, bulimia, obsessive eating disorder, obesity-related diseases, hypertension, diabetes, elevated plasma insulin level/insulin resistance, hyperlipemia, hyperlipidaemia, endometrial cancer, breast cancer, prostate cancer, colon cancer, cancer, osteoarthritis, obstructive sleep apnea, cholelithiasis, gallstone, heart disease, abnormal heartbeat, arrhythmia, myocardial infarction, congestive heart failure, heart failure, coronary heart disease, cardiovascular disease, sudden death, polycystic ovary, craniopharyngioma, Prader Willi syndrome, Froehlich syndrome, growth hormone deficiency, normal variant short stature, Turner syndrome, children suffering from acute lymphoblastic leukemia, syndrome X, reproductive hormone abnormality, decrease of fecundability, infertility, hypogonadism in men, sexual/reproductive-function dysfunction such as hirsutism in women, fetal defect associated with maternity obesity, gastrointestinal motility disorder such as obesity-related gastroesophageal reflux, obesity hypoventilation syndrome (Pickwickian syndrome), respiratory disease such as respiratory distress, inflammation such as vascular systemic inflammation, arteriosclerosis, hypercholesterolemia, hyperuricemia, low back pain, gallbladder disease, gout, renal cancer, secondary risk of obesity such as risk of left ventricle hypertrophy, migraine, headache, neuropathic pain, Parkinson's disease, psychosis, schizophrenia, facial flushing, night sweat, disease in genitalium/urinary system, disease associated with sexual function or fecundability, dysthymic disorder, bipolar disorder, bipolar I disorder, bipolar II disorder, cyclothymic disorder, acute stress disorder, agoraphobia, generalized anxiety disorder, obsessive-compulsive disorder, panic attack, panic disorder, posttraumatic stress disorder, separation anxiety disorder, social phobia, anxiety disorder, acute neurological and psychiatric disorder such as cerebral deficiency developed after heart bypass surgery or heart transplant, stroke, ischemic stroke, cerebral ischemia, spinal cord trauma, head injury, periparturient hypoxia, cardiac arrest, hypoglycemic nerve injury, Huntington's disease, amyotrophic lateral sclerosis, multiple sclerosis, eye damage, retinopathy, cognitive impairment, muscle spasm, tremor, epilepsy, disorder associated with muscle spasm, delirium, amnestic disorder, age-associated cognitive decline, schizoaffective disorder, paranoia, drug addiction, movement disorder, chronic fatigue syndrome, fatigue, medication-induced parkinsonian syndrome, Gilles de la Tourette syndrome, chorea, myoclonus, tic, restless legs syndrome, dystonia, dyskinesia, attention deficit hyperactivity disorder (ADHD), conduct disorder, urinary incontinence, withdrawal symptom, trigeminal neuralgia, hearing loss, tinnitus, nerve injury, retinopathy, macular degeneration, vomiting, cerebral edema, pain, bone pain, arthralgia, toothache, cataplexy, and traumatic brain injury.
90. Use of the compound of any one of clauses 1 to 81, or a pharmaceutically acceptable salt thereof, the compound of clause 82, or the pharmaceutical composition of clause 83, as a medicament.

### EXAMPLES

The invention is further described in the following examples, which do not limit the scope of the invention described in the claims.

### ABBREVIATIONS

Unless otherwise noted, or where the context dictates otherwise, the following abbreviations shall be understood to have the following meanings:

| Abbreviation | Meaning |
|---|---|
| NMR | Nuclear magnetic resonance |
| SFC | Supercritical fluid chromatography |
| ESI-MS | Electrospray mass spectrometry |
| LC/MS | Liquid chromatography-mass spectrometry |
| HPLC | High performance liquid chromatography |
| ESI | Electrospray ionization |
| g | Grams |
| mg | Milligrams |
| L | Liter(s) |
| mL | Milliliter(s) |
| μL | Microliter(s) |
| mmol | Millimole(s) |
| h | Hour(s) |
| min | Minute(s) |
| μm | Micrometer(s) |
| MHz | Megahertz |
| Hz | Hertz |
| N | Normal (concentration) |
| M | Molar (concentration) |
| ppm | Parts per million |
| AcOH | Acetic acid |
| AgOAc | Silver acetate |
| Bn | Benzyl |
| Boc | tert-Butyl carbonate |
| BTSA | 2,2-Bis(trimethylsilyl)acetamide |
| ⁿBuLi | n-Butyllithium |
| DABCO | 1,4-Diazabicyclo[2.2.2]octane |
| DAST | (Diethylamino)sulfur trifluoride |
| DBU | 1,8-Diazabicyclo[5.4.0]undec-7-ene |
| DCM | Dichloromethane |
| DIAD | Diisopropyl azodicarboxylate |
| DIBAL | Diisobutylaluminum hydride |
| DIPEA | N,N-Diisopropylethylamine |
| DMAP | N,N-Dimethylaminopyridine |
| DMF | N,N-Dimethylformamide |
| DMSO | Dimethyl sulfoxide |
| EDC | N-(3-Dimethylaminopropyl)-N′-ethylcarbodiimide |
| EtOAc | Ethyl acetate |
| HATU | 1-[Bis(dimethylamino)methylene]-1H-1,2,3- |
| | triazolo[4,5-b]pyridinium 3-oxide hexafluorophosphate |
| HOBt.xH₂O | 1-Hydroxybenzotriazole hydrate |
| LiOH | Lithium hydroxide |
| MeOH | Methanol |
| MeCN | Acetonitrile |
| MsCl | Methanesulfonic chloride |
| MsOH | Methanesulfonic acid |
| MTBE | Methyl tert-butyl ether |
| NaHMSD | Sodium bis(trimethylsilyl)amide |
| NBS | N-Bromosuccinimide |
| NCS | N-Chlorosuccinimide |
| NMM | 4-Methylmorpholine |
| NMP | N-Methyl-2-pyrrolidone |
| PhMe | Toluene |
| PPh₃ | Triphenylphosphine |
| ⁱPr₃SiH | Triisopropylsilane |
| psi | Pound-force per square inch |
| PTSA | p-Toluenesulfonic acid |
| PTSCl | p-Toluenesulfonyl chloride |
| TBAF | Tetrabutylammonium fluoride |
| TBHP | tert-Butyl hydroperoxide |
| TBSCl | tert-Butyldimethylsilyl chloride |
| Tf | Triflic |
| Tf₂O | Trifluoromethanesulfonic anhydride |
| TFA | Trifluoroacetic acid |
| TFAA | Trifluoroacetic anhydride |
| THF | Tetrahydrofuran |
| TMSCl | Trimethylsilyl chloride |
| TMSOTf | Trimethylsilyl trifluoromethanesulfonate |
| RT | Ambient temperature |

The compounds prepared in the following Examples were analyzed by LC/MS according to one of the following methods.

| LC/MS | |
|---|---|
| Method | Method Description |
| A | Waters ™ BEH C8 1.7 μm 2.1 × 50 mm, 60 to 95% |
| | MeCN in H₂O (0.1% TFA), 45° C., |
| | Flow rate 1.0 mL/min over 4.0 min |
| B | Waters ™ CSH C₁₈ 1.7 μm 2.1 × 50 mm, 5 to 95% |
| | MeCN in H2O (0.1% TFA), 60° C., |
| | Flow rate 1.25 mL/min over 2.25 min |
| C | Waters ™ CSH C18 1.7 μm 2.1 × 50 mm, 5 to 95% |
| | MeCN in H₂O (0.1% TFA), 45° C., |
| | Flow rate 1.0 mL/min over 4 min |

### Example 1

### N-((5a,8-trans)-(10aR,11S)-3-chloro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1,1,1-trifluoromethanesulfonamide (1)

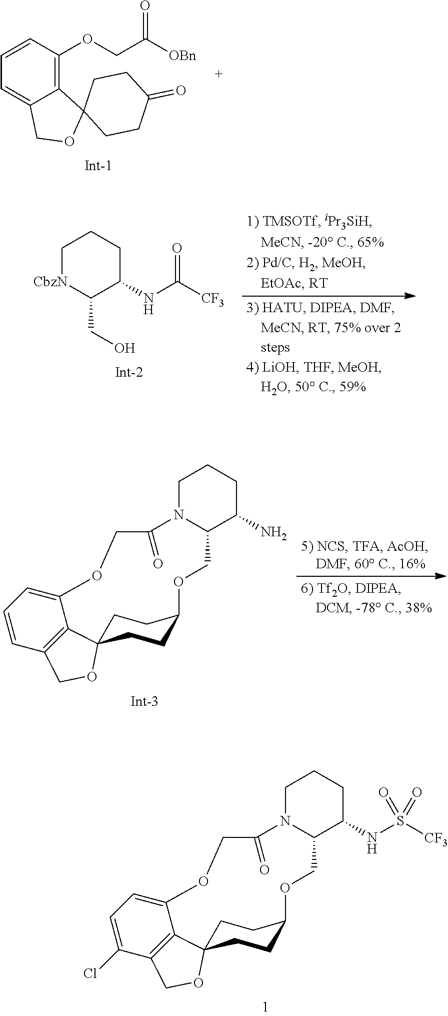

### Step 1:

Under a nitrogen atmosphere, ¹Pr₃SiH (1.5 mL, 7.294 mmol) was added to a stirred solution of benzyl 2-((4-oxo-3′H-spiro[cyclohexane-1,1′-isobenzofuran]-7′-yl)oxy)acetate (Int-1, 2.23 g, 6.086 mmol) and benzyl (2R,3S)-2-(hydroxymethyl)-3-(2,2,2-trifluoroacetamido)piperidine-1-carboxylate (Int-2, 2.7 g, 7.116 mmol) in MeCN (110 mL) at −20° C. A solution of TMSOTf (2.4 mL, 13.283 mmol) in MeCN (20 mL) was added dropwise over 3 min and the reaction mixture was stirred at −20° C. for 30 min. The reaction was quenched by addition of a saturated NaHCO₃ solution, warmed to ambient temperature and extracted with EtOAc (2×). The combined organic layers were washed with brine, dried (Na₂SO₄) and filtered. The filtrates were passed through a pad of silica, washing the pad with EtOAc. The filtrates were collected and concentrated in vacuo. Purification by flash chromatography (40 g SiO₂, 0 to 80% EtOAc in heptane) gave benzyl (2R,3S)-2-((((1,4-trans)-7′-(2-(benzyloxy)-2-oxoethoxy)-3′H-spiro[cyclohexane-1,1′-isobenzofuran]-4-yl)oxy)methyl)-3-(2,2,2-trifluoroacetamido)piperidine-1-carboxylate (2.8 g, 65%) as the predominant geometric isomer and as a yellow oil. ¹H NMR (400 MHz, Chloroform-d) δ 8.65 (s, 1H), 7.47-7.28 (m, 10H), 7.23 (t, J=7.9 Hz, 1H), 6.87 (d, J=7.4 Hz, 1H), 6.62 (d, J=8.1 Hz, 1H), 5.35-5.01 (m, 6H), 4.90-4.80 (m, 1H), 4.78-4.45 (m, 2H), 4.06 (t, J=10.1 Hz, 1H), 3.89-3.70 (m, 1H), 3.69-3.50 (m, 1H), 2.95-2.69 (m, 1H), 2.67-2.20 (m, 2H), 2.18-2.10 (m, 1H), 2.02-1.68 (m, 6H), 1.60-1.43 (m, 2H), 1.40-1.32 (m, 3H) ppm. ESI-MS m/z calc. 710.282, found 711.6 (M+1)⁺.

### Step 2:

A suspension of benzyl (2R,3S)-2-((((1,4-trans)-7′-(2-(benzyloxy)-2-oxoethoxy)-3′H-spiro[cyclohexane-1,1′-isobenzofuran]-4-yl)oxy)methyl)-3-(2,2,2-trifluoroacetamido)piperidine-1-carboxylate (40 mg, 0.025 mmol) and Pd/C (15 mg, 5 wt. % loading (dry basis) Degussa, 0.007 mmol) in a mixture of MeOH (1 mL) and EtOAc (1 mL) was placed under a hydrogen atmosphere and the reaction mixture was stirred at ambient temperature for 2 h. The reaction was filtered through a pad of Celite® and the filtrates were concentrated in vacuo to give 2-(((1,4-trans)-4-(((2R,3S)-3-(2,2,2-trifluoroacetamido)piperidin-2-yl)methoxy)-3′H-spiro[cyclohexane-1,1′-isobenzofuran]-7′-yl)oxy)acetic acid as the predominant geometric isomer, which was used without further purification in the next step.

### Step 3:

A solution of 2-(((1,4-trans)-4-(((2R,3S)-3-(2,2,2-trifluoroacetamido)piperidin-2-yl)methoxy)-3′H-spiro[cyclohexane-1,1′-isobenzofuran]-7′-yl)oxy)acetic acid in DMF (1 mL) was added dropwise over 2 min to a stirred solution of HATU (24 mg, 0.063 mmol) and DIPEA (25 μL, 0.144 mmol) in MeCN (9 mL) and the reaction mixture was stirred for 30 min at ambient temperature. The mixture was concentrated in vacuo. Purification by reverse phase HPLC (XSelect CSH C₁₈ OBD column, 0 to 100% MeCN in water with 0.2% formic acid) gave 2,2,2-trifluoro-N-((5a,8-trans)-(10aR,11S)-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)acetamide (9.4 mg, 75% over 2 steps) as a pale yellow solid. ¹H NMR (400 MHz, Methanol-d₄) δ 7.23 (t, J=7.9 Hz, 1H), 6.89 (d, J=7.5 Hz, 1H), 6.82 (d, J=8.1 Hz, 1H), 5.38 (d, J=11.6 Hz, 1H), 5.30-5.15 (m, 1H), 5.07-4.96 (m, 2H), 4.19-4.06 (m, 2H), 3.98-3.87 (m, 2H), 3.75 (s, 1H), 3.50 (t, J=13.2 Hz, 1H), 3.34 (d, J=3.5 Hz, 1H), 2.96-2.84 (m, 1H), 2.19-2.04 (m, 2H), 1.97-1.54 (m, 9H), 1.38 (d, J=14.0 Hz, 1H) ppm. ESI-MS m/z calc. 468.187, found 469.4 (M+1)⁺.

### Step 4:

LiOH monohydrate (16 mL, 2 M solution in water, 32.0 mmol) was added to a solution of 2,2,2-trifluoro-N-((5a,8-trans)-(10aR,11S)-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)acetamide (1.4 g, 2.988 mmol) in a mixture of THF (40 mL) and MeOH (40 mL) and the reaction mixture was stirred at 50° C. for 5 h. The reaction was cooled to ambient temperature, quenched by addition of a saturated NH₄Cl solution and extracted with EtOAc (2×). The combined organic layers were washed with brine, dried (Na₂SO₄), filtered and concentrated in vacuo to give (5a,8-trans)-(10aR,11S)-11-amino-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (Int-3, 730 mg, 59%) as a yellow solid, which was used without further purification in the next step. ¹H NMR (400 MHz, Methanol-d₄) δ 8.47 (s, 1H), 7.34-7.15 (m, 1H), 6.90 (d, J=7.5 Hz, 1H), 6.83 (d, J=8.0 Hz, 1H), 5.38 (d, J=11.6 Hz, 1H), 5.23 (dt, J=9.8, 4.3 Hz, 1H), 5.14-4.97 (m, 2H), 4.19 (dd, J=11.7, 1.5 Hz, 1H), 3.96 (q, J=10.0 Hz, 2H), 3.86-3.72 (m, 1H), 3.52 (t, J=13.2 Hz, 1H), 3.47-3.35 (m, 2H), 2.97-2.80 (m, 1H), 2.24-2.03 (m, 2H), 2.01-1.89 (m, 2H), 1.83-1.53 (m, 6H), 1.41 (d, J=13.9 Hz, 1H) ppm; exchangeable H not observed. ESI-MS m/z calc. 372.205, found 373.5 (M+1)⁺.

### Step 5:

NCS (40 mg, 0.3 mmol) and TFA (10 L, 0.13 mmol) were successively added to a stirred solution of (5a,8-trans)-(10aR,11S)-11-amino-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (Int-3, 50 mg, 0.121 mmol) in a mixture of DMF (1 mL) and AcOH (0.5 mL). The reaction was placed under a nitrogen atmosphere, stirred at 60° C. for 10 min and cooled to ambient temperature. Purification by reverse phase chromatography (15.5 g C₁₈ column, 0 to 100% MeCN in water with 0.2% formic acid) gave (5a,8-trans)-(10aR,11S)-11-amino-3-chloro-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (8 mg, 16%) as a yellow oil. ¹H NMR (400 MHz, Chloroform-d) δ 7.17 (d, J=8.4 Hz, 1H), 6.69 (d, J=8.5 Hz, 1H), 5.30 (s, 2H), 5.23-4.99 (m, 4H), 4.27 (d, J=11.5 Hz, 1H), 3.93-3.72 (m, 3H), 3.70-3.40 (m, 3H), 2.80-2.58 (m, 1H), 2.24-1.79 (m, 6H), 1.71-1.31 (m, 5H) ppm. ESI-MS m/z calc. 406.166, found 407.5 (M+1)⁺.

### Step 6:

Tf₂O (4 μL, 0.024 mmol) was added to a stirred solution of (5a,8-trans)-(10aR,11S)-11-amino-3-chloro-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (8 mg, 0.020 mmol) and DIPEA (15 L, 0.086 mmol) in DCM (1 mL) at −78° C. and the reaction mixture was stirred at −78° C. for 30 min. The reaction was quenched by addition of a saturated NaHCO₃ solution, warmed to ambient temperature and concentrated in vacuo. Purification by reverse phase HPLC (XSelect CSH C₁₈ OBD column, 0 to 100% MeCN in water with 0.2% formic acid) gave N-((5a,8-trans)-(10aR,11S)-3-chloro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1,1,1-trifluoromethanesulfonamide (1, 4.3 mg, 38%) as a pink solid. ¹H NMR (400 MHz, Methanol-d₄) δ 7.20 (d, J=8.4 Hz, 1H), 6.86 (d, J=8.9 Hz, 1H), 5.36 (d, J=11.4 Hz, 1H), 5.19-5.09 (m, 1H), 5.02 (d, J=7.5 Hz, 2H), 4.16 (d, J=11.9 Hz, 1H), 3.95-3.69 (m, 4H), 3.54-3.39 (m, 2H), 2.89-2.77 (m, 1H), 2.18-1.83 (m, 6H), 1.81-1.59 (m, 6H) ppm. ESI-MS m/z calc. 538.115, found 539.5 (M+1)⁺.

### Example 2

### N-((5a,8-trans)-(10aR,11S)-16-Oxo-7,8,10,10a,11,12,13,14,16,17-Decahydro-4H,6H-5a,8-ethanoisobenzofuro[7,1-hi]pyrido[2,1-c][1,7]dioxa[4]azacyclotridecin-11-yl)methanesulfonamide (2)

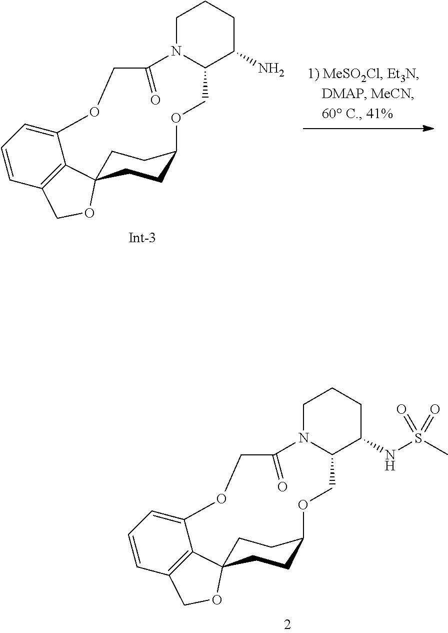

### Step 1:

Et₃N (14.26 mg, 19.64 μL, 0.141 mmol) was added to a stirred solution of (5a,8-trans)-(10aR,11S)-11-amino-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (Int-3, 15 mg, 0.040 mmol), MeSO₂Cl (2.537 mg, 1.714 μL, 0.022 mmol) and DMAP (4.920 mg, 0.040 mmol) in MeCN (1 mL). The reaction mixture was stirred at 60° C. for 15 h and concentrated in vacuo. Purification by reverse phase HPLC (XSelect CSH C₁₈ OBD column, 0 to 100% MeCN in water with 0.2% formic acid) gave N-((5a,8-trans)-(10aR,11S)-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)methanesulfonamide (2, 8 mg, 41%) as a white solid. ¹H NMR (400 MHz, DMSO-d₆) δ 7.27 (d, J=6.8 Hz, 1H), 7.24-7.19 (m, 1H), 6.89 (d, J=7.5 Hz, 1H), 6.80 (d, J=7.9 Hz, 1H), 5.35 (d, J=11.4 Hz, 1H), 4.98-4.88 (m, 3H), 4.02 (d, J=11.4 Hz, 1H), 3.84-3.67 (m, 3H), 3.48-3.43 (m, 2H), 2.96 (s, 3H), 2.80-2.71 (m, 1H), 2.07-1.99 (m, 2H), 1.80-1.71 (m, 2H), 1.63-1.56 (m, 4H), 1.55-1.41 (m, 3H), 1.31-1.20 (m, 1H) ppm. ESI-MS m/z calc. 450.182, found 451.5 (M+1)⁺.

### Example 3

### 1,1-Difluoro-N-((5a,8-trans)-(10aR,11S)-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)methanesulfonamide (3)

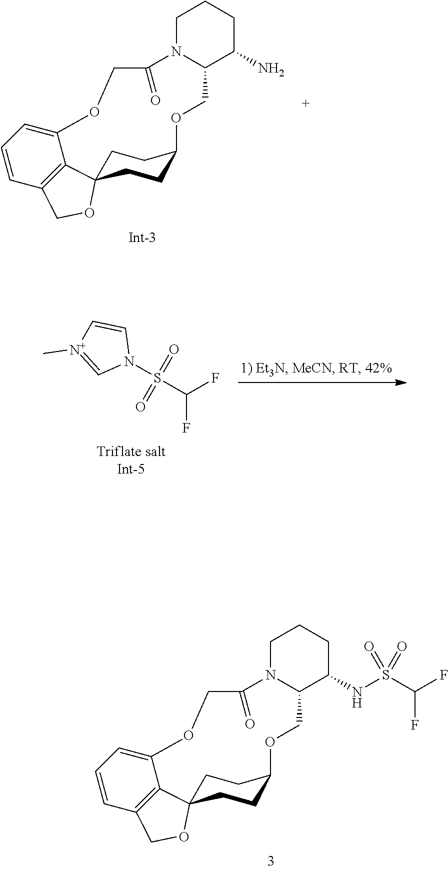

### Step 1:

1-((Difluoromethyl)sulfonyl)-3-methyl-1H-imidazol-3-ium triflate (Int-5, 20 mg, 0.064 mmol) was added to a stirred solution of (5a,8-trans)-(10aR,11S)-11-amino-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (Int-3, 20 mg, 0.048 mmol) and Et₃N (20 μL, 0.144 mmol) in MeCN (1 mL). The reaction was stirred at ambient temperature for 18 h and concentrated in vacuo. Purification by reverse phase HPLC (XSelect CSH C₁₈ OBD column, 0 to 100% MeCN in water with 0.2% formic acid) gave 1,1-difluoro-N-((5a,8-trans)-(10aR,11S)-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)methanesulfonamide (3, 11.5 mg, 42%) as a white solid. ¹H NMR (400 MHz, DMSO-d₆) δ 8.74 (d, J=8.1 Hz, 1H), 7.22 (t, J=7.7 Hz, 1H), 6.89 (d, J=7.6 Hz, 1H), 6.80 (d, J=7.9 Hz, 1H), 5.34 (d, J=11.4 Hz, 1H), 5.02-4.86 (m, 3H), 4.02 (d, J=11.4 Hz, 1H), 3.86-3.66 (m, 4H), 3.34-3.23 (m, 2H), 2.82-2.72 (m, 1H), 2.11-1.94 (m, 2H), 1.88-1.69 (m, 4H), 1.52 (dd, J=35.9, 19.5 Hz, 5H), 1.26 (d, J=13.6 Hz, 1H) ppm. ESI-MS m/z calc. 486.164, found 487.5 (M+1)⁺.

### Example 4

### 1,1,1-Trifluoro-N-((5a,8-cis)-(10aR,11S)-16-oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)methanesulfonamide (4)

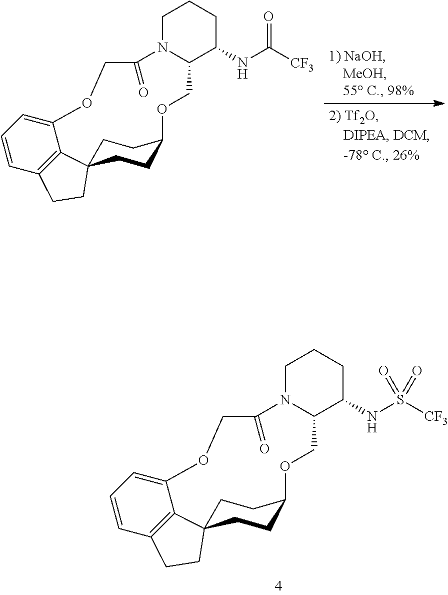

### Step 1:

NaOH (120 mL, 1 M aqueous solution, 120 mmol) was added to a slurry of 2,2,2-trifluoro-N-((5a,8-cis)-(10aR,11S)-16-oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)acetamide (7.68 g, prepared using the method described in Example 1 Steps 1 to 3 from Int-24 and Int-2, 16.08 mmol) in MeOH (110 mL) and the reaction mixture was heated to 55° C. for 2 h. The reaction was concentrated to half of its volume. The mixture was diluted with water (100 mL) and extracted with DCM (2×100 mL). The combined organic phases were washed with brine, dried (Na₂SO₄), and concentrated in vacuo to give ((5a,8-cis)-(10aR,11S)-11-amino-4,5,7,8,10,10a,11,12,13,14-decahydro-6H-5a,8-ethanoindeno[7,1-hi]pyrido[2,1-c][1,7]dioxa[4]azacyclotridecin-16(17H)-one (5.9 g, 98%) as rotamers and as a white foam. ¹H NMR (predominant rotamer)(300 MHz, DMSO-d₆) δ 7.08 (t, J=7.7 Hz, 1H), 6.80 (d, J=7.4 Hz, 1H), 6.65 (d, J=7.9 Hz, 1H), 5.29 (d, J=11.3 Hz, 1H), 4.81 (dd, J=11.1, 5.5 Hz, 1H), 3.95 (d, J=11.3 Hz, 1H), 3.84-3.56 (m, 3H), 3.48 (dd, J=9.1, 3.5 Hz, 1H), 3.35-3.20 (m, 1H), 3.01 (td, J=13.7, 4.2 Hz, 1H), 2.93-2.78 (m, 3H), 2.16 (dt, J=11.9, 5.6 Hz, 1H), 2.09-1.96 (m, 2H), 1.77-1.21 (m, 10H), 1.12 (d, J=13.1 Hz, 1H), 0.97 (d, J=13.2 Hz, 1H) ppm. ESI-MS m/z calc. 370.226, found 371.3 (M+1)⁺.

### Step 2:

Tf₂O (40 μL, 0.238 mmol) was added to a stirred solution of (5a,8-cis)-(10aR,11S)-11-amino-4,5,7,8,10,10a,11,12,13,14-decahydro-6H-5a,8-ethanoindeno[7,1-hi]pyrido[2,1-c][1,7]dioxa[4]azacyclotridecin-16(17H)-one (70 mg, 0.189 mmol) and DIPEA (144 μL, 0.827 mmol) in DCM (8.7 mL) at −78° C. and the reaction mixture was stirred at −78° C. for 30 min. The reaction was quenched by addition of a saturated aqueous NaHCO₃ solution and warmed to ambient temperature. The organic layer was separated and concentrated in vacuo. Purification by reverse phase chromatography (C₁₈ column, MeCN in Water with 0.2% formic acid) gave 1,1,1-trifluoro-N-((5a,8-cis)-(10aR,11S)-16-oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)methanesulfonamide (4, 25.3 mg, 26%) as a white solid. ¹H NMR (300 MHz, Methanol-d₄) δ 7.08 (t, J=7.7 Hz, 1H), 6.82 (dd, J=7.5, 0.9 Hz, 1H), 6.67 (dd, J=8.0, 0.9 Hz, 1H), 5.31 (d, J=11.6 Hz, 1H), 5.20-5.12 (m, 1H), 4.14 (d, J=11.6 Hz, 1H), 3.97-3.85 (m, 2H), 3.76-3.66 (m, 2H), 3.55-3.44 (m, 2H), 3.16-3.05 (m, 1H), 2.88 (dd, J=9.5, 5.6 Hz, 2H), 2.30-2.21 (m, 1H), 2.13-2.04 (m, 2H), 1.95-1.84 (m, 2H), 1.83-1.71 (m, 3H), 1.69-1.34 (m, 3H), 1.24 (d, J=13.3 Hz, 1H), 1.11 (d, J=13.0 Hz, 1H) ppm; exchangeable H not observed. ESI-MS m/z calc. 502.175, found 503.2 (M+1)⁺.

### Example 5

### dia-1,1,1-Trifluoro-N-((5a,8-cis)-(4S,10aR*,11S*)-4-hydroxy-16-oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)methanesulfonamide (5) and dia-1,1,1-trifluoro-N-((5a,8-cis)-(4S,10aR*,11S*)-4-fluoro-16-oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)methanesulfonamide (6)

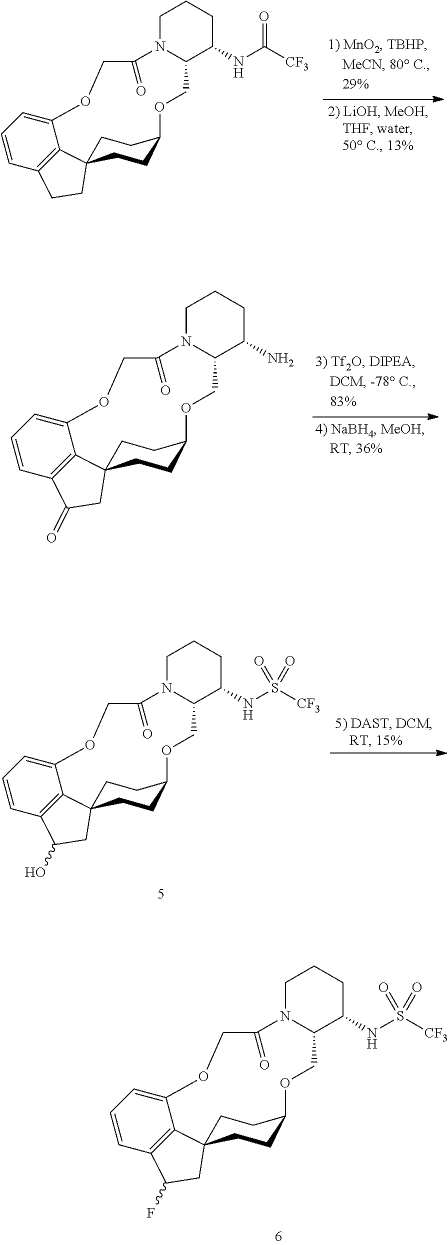

### Step 1:

MnO₂ (580 μL, 33.53 mmol) was added to a stirred solution of TBHP (1.8 mL, 5.5 M solution in decane, 9.9 mmol) and 2,2,2-trifluoro-N-((5a,8-cis)-(10aR,11S)-16-oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)acetamide (1.556 g, prepared using the method described in Example 1 Steps 1 to 3 from Int-24 and Int-2, 3.224 mmol) in MeCN (10 mL) and the reaction mixture was stirred at 80° C. for 5 h. The mixture was filtered and the filtrates were added to water. The aqueous layer was extracted with EtOAc (x 3). The combined organic phases were washed with brine, dried (MgSO₄), filtered and concentrated in vacuo to give N-((5a,8-cis)-(10aR,11S)-4,16-dioxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)-2,2,2-trifluoroacetamide (1.3 g, 29%) as a yellow oil. ESI-MS m/z calc. 480.187, found 481.3 (M+1)⁺.

### Step 2:

LiOH (608 μL, 2 M aqueous solution, 1.216 mmol) was added to a stirred solution of N-((5a,8-cis)-(10aR,11S)-4,16-dioxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)-2,2,2-trifluoroacetamide (500 mg, 0.608 mmol) in a mixture of MeOH (2.1 mL) and THF (2.1 mL) and the reaction mixture was stirred at 50° C. overnight. The reaction was concentrated in vacuo. The residue was dissolved in a minimum of water, and a 1 M aqueous HCl solution was added dropwise to the solution. The mixture was extracted with DCM. The organic layer was separated and dried by passing the solution through a phase separation cartridge. The liquors were concentrated in vacuo to give (5a,8-cis)-(10aR,11S)-11-amino-7,8,10,10a,11,12,13,14-octahydro-6H-5a,8-ethanoindeno[7,1-hi]pyrido[2,1-c][1,7]dioxa[4]azacyclotridecine-4,16(5H,17H)-dione (60 mg, 13%). ESI-MS m/z calc. 384.205, found 385.3 (M+1)⁺.

### Step 3:

Tf₂O (126 μL, 0.749 mmol) was added to a stirred solution of (5a,8-cis)-(10aR,11S)-11-amino-7,8,10,10a,11,12,13,14-octahydro-6H-5a,8-ethanoindeno[7,1-hi]pyrido[2,1-c][1,7]dioxa[4]azacyclotridecine-4,16(5H,17H)-dione (233 mg, 0.606 mmol) and DIPEA (463 μL, 2.658 mmol) in DCM (29 mL) and the reaction mixture was stirred at −78° C. for 30 min. The reaction was quenched by addition of a saturated aqueous NaHCO₃ solution and warmed to ambient temperature. The organic layer was separated and dried by passing the solution through a phase separation cartridge. The liquors were concentrated in vacuo to give N-((5a,8-cis)-(10aR,11S)-4,16-dioxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)-1,1,1-trifluoromethanesulfonamide (442 mg, 83%) as a yellow solid. ESI-MS m/z calc. 516.154, found 517.2 (M+1)⁺.

### Step 4:

NaBH₄ (5.5 mg, 0.145 mmol) was added to a stirred solution of N-((5a,8-cis)-(10aR,11S)-4,16-dioxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)-1,1,1-trifluoromethanesulfonamide (32 mg, 0.048 mmol) in MeOH (482 μL) and the reaction mixture was stirred at ambient temperature for 15 min. Three successive additional amounts of NaBH₄ were added (3×5.5 mg, 0.145 mmol) and the reaction mixture was stirred at ambient temperature for 15 min after each addition. The mixture was concentrated in vacuo. The residue was partitioned between brine (2 mL) and DCM (2 mL). The aqueous phase was separated and extracted with DCM (2×2 mL). Purification of the aqueous layer by flash chromatography (C₁₈ column, 10 to 100% MeCN in water with formic acid) gave dia-1,1,1-trifluoro-N-((5a,8-cis)-(4S,10aR*,11S*)-4-hydroxy-16-oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)methanesulfonamide (5, 9.2 mg, 36%). ESI-MS m/z calc. 518.170, found 519.3 (M+1)⁺.

### Step 5:

A solution of DAST (2.3 mg, 0.014 mmol) in DCM (189 μL) was added dropwise to a stirred solution of dia-1,1,1-trifluoro-N-((5a,8-cis)-(4S,10aR*,11S*)-4-hydroxy-16-oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)methanesulfonamide (5 mg, 0.009 mmol) in DCM (189 μL) and the reaction mixture was stirred at ambient temperature. Purification by reversed phase chromatography (C₁₈ column, MeCN in water with formic acid) gave dia-1,1,1-trifluoro-N-((5a,8-cis)-(4S,10aR*,11S*)-4-fluoro-16-oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)methanesulfonamide (6, 0.8 mg, 15%) as a white solid. ESI-MS m/z calc. 520.166, found 521.4 (M+1)⁺.

### Example 6

### 1,1,1-Trifluoro-N-((5a,8-trans)-(10aR,11S)-1-fluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)methanesulfonamide (7)

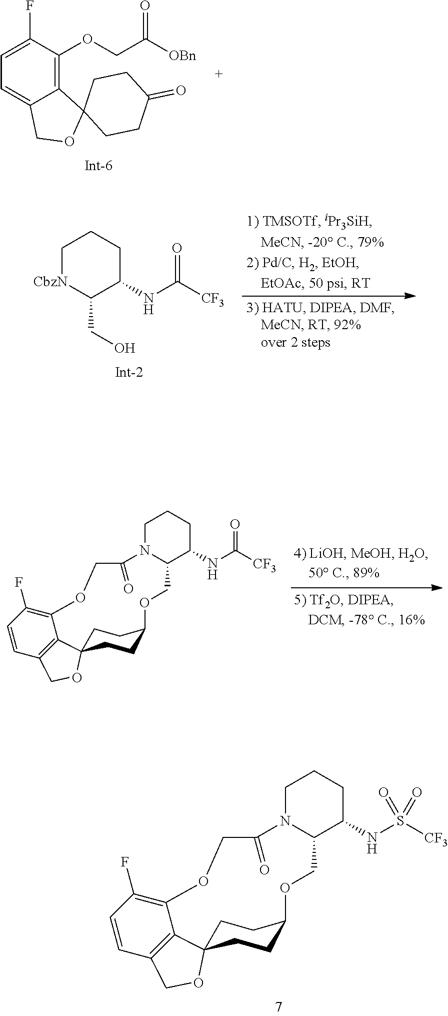

### Step 1:

A solution of TMSOTf (2.1 mL, 11.62 mmol) in MeCN (15 mL) was added over 15 min to a stirred suspension of benzyl (2R,3S)-2-(hydroxymethyl)-3-(2,2,2-trifluoroacetamido)piperidine-1-carboxylate (Int-2, 2 g, 5.550 mmol), benzyl 2-((6′-fluoro-4-oxo-3′H-spiro[cyclohexane-1,1′-isobenzofuran]-7′-yl)oxy)acetate (Int-6, 2 g, 4.943 mmol) and ⁱPr₃SiH (2.6 mL, 11.49 mmol) in MeCN (60 mL) at −20° C. and the reaction mixture was stirred at −20° C. overnight. The mixture was partitioned between DCM (50 mL) and a saturated aqueous NaHCO₃ solution (50 mL). The aqueous layer was extracted with DCM (2×50 mL) and the combined organic layers were concentrated in vacuo. Purification by flash chromatography (220 g SiO₂, 0 to 30% MTBE in DCM) gave benzyl (2R,3S)-2-((((1,4-trans)-7′-(2-(benzyloxy)-2-oxoethoxy)-6′-fluoro-3′H-spiro[cyclohexane-1,1′-isobenzofuran]-4-yl)oxy)methyl)-3-(2,2,2-trifluoroacetamido)piperidine-1-carboxylate (3 g, 79%) as the predominant geometric isomer and as a colourless oil. ESI-MS m/z calc. 728.272, found 729.6 (M+1)⁺.

### Step 2:

Benzyl (2R,3S)-2-((((1,4-trans)-7′-(2-(benzyloxy)-2-oxoethoxy)-6′-fluoro-3′H-spiro[cyclohexane-1,1′-isobenzofuran]-4-yl)oxy)methyl)-3-(2,2,2-trifluoroacetamido)piperidine-1-carboxylate was added to a suspension of Pd (200 mg, 10% w/w on carbon, 0.188 mmol) in a mixture of EtOH (5 mL) and EtOAc (5 mL) and the reaction mixture was stirred under 50 psi of hydrogen for 1 h. The mixture was filtered through a pad of Celite® and concentrated in vacuo to give 2-(((1,4-trans)-6′-fluoro-4-(((2R,3S)-3-(2,2,2-trifluoroacetamido)piperidin-2-yl)methoxy)-3′H-spiro[cyclohexane-1,1′-(5a,8-trans)-(isobenzofuran]-7′-yl)oxy)acetic acid as the predominant geometric isomer, which was used without further purification in the next step.

### Step 3:

A solution of the residue of 2-(((1,4-trans)-6′-fluoro-4-(((2R,3S)-3-(2,2,2-trifluoroacetamido)piperidin-2-yl)methoxy)-3′H-spiro[cyclohexane-1,1′-isobenzofuran]-7′-yl)oxy)acetic acid and DIPEA (1 mL, 5.741 mmol) in DMF (200 mL) was added to a solution of HATU (1.5 g, 3.945 mmol) in MeCN (1 L) at ambient temperature over 3 h and the reaction mixture was stirred at ambient temperature over 3 days. The mixture was concentrated in vacuo. The residue was partitioned between DCM (200 mL) and water (200 mL). The aqueous layer was separated and extracted with DCM (200 mL). The combined organic layers were concentrated in vacuo. Purification by reverse phase chromatography (130 g C₁₈ column, MeCN in water with 0.2% formic acid) gave 2,2,2-trifluoro-N-((5a,8-trans)-(10aR,11S)-1-fluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)acetamide (614 mg, 92% over 2 steps) as a yellow solid. ¹H NMR (300 MHz, Chloroform-d) δ 7.19 (d, J=6.7 Hz, 1H), 7.02 (dd, J=11.1, 8.2 Hz, 1H), 6.84 (dd, J=8.2, 3.9 Hz, 1H), 5.37 (dd, J=13.0, 2.2 Hz, 1H), 5.03 (t, J=5.1 Hz, 3H), 4.40-4.21 (m, 2H), 4.17-3.97 (m, 1H), 3.95-3.75 (m, 2H), 3.78-3.46 (m, 2H), 2.87-2.70 (m, 1H), 2.12-1.72 (m, 9H), 1.55 (d, J=14.5 Hz, 2H) ppm. ESI-MS m/z calc. 486.178, found 487.4 (M+1)⁺.

### Step 4:

LiOH (200 mg, 8.351 mmol) was added to a suspension of 2,2,2-trifluoro-N-(5a,8-trans)-(10aR,11S)-1-fluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)acetamide (600 mg, 1.110 mmol) in a mixture of MeOH (5 mL) and water (1 mL) and the reaction mixture was heated at 50° C. for 2 h. Additional LiOH (50 mg, 2.088 mmol) was added, and the mixture was stirred at 50° C. for a further 30 min. The reaction was quenched by addition of formic acid (300 μL, 7.952 mmol) and concentrated in vacuo. Purification by reverse phase chromatography (130 g C₁₈ column, MeCN in water with 0.2% formic acid) followed by flash chromatography (40 g SiO₂, 0 to 10% MeOH in DCM with 2% NH₄OH) gave (5a,8-trans)-(10aR,11S)-11-amino−1-fluoro-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (365.4 mg, 89%) as a colourless solid. ¹H NMR (300 MHz, Methanol-d₄) δ 7.06 (dd, J=11.3, 8.2 Hz, 1H), 6.97-6.86 (m, 1H), 5.44 (dd, J=12.9, 2.2 Hz, 1H), 5.06 (dt, J=9.8, 4.2 Hz, 1H), 5.00-4.89 (m, 2H), 4.18 (dd, J=12.8, 2.0 Hz, 1H), 4.01-3.81 (m, 2H), 3.76 (s, 1H), 3.63-3.39 (m, 2H), 3.08-2.95 (m, 1H), 2.90-2.72 (m, 1H), 2.09 (dt, J=13.7, 9.0 Hz, 2H), 1.94-1.77 (m, 2H), 1.77-1.24 (m, 7H) ppm; exchangeable H not observed. ESI-MS m/z calc. 390.196, found 391.4 (M+1)⁺.

### Step 5:

A solution of Tf₂O (60 μL, 0.357 mmol) in DCM (1 mL) was added dropwise at −78° C. to a suspension of (5a,8-trans)-(10aR,11S)-11-amino-1-fluoro-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one and DIPEA (180 μL, 1.033 mmol) in DCM (5 mL) and the reaction mixture was stirred at −78° C. for 30 min. The reaction was quenched by addition of water (5 mL). The organic layer was separated and concentrated in vacuo. Purification by reverse phase chromatography (50 g C₁₈ column, MeCN in water with 0.1% TFA) gave 1,1,1-trifluoro-N-((5a,8-trans)-(10aR,11S)-1-fluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)methanesulfonamide (7, 32.3 mg, 16%) as an off-white solid. ¹H NMR (300 MHz, Methanol-d₄) δ 7.06 (dd, J=11.3, 8.2 Hz, 1H), 6.92 (dd, J=8.2, 4.0 Hz, 1H), 5.46 (dd, J=12.9, 2.2 Hz, 1H), 5.23-5.10 (m, 1H), 4.96 (t, J=1.2 Hz, 2H), 4.19 (dd, J=12.9, 2.0 Hz, 1H), 4.00-3.84 (m, 2H), 3.86-3.62 (m, 2H), 3.58-3.37 (m, 2H), 2.80 (td, J=14.1, 5.1 Hz, 1H), 2.03 (s, 2H), 1.98-1.82 (m, 2H), 1.82-1.55 (m, 6H), 1.48 (d, J=14.4 Hz, 1H) ppm; exchangeable H not observed. ESI-MS m/z calc. 522.145, found 523.4 (M+1)⁺.

### Example 7

### N-((5a,8-trans)-(10aR,11S)-3-Chloro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1,1-difluoroethane-1-sulfonamide (8)

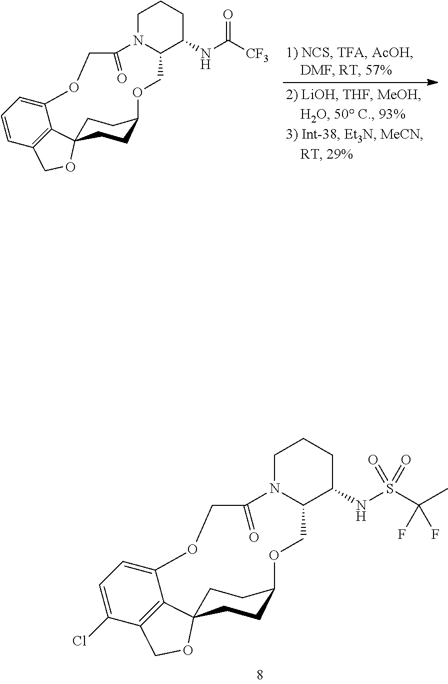

### Step 1:

NCS (35 mg, 0.262 mmol) and TFA (20 μL, 0.260 mmol) were successively added to a solution of 2,2,2-trifluoro-N-((5a,8-trans)-(10aR,11S)-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)acetamide (100 mg, prepared using the method described in Example 1 Steps 1 to 3, 0.214 mmol) in a mixture of DMF (1 mL) and acetic acid (500 μL) and the reaction mixture was stirred at ambient temperature for 15 h. The reaction was concentrated in vacuo. Purification by flash chromatography (24 g SiO₂, 0 to 90% EtOAc in heptane) gave N-((5a,8-trans)-(10aR,11S)-3-chloro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-2,2,2-trifluoroacetamide (139 mg, 57%) as a yellow solid. ESI-MS m/z calc. 502.148, found 503.3 (M+1)⁺.

### Step 2:

LiOH (4 mL, 2 M aqueous solution, 8 mmol) was added to a solution of N-((5a,8-trans)-(10aR,11S)-3-chloro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-2,2,2-trifluoroacetamide (1 g, 1.183 mmol) in a mixture of THF (15 mL) and MeOH (15 mL) and the reaction mixture was stirred at 50° C. for 15 h. The reaction was cooled to ambient temperature, quenched by addition of a saturated aqueous NH₄Cl solution and extracted with EtOAc (2×). The combined organic layers were washed with brine, dried (Na₂SO₄), filtered and concentrated in vacuo to give (5a,8-trans)-(10aR,11S)-11-amino-3-chloro-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (450 mg, 93%) as a yellow solid, which was used without further purification in the next step. ESI-MS m/z calc. 406.166, found 407.4 (M+1)⁺.

### Step 3:

1-((1,1-Difluoroethyl)sulfonyl)-3-methyl-1H-imidazol-3-ium triflate (Int-38, 110 mg, 0.305 mmol) was added to a stirred solution of (5a,8-trans)-(10aR,11S)-11-amino-3-chloro-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (100 mg, 0.246 mmol) and Et₃N (120 μL, 0.861 mmol) in MeCN (2 mL) and the reaction mixture was stirred at ambient temperature for 15 h. The reaction was neutralised by addition of formic acid and concentrated in vacuo. Purification by reverse phase HPLC (XSelect CSH C₁₈ OBD column, 0 to 100% MeCN in water with 0.2% formic acid) gave N-((5a,8-trans)-(10aR,11S)-3-chloro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1,1-difluoroethane-1-sulfonamide (8, 40.9 mg, 29%) as a white solid. ¹H NMR (400 MHz, DMSO-d₆) δ 8.68 (d, J=8.1 Hz, 1H), 7.28 (d, J=8.5 Hz, 1H), 6.89 (d, J=8.5 Hz, 1H), 5.36 (d, J=11.4 Hz, 1H), 5.04-4.89 (m, 3H), 4.02 (d, J=11.5 Hz, 1H), 3.81 (t, J=10.0 Hz, 1H), 3.73-3.61 (m, 2H), 3.51-3.43 (m, 2H), 3.27 (t, J=13.0 Hz, 1H), 2.73-2.64 (m, 1H), 2.02-1.93 (m, 3H), 1.83-1.67 (m, 4H), 1.64-1.43 (m, 6H), 1.33 (d, J=13.5 Hz, 1H) ppm. ESI-MS m/z calc. 534.140, found 535.5 (M+1)⁺.

### Example 8

### 1,1-Difluoro-N-((6a,9-trans)-(11aR,12S)-17-oxo-4,5,8,9,11,11a,12,13,14,15,17,18-dodecahydro-7H-6a,9-ethanoisochromeno[8,1-hi]pyrido[2,1-c][1,7]dioxa[4]azacyclotridecin-12-yl)ethane-1-sulfonamide (9)

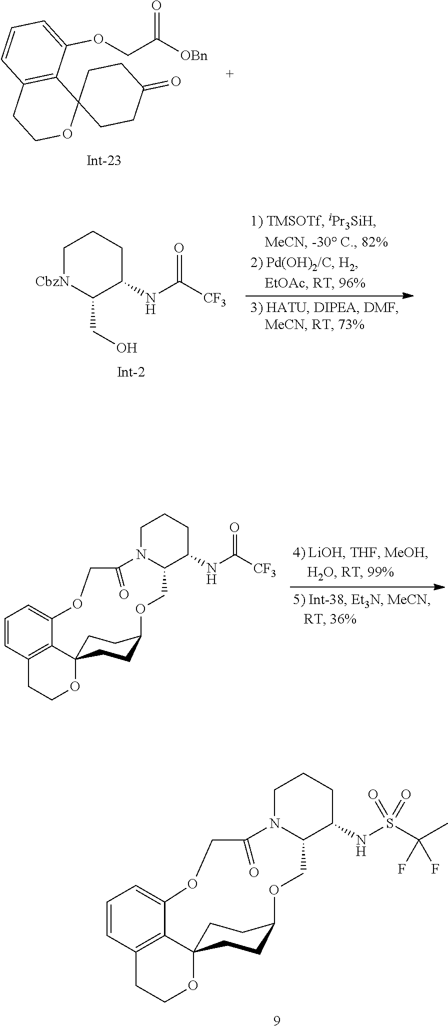

### Step 1:

A solution of TMSOTf (2.33 g, 5.684 mmol) in MeCN (17.6 mL) was added over 2 min to a stirred suspension of benzyl (2R,3S)-2-(hydroxymethyl)-3-(2,2,2-trifluoroacetamido)piperidine-1-carboxylate (Int-2, 2.64 g, 7.253 mmol), benzyl 2-((4-oxospiro[cyclohexane-1,1′-isochroman]-8′-yl)oxy)acetate (Int-23, 2.33 g, 5.684 mmol) and ⁱPr₃SiH (2.5 mL, 12.16 mmol) in MeCN (100 mL) at −30° C. and the reaction mixture was stirred between −25 and −30° C. for 30 min. The reaction was quenched by addition of a saturated aqueous NaHCO₃ solution, warmed to ambient temperature and extracted with DCM (3×). The combined organic layers were dried by passing the solution through a phase separation cartridge and the liquors were concentrated in vacuo. Purification by flash chromatography (SiO₂, 0 to 60% EtOAc in heptane) gave benzyl (2R,3S)-2-((((1,4-trans)-8′-(2-(benzyloxy)-2-oxoethoxy)spiro[cyclohexane-1,1′-isochroman]-4-yl)oxy)methyl)-3-(2,2,2-trifluoroacetamido)piperidine-1-carboxylate (3.51 g, 82%) as the predominant geometric isomer and as a white solid. ¹H NMR (400 MHz, Chloroform-d) δ 8.83 (s, 1H), 7.38-7.28 (m, 7H), 7.26-7.18 (m, 3H), 7.07 (t, J=8.0 Hz, 1H), 6.77 (d, J=7.6 Hz, 1H), 6.54 (d, J=8.2 Hz, 1H), 5.31-4.92 (m, 4H), 4.88-4.57 (m, 2H), 4.56-4.29 (m, 1H), 4.17-4.05 (m, 2H), 3.84 (t, J=5.7 Hz, 2H), 3.78-3.47 (m, 2H), 2.94-2.66 (m, 5H), 2.16-2.06 (m, 1H), 1.91 (dt, J=25.4, 13.9 Hz, 2H), 1.82-1.58 (m, 5H), 1.58-1.56 (m, 2H, overlapped with water), 1.54-1.47 (m, 1H) ppm. ESI-MS m/z calc. 724.297, found 725.6 (M+1)⁺.

### Step 2:

Under a nitrogen atmosphere, a solution of benzyl (2R,3S)-2-((((1,4-trans)-8′-(2-(benzyloxy)-2-oxoethoxy)spiro[cyclohexane-1,1′-isochroman]-4-yl)oxy)methyl)-3-(2,2,2-trifluoroacetamido)piperidine-1-carboxylate (3.51 g, 4.639 mmol) in EtOAc (61 mL) was added to a suspension of Pd(OH)₂/C (650 mg, 0.463 mmol) in EtOAc (61 mL) and the reaction mixture was stirred at ambient temperature under a hydrogen atmosphere for 1 h. The hydrogen atmosphere was replaced by a nitrogen atmosphere and the mixture was filtered through a pad of Celite®, washing the filtered cake with EtOAc. The filtrates were concentrated in vacuo to give 2-(((1,4-trans)-4-(((2R,3S)-3-(2,2,2-trifluoroacetamido)piperidin-2-yl)methoxy)spiro[cyclohexane-1,1′-isochroman]-8′-yl)oxy)acetic acid (2.40 g, 96%) as the predominant geometric isomer and as a white solid. ¹H NMR (400 MHz, Chloroform-d) δ 9.72 (s, 1H), 7.08 (t, J=7.9 Hz, 1H), 6.73 (d, J=7.6 Hz, 1H), 6.67 (d, J=8.2 Hz, 1H), 4.57-4.42 (m, 2H), 4.20 (d, J=13.5 Hz, 1H), 3.89-3.74 (m, 2H), 3.74-3.60 (m, 2H), 3.60-3.45 (m, 2H), 3.43-3.33 (m, 1H), 3.00-2.69 (m, 5H), 2.19-2.05 (m, 1H), 2.03-1.69 (m, 5H), 1.68-1.48 (m, 3H), 1.34-1.27 (m, 1H) ppm; exchangeable H not observed. ESI-MS m/z calc. 500.213, found 501.3 (M+1)⁺.

### Step 3:

2-(((1,4-trans)-4-(((2R,3S)-3-(2,2,2-Trifluoroacetamido)piperidin-2-yl)methoxy)spiro[cyclohexane-1,1′-isochroman]-8′-yl)oxy)acetic acid (2.4 g, 4.439 mmol) was treated via the method described in Example 1 Step 3 to give, after purification by reverse phase chromatography (275 g C₁₈ column, 10 to 100% MeCN in water with 0.2% formic acid), 2,2,2-trifluoro-N-((6a,9-trans)-(11aR,12S)-17-oxo-4,5,8,9,11,11a,12,13,14,15,17,18-dodecahydro-7H-6a,9-ethanoisochromeno[8,1-hi]pyrido[2,1-c][1,7]dioxa[4]azacyclotridecin-12-yl)acetamide (2.36 g, 73%) as a light yellow solid. ¹H NMR (300 MHz, Chloroform-d) δ 7.11 (t, J=7.8 Hz, 1H), 6.81-6.68 (m, 2H), 6.61 (dd, J=8.1, 1.2 Hz, 1H), 5.26-5.16 (m, 1H), 5.09 (d, J=10.2 Hz, 1H), 4.36-4.21 (m, 2H), 3.85 (t, J=5.4 Hz, 2H), 3.80-3.66 (m, 3H), 3.65-3.44 (m, 2H), 2.88-2.69 (m, 3H), 2.50-2.35 (m, 1H), 1.95-1.67 (m, 8H), 1.66-1.43 (m, 2H) ppm. ESI-MS m/z calc. 482.203, found 483.3 (M+1)⁺.

### Step 4:

LiOH (8.3 mL, 2 M aqueous solution, 16.60 mmol) was added to a solution of 2,2,2-trifluoro-N-((6a,9-trans)-(11aR,12S)-17-oxo-4,5,8,9,11,11a,12,13,14,15,17,18-dodecahydro-7H-6a,9-ethanoisochromeno[8,1-hi]pyrido[2,1-c][1,7]dioxa[4]azacyclotridecin-12-yl)acetamide (1.00 g, 1.652 mmol) in a mixture of MeOH (8.3 mL) and THF (25 mL) and the reaction mixture was stirred at ambient temperature overnight. The reaction was partitioned between EtOAc and a saturated aqueous NH₄Cl solution. The aqueous phase was separated and extracted with EtOAc (2×). The combined organic phases were dried (Na₂SO₄), filtered, and concentrated in vacuo. Purification by reverse phase chromatography (50 g C₁₈ column, 10 to 100% MeCN in water with 0.2% formic acid) gave (6a,9-trans)-(11aR,12S)-12-amino-4,5,8,9,11,11a,12,13,14,15-decahydro-7H-6a,9-ethanoisochromeno[8,1-hi]pyrido[2,1-c][1,7]dioxa[4]azacyclotridecin-17(18H)-one (773 mg, 99%) as a white solid. ¹H NMR (300 MHz, Methanol-d₄) δ 7.10 (t, J=7.9 Hz, 1H), 6.77-6.67 (m, 2H), 5.25 (d, J=10.2 Hz, 1H), 5.09 (dt, J=10.4, 4.8 Hz, 1H), 4.06 (d, J=10.3 Hz, 1H), 3.99-3.64 (m, 5H), 3.55-3.34 (m, 2H), 3.11-2.99 (m, 1H), 2.89 (td, J=13.7, 4.9 Hz, 1H), 2.74 (t, J=5.5 Hz, 2H), 2.60 (td, J=13.6, 4.5 Hz, 1H), 2.05-1.92 (m, 1H), 1.87-1.38 (m, 9H) ppm; exchangeable H not observed. ESI-MS m/z calc. 386.221, found 387.4 (M+1)⁺.

### Step 5:

(6a,9-trans)-(11aR,12S)-12-Amino-4,5,8,9,11,11a,12,13,14,15-decahydro-7H-6a,9-ethanoisochromeno[8,1-hi]pyrido[2,1-c][1,7]dioxa[4]azacyclotridecin-17(18H)-one (30 mg, 0.068 mmol) and 1-((1,1-difluoroethyl)sulfonyl)-3-methyl-1H-imidazol-3-ium triflate (Int-38, 49 mg, 0.136 mmol) were treated via the method described in Example 3 Step 1 to give, after purification by reverse phase chromatography (XSelect CSH C₁₈ OBD column, MeCN in water with 0.2% formic acid), 1,1-difluoro-N-((6a,9-trans)-(11aR,12S)-17-oxo-4,5,8,9,11,11a,12,13,14,15,17,18-dodecahydro-7H-6a,9-ethanoisochromeno[8,1-hi]pyrido[2,1-c][1,7]dioxa[4]azacyclotridecin-12-yl)ethane-1-sulfonamide (9, 13.6 mg, 36%) as a white solid. ¹H NMR (300 MHz, Chloroform-d) δ 7.11 (t, J=7.8 Hz, 1H), 6.76 (dd, J=7.6, 1.1 Hz, 1H), 6.60 (d, J=8.3 Hz, 1H), 5.25 (dt, J=9.8, 5.0 Hz, 1H), 5.06 (d, J=10.2 Hz, 1H), 4.92 (d, J=8.4 Hz, 1H), 4.25 (d, J=10.2 Hz, 1H), 3.96-3.76 (m, 4H), 3.73 (s, 1H), 3.69-3.57 (m, 2H), 3.53-3.36 (m, 1H), 2.78 (q, J=5.7 Hz, 2H), 2.54-2.37 (m, 1H), 2.02 (t, J=18.5 Hz, 3H), 1.89-1.46 (m, 11H) ppm. ESI-MS m/z calc. 514.195, found 515.6 (M+1)⁺.

### Example 9

### N-((5a,8-trans)-(10aR,11S)-2,3-Difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1-fluorocyclopropane-1-sulfonamide (10)

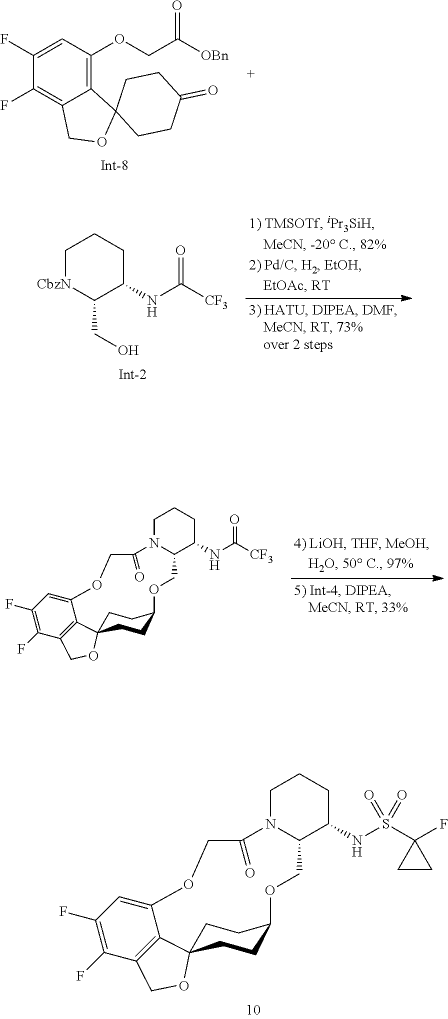

### Step 1:

Under a nitrogen atmosphere, TMSOTf (703 μL, 3.891 mmol) was added dropwise to a stirred mixture of benzyl 2-((4′,5′-difluoro-4-oxo-3′H-spiro[cyclohexane-1,1′-isobenzofuran]-7′-yl)oxy)acetate (Int-8, 760 mg, 1.700 mmol), benzyl (2R,3S)-2-(hydroxymethyl)-3-(2,2,2-trifluoroacetamido)piperidine-1-carboxylate (Int-2, 805 mg, 2.212 mmol) and ⁱPr3SiH (804 μL, 3.909 mmol) in MeCN (34.2 mL) at −20° C. and the reaction mixture was stirred at −20° C. for 30 min. The reaction was quenched by addition of a saturated aqueous NaHCO₃ solution and diluted with water (50 mL). The mixture was warmed to ambient temperature and extracted with EtOAc (2×50 mL). The combined organic layers were washed with brine, dried (Na₂SO₄), filtered by passing the solution through a pad of silica, washing the filtered cake with EtOAc and concentrated in vacuo. Purification by flash chromatography (40 g SiO₂, 0 to 80% EtOAc in heptane) gave benzyl (2R,3S)-2-((((1,4-trans)-7′-(2-(benzyloxy)-2-oxoethoxy)-4′,5′-difluoro-3′H-spiro[cyclohexane-1,1′-isobenzofuran]-4-yl)oxy)methyl)-3-(2,2,2-trifluoroacetamido)piperidine-1-carboxylate (1.040 g, 82%) as the predominant geometric isomer and as a white foam. ¹H NMR (400 MHz, Chloroform-d) δ 8.45 (s, 1H), 7.34-7.21 (m, 10H), 6.33 (dd, J=12.1, 5.3 Hz, 1H), 5.15-4.85 (m, 6H), 4.78-4.21 (m, 3H), 4.10-3.76 (m, 3H), 3.55 (d, J=55.7 Hz, 2H), 2.71 (t, J=13.3 Hz, 1H), 2.36 (dd, J=29.8, 16.3 Hz, 2H), 2.07-1.54 (m, 8H), 1.42 (d, J=14.6 Hz, 2H) ppm.

### Step 2:

A suspension of benzyl (2R,3S)-2-((((1,4-trans)-7′-(2-(benzyloxy)-2-oxoethoxy)-4′,5′-difluoro-3′H-spiro[cyclohexane-1,1′-isobenzofuran]-4-yl)oxy)methyl)-3-(2,2,2-trifluoroacetamido)piperidine-1-carboxylate (1 g, 1.339 mmol) and Pd (143 mg, 10% w/w on carbon, 0.134 mmol) in a mixture of EtOH (10 mL) and EtOAc (10 mL) was placed under a hydrogen atmosphere and stirred at ambient temperature for 6 h. The reaction was filtered through a pad of Celite® and concentrated in vacuo to give 2-(((1,4-trans)-4′,5′-difluoro-4-(((2R,3S)-3-(2,2,2-trifluoroacetamido)piperidin-2-yl)methoxy)-3′H-spiro[cyclohexane-1,1′-isobenzofuran]-7′-yl)oxy)acetic acid as the predominant geometric isomer, which was used without further purification in the next step.

### Step 3:

2-(((1,4-trans)-4′,5′-Difluoro-4-(((2R,3S)-3-(2,2,2-trifluoroacetamido)piperidin-2-yl)methoxy)-3′H-spiro[cyclohexane-1,1′-isobenzofuran]-7′-yl)oxy)acetic acid was treated via the method described in Example 1 Step 3 to give, after purification by reverse phase HPLC (130 g C₁₈ column, 0 to 100% MeCN in water with 0.1% TFA), N-((5a,8-trans)-(10aR,11S)-2,3-difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-2,2,2-trifluoroacetamide (500 mg, 73% over 2 steps) as a yellow solid. ESI-MS m/z calc. 504.168, found 505.5 (M+1)⁺.

### Step 4:

N-((5a,8-trans)-(10aR,11S)-2,3-Difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-2,2,2-trifluoroacetamide (500 mg, 0.991 mmol) was treated via the method described in Example 1 Step 4 to give (5a,8-trans)-(10aR,11S)-11-amino-2,3-difluoro-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (400 mg, 97%) as a colourless foam, which was used without further purification in the next step. ESI-MS m/z calc. 408.186, found 409.5 (M+1)⁺.

### Step 5:

Under a nitrogen atmosphere, 1-((1-fluorocyclopropyl)sulfonyl)-3-methyl-1H-imidazol-3-ium triflate (Int-4, 51.1 mg, 0.144 mmol) was added to a stirred solution of (5a,8-trans)-(10aR,11S)-11-amino-2,3-difluoro-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (30 mg, 0.072 mmol) and DIPEA (24.8 μL, 0.142 mmol) in MeCN (1.5 mL) at ambient temperature and the reaction mixture was stirred at ambient temperature for 30 min. The mixture was diluted with a saturated aqueous NH₄Cl solution (5 mL) and water (5 mL) and extracted with DCM (2×10 mL). The combined organic layers were concentrated in vacuo. Purification by reverse phase HPLC (2×XSelect CSH C₁₈ OBD column, 0 to 100% MeCN in water with 0.2% formic acid) gave N-((5a,8-trans)-(10aR,11S)-2,3-difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1-fluorocyclopropane-1-sulfonamide (10, 12.7 mg, 33%). ¹H NMR (300 MHz, Methanol-d₄) δ 6.99-6.81 (m, 1H), 5.32 (d, J=11.7 Hz, 1H), 5.20-5.03 (m, 3H), 4.15 (d, J=11.7 Hz, 1H), 3.97-3.35 (m, 6H), 2.82 (dd, J=15.5, 4.8 Hz, 1H), 2.19-1.31 (m, 15H) ppm; exchangeable H not observed. ESI-MS m/z calc. 530.170, found 531.5 (M+1)⁺.

### Example 10

### N-((5a,8-trans)-(10aR,11S)-3-Fluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)propane-2-sulfonamide (11)

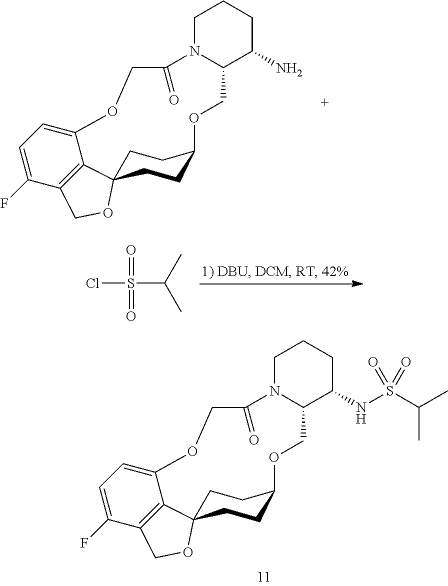

### Step 1:

A solution of propane-2-sulfonyl chloride (42 mg, 0.295 mmol) in DCM (1 mL) was added to a solution of (5a,8-trans)-(10aR,11S)-11-amino-3-fluoro-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (40 mg, prepared using the method described in Example 1 Steps 1 to 4 from Int-7 and Int-2, 0.097 mmol) and DBU (78 μL, 0.522 mmol) in DCM (2 mL) and the reaction mixture was stirred at ambient temperature for 2 h. The reaction was concentrated in vacuo. Purification by reverse phase HPLC (XBridge C₈ column, MeCN in water with 0.2% formic acid) gave N-((5a,8-trans)-(10aR,11S)-3-fluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)propane-2-sulfonamide (11, 22.2 mg, 42%) as a white solid. ¹H NMR (300 MHz, Methanol-d₄) δ 7.03-6.84 (m, 2H), 5.34 (d, J=11.8 Hz, 1H), 5.18-5.03 (m, 3H), 4.14 (d, J=11.8 Hz, 1H), 3.97-3.73 (m, 3H), 3.64-3.41 (m, 3H), 3.27-3.19 (m, 1H), 2.96-2.75 (m, 1H), 2.20-2.01 (m, 2H), 1.94-1.54 (m, 8H), 1.35 (dd, J=6.8, 5.5 Hz, 6H), 1.29 (dd, J=6.8, 3.6 Hz, 1H) ppm; exchangeable H not observed. ESI-MS m/z calc. 496.204, found 497.5 (M+1)⁺.

### Example 11

### rac-N-((5a,8-trans)-(10aR,11R)-12,12-Difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1-fluoromethanesulfonamide (12)

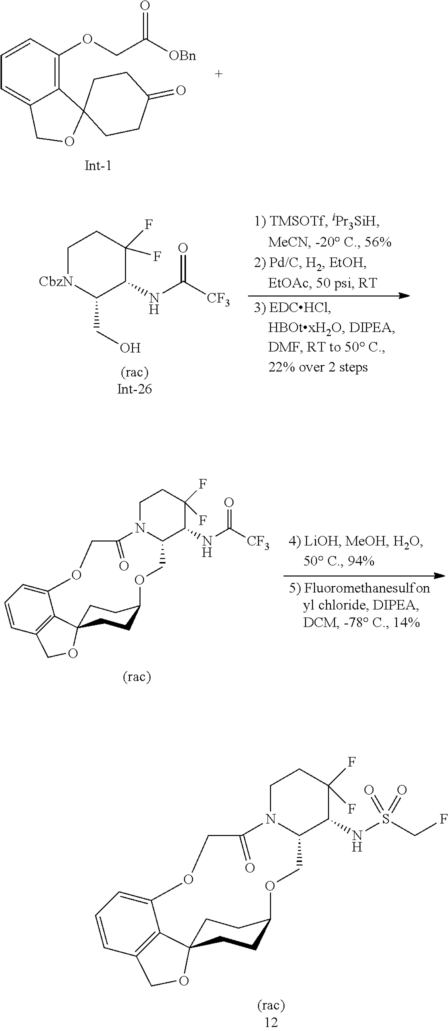

### Step 1:

Benzyl 2-((4-oxo-3′H-spiro[cyclohexane-1,1′-isobenzofuran]-7′-yl)oxy)acetate (Int-1, 400 mg, 1.092 mmol) and benzyl rac-(2R,3R)-4,4-difluoro-2-(hydroxymethyl)-3-(2,2,2-trifluoroacetamido)piperidine-1-carboxylate (Int-26, 400 mg, 1.009 mmol) were treated via the method described in Example 1 Step 1 to give, after purification by flash chromatography (40 g SiO₂, 0 to 30% MTBE in DCM), benzyl rac-(2R,3R)-2-((((1,4-trans)-7′-(2-(benzyloxy)-2-oxoethoxy)-3′H-spiro[cyclohexane-1,1′-isobenzofuran]-4-yl)oxy)methyl)-4,4-difluoro-3-(2,2,2-trifluoroacetamido)piperidine-1-carboxylate (530 mg, 56%) as the predominant geometric isomers and as a colourless oil. ESI-MS m/z calc. 746.263, found 747.5 (M+1)⁺.

### Step 2:

Benzyl rac-(2R,3R)-2-((((1,4-trans)-7′-(2-(benzyloxy)-2-oxoethoxy)-3′H-spiro[cyclohexane-1,1′-isobenzofuran]-4-yl)oxy)methyl)-4,4-difluoro-3-(2,2,2-trifluoroacetamido)piperidine-1-carboxylate (530 mg, 0.568 mmol) was treated via the method described in Example 6 Step 2 to give rac-2-(((1,4-trans)-4-(((2R,3R)-4,4-difluoro-3-(2,2,2-trifluoroacetamido)piperidin-2-yl)methoxy)-3′H-spiro[cyclohexane-1,1′-isobenzofuran]-7′-yl)oxy)acetic acid as the predominant geometric isomers, which were used without further purification in the next step.

### Step 3:

A solution of rac-2-(((1,4-trans)-4-(((2R,3R)-4,4-difluoro-3-(2,2,2-trifluoroacetamido)piperidin-2-yl)methoxy)-3′H-spiro[cyclohexane-1,1′-isobenzofuran]-7′-yl)oxy)acetic acid and DIPEA (700 μL, 4.019 mmol) in DMF (100 mL) was added dropwise over 1 h to a stirred suspension of EDC·HCl (330 mg, 1.721 mmol) and HOBt·xH₂O (230 mg, 1.702 mmol) in DMF (1 L) and the reaction mixture was stirred at ambient temperature for 3 days. Additional EDC·HCl (330 mg, 1.721 mmol), DIPEA (700 μL, 4.019 mmol) and HOBt·xH₂O (230 mg, 1.702 mmol) were added and the mixture was heated to 50° C. for 2 h. The reaction was concentrated in vacuo. Purification by reverse phase HPLC (130 g C₁₈ column, MeCN in water with 0.2% formic acid) gave rac-N-((5a,8-trans)-(10aR,11R)-12,12-difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-2,2,2-trifluoroacetamide (70 mg, 22% over 2 steps) as a pale yellow solid. ESI-MS m/z calc. 504.168, found 505.4 (M+1)⁺.

### Step 4:

rac-N-((5a,8-trans)-(10aR,11R)-12,12-Difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-2,2,2-trifluoroacetamide (70 mg, 0.125 mmol) was treated via the method described in Example 6 Step 4 to give, after purification by flash chromatography (24 g SiO₂, 0 to 10% MeOH in DCM with 2% NH₄OH), rac-(5a,8-trans)-(10aR,11R)-11-amino-12,12-difluoro-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (50 mg, 94%) as a colourless solid. ¹H NMR (300 MHz, Methanol-d₄) δ 7.26 (t, J=7.8 Hz, 1H), 6.97-6.80 (m, 2H), 5.42 (d, J=11.8 Hz, 1H), 5.24 (td, J=7.3, 3.6 Hz, 1H), 5.04 (dt, J=3.0, 0.9 Hz, 2H), 4.21 (d, J=11.8 Hz, 1H), 4.10 (d, J=14.5 Hz, 1H), 3.82 (dt, J=15.4, 4.6 Hz, 2H), 3.73-3.52 (m, 2H), 3.43-3.25 (m, 1H), 2.95-2.78 (m, 1H), 2.29-2.00 (m, 4H), 1.79-1.54 (m, 4H), 1.53-1.32 (m, 1H) ppm; exchangeable H not observed. ESI-MS m/z calc. 408.186, found 409.4 (M+1)⁺.

### Step 5:

rac-(5a,8-trans)-(10aR,11R)-11-Amino-12,12-difluoro-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (10 mg, 0.023 mmol) and fluoromethanesulfonyl chloride (5 mg, 0.038 mmol) were treated via the method described in Example 6 Step 5 to give, after purification by reverse phase HPLC (XSelect CSH C₁₈ OBD column, MeCN in water with 0.1% TFA), rac-N-((5a,8-trans)-(10aR,11R)-12,12-difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1-fluoromethanesulfonamide trifluoroacetate (12, 2 mg, 14%) as a colourless solid. ¹H NMR (300 MHz, Methanol-d₄) δ 7.14 (td, J=7.7, 2.8 Hz, 1H), 6.85-6.66 (m, 2H), 5.35-5.03 (m, 3H), 4.93 (d, J=3.0 Hz, 2H), 4.15-3.90 (m, 3H), 3.85-3.67 (m, 2H), 3.50 (dd, J=9.4, 3.3 Hz, 2H), 2.82-2.66 (m, 1H), 2.29-1.80 (m, 5H), 1.70-1.43 (m, 4H), 1.30 (d, J=14.0 Hz, 1H) ppm; exchangeable H not observed. ESI-MS m/z calc. 504.154, found 505.4 (M+1)⁺.

### Example 12

### N-((5a,8-trans)-(10aR,11S)-16-Oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)propane-1-sulfonamide (13)

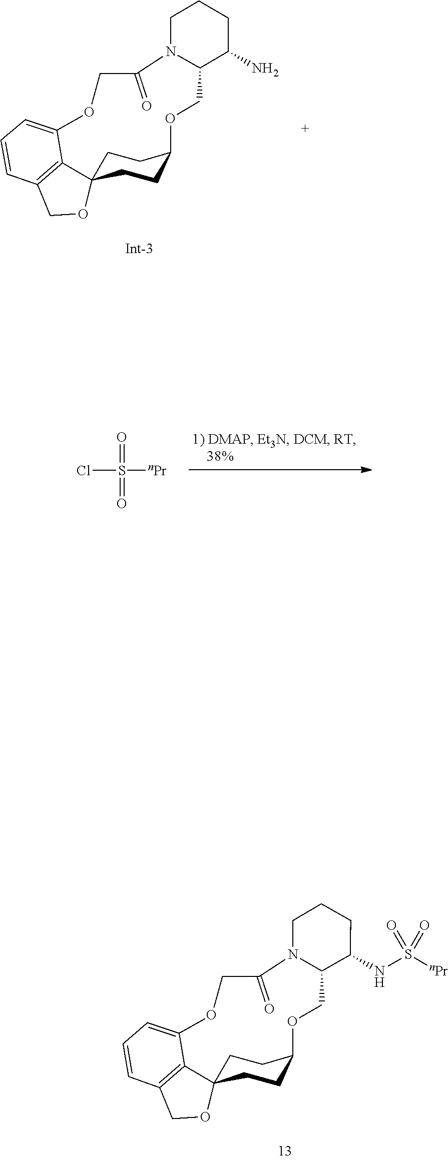

### Step 1:

Propane-1-sulfonyl chloride (38.27 mg, 30.09 μL, 0.268 mmol) was added to a stirred solution of (5a,8-trans)-(10aR,11S)-1l-amino-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (Int-3, 50 mg, 0.134 mmol), Et₃N (40.74 mg, 56.12 μL, 0.403 mmol) and DMAP (16.40 mg, 0.134 mmol) in DCM (1 mL) and the reaction mixture was stirred at ambient temperature for 3 h. The mixture was acidified by addition of formic acid and concentrated in vacuo. Purification by reverse phase HPLC (XSelect CSH C₁₈ OBD column, 0 to 100% MeCN in water with 0.2% formic acid) gave N-((5a,8-trans)-(10aR,11S)-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)propane-1-sulfonamide (13, 25.6 mg, 38%) as a solid. ESI-MS m/z calc. 478.214, found 479.5 (M+1).

### Example 13

### rel-1,1,1-Trifluoro-N-((5a,8-trans)-(4R,10aR*,11S*)-4-methyl-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)methanesulfonamide (14)

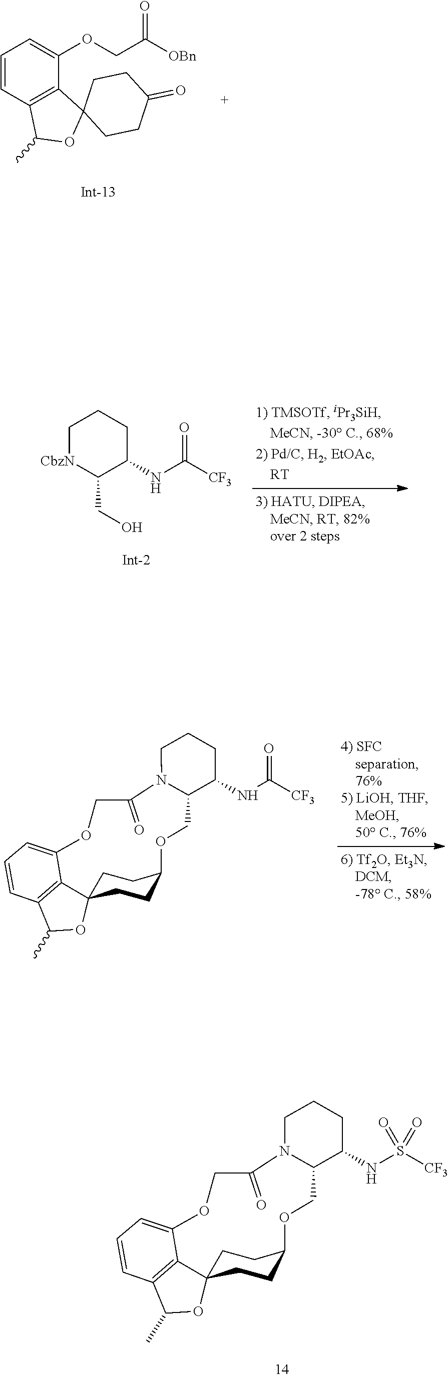

### Step 1:

Benzyl rac-2-((3′-methyl-4-oxo-3′H-spiro[cyclohexane-1,1′-isobenzofuran]-7′-yl)oxy)acetate (Int-13, 2.83 g, 6.734 mmol) and benzyl (2R,3S)-2-(hydroxymethyl)-3-(2,2,2-trifluoroacetamido)piperidine-1-carboxylate (Int-2, 2.74 g, 7.528 mmol) were treated via the method described in Example 8 Step 1 to give, after purification by flash column chromatography (80 g SiO₂, 0 to 100% EtOAc in heptane), benzyl dia-(2R*,3S*)-2-((((1,4-trans)-(3′R)-7′-(2-(benzyloxy)-2-oxoethoxy)-3′-methyl-3′H-spiro[cyclohexane-1,1′-isobenzofuran]-4-yl)oxy)methyl)-3-(2,2,2-trifluoroacetamido)piperidine-1-carboxylate (3.665 g, 68%) as the predominant geometric isomers and as a white foam. ¹H NMR (300 MHz, Chloroform-d) δ 8.51 (s, 1H), 7.29-7.17 (m, 10H), 7.10 (t, J=7.8 Hz, 1H), 6.66 (d, J=7.5 Hz, 1H), 6.48 (d, J=8.1 Hz, 1H), 5.23-4.86 (m, 5H), 4.74-4.65 (m, 1H), 4.48 (s, 3H), 4.13-3.85 (m, 4H), 3.63 (s, 1H), 3.49 (s, 1H), 2.82-2.63 (m, 1H), 2.63-2.48 (m, 1H), 2.41-2.18 (m, 1H), 2.05-1.71 (m, 4H), 1.71-1.55 (m, 3H), 1.43-1.30 (m, 4H) ppm.

### Step 2:

Pd/C (970 mg, 0.912 mmol) was added to a stirred solution of benzyl dia-(2R*,3S*)-2-((((1,4-trans)-(3′R)-7′-(2-(benzyloxy)-2-oxoethoxy)-3′-methyl-3′H-spiro[cyclohexane-1,1′-isobenzofuran]-4-yl)oxy)methyl)-3-(2,2,2-trifluoroacetamido)piperidine-1-carboxylate (3.66 g, 4.545 mmol) in EtOAc (66 mL) and the suspension was stirred under an atmospheric pressure of hydrogen for 4 h. The reaction was filtered through a pad of Celite®, washing the filtered cake with EtOAc. The mother liquors were concentrated in vacuo and the solid was dried under high vacuum to give dia-2-(((1,4-trans)-(3′R)-3′-methyl-4-(((2R*,3S)-3-(2,2,2-trifluoroacetamido)piperidin-2-yl)methoxy)-3′H-spiro[cyclohexane-1,1′-isobenzofuran]-7′-yl)oxy)acetic acid as the predominant geometric isomers and as a light grey solid, which was used without further purification in the next step. ESI-MS m/z calc. 500.213, found 501.4 (M+1)⁺.

### Step 3:

A solution of dia-2-(((1,4-trans)-(3′R)-3′-methyl-4-(((2R*,3S*)-3-(2,2,2-trifluoroacetamido)piperidin-2-yl)methoxy)-3′H-spiro[cyclohexane-1,1′-isobenzofuran]-7′-yl)oxy)acetic acid in MeCN (325 mL) was added dropwise over 3 h to a mixture of HATU (2.6 g, 6.838 mmol) and DIPEA (2.4 mL, 13.78 mmol) in MeCN (3 L) and the reaction mixture was stirred at ambient temperature overnight. The reaction was concentrated in vacuo. Purification by reverse phase chromatography (100 g Cis, 0 to 100% MeCN in water with 0.2% formic acid) followed by flash chromatography (40 g SiO₂, 0 to 100% EtOAc in heptane, then 100% EtOAc:EtOH (3:1)) gave dia-2,2,2-trifluoro-N-((5a,8-trans)-(4R,10aR*,11S*)-4-methyl-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)acetamide (1.86 g, 82% over 2 steps) as an off-white solid. ¹H NMR (300 MHz, Chloroform-d) δ 7.43-7.13 (m, 2H), 6.83-6.69 (m, 2H), 5.40-5.15 (m, 2H), 5.08-4.78 (m, 1H), 4.59-4.21 (m, 2H), 4.06-3.80 (m, 3H), 3.79-3.52 (m, 2H), 2.95-2.57 (m, 1H), 2.27-1.55 (m, 10H), 1.48 (dd, J=8.4, 6.4 Hz, 4H) ppm. ESI-MS m/z calc. 482.203, found 483.4 (M+1)⁺.

### Step 4:

The diastereomers of dia-2,2,2-trifluoro-N-((5a,8-trans)-(4R,10aR*,11S*)-4-methyl-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)acetamide (1.86 g) were separated by chiral SFC using a Lux® Cellulose-2 column, 5 mm particle size, 25 cm×21 mm from Phenomenex (Mobile phase: 12% 3:1 MeCN:MeOH, 88% CO₂; Flow rate 80 mL/min) to give:

First Eluting Isomer: rel-1,1,1-trifluoro-N-((5a,8-trans)-(4R,10aR*,11S*)-4-methyl-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)methanesulfonamide (645.1 mg, 36%) as a white solid. ¹H NMR (300 MHz, Chloroform-d) δ 7.25-7.12 (m, 2H), 6.84-6.68 (m, 2H), 5.29-5.16 (m, 2H), 5.07-4.77 (m, 1H), 4.61-4.22 (m, 2H), 4.09-3.88 (m, 1H), 3.85 (s, 1H), 3.81-3.53 (m, 2H), 2.96-2.58 (m, 1H), 2.04-1.70 (m, 8H), 1.70-1.54 (m, 4H), 1.50 (d, J=6.3 Hz, 3H) ppm. ESI-MS m/z calc. 482.203, found 483.4 (M+1)⁺.

Second Eluting Isomer: rel-1,1,1-trifluoro-N-((5a,8-trans)-(4S,10aR*,11S*)-4-methyl-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)methanesulfonamide (705.1 mg, 40%) as a white solid. ¹H NMR (300 MHz, Chloroform-d) δ 7.23 (s, 1H), 6.99-6.67 (m, 2H), 5.36 (q, J=6.4 Hz, 1H), 5.19 (d, J=11.3 Hz, 1H), 5.06-4.84 (m, 1H), 4.60-4.21 (m, 2H), 4.08-3.76 (m, 3H), 3.73-3.53 (m, 2H), 2.79-2.34 (m, 1H), 2.25-1.55 (m, 11H), 1.48 (dd, J=8.5, 6.4 Hz, 4H) ppm. ESI-MS m/z calc. 482.203, found 483.4 (M+1)⁺.

### Step 5:

LiOH (432.5 mg, 18.06 mmol) was added to a stirred solution of rel-1,1,1-trifluoro-N-((5a,8-trans)-(4R,10aR*, 11S*)-4-methyl-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)methanesulfonamide (First Eluting Isomer from Step 4, 628.5 mg, 1.290 mmol) in a mixture of THF (12 mL) and MeOH (12 mL) and the reaction mixture was heated and stirred at 50° C. overnight. Additional LiOH (150 mg, 6.264 mmol) was added and the reaction was stirred at 50° C. for 2 h. The reaction was quenched by addition of a saturated aqueous NH₄Cl solution and extracted with DCM (3×). The combined organic layers were filtered through a phase separation cartridge and the mother liquors were concentrated in vacuo. Purification by flash column chromatography (40 g SiO₂, 0 to 20% MeOH in DCM with 0.1% NH₄OH) gave rel-(5a,8-trans)-(4R,10aR*,11S*)-11-amino-4-methyl-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (380.3 mg, 76%) as a white solid, which was used without further purification in the next step. ESI-MS m/z calc. 386.221, found 385.4 (M+1)⁺.

### Step 6:

rel-(5a,8-trans)-(4R,10aR*,11S*)-11-Amino-4-methyl-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (25 mg, 0.064 mmol) was treated via the method described in Example 24 Step 6 to give, after purification by reverse phase chromatography (C₁₈ column, 0 to 100% MeCN in water with 0.2% formic acid), rel-1,1,1-trifluoro-N-((5a,8-trans)-(4R,10aR*,11S*)-4-methyl-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)methanesulfonamide (14, 19.8 mg, 58%) as a white solid. ¹H NMR (300 MHz, Chloroform-d) δ 7.22 (t, J=7.8 Hz, 1H), 6.84-6.67 (m, 2H), 6.47-6.10 (m, 1H), 5.35-5.03 (m, 3H), 4.90-4.25 (m, 1H), 4.04-3.65 (m, 5H), 3.54 (t, J=13.1 Hz, 1H), 2.97-2.61 (m, 1H), 2.06-1.56 (m, 10H), 1.55-1.38 (m, 4H) ppm. ESI-MS m/z calc. 518.170, found 519.3 (M+1)⁺.

### Example 14

### rel-2,2,2-Trifluoro-N-((5a,8-trans)-(4R,10aR*,11S*)-4-methyl-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)ethane-1-sulfonamide (15)

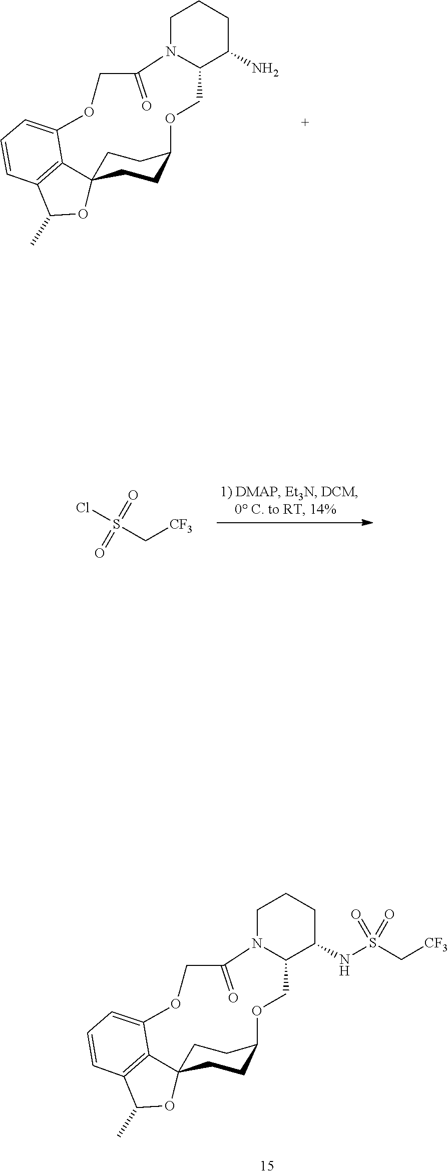

### Step 1:

2,2,2-Trifluoroethane-1-sulfonyl chloride (7.765 mg, 4.703 μL, 0.043 mmol) was added to a stirred solution of rel-(5a,8-trans)-(4R,10aR*,11S*)-11-amino-4-methyl-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (15 mg, prepared using the method described in Example 13 Steps 1 to 5 from Int-13 and Int-2, 0.039 mmol), DMAP (5.197 mg, 0.043 mmol) and Et₃N (11 μL, 0.079 mmol) in DCM (3.3 mL) at 0° C. and the reaction mixture was warmed and stirred at ambient temperature for 2 h. The reaction was concentrated in vacuo. Purification by reverse phase HPLC (XSelect CSH C₁₈ OBD column, MeCN in water with 0.2% formic acid) gave rel-2,2,2-trifluoro-N-((5a,8-trans)-(4R,10aR*,11S)-4-methyl-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)ethane-1-sulfonamide (15, 3.1 mg, 14%). ESI-MS m/z calc. 532.186, found 533.5 (M+1)⁺.

### Example 15

### 1,1,1-Trifluoro-N-((5a,8-trans)-(10aR,11S,13R)-13-methyl-15-oxo-7,8,10a,11,12,13,15,16-octahydro-4H,6H,10H-5a,8-ethanoisobenzofuro[7,1-hi]pyrrolo[2,1-c][1,7]dioxa[4]azacyclotridecin-11-yl)methanesulfonamide (16)

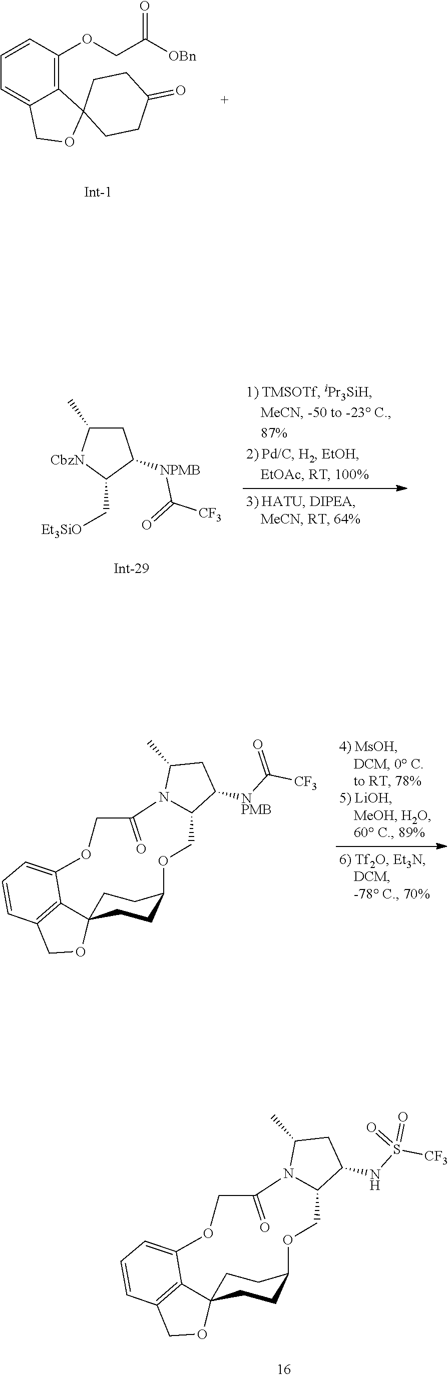

### Step 1:

A solution of TMSOTf (4.1 mL, 22.69 mmol) in MeCN (100 mL) was added over 1.25 h to a stirred solution of benzyl (2R,3S,5R)-5-methyl-2-(((triethylsilyl)oxy)methyl)-3-(2,2,2-trifluoro-N-(4-methoxybenzyl)acetamido)pyrrolidine-1-carboxylate (Int-29, 9.97 g, 15.09 mmol), benzyl 2-((4-oxo-3′H-spiro[cyclohexane-1,1′-isobenzofuran]-7′-yl)oxy)acetate (Int-1, 7 g, 18.82 mmol) and ⁱPr3SiH (7.7 mL, 37.59 mmol) in MeCN (300 mL) at −50° C. and the reaction mixture was stirred for an additional 30 min post addition, during which time, the internal temperature reached −23° C. The reaction was quenched by addition of a saturated aqueous NaHCO₃ solution (200 mL) and concentrated in vacuo to remove most of the MeCN. The mixture was partitioned between MTBE (400 mL) and water (100 mL). The organic layer was separated, washed with a saturated aqueous NaHCO₃ solution (300 mL) and brine, dried (MgSO₄) filtered and concentrated in vacuo. Purification by flash chromatography (330 g SiO₂, 0 to 70% EtOAc in heptane) gave benzyl (2R,3S,5R)-2-((((1,4-trans)-7′-(2-(benzyloxy)-2-oxoethoxy)-3′H-spiro[cyclohexane-1,1′-isobenzofuran]-4-yl)oxy)methyl)-5-methyl-3-(2,2,2-trifluoro-N-(4-methoxybenzyl)acetamido)pyrrolidine-1-carboxylate (10.86 g, 87%) as the predominant geometric isomer and as a clear viscous oil. ESI-MS m/z calc. 830.339, found 831.4 (M+1)⁺.

### Step 2:

Pd (2.12 g, 2.5% w/w on carbon, wet, Degussa, 0.498 mmol) was added to a stirred solution of benzyl (2R,3S,5R)-2-((((1,4-trans)-7′-(2-(benzyloxy)-2-oxoethoxy)-3′H-spiro[cyclohexane-1,1′-isobenzofuran]-4-yl)oxy)methyl)-5-methyl-3-(2,2,2-trifluoro-N-(4-methoxybenzyl)acetamido)pyrrolidine-1-carboxylate (10.85 g, 13.06 mmol) in a mixture of EtOH (165 mL) and EtOAc (165 mL) and the reaction mixture was stirred under an atmospheric pressure of hydrogen at 20° C. for 18 h. The reaction was filtered through a pad of Celite®, washing the filtered cake with MeOH (200 mL). The filtrates were concentrated in vacuo to give 2-(((1,4-trans)-4-(((2R,3S,5R)-5-methyl-3-(2,2,2-trifluoro-N-(4-methoxybenzyl)acetamido)pyrrolidin-2-yl)methoxy)-3′H-spiro[cyclohexane-1,1′-isobenzofuran]-7′-yl)oxy)acetic acid (7.9 g, 100%) as the predominant geometric isomer and as a tan foam. ¹H NMR (300 MHz, Chloroform-d) δ 7.25-7.01 (m, 4H), 6.97-6.83 (m, 2H), 6.84-6.73 (m, 1H), 6.69 (dd, J=8.1, 4.3 Hz, 1H), 5.12 (s, 1H), 5.05 (s, 2H), 4.69 (s, 2H), 4.54-4.28 (m, 3H), 4.19-4.01 (m, 1H), 3.80 (s, 3H), 3.64 (s, 1H), 3.36 (dd, J=10.4, 4.9 Hz, 1H), 2.92-2.47 (m, 3H), 2.33 (dd, J=21.5, 8.2 Hz, 1H), 2.17-1.66 (m, 7H), 1.44 (d, J=6.4 Hz, 4H) ppm. ESI-MS m/z calc. 606.255, found 607.4 (M+1)⁺.

### Step 3:

2-(((1,4-trans)-4-(((2R,3S,5R)-5-Methyl-3-(2,2,2-trifluoro-N-(4-methoxybenzyl)acetamido)pyrrolidin-2-yl)methoxy)-3′H-spiro[cyclohexane-1,1′-isobenzofuran]-7′-yl)oxy)acetic acid (6.35 g, 10.41 mmol) was treated via the method described in Example 13 Step 3 to give, after purification by flash column chromatography (220 g SiO₂, 0 to 100% EtOAc in heptane), 2,2,2-trifluoro-N-(4-methoxybenzyl)-N-((5a,8-trans)-(10aR,11S,13R)-13-methyl-15-oxo-7,8,10a,11,12,13,15,16-octahydro-4H,6H,10H-5a,8-ethanoisobenzofuro[7,1-hi]pyrrolo[2,1-c][1,7]dioxa[4]azacyclotridecin-11-yl)acetamide (3.984 g, 64%) as an off-white solid. ESI-MS m/z calc. 588.245, found 291.9 (M+1)⁺.

### Step 4:

MsOH (6.4 mL, 98.63 mmol) was added to a stirred solution of 2,2,2-trifluoro-N-(4-methoxybenzyl)-N-((5a,8-trans)-(10aR,11S,13R)-13-methyl-15-oxo-7,8,10a,11,12,13,15,16-octahydro-4H,6H,10H-5a,8-ethanoisobenzofuro[7,1-hi]pyrrolo[2,1-c][1,7]dioxa[4]azacyclotridecin-11-yl)acetamide (3.98 g, 6.621 mmol) in DCM (60 mL) at 0° C. and the reaction mixture was warmed and stirred at ambient temperature for 5 min, then at 35° C. for 70 min. The reaction was cooled to 0° C., quenched by dropwise addition of a saturated NaHCO₃ solution (100 mL) and stirred at ambient temperature for 15 min. The aqueous layer was separated and extracted with DCM (2×). The combined organic layers were washed with brine, passed through a phase separation cartridge and the filtrates were concentrated in vacuo. Purification by reverse phase chromatography (275 g C₁₈ column, 0 to 100% MeCN in water with 0.2% formic acid) gave 2,2,2-trifluoro-N-((5a,8-trans)-(10aR,11S,13R)-13-methyl-15-oxo-7,8,10a,11,12,13,15,16-octahydro-4H,6H,10H-5a,8-ethanoisobenzofuro[7,1-hi]pyrrolo[2,1-c][1,7]dioxa[4]azacyclotridecin-11-yl)acetamide (2.478 g, 78%) as a white solid. ¹H NMR (300 MHz, Methanol-d₄) δ 7.25 (t, J=7.8 Hz, 1H), 6.93-6.78 (m, 2H), 5.22 (d, J=10.8 Hz, 1H), 5.10-4.96 (m, 2H), 4.85 (s, 3H), 4.62 (d, J=6.7 Hz, 1H), 4.49 (m, 2H), 4.28 (d, J=10.8 Hz, 1H), 3.94 (s, 1H), 3.42 (d, J=9.8 Hz, 1H), 3.10 (td, J=14.4, 4.9 Hz, 1H), 2.38 (dt, J=14.2, 7.4 Hz, 1H), 2.18-1.27 (m, 9H) ppm; exchangeable H not observed. ESI-MS m/z calc. 468.187, found 469.2 (M+1)⁺.

### Step 5:

A solution of LiOH (1.2 g, 50.11 mmol) in water (12 mL) was added to a stirred solution of 2,2,2-trifluoro-N-((5a,8-trans)-(10aR,11S,13R)-13-methyl-15-oxo-7,8,10a,11,12,13,15,16-octahydro-4H,6H,10H-5a,8-ethanoisobenzofuro[7,1-hi]pyrrolo[2,1-c][1,7]dioxa[4]azacyclotridecin-11-yl)acetamide (2.48 g, 5.193 mmol) in MeOH (36 mL) and the reaction mixture was stirred at 60° C. for 90 min. The reaction was diluted with DCM. The aqueous phase was separated and extracted with DCM (2×). The combined organic layers were washed with brine, passed through a phase separation cartridge. The filtrates were concentrated in vacuo to give (5a,8-trans)-(10aR,11S,13R)-11-amino-13-methyl-7,8,10a,11,12,13-hexahydro-4H,6H,10H-5a,8-ethanoisobenzofuro[7,1-hi]pyrrolo[2,1-c][1,7]dioxa[4]azacyclotridecin-15(16H)-one (1.817 g, 89%) as an off-white solid. ESI-MS m/z calc. 372.205, found 373.2 (M+1)⁺.

### Step 6:

(5a,8-trans)-(10aR,11S,13R)-11-Amino-13-methyl-7,8,10a,11,12,13-hexahydro-4H,6H,10H-5a,8-ethanoisobenzofuro[7,1-hi]pyrrolo[2,1-c][1,7]dioxa[4]azacyclotridecin-15(16H)-one (120 mg, 0.322 mmol) was treated via the method described in Example 24 Step 6 to give, after purification by reverse phase chromatography (C₁₈ column, MeCN in water with 0.2% formic acid), 1,1,1-trifluoro-N-((5a,8-trans)-(10aR,11S,13R)-13-methyl-15-oxo-7,8,10a,11,12,13,15,16-octahydro-4H,6H,10H-5a,8-ethanoisobenzofuro[7,1-hi]pyrrolo[2,1-c][1,7]dioxa[4]azacyclotridecin-11-yl)methanesulfonamide (16, 115 mg, 70%) as a white solid. ¹H NMR (300 MHz, Chloroform-d) δ 7.76 (s, 1H), 7.23 (t, J=7.8 Hz, 1H), 6.86 (dd, J=7.5, 0.9 Hz, 1H), 6.74 (d, J=8.0 Hz, 1H), 5.18-4.89 (m, 3H), 4.52 (td, J=13.4, 6.8 Hz, 2H), 4.34 (dd, J=13.1, 6.9 Hz, 2H), 3.98 (s, 1H), 3.40 (d, J=9.4 Hz, 1H), 2.97 (td, J=13.8, 5.4 Hz, 1H), 2.38-0.52 (m, 13H) ppm. ESI-MS m/z calc. 504.154, found 505.4 (M+1)⁺.

### Example 16

### 1-Methyl-N-((5a,8-trans)-(10a′R,11′S)-16′-oxo-7′,8′,10′,10a′,11′,12′,13′,14′-octahydro-4′H,6′H,16′H-spiro[cyclopropane-1,17′-[5a,8]ethanoisobenzofuro[7,1-hi]pyrido[2,1-c][1,7]dioxa[4]azacyclotridecin]-11′-yl)cyclopropane-1-sulfonamide (17)

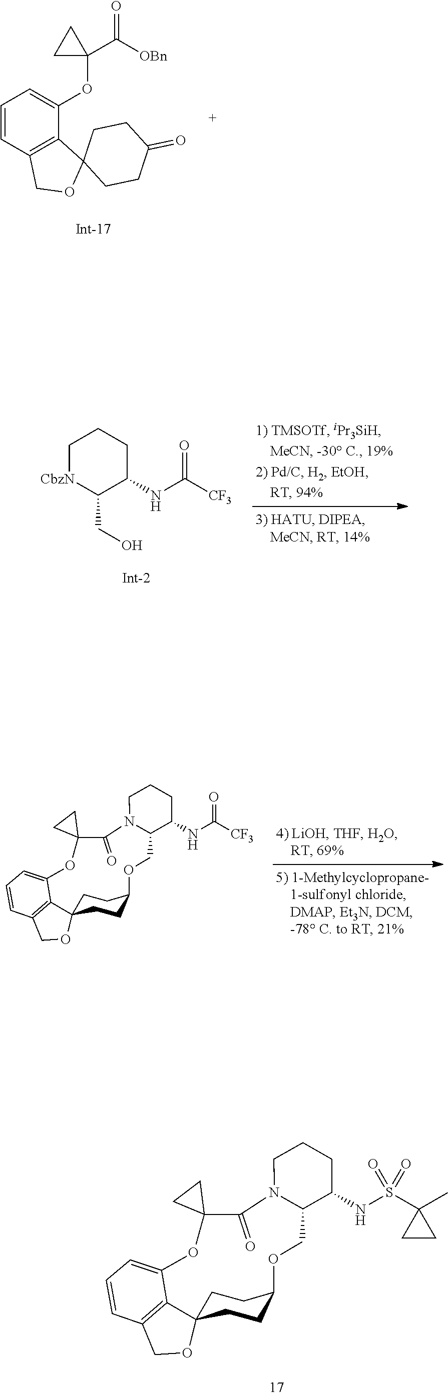

### Step 1:

Benzyl 1-((4-oxo-3′H-spiro[cyclohexane-1,1′-isobenzofuran]-7′-yl)oxy)cyclopropane-1-carboxylate (Int-17, 160 mg, 0.408 mmol) and benzyl (2R,3S)-2-(hydroxymethyl)-3-(2,2,2-trifluoroacetamido)piperidine-1-carboxylate (Int-2, 176 mg, 0.484 mmol) were treated via the method described in Example 8 Step 1 to give, after purification by flash chromatography (SiO₂, 0 to 30% EtOAc in heptane), benzyl (2R,3S)-2-((((1,4-trans)-7′-(1-((benzyloxy)carbonyl)cyclopropoxy)-3′H-spiro[cyclohexane-1,1′-isobenzofuran]-4-yl)oxy)methyl)-3-(2,2,2-trifluoroacetamido)piperidine-1-carboxylate (141.4 mg, 19%) as the predominant geometric isomer and as a white foam. ESI-MS m/z calc. 736.297, found 737.6 (M+1)⁺.

### Step 2:

Pd (20 mg, 10% w/w on carbon, 0.019 mmol) was added to a stirred solution of benzyl (2R,3S)-2-((((1,4-trans)-7′-(1-((benzyloxy)carbonyl)cyclopropoxy)-3′H-spiro[cyclohexane-1,1′-isobenzofuran]-4-yl)oxy)methyl)-3-(2,2,2-trifluoroacetamido)piperidine-1-carboxylate (141.4 mg) in EtOH (4 mL) and the reaction mixture was stirred under an atmospheric pressure of hydrogen at ambient temperature for 2 h. The mixture was filtered through a glass paper and concentrated in vacuo. Purification by reverse phase chromatography (C₁₈ column, 0 to 60% MeCN in water with 0.2% formic acid) gave 1-(((1,4-trans)-4-(((2R,3S)-3-(2,2,2-trifluoroacetamido)piperidin-2-yl)methoxy)-3′H-spiro[cyclohexane-1,1′-isobenzofuran]-7′-yl)oxy)cyclopropane-1-carboxylic acid (87 mg, 94%) as the predominant geometric isomer and as a pale yellow semi-solid. ESI-MS m/z calc. 512.213, found 513.3 (M+1)⁺.

### Step 3:

1-(((1,4-trans)-4-(((2R,3S)-3-(2,2,2-Trifluoroacetamido)piperidin-2-yl)methoxy)-3′H-spiro[cyclohexane-1,1′-isobenzofuran]-7′-yl)oxy)cyclopropane-1-carboxylic acid (87 mg) was treated via the method described in Example 13 Step 3 to give, after purification by reverse phase chromatography (C₁₈ column, 0 to 80% MeCN in water with 0.2% formic acid), 2,2,2-trifluoro-N-((5a′,8′-trans)-(10a′R,11′S)-16′-oxo-7′,8′,10′,10a′,11′,12′,13′,14′-octahydro-4′H,6′H,16′H-spiro[cyclopropane-1,17′-[5a,8]ethanoisobenzofuro[7,1-hi]pyrido[2,1-c][1,7]dioxa[4]azacyclotridecin]-11′-yl)acetamide (6 mg, 14%) as a white solid. ESI-MS m/z calc. 494.203, found 495.4 (M+1)⁺.

### Step 4:

LiOH (3.0 mg, 0.125 mmol) was added to a stirred solution of 2,2,2-trifluoro-N-((5a′,8′-trans)-(10a′R,11′S)-16′-oxo-7′,8′,10′,10a′,11′,12′,13′,14′-octahydro-4′H,6′H,16′H-spiro[cyclopropane-1,17′-[5a,8]ethanoisobenzofuro[7,1-hi]pyrido[2,1-c][1,7]dioxa[4]azacyclotridecin]-11′-yl)acetamide (6 mg) in a mixture of THF (96 μL) and water (32 μL) and the reaction mixture was stirred at ambient temperature for 18 h. The mixture was concentrated in vacuo and the residue was partitioned between water and a 3:1 mixture of CHCl₃ and ⁱPrOH. The aqueous phase was separated and extracted with a 3:1 mixture of CHCl₃ and ⁱPrOH (3×). The combined organic phases were passed through a phase separation cartridge and the filtrates were concentrated in vacuo to give (5a′,8′-trans)-(10a′R,11′S)-11′-amino-7′,8′,10′,10a′,11′,12′,13′,14′-octahydro-4′H,6′H,16′H-spiro[cyclopropane-1,17′-[5a,8]ethanoisobenzofuro[7,1-hi]pyrido[2,1-c][1,7]dioxa[4]azacyclotridecin]-16′-one (3.7 mg, 69%) as a colourless solid. ESI-MS m/z calc. 398.221, found 399.3 (M+1)⁺.

### Step 5:

A solution of 1-methylcyclopropane-1-sulfonyl chloride (5 μL, 0.049 mmol) in DCM (0.1 mL) was added dropwise to a stirred solution of (5a′,8′-trans)-(10a′R,11′5)-11′-amino-7′,8′,10′,10a′,11′,12′,13′,14′-octahydro-4′H,6′H,16′H-spiro[cyclopropane-1,17′-[5a,8]ethanoisobenzofuro[7,1-hi]pyrido[2,1-c][1,7]dioxa[4]azacyclotridecin]-16′-one (3.7 mg), DMAP (6 mg, 0.049 mmol) and Et₃N (10 μL, 0.072 mmol) in DCM (0.4 mL) at −78° C. and the reaction mixture was stirred at ambient temperature for 18 h. The reaction was diluted with water and DCM. The aqueous phase was separated and extracted with DCM (3×). The combined organic phases were passed through a phase separation cartridge and the filtrates were concentrated in vacuo. Purification by reverse phase chromatography (C₁₈ column, 0 to 60% MeCN in water with 0.1% TFA) gave 1-methyl-N-((5a′,8′-trans)-(10a′R,11′S)-16′-oxo-7′,8′,10′,10a′,11′,12′,13′,14′-octahydro-4′H,6′H,16′H-spiro[cyclopropane-1,17′-[5a,8]ethanoisobenzofuro[7,1-hi]pyrido[2,1-c][1,7]dioxa[4]azacyclotridecin]-11′-yl)cyclopropane-1-sulfonamide (17, 0.8 mg, 21%) as a white solid. ¹H NMR (300 MHz, Chloroform-d) δ 7.07 (t, J=7.7 Hz, 1H), 6.89 (dd, J=7.4, 0.9 Hz, 1H), 6.38 (d, J=7.9 Hz, 1H), 5.34-5.07 (m, 1H), 4.97 (s, 2H), 4.44-4.23 (m, 1H), 4.17-4.01 (m, 1H), 3.81-3.65 (m, 2H), 3.67-3.53 (m, 1H), 3.53-3.45 (m, 1H), 3.44-3.27 (m, 1H), 2.74-2.54 (m, 1H), 2.07-1.91 (m, 4H), 1.89-1.54 (m, 5H), 1.50 (d, J=5.5 Hz, 4H), 1.49-1.44 (m, 1H), 1.42-1.33 (m, 3H), 1.32-1.16 (m, 1H), 1.14-0.98 (m, 1H), 0.92-0.67 (m, 2H) ppm; exchangeable H not observed. ESI-MS m/z calc. 516.229, found 517.3 (M+1)⁺.

### Example 17

### N-((5a,8-trans)-(10aR,11S)-4,4-Dimethyl-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1-methylcyclopropane-1-sulfonamide (18)

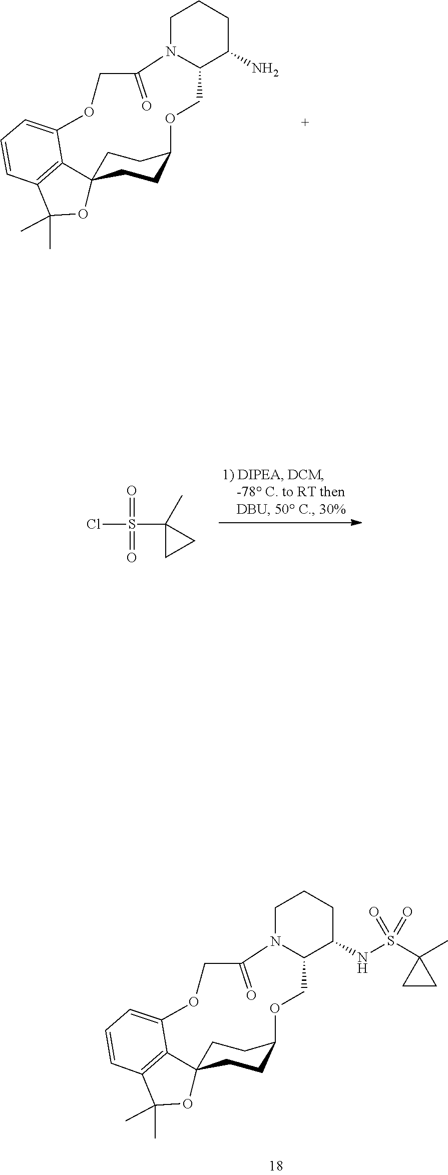

### Step 1:

Starting material (5a,8-trans)-(10aR,11S)-11-amino-4,4-dimethyl-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one was prepared in 4 steps from Int-14 and Int-2 using the method described in Example 13 Steps 1 to 3 followed by the method described in Example 6 Step 4.

1-Methylcyclopropane-1-sulfonyl chloride (4.516 mg, 0.029 mmol) was added to a stirred mixture of (5a,8-trans)-(10aR,11S)-11-amino-4,4-dimethyl-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (9 mg, 0.022 mmol) and DIPEA (8.713 mg, 11.74 μL, 0.067 mmol) in DCM (0.5 mL) at −78° C. and the reaction mixture was stirred at −78° C. for 5 min. The reaction was warmed to ambient temperature. DBU (3.421 mg, 3.361 μL, 0.022 mmol) was added and the reaction mixture was stirred and heated at 50° C. over the weekend. The reaction was diluted with DCM (2 mL) and 0.1 N aqueous HCl (2 mL) and the phases were separated. The organic layer was passed through a phase separation cartridge and the filtrates were concentrated in vacuo. Purification by reverse phase HPLC (C₁₈ Sunfire column, MeCN in water with 0.2% formic acid) gave N-((5a,8-trans)-(10aR,11S)-4,4-dimethyl-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1-methylcyclopropane-1-sulfonamide (18, 3.89 mg, 30%) as a white solid. ESI-MS m/z calc. 518.245, found 519.5 (M+1)⁺.

### Example 18

### 1,1,1-Trifluoro-N-((5a,8-trans)-(10aR,11S,17R)-17-methyl-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)methanesulfonamide (19)

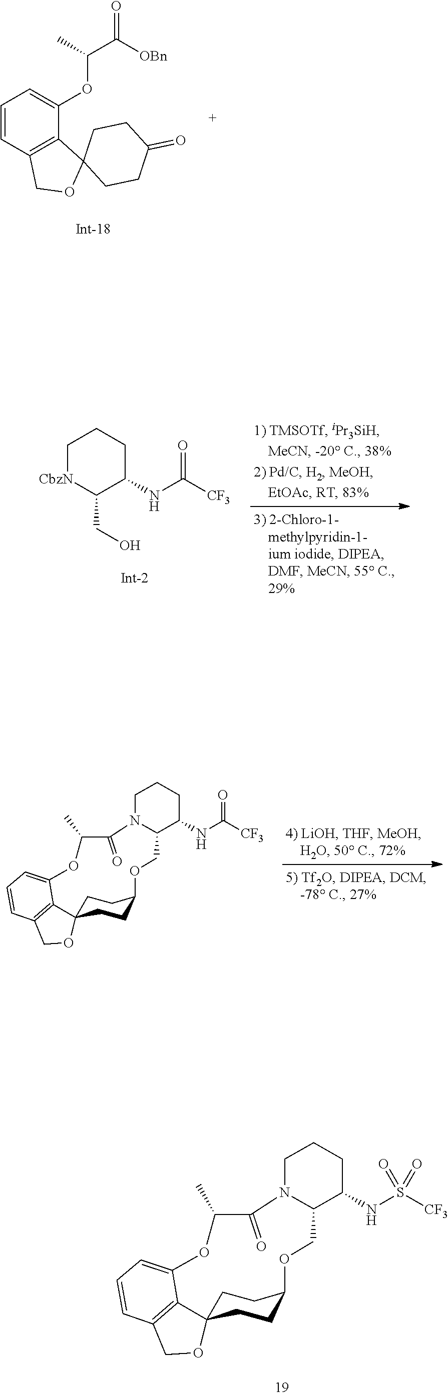

### Step 1:

Benzyl (R)-2-((4-oxo-3′H-spiro[cyclohexane-1,1′-isobenzofuran]-7′-yl)oxy)propanoate (Int-18, 1.5 g, 3.549 mmol) and benzyl (2R,3S)-2-(hydroxymethyl)-3-(2,2,2-trifluoroacetamido)piperidine-1-carboxylate (Int-2, 17 mL, 0.278 M in MeCN, 4.718 mmol) were treated via the method described in Example 1 Step 1 to give, after purification by flash chromatography (SiO₂, 0 to 55% EtOAc in heptane), benzyl (2R,3S)-2-((((1,4-trans)-7′-(((R)-1-(benzyloxy)-1-oxopropan-2-yl)oxy)-3′H-spiro[cyclohexane-1,1′-isobenzofuran]-4-yl)oxy)methyl)-3-(2,2,2-trifluoroacetamido)piperidine-1-carboxylate (1.1 g, 38%) as the predominant geometric isomer and as a colourless oil. ESI-MS m/z calc. 724.297, found 725.5 (M+1)⁺.

### Step 2:

Benzyl (2R,3S)-2-((((1,4-trans)-7′-(((R)-1-(benzyloxy)-1-oxopropan-2-yl)oxy)-3′H-spiro[cyclohexane-1,1′-isobenzofuran]-4-yl)oxy)methyl)-3-(2,2,2-trifluoroacetamido)piperidine-1-carboxylate (1.1 g, 1.518 mmol) was treated via the method described in Example 1 Step 2 to give (R)-2-(((1,4-trans)-4-(((2R,3S)-3-(2,2,2-trifluoroacetamido)piperidin-2-yl)methoxy)-3′H-spiro[cyclohexane-1,1′-isobenzofuran]-7′-yl)oxy)propanoic acid (750 mg, 83%) as the predominant geometric isomer and as a grey solid, which was used without further purification in the next step. ESI-MS m/z calc. 500.213, found 501.4 (M+1)⁺.

### Step 3:

A solution of (R)-2-(((1,4-trans)-4-(((2R,3S)-3-(2,2,2-trifluoroacetamido)piperidin-2-yl)methoxy)-3′H-spiro[cyclohexane-1,1′-isobenzofuran]-7′-yl)oxy)propanoic acid (600 mg, 1.199 mmol) in DMF (25 mL) was added dropwise to a stirred solution of 2-chloro-1-methylpyridin-1-ium iodide (740 mg, 2.896 mmol) and DIPEA (1.3 mL, 7.463 mmol) in MeCN (600 mL) at 55° C. and the reaction mixture was stirred at 55° C. for 30 min. The reaction was cooled to ambient temperature, concentrated in vacuo and the residue was partitioned between EtOAc (100 mL) and water (100 mL). The aqueous layer was separated and extracted with EtOAc (2×). The combined organic layers were washed with water and brine, dried (Na₂SO₄), filtered, and concentrated in vacuo. Purification by flash chromatography (120 g SiO₂, 0 to 75% EtOAc in heptane) gave 2,2,2-trifluoro-N-((5a,8-trans)-(10aR,11S,17R)-17-methyl-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)acetamide (200 mg, 29%) as a pale yellow solid. ESI-MS m/z calc. 482.203, found 483.4 (M+1)⁺.

### Step 4:

2,2,2-Trifluoro-N-((5a,8-trans)-(10aR,11S,17R)-17-methyl-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)acetamide (200 mg, 0.415 mmol) was treated via the method described in Example 1 Step 4 to give (5a,8-trans)-(10aR,11S,17R)-11-amino-17-methyl-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (150 mg, 72%) which was used without further purification in the next step. ESI-MS m/z calc. 386.221, found 387.4 (M+1)⁺.

### Step 5:

(5a,8-trans)-(10aR,11S,17R)-11-Amino-17-methyl-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (45 mg, 0.116 mmol) was treated via the method described in Example 1 Step 6 to give, after purification by reverse phase HPLC (XSelect CSH C₁₈ OBD column, 0 to 100% MeCN in water with 0.2% formic acid), 1,1,1-trifluoro-N-((5a,8-trans)-(10aR,11S,17R)-17-methyl-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)methanesulfonamide (19, 17.4 mg, 27%) as a white solid. ¹H NMR (300 MHz, Methanol-d₄) δ 7.23 (dd, J=8.1, 7.4 Hz, 1H), 7.02-6.96 (m, 2H), 5.18 (dt, J=10.3, 4.6 Hz, 1H), 4.99 (d, J=0.9 Hz, 2H), 4.49 (q, J=7.1 Hz, 1H), 4.29 (d, J=14.2 Hz, 1H), 3.90 (dd, J=10.9, 9.0 Hz, 1H), 3.84-3.65 (m, 2H), 3.51 (dd, J=8.9, 3.7 Hz, 1H), 3.41 (d, J=12.3 Hz, 1H), 2.96-2.83 (m, 1H), 2.11 (d, J=14.1 Hz, 1H), 1.95-1.40 (m, 13H) ppm; exchangeable H not observed. ESI-MS m/z calc. 518.170, found 519.4 (M+1)⁺.

### Example 19

### dia-N-((5a,8-trans)-(10aR*,11S*,17R)-17-Ethyl-6-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1,1-difluoroethane-1-sulfonamide (20)

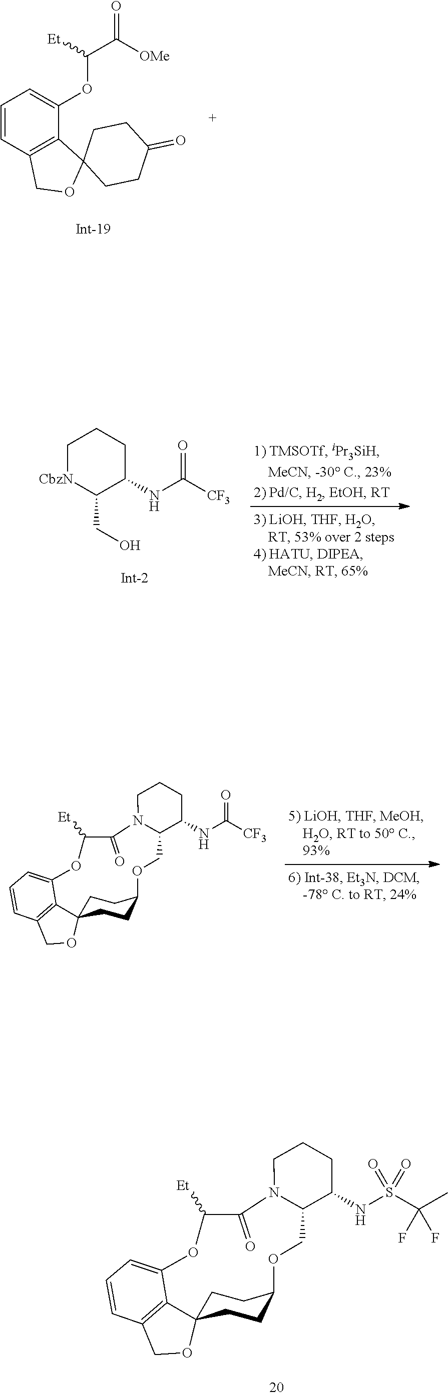

### Step 1:

Methyl rac-2-((4-oxo-3′H-spiro[cyclohexane-1,1′-isobenzofuran]-7′-yl)oxy)butanoate (Int-19, 1.030 g, 2.832 mmol) and benzyl (2R,3S)-2-(hydroxymethyl)-3-(2,2,2-trifluoroacetamido)piperidine-1-carboxylate (Int-2, 1.15 g, 3.160 mmol) were treated via the method described in Example 8 Step 1 to give, after purification by flash chromatography (SiO₂, 0 to 30% EtOAc in heptane), benzyl dia-(2R*,3S*)-2-((((1,4-trans)-7′-(((R)-1-methoxy-1-oxobutan-2-yl)oxy)-3′H-spiro[cyclohexane-1,1′-isobenzofuran]-4-yl)oxy)methyl)-3-(2,2,2-trifluoroacetamido)piperidine-1-carboxylate (431.7 mg, 23%) as the predominant geometric isomers and as a white foam. ¹H NMR (300 MHz, Chloroform-d) δ 8.68 (s, 1H), 7.42-7.22 (m, 5H), 7.09 (t, 1H), 6.72 (d, J=7.5 Hz, 1H), 6.45 (d, J=8.1 Hz, 1H), 5.24-5.00 (m, 2H), 4.97 (s, 2H), 4.70-4.43 (m, 2H), 4.15-3.97 (m, 2H), 3.90 (dt, 1H), 3.79-3.42 (m, 5H), 2.84-2.61 (m, 1H), 2.61-2.46 (m, 1H), 2.38 (td, J=12.8, 12.8 Hz, 1H), 2.17-2.02 (m, 1H), 2.00-1.49 (m, 11H), 1.12-0.84 (m, 3H) ppm. ESI-MS m/z calc. 662.282, found 663.6 (M+1)⁺.

### Step 2:

Benzyl dia-(2R*,3S*)-2-((((1,4-trans)-7′-(((R)-1-methoxy-1-oxobutan-2-yl)oxy)-3′H-spiro[cyclohexane-1,1′-isobenzofuran]-4-yl)oxy)methyl)-3-(2,2,2-trifluoroacetamido)piperidine-1-carboxylate (430 mg, 0.635 mmol) was treated via the method described in Example 16 Step 2 to give methyl dia-(R)-2-(((1,4-trans)-4-(((2R*,3S*)-3-(2,2,2-trifluoroacetamido)piperidin-2-yl)methoxy)-3′H-spiro[cyclohexane-1,1′-isobenzofuran]-7′-yl)oxy)butanoate as the predominant geometric isomers. ESI-MS m/z calc. 528.245, found 529.4 (M+1)⁺.

### Step 3:

LiOH (80 mg, 3.341 mmol) was added to a stirred solution of methyl dia-(R)-2-(((1,4-trans)-4-(((2R*,3S*)-3-(2,2,2-trifluoroacetamido)piperidin-2-yl)methoxy)-3′H-spiro[cyclohexane-1,1′-isobenzofuran]-7′-yl)oxy)butanoate in a mixture of THF (8 mL) and water (0.8 mL) and the reaction mixture was stirred at ambient temperature for 28 h. The reaction was concentrated in vacuo and the residue was partitioned between water and a 3:1 mixture of CHCl₃ and ⁱPrOH. The aqueous phase was separated and extracted with a 3:1 mixture of CHCl₃ and ⁱPrOH (3×). The combined organic phases were passed through a phase separation cartridge and the filtrates were concentrated in vacuo. Purification by reverse phase chromatography (C₁₈ column, 0 to 50% MeCN in water with 0.2% formic acid) gave dia-(R)-2-(((1,4-trans)-4-(((2R*,3S*)-3-(2,2,2-trifluoroacetamido)piperidin-2-yl)methoxy)-3′H-spiro[cyclohexane-1,1′-isobenzofuran]-7′-yl)oxy)butanoic acid (175 mg, 53% over 2 steps) as a white solid. ¹H NMR (300 MHz, Chloroform-d) δ 9.85 (d, J=9.0 Hz, 1H), 8.39 (s, 1H), 8.26 (s, 1H), 7.09 (td, J=7.9, 1.0 Hz, 1H), 6.76-6.67 (m, 1H), 6.64 (dd, J=8.1, 3.1 Hz, 1H), 5.03-4.82 (m, 2H), 4.53 (t, J=5.8 Hz, 2H), 3.74-3.58 (m, 1H), 3.58-3.46 (m, 2H), 3.45-3.25 (m, 2H), 2.82 (td, J=12.9, 2.9 Hz, 1H), 2.51 (td, J=13.5, 4.7 Hz, 1H), 2.34 (td, J=13.2, 5.4 Hz, 1H), 2.09-1.84 (m, 5H), 1.83-1.62 (m, 5H), 1.53-1.38 (m, 1H), 1.36-1.18 (m, 1H), 1.03-0.86 (m, 3H) ppm. ESI-MS m/z calc. 514.229, found 515.4 (M+1)⁺.

### Step 4:

dia-(R)-2-(((1,4-trans)-4-(((2R*,3S)-3-(2,2,2-Trifluoroacetamido)piperidin-2-yl)methoxy)-3′H-spiro[cyclohexane-1,1′-isobenzofuran]-7′-yl)oxy)butanoic acid (175 mg, 0.340 mmol) was treated via the method described in Example 13 Step 3 to give, after purification by reverse phase chromatography (Cis column, 0 to 60% MeCN in water with 0.2% formic acid), dia-N-((5a,8-trans)-(10aR*,11S*,17R)-17-ethyl-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-2,2,2-trifluoroacetamide (113.2 mg, 65%) as a white solid. ESI-MS m/z calc. 496.219, found 497.3 (M+1)⁺.

### Step 5:

LiOH (105 mg, 4.385 mmol) was added to a stirred suspension of dia-N-((5a,8-trans)-(10aR*,11S*,17R)-17-ethyl-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-2,2,2-trifluoroacetamide (113.2 mg, 0.222 mmol) in a mixture of THF (1.1 mL), MeOH (330 μL) and water (330 μL) and the reaction mixture was stirred at ambient temperature for 4 h and at 50° C. for 3 h. The mixture was cooled to ambient temperature, concentrated in vacuo and the residue was partitioned between water and a 3:1 mixture of CHCl₃ and ⁱPrOH. The aqueous phase was separated and extracted with a 3:1 mixture of CHCl₃ and ⁱPrOH (3×). The combined organic phases were passed through a phase separation cartridge and the filtrates were concentrated in vacuo to give dia-(5a,8-trans)-(10aR*,11S*,17R)-11-amino-17-ethyl-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (95 mg, 93%) as a clear semi-solid. ¹H NMR (300 MHz, Chloroform-d) δ 7.16-7.06 (m, 1H), 6.90-6.53 (m, 2H), 5.33-5.21 (m, 1H), 5.07-4.83 (m, 2H), 4.62-4.51 (m, 1H), 3.90-3.52 (m, 5H), 3.47 (d, J=13.6 Hz, 1H), 3.15-2.95 (m, 1H), 2.93-2.72 (m, 1H), 2.34-2.01 (m, 2H), 1.92-1.60 (m, 8H), 1.49-1.32 (m, 4H), 1.17-0.98 (m, 1H), 0.90 (td, J=7.5, 3.8 Hz, 2H) ppm. ESI-MS m/z calc. 400.236, found 401.4 (M+1)⁺.

### Step 6:

1-((1,1-Difluoroethyl)sulfonyl)-3-methyl-1H-imidazol-3-ium triflate (50 mg, 0.139 mmol) was added dropwise to a stirred solution of dia-(5a,8-trans)-(10aR*,11S*,17R)-11-amino-17-ethyl-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (45 mg, 0.112 mmol) and Et₃N (65 μL, 0.466 mmol) in DCM (1.0 mL) at −78° C. and the reaction mixture was warmed to ambient temperature and stirred for 18 h. The mixture was diluted with water and DCM and the aqueous phase was separated and extracted with DCM (3×). The combined organic phases were passed through a phase separation cartridge and the filtrates were concentrated in vacuo. Purification by reverse phase chromatography (C₁₈ column, 0 to 60% MeCN in water) gave dia-N-((5a,8-trans)-(10aR*,11S*,17R)-17-ethyl-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1,1-difluoroethane-1-sulfonamide (20, 14.3 mg, 24%) as an off-white solid. ¹H NMR (300 MHz, Acetonitrile-d₃) δ 7.12 (dd, J=8.1, 7.3 Hz, 1H), 6.96-6.65 (m, 2H), 6.11 (d, J=8.3 Hz, 1H), 5.25-5.13 (m, 1H), 4.98-4.82 (m, 2H), 4.63 (t, J=6.7 Hz, 1H), 3.76-3.60 (m, 2H), 3.60-3.47 (m, 2H), 3.45 (dd, J=9.4, 3.4 Hz, 1H), 2.98 (td, J=13.1, 2.7 Hz, 1H), 2.76 (td, J=13.5, 5.4 Hz, 1H), 2.04-1.89 (m, 5H, overlapped with solvent), 1.82-1.24 (m, 11H), 0.84 (t, J=7.5 Hz, 3H, overlapped with impurity) ppm. ESI-MS m/z calc. 528.211, found 529.3 (M+1)⁺.

### Example 20

### N-((5a,8-trans)-(10aR,11S)-17,17-Difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)cyclopropanesulfonamide (21)

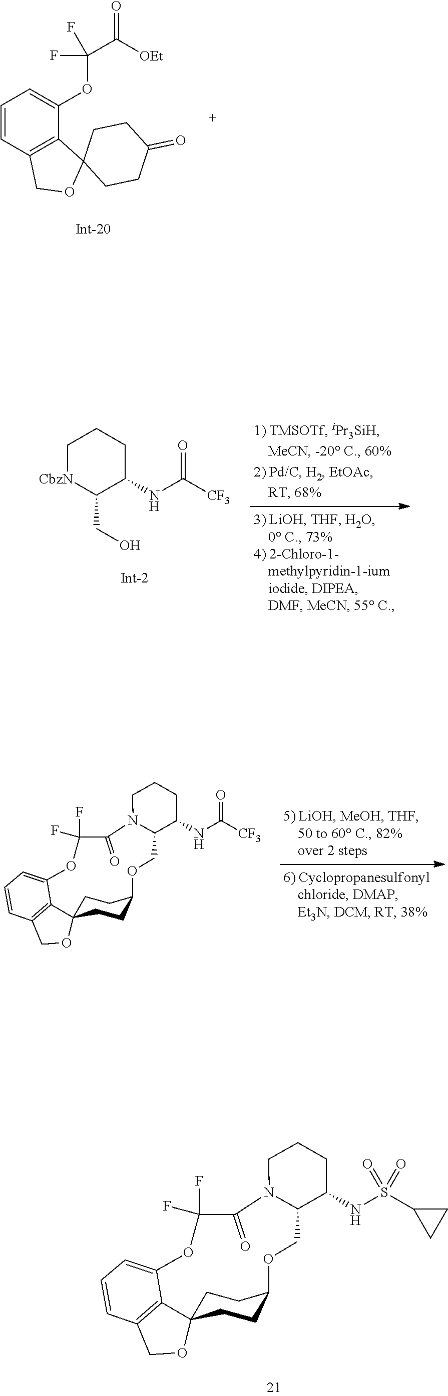

### Step 1:

Ethyl 2,2-difluoro-2-((4-oxo-3′H-spiro[cyclohexane-1,1′-isobenzofuran]-7′-yl)oxy)acetate (Int-20, 999 mg, 2.836 mmol) and benzyl (2R,3S)-2-(hydroxymethyl)-3-(2,2,2-trifluoroacetamido)piperidine-1-carboxylate (Int-2, 12 mL, 3.330 mmol) were treated via the method described in Example 1 Step 1 to give, after purification by flash chromatography (40 g SiO₂, 0 to 100% EtOAc in heptane), benzyl (2R,3S)-2-((((1,4-trans)-7′-(2-ethoxy-1,1-difluoro-2-oxoethoxy)-3′H-spiro[cyclohexane-1,1′-isobenzofuran]-4-yl)oxy)methyl)-3-(2,2,2-trifluoroacetamido)piperidine-1-carboxylate (1.476 g, 60%) as the predominant geometric isomer and as a clear oil. ESI-MS m/z calc. 684.247, found 685.4 (M+1)⁺.

### Step 2:

Benzyl (2R,3S)-2-((((1,4-trans)-7′-(2-ethoxy-1,1-difluoro-2-oxoethoxy)-3′H-spiro[cyclohexane-1,1′-isobenzofuran]-4-yl)oxy)methyl)-3-(2,2,2-trifluoroacetamido)piperidine-1-carboxylate (450 mg, 0.657 mmol) was treated via the method described in Example 13 Step 2 to give ethyl 2,2-difluoro-2-(((1,4-trans)-4-(((2R,3S)-3-(2,2,2-trifluoroacetamido)piperidin-2-yl)methoxy)-3′H-spiro[cyclohexane-1,1′-isobenzofuran]-7′-yl)oxy)acetate (343.5 mg, 68%) as the predominant geometric isomer and as a pale grey solid, which was used without further purification in the next step. ESI-MS m/z calc. 550.210, found 551.4 (M+1)⁺.

### Step 3:

LiOH (17.31 mg, 0.723 mmol) was added to a stirred solution of ethyl 2,2-difluoro-2-(((1,4-trans)-4-(((2R,3S)-3-(2,2,2-trifluoroacetamido)piperidin-2-yl)methoxy)-3′H-spiro[cyclohexane-1,1′-isobenzofuran]-7′-yl)oxy)acetate (343.5 mg) in THF (9 mL) and water (900 μL) at 0° C. and the reaction mixture was stirred at 0° C. for 3 h. The reaction was quenched by addition of a saturated aqueous NH₄Cl solution to pH ˜7 and the mixture was extracted with DCM (3×). The combined organic layers were passed through a phase separation cartridge and the filtrates were concentrated in vacuo. Purification by reverse phase chromatography (C₁₈ column, 0 to 100% MeCN in water with 0.2% formic acid) gave 2,2-difluoro-2-(((1,4-trans)-4-(((2R,3S)-3-(2,2,2-trifluoroacetamido)piperidin-2-yl)methoxy)-3′H-spiro[cyclohexane-1,1′-isobenzofuran]-7′-yl)oxy)acetic acid (249.3 mg, 73%) as a white solid. ¹H NMR (300 MHz, DMSO-d₆) δ 10.13 (s, 1H), 9.37-9.10 (m, 2H), 7.38-7.27 (m, 1H), 7.22-7.08 (m, 2H), 5.00 (s, 2H), 4.43 (s, 1H), 3.73-3.44 (m, 5H), 3.21-3.04 (m, 1H), 2.54 (m, 2H), 2.03 (s, 1H), 1.92-1.66 (m, 7H), 1.36 (d, J=13.1 Hz, 2H) ppm. ESI-MS m/z calc. 522.179, found 523.4 (M+1)⁺.

### Step 4:

2,2-Difluoro-2-(((1,4-trans)-4-(((2R,3S)-3-(2,2,2-trifluoroacetamido)piperidin-2-yl)methoxy)-3′H-spiro[cyclohexane-1,1′-isobenzofuran]-7′-yl)oxy)acetic acid (74 mg, 0.119 mmol) was treated via the method described in Example 18 Step 3 to give N-((5a,8-trans)-(10aR,11S)-17,17-difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-2,2,2-trifluoroacetamide (60.03 mg).

### Step 5:

LiOH (84 mg, 3.508 mmol) was added to a stirred solution of N-((5a,8-trans)-(10aR,11S)-17,17-difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-2,2,2-trifluoroacetamide (183 mg, 0.351 mmol) in a mixture of THF (3 mL) and MeOH (3 mL) and the reaction mixture was heated at 50° C. overnight. Additional LiOH (45 mg, 1.879 mmol) was added and the reaction was stirred at 60° C. for 4 h. The reaction was quenched by addition of a saturated aqueous NH₄Cl solution and extracted with DCM (2×), then with a 3:1 mixture of CHCl₃ and ⁱPrOH (1×). The combined organic layers were washed with brine, filtered through a phase separation cartridge and the filtrates were concentrated in vacuo. Purification by flash column chromatography (40 g SiO₂, 0 to 20% MeOH in DCM with 0.1% NH₄₀₁H) gave (5a,8-trans)-(10aR,11S)-11-amino-17,17-difluoro-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (144.5 mg, 82% over 2 steps) as a white solid, which was used without further purification in the next step. ESI-MS m/z calc. 408.186, found 409.3 (M+1)⁺.

### Step 6:

(5a,8-trans)-(10aR,11S)-11-Amino-17,17-difluoro-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (15 mg, 0.030 mmol) and cyclopropanesulfonyl chloride (6.315 mg, 0.045 mmol) were treated via the method described in Example 12 Step 1 to give, after purification by reverse phase chromatography (XSelect CSH C₁₈ OBD column, 0 to 100% MeCN in water with 0.2% formic acid), N-((5a,8-trans)-(10aR,11S)-17,17-difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)cyclopropanesulfonamide (21, 6.2 mg, 38%) as a colourless sticky oil.

### Example 21

### 1,1,1-Trifluoro-N-(6a,9-trans)-(11aR,12S,14R)-14-methyl-16-oxo-4,5,8,9,11a,12,13,14,16,17-decahydro-7H,11H-6a,9-ethanoisochromeno[8,1-hi]pyrrolo[2,1-c][1,7]dioxa[4]azacyclotridecin-12-yl)methanesulfonamide (22)

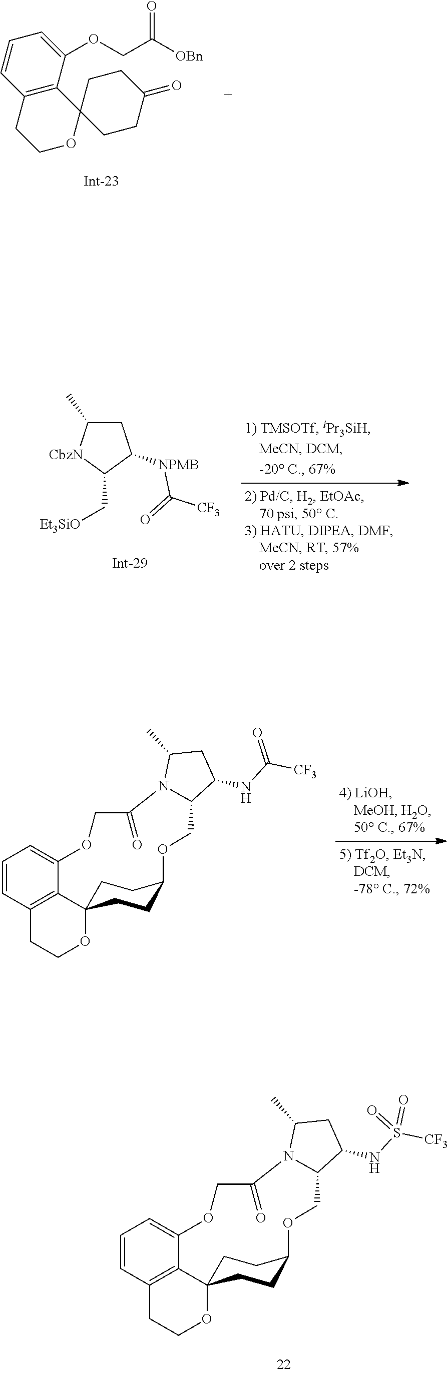

### Step 1:

A solution of TMSOTf (500 μL, 2.767 mmol) in DCM (2 mL) was added over 5 min to a stirred mixture of benzyl 2-((4-oxospiro[cyclohexane-1,1′-isochroman]-8′-yl)oxy)acetate (Int-23, 470 mg, 1.112 mmol), benzyl (2R,3S,5R)-5-methyl-2-(((triethylsilyl)oxy)methyl)-3-(2,2,2-trifluoro-N-(4-methoxybenzyl)acetamido)pyrrolidine-1-carboxylate (Int-29, 800 mg, 1.345 mmol) and ⁱPr₃SiH (700 μL, 3.094 mmol) in a mixture of MeCN (10 mL) and DCM (2 mL) at −20° C. and the reaction mixture was stirred at −20° C. for 1 h. The mixture was diluted with DCM (50 mL) and a saturated aqueous NaHCO₃ solution (50 mL). The aqueous layer was separated and extracted with DCM (2×50 mL). The combined organic layers were concentrated in vacuo. Purification by flash chromatography (220 g SiO₂, 0 to 30% MTBE in DCM) gave benzyl (2R,3S,5R)-2-((((1,4-trans)-8′-(2-(benzyloxy)-2-oxoethoxy)spiro[cyclohexane-1,1′-isochroman]-4-yl)oxy)methyl)-5-methyl-3-(2,2,2-trifluoro-N-(4-methoxybenzyl)acetamido)pyrrolidine-1-carboxylate (700 mg, 67%) as the predominant geometric isomer and as a colourless oil. ESI-MS m/z calc. 844.355, found 845.7 (M+1)⁺.

### Step 2:

A suspension of benzyl (2R,3S,5R)-2-((((1,4-trans)-8′-(2-(benzyloxy)-2-oxoethoxy)spiro[cyclohexane-1,1′-isochroman]-4-yl)oxy)methyl)-5-methyl-3-(2,2,2-trifluoro-N-(4-methoxybenzyl)acetamido)pyrrolidine-1-carboxylate (700 mg, 0.746 mmol) and Pd (190 mg, 10% w/w on carbon, 0.179 mmol) in EtOAc (20 mL) was stirred under 70 psi of hydrogen at 50° C. for 3 days. The reaction was cooled to ambient temperature, filtered through a pad of Celite® and the filtered cake was washed with DMF (100 mL). The filtrates were concentrated in vacuo to give 2-(((1,4-trans)-4-(((2R,3S,5R)-5-methyl-3-(2,2,2-trifluoroacetamido)pyrrolidin-2-yl)methoxy)spiro[cyclohexane-1,1′-isochroman]-8′-yl)oxy)acetic acid as the predominant geometric isomer.

### Step 3:

2-(((1,4-trans)-4-(((2R,3S,5R)-5-Methyl-3-(2,2,2-trifluoroacetamido)pyrrolidin-2-yl)methoxy)spiro[cyclohexane-1,1′-isochroman]-8′-yl)oxy)acetic acid was treated via the method described in Example 1 Step 3 to give, after purification by reverse phase chromatography (130 g Cis column, MeCN in water with 0.1% TFA), 2,2,2-trifluoro-N-((6a,9-trans)-(11aR,12S,14R)-14-methyl-16-oxo-4,5,8,9,11a,12,13,14,16,17-decahydro-7H,11H-6a,9-ethanoisochromeno[8,1-hi]pyrrolo[2,1-c][1,7]dioxa[4]azacyclotridecin-12-yl)acetamide (210 mg, 57% over 2 steps) as a yellow oil. ESI-MS m/z calc. 482.203, found 483.4 (M+1)⁺.

### Step 4:

2,2,2-Trifluoro-N-((6a,9-trans)-(11aR,12S,14R)-14-methyl-16-oxo-4,5,8,9,11a,12,13,14,16,17-decahydro-7H,11H-6a,9-ethanoisochromeno[8,1-hi]pyrrolo[2,1-c][1,7]dioxa[4]azacyclotridecin-12-yl)acetamide (210 mg, 0.429 mmol) was treated via the method described in Example 6 Step 4 to give, after purification by flash chromatography (40 g SiO₂, 0 to 10% MeOH in DCM with 2% NH₄OH), (6a,9-trans)-(11aR,12S,14R)-12-amino-14-methyl-4,5,8,9,11a,12,13,14-octahydro-7H,11H-6a,9-ethanoisochromeno[8,1-hi]pyrrolo[2,1-c][1,7]dioxa[4]azacyclotridecin-16(17H)-one (112.0 mg, 67%) as a colourless solid. ¹H NMR (300 MHz, Methanol-d₄) δ 7.16-7.05 (m, 1H), 6.98 (s, 1H), 6.74 (d, J=7.5 Hz, 1H), 4.67-4.54 (m, 1H), 4.44 (s, 1H), 4.35-4.04 (m, 2H), 3.83 (t, J=5.4 Hz, 2H), 3.80-3.61 (m, 2H), 2.86 (s, 1H), 2.74 (t, J=5.4 Hz, 2H), 2.69-2.55 (m, 1H), 2.57-2.31 (m, 1H), 2.02 (d, J=16.4 Hz, 3H), 1.94-1.60 (m, 5H), 1.63-1.32 (m, 4H) ppm; exchangeable H not observed. ESI-MS m/z calc.
386.221, found 387.4 (M+1)⁺.

### Step 5:

(6a,9-trans)-(11aR,12S,14R)-12-Amino-14-methyl-4,5,8,9,11a,12,13,14-octahydro-7H,11H-6a,9-ethanoisochromeno[8,1-hi]pyrrolo[2,1-c][1,7]dioxa[4]azacyclotridecin-16(17H)-one (70 mg, 0.170 mmol) was treated via the method described in Example 24 Step 6 to give, after purification by flash chromatography (24 g SiO₂, 20 to 100% MTBE in heptane), 1,1,1-trifluoro-N-((6a,9-trans)-(11aR,12S,14R)-14-methyl-16-oxo-4,5,8,9,11a,12,13,14,16,17-decahydro-7H,11H-6a,9-ethanoisochromeno[8,1-hi]pyrrolo[2,1-c][1,7]dioxa[4]azacyclotridecin-12-yl)methanesulfonamide (22, 66.9 mg, 72%) as a colourless solid. ¹H NMR (300 MHz, Chloroform-d) δ 7.82 (d, J=6.5 Hz, 1H), 7.14 (t, J=7.8 Hz, 1H), 6.79 (d, J=7.4 Hz, 1H), 6.64 (d, J=8.0 Hz, 1H), 4.96 (d, J=9.0 Hz, 1H), 4.65 (d, J=9.6 Hz, 1H), 4.56 (dd, J=7.7, 3.6 Hz, 1H), 4.49 (t, J=6.4 Hz, 1H), 4.33 (t, J=7.8 Hz, 2H), 3.98-3.80 (m, 3H), 3.36 (d, J=9.6 Hz, 1H), 2.98-2.74 (m, 3H), 2.55-2.26 (m, 2H), 2.12 (d, J=13.8 Hz, 1H), 2.07-1.86 (m, 2H), 1.84-1.68 (m, 1H), 1.62 (d, J=6.9 Hz, 6H) ppm. ESI-MS m/z calc. 518.170, found 519.3 (M+1)⁺.

### Example 22

### 1,1,1-Trifluoro-N-((5a,8-trans)-(10aR,11S,13R)-13-methyl-15-oxo-7,8,10a,11,12,13,16,17-octahydro-4H,6H,10H,15H-5a,8-ethanoisobenzofuro[7,1-ij]pyrrolo[2,1-c][1,8]dioxa[4]azacyclotetradecin-11-yl)methanesulfonamide (23)

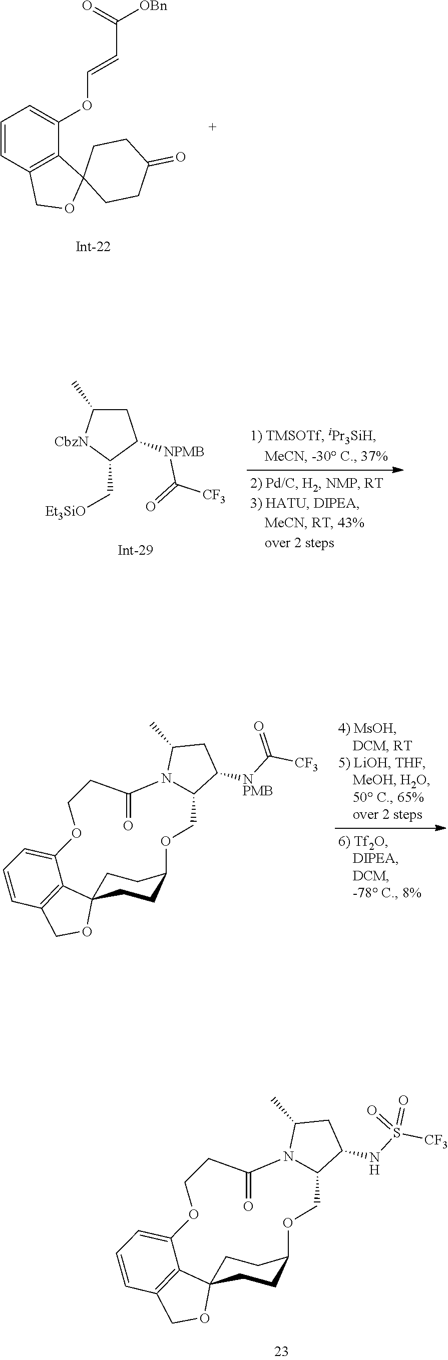

### Step 1:

Benzyl (E)-3-((4-oxo-3′H-spiro[cyclohexane-1,1′-isobenzofuran]-7′-yl)oxy)acrylate (Int-22, 270 mg, 0.668 mmol) and benzyl (2R,3S,5R)-5-methyl-2-(((triethylsilyl)oxy)methyl)-3-(2,2,2-trifluoro-N-(4-methoxybenzyl)acetamido)pyrrolidine-1-carboxylate (Int-29, 650 mg, 1.093 mmol) were treated via the method described in Example 8 Step 1 to give, after purification by reverse phase chromatography (Cis column, 30 to 100% MeCN in water with 0.1% TFA), benzyl (2R,3S,5R)-2-((((1,4-trans)-7′-(((E)-3-(benzyloxy)-3-oxoprop-1-en-1-yl)oxy)-3′H-spiro[cyclohexane-1,1′-isobenzofuran]-4-yl)oxy)methyl)-5-methyl-3-(2,2,2-trifluoro-N-(4-methoxybenzyl)acetamido)pyrrolidine-1-carboxylate (304 mg, 37%) as the predominant geometric isomer and as a white solid. ESI-MS m/z calc. 842.339, found 843.4 (M+1)⁺.

### Step 2:

A mixture of benzyl (2R,3S,5R)-2-((((1,4-trans)-7′-(((E)-3-(benzyloxy)-3-oxoprop−1-en-1-yl)oxy)-3′H-spiro[cyclohexane-1,1′-isobenzofuran]-4-yl)oxy)methyl)-5-methyl-3-(2,2,2-trifluoro-N-(4-methoxybenzyl)acetamido)pyrrolidine-1-carboxylate (304 mg, 0.361 mmol) and Pd (75 mg, 5% w/w on carbon, 0.035 mmol) in NMP (6 mL) was stirred at ambient temperature for 4 h under an atmospheric pressure of hydrogen. The reaction mixture was filtered through a pad of Celite®, washing the filtered cake with NMP (4 mL) and the mother liquors were concentrated in vacuo to give 3-(((1,4-trans)-4-(((2R,3S,5R)-5-methyl-3-(2,2,2-trifluoro-N-(4-methoxybenzyl)acetamido)pyrrolidin-2-yl)methoxy)-3′H-spiro[cyclohexane-1,1′-isobenzofuran]-7′-yl)oxy)propanoic acid as the predominant geometric isomer, which was used without further purification in the next step.

### Step 3:

3-(((1,4-trans)-4-(((2R,3S,5R)-5-Methyl-3-(2,2,2-trifluoro-N-(4-methoxybenzyl)acetamido)pyrrolidin-2-yl)methoxy)-3′H-spiro[cyclohexane-1,1′-isobenzofuran]-7′-yl)oxy)propanoic acid was treated via the method described in Example 13 Step 3 to give, after purification by reverse phase chromatography (C₁₈ column, 10 to 100% MeCN in water with 0.1% TFA), 2,2,2-trifluoro-N-(4-methoxybenzyl)-N-((5aR,8R,10aR,11S,13R)-13-methyl-15-oxo-7,8,10a,11,12,13,16,17-octahydro-4H,6H,10H,15H-5a,8-ethanoisobenzofuro[7,1-ij]pyrrolo[2,1-c][1,8]dioxa[4]azacyclotetradecin-11-yl)acetamide (109.5 mg, 43% over 2 steps) as a pale yellow oil. ESI-MS m/z calc. 602.260, found 603.4 (M+1)⁺.

### Step 4:

MsOH (250 μL, 3.853 mmol) was added to a stirred solution of 2,2,2-trifluoro-N-(4-methoxybenzyl)-N-((5a,8-trans)-(10aR,11S,13R)-13-methyl-15-oxo-7,8,10a,11,12,13,16,17-octahydro-4H,6H,10H,15H-5a,8-ethanoisobenzofuro[7,1-ij]pyrrolo[2,1-c][1,8]dioxa[4]azacyclotetradecin-11-yl)acetamide (100 mg, 0.153 mmol) in DCM (1 mL) and the reaction mixture was stirred at ambient temperature for 2 h. The mixture was diluted with DCM (5 mL) and a saturated aqueous NaHCO₃ solution (5 mL). The organic layer was separated, passed through a phase separation cartridge and concentrated in vacuo to give 2,2,2-trifluoro-N-((5a,8-trans)-(10aR,11S,13R)-13-methyl-15-oxo-7,8,10a,11,12,13,16,17-octahydro-4H,6H,10H,15H-5a,8-ethanoisobenzofuro[7,1-ij]pyrrolo[2,1-c][1,8]dioxa[4]azacyclotetradecin-11-yl)acetamide, which was used without further purification in the next step.

### Step 5:

2,2,2-Trifluoro-N-((5a,8-trans)-(10aR,11S,13R)-13-methyl-15-oxo-7,8,10a,11,12,13,16,17-octahydro-4H,6H,10H,15H-5a,8-ethanoisobenzofuro[7,1-ij]pyrrolo[2,1-c][1,8]dioxa[4]azacyclotetradecin-11-yl)acetamide was treated via the method described in Example 1 Step 4 to give (5a,8-trans)-(10aR,11S,13R)-11-amino-13-methyl-7,8,10a,11,12,13,16,17-octahydro-4H,6H,10H,15H-5a,8-ethanoisobenzofuro[7,1-ij]pyrrolo[2,1-c][1,8]dioxa[4]azacyclotetradecin-15-one (67 mg, 65% over 2 steps) as an off-white solid, which was used without further purification in the next step. ESI-MS m/z calc. 386.221, found 387.3 (M+1)⁺.

### Step 6:

(5a,8-trans)-(10aR,11S,13R)-11-Amino-13-methyl-7,8,10a,11,12,13,16,17-octahydro-4H,6H,10H,15H-5a,8-ethanoisobenzofuro[7,1-ij]pyrrolo[2,1-c][1,8]dioxa[4]azacyclotetradecin-15-one (65 mg, 0.168 mmol) was treated via the method described in Example 1 Step 6 to give, after purification by reverse phase chromatography (C₁₈ column, 10 to 100% MeCN in water with 0.2% formic acid) and lyophilisation from MeCN (0.5 mL) and water (1 ml), 1,1,1-trifluoro-N-((5a,8-trans)-(10aR,11S,13R)-13-methyl-15-oxo-7,8,10a,11,12,13,16,17-octahydro-4H,6H,10H,15H-5a,8-ethanoisobenzofuro[7,1-ij]pyrrolo[2,1-c][1,8]dioxa[4]azacyclotetradecin-11-yl)methanesulfonamide (23, 7.4 mg, 8%) as a white solid. ¹H NMR (300 MHz, Chloroform-d) δ 7.72 (s, 1H), 7.24-7.18 (m, 1H), 6.85 (d, J=8.1 Hz, 1H), 6.83-6.74 (m, 1H), 5.15-4.96 (m, 2H), 4.71 (dd, J=9.5, 2.7 Hz, 1H), 4.55-4.37 (m, 3H), 4.33-4.18 (m, 2H), 3.92 (s, 1H), 3.32 (d, J=9.4 Hz, 1H), 3.08 (ddd, J=14.6, 12.2, 4.5 Hz, 1H), 2.58 (td, J=13.6, 4.5 Hz, 1H), 2.51-2.40 (m, 1H), 2.39-2.13 (m, 2H), 2.09-1.69 (m, 6H), 1.53 (s, 3H), 1.36 (d, J=13.8 Hz, 1H) ppm. ESI-MS m/z calc. 518.170, found 519.2 (M+1)⁺.

### Example 23

### 1,1,1-Trifluoro-N-((5a,8-cis)-(10aR,11S)-16-oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl-4,4-d₂)methanesulfonamide (24)

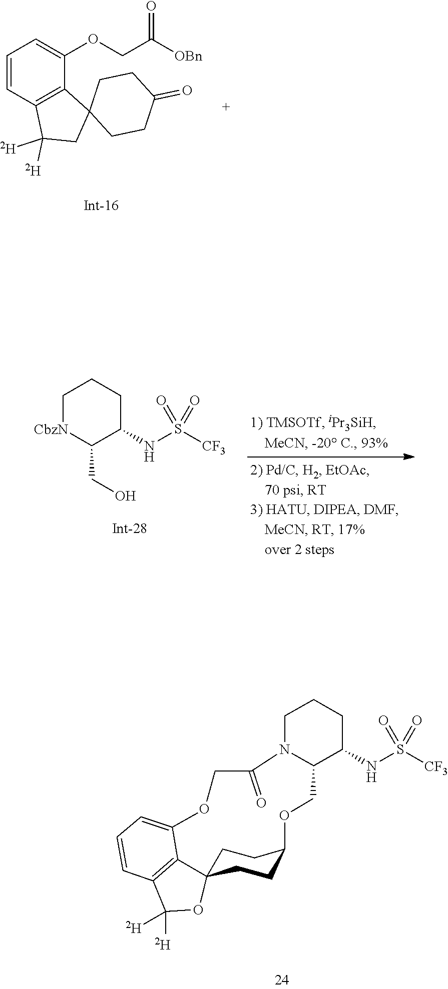

### Step 1:

Benzyl 2-((4-oxo-2′,3′-dihydrospiro[cyclohexane-1,1′-inden]-7′-yl-3′,3′-d₂)oxy)acetate (Int-16, 400 mg, 1.037 mmol) and benzyl (2R,3S)-2-(hydroxymethyl)-3-((trifluoromethyl)sulfonamido)piperidine-1-carboxylate (Int-28, 600 mg, 1.499 mmol) were treated via the method described in Example 1 Step 1 to give, after purification by flash chromatography (120 g SiO₂, 0 to 30% MTBE in DCM), benzyl (2R,3S)-2-((((1,4-cis)-7′-(2-(benzyloxy)-2-oxoethoxy)-2′,3′-dihydrospiro[cyclohexane-1,1′-inden]-4-yl-3′,3′-d₂)oxy)methyl)-3-((trifluoromethyl)sulfonamido)piperidine-1-carboxylate (900 mg, 93%) as the predominant geometric isomer and as a colourless oil. ESI-MS m/z calc. 746.281, found 747.6 (M+1)⁺.

### Step 2:

A suspension of benzyl (2R,3S)-2-((((1,4-cis)-7′-(2-(benzyloxy)-2-oxoethoxy)-2′,3′-dihydrospiro[cyclohexane-1,1′-inden]-4-yl-3′,3′-d2)oxy)methyl)-3-((trifluoromethyl)sulfonamido)piperidine-1-carboxylate (900 mg, 0.964 mmol) and Pd (250 mg, 10% w/w on carbon, 0.235 mmol) in EtOAc (30 mL) was stirred under 70 psi of hydrogen at ambient temperature for 3 h. The reaction was diluted with DMF (50 mL), and Celite® (20 mL) was added to the mixture. The suspension was filtered through a pad of Celite®, washing the filtered cake with DMF (500 mL) and the filtrates were concentrated in vacuo to give 2-(((1,4-cis)-4-(((2R,3S)-3-((trifluoromethyl)sulfonamido)piperidin-2-yl)methoxy)-2′,3′-dihydrospiro[cyclohexane-1,1′-inden]-7′-yl-3′,3′-d₂)oxy)acetic acid as the predominant geometric isomer, which was used without further purification in the next step.

### Step 3:

2-(((1,4-cis)-4-(((2R,3S)-3-((Trifluoromethyl)sulfonamido)piperidin-2-yl)methoxy)-2′,3′-dihydrospiro[cyclohexane-1,1′-inden]-7′-yl-3′,3′-d₂)oxy)acetic acid was treated via the method described in Example 1 Step 3 to give, after purification by reverse phase chromatography (150 g C₁₈ column, MeCN in water with 0.1% TFA) followed by flash chromatography (80 g SiO₂, 20 to 100% MTBE in heptane), 1,1,1-trifluoro-N-((5a,8-cis)-(10aR,11S)-16-oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl-4,4-d₂)methanesulfonamide (24, 88.4 mg, 17% over 2 steps) as a colourless solid. ¹H NMR (300 MHz, Methanol-d₄) δ 7.08 (t, J=7.7 Hz, 1H), 6.82 (dd, J=7.5, 0.9 Hz, 1H), 6.67 (dd, J=8.0, 0.9 Hz, 1H), 5.31 (d, J=11.6 Hz, 1H), 5.16 (dt, J=10.2, 4.2 Hz, 1H), 4.14 (d, J=11.6 Hz, 1H), 4.04-3.80 (m, 2H), 3.71 (d, J=11.0 Hz, 2H), 3.54-3.42 (m, 2H), 3.10 (td, J=13.9, 4.6 Hz, 1H), 2.24 (d, J=12.7 Hz, 1H), 2.05 (d, J=11.7 Hz, 2H), 2.00-1.33 (m, 8H), 1.32-0.81 (m, 2H) ppm; exchangeable H not observed. ESI-MS m/z calc. 504.187 found 505.4 (M+1)⁺.

### Example 24

### 1,1,1-Trifluoro-N-((5a,8-cis)-(10aR,11S,13R)-13-methyl-15-oxo-4,5,7,8,10a,11,12,13,15,16-decahydro-6H,10H-5a,8-ethanoindeno[7,1-hi]pyrrolo[2,1-c][1,7]dioxa[4]azacyclotridecin-11-yl)methanesulfonamide (25)

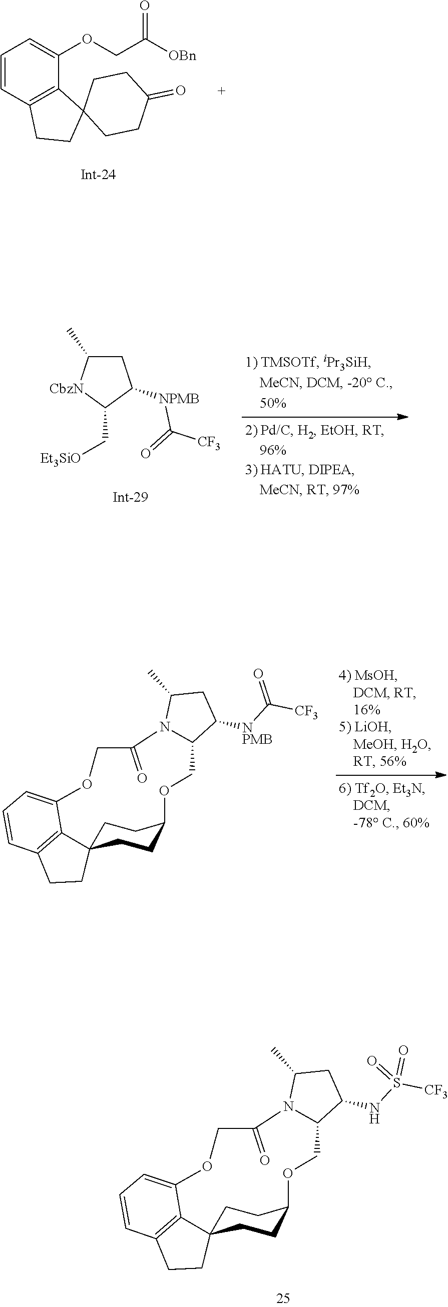

### Step 1:

Benzyl 2-((4-oxo-2′,3′-dihydrospiro[cyclohexane-1,1′-inden]-7′-yl)oxy)acetate (Int 24, 1.62 g, 4.299 mmol) and benzyl (2R,3S,5R)-5-methyl-2-(((triethylsilyl)oxy)methyl)-3-(2,2,2-trifluoro-N-(4-methoxybenzyl)acetamido)pyrrolidine-1-carboxylate (Int-29, 2.8 g, 4.708 mmol) were treated via the method described in Example 21 Step 1 to give, after purification by flash chromatography (SiO₂, 0 to 30% EtOAc in heptane), benzyl (2R,3S,5R)-2-((((1,4-cis)-7′-(2-(benzyloxy)-2-oxoethoxy)-2′,3′-dihydrospiro[cyclohexane-1,1′-inden]-4-yl)oxy)methyl)-5-methyl-3-(2,2,2-trifluoro-N-(4-methoxybenzyl)acetamido)pyrrolidine-1-carboxylate (1.83 g, 50%) as the predominant geometric isomer and as a clear semi-solid. ¹H NMR (300 MHz, Chloroform-d) δ 7.30-7.19 (m, 8H), 7.14-7.04 (m, 2H), 7.01-6.89 (m, 3H), 6.84-6.69 (m, 3H), 6.38 (d, J=8.2 Hz, 1H), 5.18-4.76 (m, 6H), 4.57-4.35 (m, 3H), 3.86-3.37 (m, 6H), 3.35-3.04 (m, 1H), 2.80 (t, J=7.4 Hz, 2H), 2.51 (t, J=13.6 Hz, 2H), 1.98 (dd, J=7.4, 7.4 Hz, 2H), 1.90-1.71 (m, 2H), 1.70-1.55 (m, 2H), 1.34-1.14 (m, 8H) ppm. ESI-MS m/z calc. 828.360, found 829.6 (M+1)⁺.

### Step 2:

Benzyl (2R,3S,5R)-2-((((1,4-cis)-7′-(2-(benzyloxy)-2-oxoethoxy)-2′,3′-dihydrospiro[cyclohexane-1,1′-inden]-4-yl)oxy)methyl)-5-methyl-3-(2,2,2-trifluoro-N-(4-methoxybenzyl)acetamido)pyrrolidine-1-carboxylate (1.83 g, 2.142 mmol) was treated via the method described in Example 16 Step 2 to give 2-(((1,4-cis)-4-(((2R,3S,5R)-5-methyl-3-(2,2,2-trifluoro-N-(4-methoxybenzyl)acetamido)pyrrolidin-2-yl)methoxy)-2′,3′-dihydrospiro[cyclohexane-1,1′-inden]-7′-yl)oxy)acetic acid (1.33 g, 96%) as the predominant geometric isomer and as a white semi-solid. ¹H NMR (300 MHz, Chloroform-d) δ 7.08-6.93 (m, 3H), 6.88-6.78 (m, 2H), 6.78-6.68 (m, 1H), 6.58 (d, J=8.0 Hz, 1H), 4.68-4.50 (m, 2H), 4.36 (d, J=12.9 Hz, 1H), 4.29-4.16 (m, 2H), 4.06-3.91 (m, 1H), 3.81-3.70 (m, 4H), 3.56-3.44 (m, 2H), 3.25 (dd, J=10.2, 5.6 Hz, 1H), 2.96-2.75 (m, 3H), 2.73-2.53 (m, 1H), 2.09-1.94 (m, 2H), 1.94-1.81 (m, 2H), 1.83-1.64 (m, 2H), 1.67-1.54 (m, 1H), 1.53-1.42 (m, 1H), 1.39 (d, J=6.4 Hz, 3H), 1.28-1.06 (m, 2H) ppm; exchangeable H not observed. ESI-MS m/z calc. 604.276, found 605.4 (M+1)⁺.

### Step 3:

2-(((1,4-cis)-4-(((2R,3S,5R)-5-Methyl-3-(2,2,2-trifluoro-N-(4-methoxybenzyl)acetamido)pyrrolidin-2-yl)methoxy)-2′,3′-dihydrospiro[cyclohexane-1,1′-inden]-7′-yl)oxy)acetic acid (1.33 g, 2.046 mmol) was treated via the method described in Example 13 Step 3 to give, after purification by flash chromatography (SiO₂, 0 to 50% EtOAc in heptane), 2,2,2-trifluoro-N-(4-methoxybenzyl)-N-((5a,8-cis)-(10aR,11S,13R)-13-methyl-15-oxo-4,5,7,8,10a,11,12,13,15,16-decahydro-6H,10H-5a,8-ethanoindeno[7,1-hi]pyrrolo[2,1-c][1,7]dioxa[4]azacyclotridecin-11-yl)acetamide (1.180 g, 97%) as a white solid. ESI-MS m/z calc. 586.265, found 587.4 (M+1)⁺.

### Step 4:

2,2,2-Trifluoro-N-(4-methoxybenzyl)-N-((5a,8-cis)-(10aR,11S,13R)-13-methyl-15-oxo-4,5,7,8,10a,11,12,13,15,16-decahydro-6H,10H-5a,8-ethanoindeno[7,1-hi]pyrrolo[2,1-c][1,7]dioxa[4]azacyclotridecin-11-yl)acetamide (1.08 g, 1.832 mmol) was treated via the method described in Example 22 Step 4 to give, after purification by reverse phase chromatography (C₁₈ column, 0 to 70% MeCN in water with 0.1% TFA), 2,2,2-trifluoro-N-((5a,8-cis)-(10aR,11S,13R)-13-methyl-15-oxo-4,5,7,8,10a,11,12,13,15,16-decahydro-6H,10H-5a,8-ethanoindeno[7,1-hi]pyrrolo[2,1-c][1,7]dioxa[4]azacyclotridecin-11-yl)acetamide (137.5 mg, 16%) as a white solid. ¹H NMR (300 MHz, Chloroform-d) δ 9.12 (d, 1H), 7.04 (t, J=7.7 Hz, 1H), 6.82-6.76 (m, 1H), 6.57 (d, J=8.0 Hz, 1H), 4.96 (d, J=10.3 Hz, 1H), 4.58 (q, J=6.7 Hz, 1H), 4.51 (dd, J=9.6, 4.0 Hz, 1H), 4.43 (dd, J=7.7, 3.8 Hz, 1H), 4.35-4.22 (m, 2H), 3.88-3.78 (m, 1H), 3.23 (d, J=9.5 Hz, 1H), 3.08 (td, J=13.6, 4.6 Hz, 1H), 2.89-2.78 (m, 2H), 2.25 (ddd, J=14.1, 8.1, 6.1 Hz, 1H), 2.09 (ddd, J=12.4, 7.8, 4.4 Hz, 1H), 2.01-1.86 (m, 3H), 1.82-1.66 (m, 2H), 1.59 (ddd, J=13.9, 4.1, 1.9 Hz, 1H), 1.50 (d, J=6.9 Hz, 3H), 1.46-1.32 (m, 1H), 1.25-1.14 (m, 1H), 1.14-1.01 (m, 1H) ppm. ESI-MS m/z calc. 466.208, found 467.2 (M+1)⁺.

### Step 5:

LiOH (70 mg, 2.923 mmol) was added to a solution of 2,2,2-trifluoro-N-((5a,8-cis)-(10aR,11S,13R)-13-methyl-15-oxo-4,5,7,8,10a,11,12,13,15,16-decahydro-6H,10H-5a,8-ethanoindeno[7,1-hi]pyrrolo[2,1-c][1,7]dioxa[4]azacyclotridecin-11-yl)acetamide (137.5 mg, 0.294 mmol) in a mixture of MeOH (2.1 mL) and water (700 μL) and the reaction mixture was stirred at ambient temperature for 24 h. The reaction was concentrated in vacuo and the residue was partitioned between water and a 3:1 mixture of CHCl₃ and ⁱPrOH. The aqueous phase was separated and extracted with a 3:1 mixture of CHCl₃ and ⁱPrOH (3×). The combined organic phases were passed through a phase separation cartridge and the filtrates were concentrated in vacuo. Purification by reverse phase chromatography (C₁₈ column, 0 to 50% MeCN in water with 0.2% formic acid) gave (5a,8-cis)-(10aR,11S,13R)-11-amino-13-methyl-4,5,7,8,10a,11,12,13-octahydro-6H,10H-5a,8-ethanoindeno[7,1-hi]pyrrolo[2,1-c][1,7]dioxa[4]azacyclotridecin-15(16H)-one (64.3 mg, 56%) as a white solid. ESI-MS m/z calc. 370.226, found 371.3 (M+1)⁺.

### Step 6:

A solution of Tf₂O (12 μL, 0.071 mmol) in DCM (200 μL) was added dropwise to a stirred solution of (5a,8-cis)-(10aR,11S,13R)-11-amino-13-methyl-4,5,7,8,10a,11,12,13-octahydro-6H,10H-5a,8-ethanoindeno[7,1-hi]pyrrolo[2,1-c][1,7]dioxa[4]azacyclotridecin-15(16H)-one (20 mg, 0.054 mmol) and Et₃N (15 μL, 0.108 mmol) in DCM (1.6 mL) at −78° C. and the reaction mixture was stirred at −78° C. for 65 min. Additional Et₃N (15 μL, 0.108 mmol) and a solution of Tf₂O (12 μL, 0.071 mmol) in DCM (200 μL) were added and the reaction was stirred at −78° C. for an additional 10 min. The cold bath was removed. A saturated aqueous Na₂CO₃ solution was added and the reaction was stirred for 10 min. The aqueous phase was separated and extracted with DCM (3×). The combined organic phases were passed through a phase separation cartridge and the filtrates were concentrated in vacuo. Purification by reverse chromatography (C₁₈ column, 0 to 70% MeCN in water with 0.1% TFA) gave 1,1,1-trifluoro-N-((5a,8-cis)-(10aR,11S,13R)-13-methyl-15-oxo-4,5,7,8,10a,11,12,13,15,16-decahydro-6H,10H-5a,8-ethanoindeno[7,1-hi]pyrrolo[2,1-c][1,7]dioxa[4]azacyclotridecin-11-yl)methanesulfonamide (25, 16.9 mg, 60%) as a white solid. ¹H NMR (300 MHz, Chloroform-d) δ 7.05 (t, J=7.7 Hz, 1H), 6.78 (dd, 1H), 6.57 (d, J=8.0 Hz, 1H), 4.95 (d, J=10.2 Hz, 1H), 4.60-4.48 (m, 1H), 4.48-4.33 (m, 2H), 4.34-4.17 (m, 2H), 3.84 (s, 1H), 3.29 (d, J=9.6 Hz, 1H), 3.05 (td, J=13.6, 4.6 Hz, 1H), 2.90-2.77 (m, 2H), 2.33-2.16 (m, 1H), 2.16-1.83 (m, 5H), 1.82-1.65 (m, 2H), 1.64-1.49 (m, 4H), 1.40 (td, J=14.3, 4.5 Hz, 1H), 1.27-1.16 (m, 1H), 1.15-1.03 (m, 1H) ppm. ESI-MS m/z calc. 502.175, found 503.3 (M+1)⁺.

### Example 25

### N-((5a,8-trans)-(10aR,11S)-3-Chloro-1-fluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1,1,1-trifluoromethanesulfonamide (26)

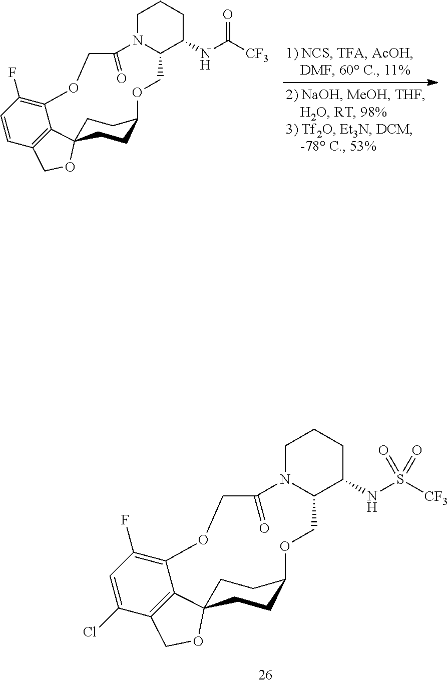

### Step 1:

2,2,2-Trifluoro-N-((5a,8-trans)-(10aR,11S)-1-fluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)acetamide (626 mg, prepared using the method described in Example 6 Steps 1 to 3 from Int-6 and Int-2, 1.287 mmol) was treated via the method described in Example 1 Step 5 to give, after purification by reverse phase chromatography (15.5 g C₁₈ column, 10 to 100% MeCN in water with 0.2% formic acid), N-((5a,8-trans)-(10aR,11S)-3-chloro-1-fluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-2,2,2-trifluoroacetamide (71 mg, 11%) as a white solid. ¹H NMR (400 MHz, Chloroform-d) δ 7.08 (d, J=6.5 Hz, 1H), 7.01 (dd, J=10.4, 0.9 Hz, 1H), 5.37-5.29 (m, 1H), 5.06-4.92 (m, 3H), 4.31-4.22 (m, 2H), 4.01 (dd, J=14.5, 5.6 Hz, 1H), 3.87-3.82 (m, 1H), 3.75 (dd, J=9.0, 4.0 Hz, 1H), 3.64 (dd, J=9.0, 6.4 Hz, 1H), 3.52 (ddd, J=14.9, 11.2, 4.5 Hz, 1H), 2.71 (td, J=12.9, 4.6 Hz, 1H), 1.95-1.76 (m, 7H), 1.76-1.66 (m, 3H), 1.56-1.50 (m, 1H) ppm. ESI-MS m/z calc. 520.139, found 521.1 (M+1)⁺.

### Step 2:

NaOH (580 μL, 2 M aqueous solution, 1.160 mmol) was added to a stirred solution of N-((5a,8-trans)-(10aR,11S)-3-chloro-1-fluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-2,2,2-trifluoroacetamide (60 mg, 0.115 mmol) in a mixture of MeOH (580 μL) and THF (1.75 mL) and the reaction mixture was stirred at ambient temperature for 18 h. The reaction was partitioned between EtOAc and a saturated aqueous NH₄Cl solution. The aqueous phase was separated and extracted with EtOAc (2×). The combined organic layers were dried (Na₂SO₄), filtered, and concentrated in vacuo. Purification by reverse phase chromatography (15.5 g C₁₈ column, 10 to 100% MeCN in water with 0.2% formic acid) gave (5a,8-trans)-(10aR,11S)-11-amino-3-chloro-1-fluoro-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (50 mg, 98%) as a white solid. ¹H NMR (300 MHz, Chloroform-d) δ 7.01 (d, J=10.5 Hz, 1H), 5.37-5.31 (m, 1H), 5.11 (dt, J=10.1, 4.6 Hz, 1H), 5.06-4.93 (m, 2H), 4.30 (dd, J=12.8, 2.0 Hz, 1H), 3.92-3.77 (m, 3H), 3.69 (dd, J=8.9, 3.7 Hz, 1H), 3.47-3.34 (m, 1H), 3.20 (dt, J=11.9, 4.7 Hz, 1H), 2.79 (td, J=14.4, 5.1 Hz, 1H), 2.05 (td, J=13.5, 4.6 Hz, 2H), 1.89-1.77 (m, 3H), 1.74-1.67 (m, 2H), 1.60-1.44 (m, 4H), 1.28 (s, 2H) ppm. ESI-MS m/z calc. 424.157, found 425.1 (M+1)⁺.

### Step 3:

(5a,8-trans)-(10aR,11S)-11-Amino-3-chloro-1-fluoro-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (30 mg, 0.071 mmol) was treated via the method described in Example 24 Step 6 to give, after purification by reverse phase HPLC (XSelect CSH C₁₈ OBD column, MeCN in water with 0.2% formic acid), N-((5a,8-trans)-(10aR,11S)-3-chloro-1-fluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1,1,1-trifluoromethanesulfonamide (26, 21.2 mg, 53%) as a white solid. ¹H NMR (400 MHz, Chloroform-d) δ 7.01 (dd, J=10.3, 2.8 Hz, 1H), 5.68 (d, J=7.6 Hz, 1H), 5.31 (dd, J=13.0, 2.4 Hz, 1H), 5.08 (dd, J=6.8, 4.2 Hz, 1H), 4.99 (q, J=13.5 Hz, 2H), 4.27 (dd, J=13.0, 2.4 Hz, 1H), 4.00-3.90 (m, 2H), 3.83 (s, 1H), 3.76 (dd, J=9.0, 3.9 Hz, 1H), 3.70 (t, J=8.0 Hz, 1H), 3.54-3.44 (m, 1H), 2.69 (td, J=14.5, 5.1 Hz, 1H), 1.97-1.79 (m, 6H), 1.78-1.65 (m, 4H), 1.31-1.19 (m, 1H) ppm. ESI-MS m/z calc. 556.106, found 557.0 (M+1)⁺.

### Example 26

### N-((5a,8-trans)-(10aR,11S,13R)-3,13-Dimethyl-15-oxo-7,8,10a,11,12,13,15,16-octahydro-4H,6H,10H-5a,8-ethanoisobenzofuro[7,1-hi]pyrrolo[2,1-c][1,7]dioxa[4]azacyclotridecin-11-yl)-1,1,1-trifluoromethanesulfonamide (27)

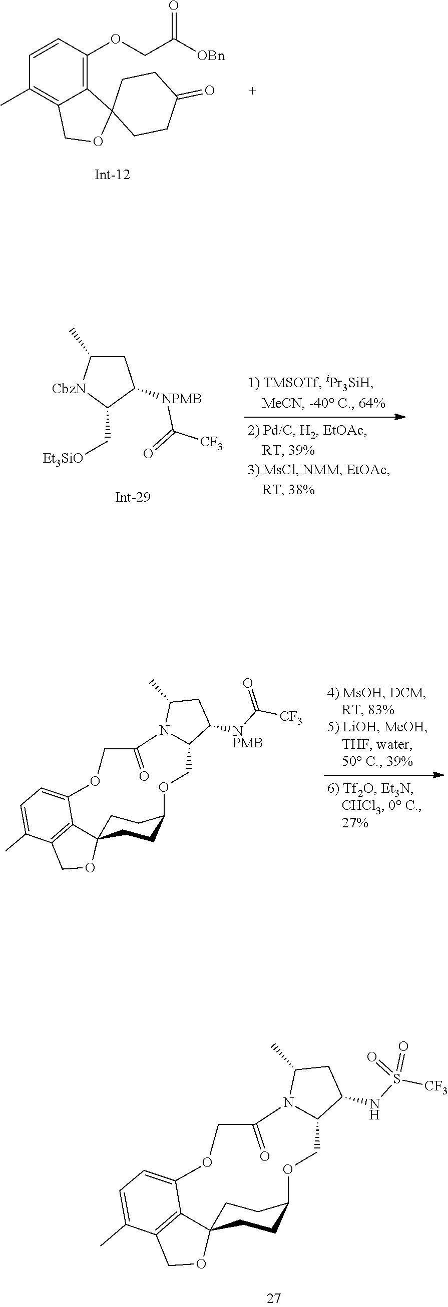

### Step 1:

ⁱPr3SiH (270 μL, 1.318 mmol) was added to a stirred solution of benzyl 2-((4′-methyl-4-oxo-3′H-spiro[cyclohexane-1,1′-isobenzofuran]-7′-yl)oxy)acetate (Int-12, 250 mg, 0.651 mmol) and benzyl (2R,3S,5R)-5-methyl-2-(((triethylsilyl)oxy)methyl)-3-(2,2,2-trifluoro-N-(4-methoxybenzyl)acetamido)pyrrolidine-1-carboxylate (Int-29, 427.5 mg, 0.719 mmol) in MeCN (10 mL) at −40° C. and the reaction mixture was stirred at −40° C. for 3 min. A solution of TMSOTf (240 μL, 1.328 mmol) in MeCN (500 μL) was added dropwise over 1.5 min and the reaction was stirred at −40° C. for 25 min. The reaction was quenched by addition of a saturated aqueous NaHCO₃ solution (˜15 mL), warmed to ambient temperature and diluted with EtOAc (˜25 mL). The organic layer was separated, washed with brine (1×25 mL), dried (Na₂SO₄), filtered, and concentrated in vacuo. Purification by flash chromatography (24 g SiO₂, 0 to 70% EtOAc in heptane) gave benzyl (2R,3S,5R)-2-(((7′-(2-(benzyloxy)-2-oxoethoxy)-4′-methyl-3′H-spiro[cyclohexane-1,1′-isobenzofuran]-4-yl)oxy)methyl)-5-methyl-3-(2,2,2-trifluoro-N-(4-methoxybenzyl)acetamido)pyrrolidine-1-carboxylate (359 mg, 64%) as a 5:1 mixture of trans and cis isomers and as a clear oil. ESI-MS m/z calc. 844.355, found 845.5 (M+1)⁺.

### Step 2:

A 5:1 mixture of trans and cis isomers of benzyl (2R,3S,5R)-2-(((7′-(2-(benzyloxy)-2-oxoethoxy)-4′-methyl-3′H-spiro[cyclohexane-1,1′-isobenzofuran]-4-yl)oxy)methyl)-5-methyl-3-(2,2,2-trifluoro-N-(4-methoxybenzyl)acetamido)pyrrolidine-1-carboxylate (359 mg, 0.414 mmol) was treated via the method described in Example 13 Step 2 to give, after purification by reverse phase chromatography (15.5 g C₁₈ column, 0 to 100% MeCN in water with 0.2% formic acid), 2-(((1,4-trans)-4′-methyl-4-(((2R,3S,5R)-5-methyl-3-(2,2,2-trifluoro-N-(4-methoxybenzyl)acetamido)pyrrolidin-2-yl)methoxy)-3′H-spiro[cyclohexane-1,1′-isobenzofuran]-7′-yl)oxy)acetic acid (108.7 mg, 39%) as a light brown oil. ESI-MS m/z calc. 620.271, found 621.3 (M+1)⁺. 2-(((1,4-cis)-4′-Methyl-4-(((2R,3S,5R)-5-methyl-3-(2,2,2-trifluoro-N-(4-methoxybenzyl)acetamido)pyrrolidin-2-yl)methoxy)-3′H-spiro[cyclohexane-1,1′-isobenzofuran]-7′-yl)oxy)acetic acid (55.2 mg, 21%) was also collected.

### Step 3:

MsCl (25 μL, 0.323 mmol) and NMM (50 μL, 0.455 mmol) were successively added to a stirred solution of 2-(((1,4-trans)-4′-methyl-4-(((2R,3S,5R)-5-methyl-3-(2,2,2-trifluoro-N-(4-methoxybenzyl)acetamido)pyrrolidin-2-yl)methoxy)-3′H-spiro[cyclohexane-1,1′-isobenzofuran]-7′-yl)oxy)acetic acid (100 mg, 0.150 mmol) in EtOAc (10 mL) and the reaction mixture was stirred at ambient temperature for 3 h. The reaction was diluted with EtOAc (40 mL) washed with 0.5 N aqueous HCl (50 mL), a saturated aqueous NaHCO₃ solution (50 mL) and brine (50 mL), dried (Na₂SO₄), filtered, and concentrated in vacuo. Purification by flash chromatography (4 g SiO₂, 0 to 100% EtOAc in heptane) gave N-((5a,8-trans)-(10aR,11S,13R)-3,13-dimethyl-15-oxo-7,8,10a,11,12,13,15,16-octahydro-4H,6H,10H-5a,8-ethanoisobenzofuro[7,1-hi]pyrrolo[2,1-c][1,7]dioxa[4]azacyclotridecin-11-yl)-2,2,2-trifluoro-N-(4-methoxybenzyl)acetamide (35.3 mg, 38%) as a clear oil. ESI-MS m/z calc. 602.260, found 603.5 (M+1)⁺.

### Step 4:

N-((5a,8-trans)-(10aR,11S,13R)-3,13-Dimethyl-15-oxo-7,8,10a,11,12,13,15,16-octahydro-4H,6H,10H-5a,8-ethanoisobenzofuro[7,1-hi]pyrrolo[2,1-c][1,7]dioxa[4]azacyclotridecin-11-yl)-2,2,2-trifluoro-N-(4-methoxybenzyl)acetamide (35 mg, 0.056 mmol) was treated via the method described in Example 22 Step 4 to give N-((5a,8-trans)-(10aR,11S,13R)-3,13-dimethyl-15-oxo-7,8,10a,11,12,13,15,16-octahydro-4H,6H,10H-5a,8-ethanoisobenzofuro[7,1-hi]pyrrolo[2,1-c][1,7]dioxa[4]azacyclotridecin-11-yl)-2,2,2-trifluoroacetamide (26.5 mg, 83%) as a clear oil, which was used without further purification in the next step. ESI-MS m/z calc. 482.203, found 483.0 (M+1)⁺.

### Step 5:

N-((5a,8-trans)-(10aR,11S,13R)-3,13-Dimethyl-15-oxo-7,8,10a,11,12,13,15,16-octahydro-4H,6H,10H-5a,8-ethanoisobenzofuro[7,1-hi]pyrrolo[2,1-c][1,7]dioxa[4]azacyclotridecin-11-yl)-2,2,2-trifluoroacetamide (26.5 mg) was treated via the method described in Example 1 Step 4 to give, after purification by reverse phase chromatography (5.5 g C₁₈ column, 0 to 100% MeCN in water with 0.2% formic acid), (5a,8-trans)-(10aR,11S,13R)-11-amino-3,13-dimethyl-7,8,10a,11,12,13-hexahydro-4H,6H,10H-5a,8-ethanoisobenzofuro[7,1-hi]pyrrolo[2,1-c][1,7]dioxa[4]azacyclotridecin-15(16H)-one (9.1 mg, 39%) as a clear oil. ESI-MS m/z calc. 386.221, found 387.0 (M+1)⁺.

### Step 6:

A solution of Tf₂O (3.5 μL, 0.021 mmol) in CHCl₃ (40 μL) was added dropwise over 1 min to a stirred solution of (5a,8-trans)-(10aR,11S,13R)-11-amino-3,13-dimethyl-7,8,10a,11,12,13-hexahydro-4H,6H,10H-5a,8-ethanoisobenzofuro[7,1-hi]pyrrolo[2,1-c][1,7]dioxa[4]azacyclotridecin-15(16H)-one (7.5 mg, 0.018 mmol) and Et₃N (7.5 μL, 0.054 mmol) in CHCl₃ (170 μL) at 0° C. and the reaction mixture was stirred at 0° C. for 20 min. An additional solution of Tf₂O (1.75 μL, 0.010 mmol) in CHCl₃ (20 μL) was added and the reaction was stirred at 0° C. for a further 20 min. The reaction was quenched by addition of a saturated NaHCO₃ solution (˜2 mL) and diluted with DCM (˜2 mL). The aqueous layer was separated and extracted with DCM (2×5 mL). The combined organic layers were concentrated in vacuo. Purification by reverse phase chromatography (5.5 g C₁₈ column, 0 to 100% MeCN in water with 0.2% formic acid) gave N-((5a,8-trans)-(10aR,11S,13R)-3,13-dimethyl-15-oxo-7,8,10a,11,12,13,15,16-octahydro-4H,6H,10H-5a,8-ethanoisobenzofuro[7,1-hi]pyrrolo[2,1-c][1,7]dioxa[4]azacyclotridecin-11-yl)-1,1,1-trifluoromethanesulfonamide (27, 2.7 mg, 27%) as a clear oil. ESI-MS m/z calc. 518.170, found 518.9 (M+1)⁺.

### Example 27

### N-((5a,8-trans)-(10aR,11S)-1,3-Difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1,1-difluoromethanesulfonamide (28)

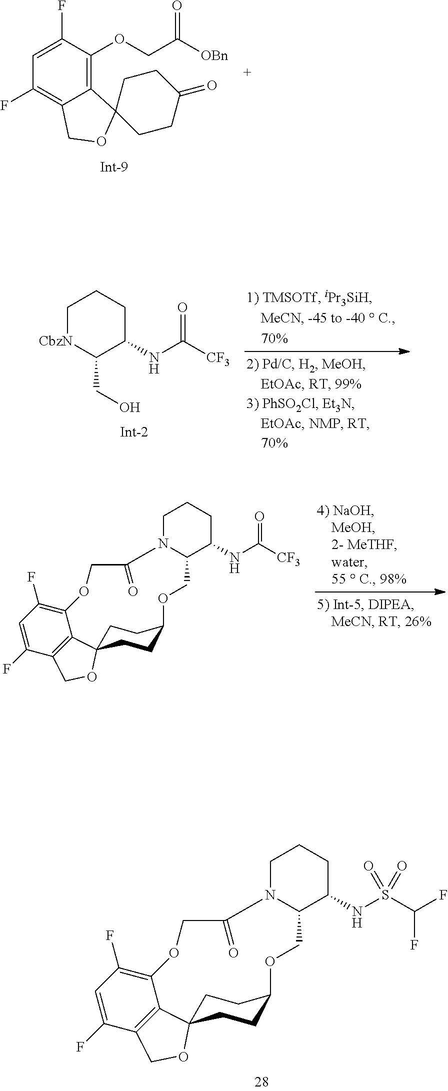

### Step 1:

Under a nitrogen atmosphere, ⁱPr₃SiH (18.0 mL, 87.86 mmol) was added to a solution of benzyl 2-((4′,6′-difluoro-4-oxo-3′H-spiro[cyclohexane-1,1′-isobenzofuran]-7′-yl)oxy)acetate (Int-9, 13.83 g, 34.37 mmol) and benzyl (2R,3S)-2-(hydroxymethyl)-3-(2,2,2-trifluoroacetamido)piperidine-1-carboxylate (Int-2, 12.7 g, 34.19 mmol) in MeCN (300 mL) at −45° C. A solution TMSOTf (9.5 mL, 52.58 mmol) in MeCN (100 mL) was added dropwise over 30 min, keeping the internal temperature below −40° C., and the reaction mixture was stirred below −40° C. for 65 min. The reaction was quenched by addition of a saturated aqueous NaHCO₃ solution (200 mL), warmed to ambient temperature and partially concentrated in vacuo to remove most of the MeCN. The mixture was partitioned with MTBE (300 mL) and filtered. The aqueous phase was separated and extracted with MTBE (200 mL). The combined organic layers were washed with a half saturated brine solution (400 mL), dried (Na₂SO₄), filtered and concentrated in vacuo. Purification by flash chromatography (2×330 g SiO₂, 0 to 40% EtOAc in heptane) gave benzyl (2R,3S)-2-((((1,4-trans)-7′-(2-(benzyloxy)-2-oxoethoxy)-4′,6′-difluoro-3′H-spiro[cyclohexane-1,1′-isobenzofuran]-4-yl)oxy)methyl)-3-(2,2,2-trifluoroacetamido)piperidine-1-carboxylate (18.83 g, 70%) as the predominant geometric isomer and as a colourless foam. ¹H NMR (300 MHz, Chloroform-d) δ 8.57 (s, 1H), 7.41-7.23 (m, 10H), 6.73 (dd, J=12.1, 7.9 Hz, 1H), 5.38-4.95 (m, 6H), 4.85-4.60 (m, 3H), 4.25-3.94 (m, 3H), 3.80-3.50 (m, 2H), 2.80 (t, J=12.7 Hz, 1H), 2.56-2.22 (m, 2H), 2.15-2.04 (m, 1H), 1.98-1.44 (m, 9H) ppm. ESI-MS m/z calc. 746.263, found 747.2 (M+1)⁺.

### Step 2:

A suspension of benzyl (2R,3S)-2-((((1,4-trans)-7′-(2-(benzyloxy)-2-oxoethoxy)-4′,6′-difluoro-3′H-spiro[cyclohexane-1,1′-isobenzofuran]-4-yl)oxy)methyl)-3-(2,2,2-trifluoroacetamido)piperidine-1-carboxylate (3.18 g, 4.046 mmol) and Pd/C (340 mg, 0.160 mmol) in a mixture of EtOAc (25 mL) and MeOH (25 mL) was stirred for 20 h under a hydrogen atmosphere at ambient temperature. The mixture was filtered through a pad of Celite®, washing the filtered cake with a mixture of MeOH and EtOAc (1:1, 500 mL). The mother liquors were concentrated in vacuo. The residue was co-evaporated with heptane (2×50 mL) to give 2-(((1,4-trans)-4′,6′-difluoro-4-(((2R,3S)-3-(2,2,2-trifluoroacetamido)piperidin-2-yl)methoxy)-3′H-spiro[cyclohexane-1,1′-isobenzofuran]-7′-yl)oxy)acetic acid (2.26 g, 99%) as as the predominant geometric isomer and a colourless solid. ¹H NMR (300 MHz, Chloroform-d) δ 10.68-8.94 (m, 2H), 6.75 (dd, J=11.1, 8.0 Hz, 1H), 5.03-4.83 (m, 2H), 4.78 (d, J=14.8 Hz, 1H), 4.55-4.44 (m, 1H), 4.03 (d, J=14.7 Hz, 1H), 3.78 (dd, J=10.9, 9.1 Hz, 1H), 3.68-3.56 (m, 2H), 3.52-3.30 (m, 2H), 2.96 (td, J=13.0, 3.0 Hz, 1H), 2.61-2.39 (m, 2H), 2.17-1.96 (m, 2H), 1.92-1.64 (m, 6H), 1.60-1.49 (m, 1H), 1.35-1.15 (m, 2H) ppm. ESI-MS m/z calc. 522.179, found 523.8 (M+1).

### Step 3:

PhSO₂Cl (0.44 mL, 3.448 mmol) was added to a solution of 2-(((1,4-trans)-4′,6′-difluoro-4-(((2R,3S)-3-(2,2,2-trifluoroacetamido)piperidin-2-yl)methoxy)-3′H-spiro[cyclohexane-1,1′-isobenzofuran]-7′-yl)oxy)acetic acid (1.00 g, 1.723 mmol) and Et₃N (0.72 mL, 5.166 mmol) in a mixture of EtOAc (36 mL) and NMP (36 mL) and the reaction mixture was stirred at 20° C. for 2 h. 1-Methylpiperazine (0.25 mL, 2.254 mmol) was added and the reaction was stirred for 15 min. The mixture was partitioned between MTBE (50 mL) and water (50 mL). The organic layer was separated, washed with a 1 M aqueous HCl solution (50 mL), a saturated aqueous NaHCO₃ solution and brine, dried (MgSO₄), filtered and concentrated in vacuo. Purification by trituration from MeOH (5 mL), followed by trituration from a 1:1 mixture of EtOAc and heptane (5 mL), gave N-((5a,8-trans)-(10aR,11S)-1,3-difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-2,2,2-trifluoroacetamide (609 mg, 70%) as a white solid. ¹H NMR (300 MHz, Chloroform-d) δ 7.15 (d, J=6.9 Hz, 1H), 6.75 (dd, J=10.7, 8.0 Hz, 1H), 5.32 (dd, J=13.0, 2.0 Hz, 1H), 5.14-4.94 (m, 3H), 4.33-4.20 (m, 2H), 4.01 (dd, J=14.5, 5.4 Hz, 1H), 3.84 (d, J=3.6 Hz, 1H), 3.70 (qd, J=8.9, 5.4 Hz, 2H), 3.60-3.43 (m, 1H), 2.74 (td, J=14.7, 4.9 Hz, 1H), 2.00-1.61 (m, 10H), 1.59-1.47 (m, 1H) ppm. ESI-MS m/z calc. 504.168, found 505.3 (M+1)⁺.

### Step 4:

NaOH (20 mL, 1 M aqueous solution, 20 mmol) was added to a suspension of N-((5a,8-trans)-(10aR, 11S)-1,3-difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-2,2,2-trifluoroacetamide (0.94 g, 1.863 mmol) in a mixture of 2-MeTHF (20 mL) and MeOH (3 mL) and the reaction mixture was heated at 55° C. for 2 h. The reaction was cooled to ambient temperature and extracted with EtOAc (3×20 mL). The combined organic layers were washed with a saturated aqueous NaHCO₃ solution (3×20 mL) and brine (20 mL), dried (Na₂SO₄) and filtered through a pad of Celite®, washing the filtered cake with EtOAc (3×50 mL). The mother liquors were concentrated in vacuo and dried under vacuum to give (5a,8-trans)-(10aR,11S)-11-amino-1,3-difluoro-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (810 mg, 98%) as a colourless foam. ¹H NMR (300 MHz, Methanol-d₄) δ 6.93 (dd, J=11.0, 8.3 Hz, 1H), 5.42 (dd, J=12.9, 1.9 Hz, 1H), 5.18-4.93 (m, 3H), 4.17 (dd, J=12.9, 1.9 Hz, 1H), 4.01-3.80 (m, 2H), 3.76 (d, J=3.1 Hz, 1H), 3.56 (dd, J=9.0, 3.5 Hz, 1H), 3.49-3.31 (m, 1H), 3.00 (ddd, J=11.9, 5.6, 3.9 Hz, 1H), 2.78 (td, J=15.3, 5.0 Hz, 1H), 2.22-2.04 (m, 2H), 1.94-1.46 (m, 9H) ppm; exchangeable H not observed. ESI-MS m/z calc. 408.186, found 408.5 (M+1)⁺.

### Step 5:

Int-5 (50.7 mg, 0.146 mmol) was added to a solution of (5a,8-trans)-(10aR,11S)-11-amino-1,3-difluoro-7,8,10,10a,11,12,13,14-octahydro-4H,6H-5a,8-ethanoisobenzofuro[7,1-hi]pyrido[2,1-c][1,7]dioxa[4]azacyclotridecin-16(17H)-one (30 mg, 0.073 mmol) and DIPEA (25.0 μL, 0.144 mmol) in MeCN (1.53 mL) and the reaction mixture was stirred at ambient temperature for 16 h. The reaction was diluted with a saturated NH₄Cl solution (5 mL) and water (5 mL), extracted with DCM (2×10 mL) and the organic layer was concentrated in vacuo. Purification by reverse phase HPLC (XSelect CSH C₁₈ OBD column, MeCN in water with 0.2% formic acid) gave N-((5a,8-trans)-(10aR,11S)-1,3-difluoro-16-oxo-7,8,10,10a,11,12,13,14,16,17-decahydro-4H,6H-5a,8-ethanoisobenzofuro[7,1-hi]pyrido[2,1-c][1,7]dioxa[4]azacyclotridecin-11-yl)-1,1-difluoromethanesulfonamide (28, 10.3 mg, 26%) as a white solid. ¹H NMR (300 MHz, Chloroform-d) δ 6.77 (dd, J=10.8, 8.1 Hz, 1H), 6.25 (t, J=54.0 Hz, 1H), 5.44-4.93 (m, 6H), 4.28 (dd, J=13.0, 2.0 Hz, 1H), 3.95 (s, 1H), 3.86 (s, 1H), 3.76 (d, J=5.9 Hz, 2H), 2.75 (d, J=5.0 Hz, 1H), 1.94 (t, J=13.2 Hz, 3H), 1.83-1.67 (m, 3H), 1.54 (s, 5H) ppm; exchangeable H not observed. ESI-MS m/z calc. 522.145, found 523.5 (M+1)⁺.

### Example 28

### N-((5a,8-cis)-(10aR,11S)-16-Oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)azetidine-3-sulfonamide (29)

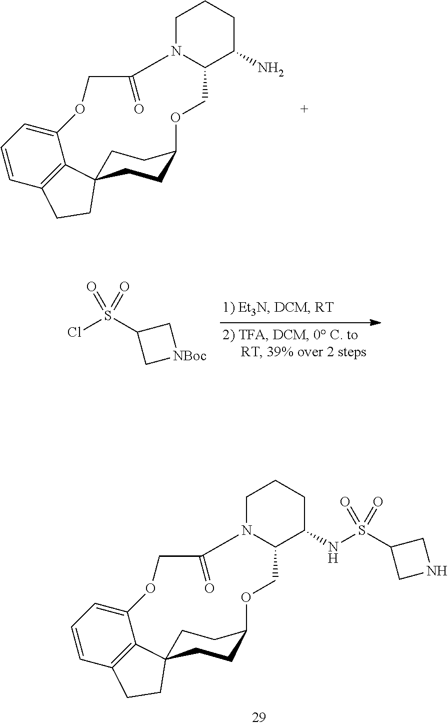

### Step 1:

tert-Butyl 3-(chlorosulfonyl)azetidine-1-carboxylate (15.9 mg, 0.062 mmol) was added to a stirred mixture of (5a,8-cis)-(10aR,11S)-11-amino-4,5,7,8,10,10a,11,12,13,14-decahydro-6H-5a,8-ethanoindeno[7,1-hi]pyrido[2,1-c][1,7]dioxa[4]azacyclotridecin-16(17H)-one (20 mg, prepared using the method described in Example 4 Step 1, 0.054 mmol) and Et₃N (15.06 μL, 0.108 mmol) in DCM (400 μL) and the reaction mixture was stirred at ambient temperature overnight. Additional tert-butyl 3-(chlorosulfonyl)azetidine-1-carboxylate (6.9 mg, 0.027 mmol) was added and the mixture was stirred for at ambient temperature for a further 5 h. The reaction was quenched by addition of a saturated aqueous NaHCO₃ solution and extracted with DCM (2×). The combined organic layers were dried by passing the solution through a phase separator cartridge and the mother liquors were concentrated in vacuo to give tert-butyl 3-(N-((5a,8-cis)-(10aR,11S)-16-oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)sulfamoyl)azetidine-1-carboxylate. ESI-MS m/z calc. 589.282, found 623.3 (M+1)⁺.

### Step 2:

TFA (21 μL, 0.273 mmol) was added to a stirred solution of tert-butyl 3-(N-((5a,8-cis)-(10aR,11S)-16-oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)sulfamoyl)azetidine-1-carboxylate (31.83 mg, 0.054 mmol) in DCM (640 μL) at 0° C. and the reaction mixture was warmed and stirred at ambient temperature for 2.5 h. Additional TFA (21 μL, 0.273 mmol) was added and the mixture was stirred at ambient temperature overnight. The reaction was quenched by addition of a saturated aqueous NaHCO₃ solution and stirred for 10 min. The solution was extracted with DCM (4×) and a 1:3 solution of ⁱPrOH in CHCl₃ (2×). The combined organic layers were dried by passing the solution through a phase separator cartridge and concentrated in vacuo. Purification by reverse phase HPLC (XSelect CSH C₁₈ OBD column, MeCN in water with 0.2% formic acid) gave N-((5a,8-cis)-(10aR,11S)-16-oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)azetidine-3-sulfonamide (29, 10.9 mg, 39% over 2 steps) as a white solid. ¹H NMR (300 MHz, Chloroform-d) δ 7.08 (t, J=7.6 Hz, 1H), 6.85 (d, J=7.5 Hz, 1H), 6.57 (d, J=7.9 Hz, 1H), 5.18-4.98 (m, 2H), 4.57-4.29 (m, 5H), 4.22 (d, J=11.5 Hz, 1H), 3.85-3.68 (m, 2H), 3.68-3.56 (m, 2H), 3.56-3.46 (m, 1H), 3.46-3.29 (m, 1H), 3.04-2.73 (m, 3H), 2.28-2.07 (m, 2H), 2.07-1.89 (m, 3H), 1.83-1.70 (m, 4H), 1.57-1.40 (m, 2H), 1.40-1.30 (m, 1H), 1.30-1.17 (m, 2H), 1.10 (d, J=12.4 Hz, 1H) ppm. ESI-MS m/z calc. 489.230, found 490.5 (M+1)⁺.

### Example 29

### 2-Hydroxy-N-((5a,8-cis)-(10aR,11S)-16-oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)ethane-1-sulfonamide (30)

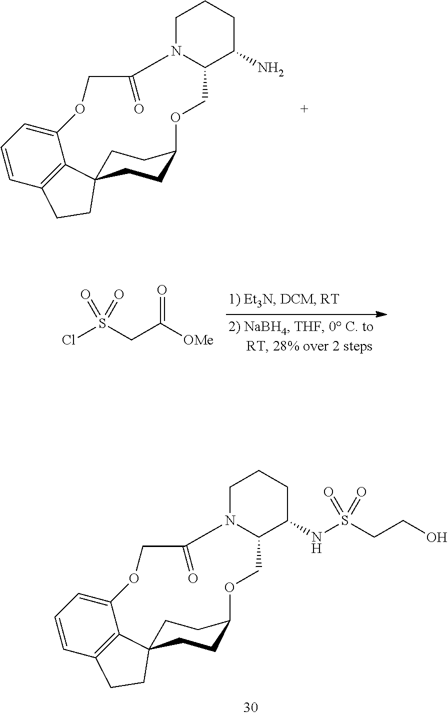

### Step 1:

Methyl 2-(chlorosulfonyl)acetate (11.3 mg, 0.065 mmol) was added to a stirred mixture of (5a,8-cis)-(10aR,11S)-11-amino-4,5,7,8,10,10a,11,12,13,14-decahydro-6H-5a,8-ethanoindeno[7,1-hi]pyrido[2,1-c][1,7]dioxa[4]azacyclotridecin-16(17H)-one (20 mg, prepared using the method described in Example 4 Step 1, 0.054 mmol) and Et₃N (15.06 μL, 0.108 mmol) in DCM (400 μL) and the reaction mixture was stirred at ambient temperature overnight. The reaction was quenched by addition of a saturated aqueous NaHCO₃ solution and extracted with DCM (2×). The combined organic layers were dried by passing the solution through a phase separator cartridge and the mother liquors were concentrated in vacuo to give methyl 2-(N-((5a,8-cis)-(10aR,11S)-16-oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)sulfamoyl)acetate. ESI-MS m/z calc. 506.209, found 506.8 (M+1)⁺.

### Step 2:

NaBH₄ (10.2 mg, 0.270 mmol) was added to a stirred solution of methyl 2-(N-((5a,8-cis)-(10aR,11S)-16-oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)sulfamoyl)acetate (23.75 mg, 0.047 mmol) in THF (340 μL) at 0° C. and the reaction mixture was warmed to ambient temperature and stirred for 4 h. The reaction was quenched by addition of a saturated aqueous NH₄Cl solution and extracted with DCM (2×). The combined organic phases were dried by passing the solution through a phase separator cartridge and the mother liquors were concentrated in vacuo. Purification by reverse phase HPLC (XSelect CSH Cis OBD column, MeCN in water with 0.1% TFA) gave 2-hydroxy-N-((5a,8-cis)-(10aR,11S)-16-oxo-4,5,7,8,10,10a,11,12,13,14,16,17-dodecahydro-6H-5a,8-ethanoindeno[7,1-hi]pyrido[2,1-c][1,7]dioxa[4]azacyclotridecin-11-yl)ethane-1-sulfonamide (30, 6.5 mg, 28% over 2 steps) as a colourless oil. ¹H NMR (300 MHz, Chloroform-d) δ 7.11 (t, J=7.7 Hz, 1H), 6.87 (d, J=7.5 Hz, 1H), 6.61 (d, J=7.9 Hz, 1H), 5.28-5.13 (m, 2H), 5.02 (s, 3H), 4.83-4.66 (m, 1H), 4.36 (dd, J=11.8, 4.6 Hz, 1H), 4.12 (t, J=5.2 Hz, 1H), 3.89-3.77 (m, 2H), 3.72 (s, 2H), 3.59-3.44 (m, 2H), 3.36 (t, J=5.2 Hz, 1H), 3.03-2.85 (m, 3H), 2.20 (dt, J=11.8, 5.6 Hz, 1H), 2.11-1.86 (m, 4H), 1.84-1.59 (m, 4H), 1.58-1.35 (m, 2H), 1.28 (d, J=13.5 Hz, 1H), 1.17 (d, J=13.3 Hz, 1H) ppm. ESI-MS m/z calc. 478.214, found 479.3 (M+1)⁺.

### Example 30

### N-((5a,8-trans)-(10aR,11S,13R)-3-Bromo-13-methyl-15-oxo-7,8,10a,11,12,13,16,17-octahydro-4H,6H,10H,15H-5a, 8-ethanoisobenzofuro[7,1-ij]pyrrolo[2,1-c][1,8]dioxa[4]azacyclotetradecin-11-yl)-1,1,1-trifluoromethanesulfonamide (31)

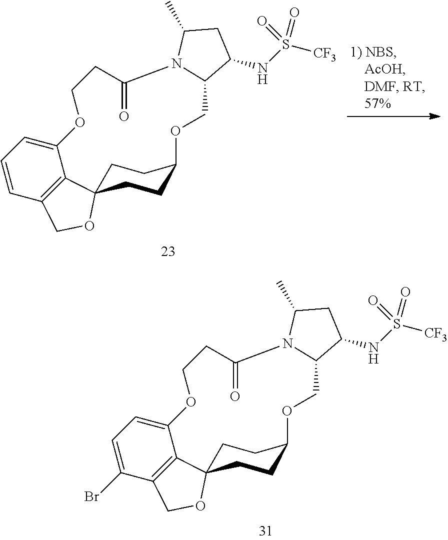

### Step 1:

NBS (34 mg, 0.191 mmol) was added to a solution of 1,1,1-trifluoro-N-((5a,8-trans)-(10aR,11S,13R)-13-methyl-15-oxo-7,8,10a,11,12,13,16,17-octahydro-4H,6H,10H,15H-5a,8-ethanoisobenzofuro[7,1-ij]pyrrolo[2,1-c][1,8]dioxa[4]azacyclotetradecin-11-yl)methanesulfonamide (23, 50 mg, 0.096 mmol) in a mixture of DMF (0.7 mL) and acetic acid (0.3 mL) and the reaction mixture was stirred at ambient temperature for 18 h. Purification by reverse phase HPLC (XSelect CSH C₁₈ OBD column, MeCN in water with 0.1% TFA) gave N-((5a,8-trans)-(10aR,11S,13R)-3-bromo-13-methyl-15-oxo-7,8,10a,11,12,13,16,17-octahydro-4H,6H,10H,15H-5a,8-ethanoisobenzofuro[7,1-ij]pyrrolo[2,1-c][1,8]dioxa[4]azacyclotetradecin-11-yl)-1,1,1-trifluoromethanesulfonamide (31, 33 mg, 57%) as a white solid. ¹H NMR (300 MHz, Chloroform-d) δ 7.64 (d, J=5.6 Hz, 1H), 7.32 (d, J=8.6 Hz, 1H), 6.78 (d, J=8.6 Hz, 1H), 5.10-4.93 (m, 2H), 4.70 (dd, J=9.4, 2.8 Hz, 1H), 4.47 (tt, J=4.7, 2.4 Hz, 3H), 4.34-4.17 (m, 2H), 3.94 (s, 1H), 3.35 (d, J=9.4 Hz, 1H), 3.20-3.02 (m, 1H), 2.62-2.46 (d, 2H), 2.34 (ddd, J=14.1, 8.1, 6.1 Hz, 1H), 2.231-1.88 (i, 4H), 1.871-1.70 (i, 2H), 1.56 (d, J=6.8 Hz, 4H), 1.41 (d, J=13.8 Hz, 1H) ppm. ESI-MS m/z calc. 596.080, found 597.4 (M+1)-.

The following compounds were made using methods analogous to those disclosed in Examples 1-30.

| | | Starting | | |
|---|---|---|---|---|
| Cmpd | Compound Structure | Materials/ | | |
| No. | Compound Name | Reagents | LC/MS | NMR (shifts in ppm) |
| 32 | 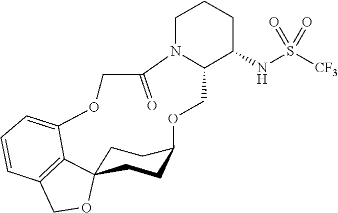 | Int-1/Int-2/ Tf₂O | ESI-MS m/z calc. 504.154, found 505.2 (M + 1)⁺. | ¹H NMR (400 MHz, Methanol- d₄) δ 7.23 (t, J = 7.8 Hz, 1H), 6.89 (d, J = 7.6 Hz, 1H), 6.81 (d, J = 8.0 Hz, 1H), 5.36 (d, J = 11.6 Hz, 1H), 5.19-5.09 (m, 1H), 5.08-4.96 (m, 2H), 4.17 (d, J = 11.6 Hz, 1H), 3.99-3.82 (m, 2H), 3.78 (s, 1H), 3.75-3.63 (m, 1H), 3.55-3.38 (m, 2H), 2.97- 2.80 (m, 1H), 2.19-1.99 (m, 2H), 1.97-1.53 (m, 8H), 1.39 (d, J = 14.1 Hz, 1H) ppm; exchangeable H not observed. |
| | 1,1,1-Trifluoro-N-((5a,8- | | | |
| | trans)-(10aR,11S)-16-oxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)methanesulfonamide | | | |
| 33 | 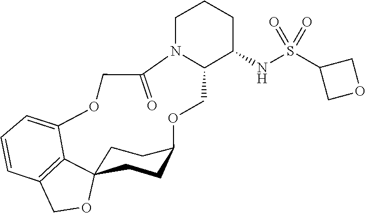 | Int-1/Int-2/ oxetane-3- sulfonyl chloride | ESI-MS m/z calc. 492.193, found 493.5 (M + 1)⁺. | |
| | N-((5a,8-trans)- | | | |
| | (10aR,11S)-16-Oxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)oxetane-3- | | | |
| | sulfonamide | | | |
| 34 | 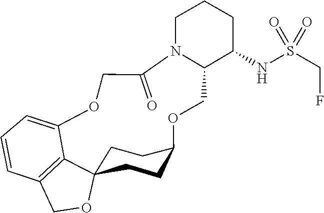 | Int-1/Int-2/ FCH₂SO₂Cl | ESI-MS m/z calc. 468.173, found 469.5 (M + 1)⁺. | |
| | 1-Fluoro-N-((5a,8-trans)- | | | |
| | (10aR,11S)-16-oxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11- | | | |
| | yl)methanesulfonamide | | | |
| 35 | 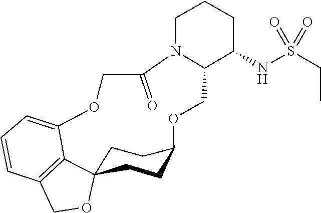 | Int-1/Int-2/ EtSO₂Cl | ESI-MS m/z calc. 464.198, found 465.5 (M + 1)⁺. | |
| | N-((5a,8-trans)- | | | |
| | (10aR,11S)-16-Oxo- | | | |
| | 7,8,10,10a,11,12,13,14,16, | | | |
| | 17-decahydro-4H,6H-5a,8- | | | |
| | ethanoisobenzofuro[7,1-hi] | | | |
| | pyrido[2,1-c][1,7]dioxa[4] | | | |
| | azacyclotridecin-11- | | | |
| | yl)ethanesulfonamide | | | |
| 36 | 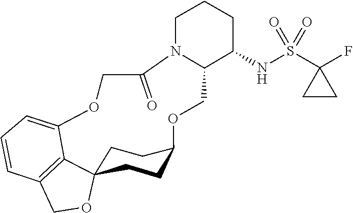 | Int-1/Int-2/ Int-4 | ESI-MS m/z calc. 494.189, found 495.4 (M + 1)⁺. | |
| | 1-Fluoro-N-((5a,8-trans)- | | | |
| | (10aR,11S)-16-Oxo- | | | |
| | 7,8,10,10a,11,12,13,14,16,17- | | | |
| | decahydro-4H,6H-5a,8- | | | |
| | ethanoisobenzofuro[7,1-hi]pyrido | | | |
| | [2,1-c][1,7]dioxa[4]azacyclotridecin- | | | |
| | 11-yl)cyclopropane-1-sulfonamide | | | |
| 37 | 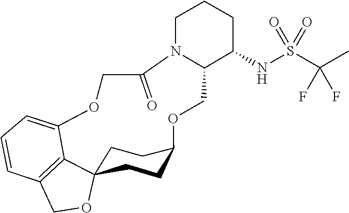 | Int-1/Int-2/ Int-38 | ESI-MS m/z calc. 500.179, found 501.5 (M + 1)⁺. | ¹H NMR (400 MHz, Methanol- d₄) δ 7.23 (t, J = 8.0 Hz, 1H), 6.89 (d, J = 7.7 Hz, 1H), 6.81 (d, J = 8.0 Hz, 1H), 5.35 (d, J = 11.6 Hz, 1H), 5.19- 5.10 (m, 1H), 5.06-4.97 (m, 2H), 4.21-4.11 (m, 1H), 3.96- 3.83 (m, 2H), 3.78 (s, 1H), 3.72-3.65 (m, 1H), 3.60-3.52 (m, 1H), 3.45 (t, J = 12.9 Hz, 1H), 2.95-2.80 (m, 1H), 2.20-2.05 (m, 2H), 2.02-1.59 (m, 12H), 1.39 (d, J = 13.9 Hz, 1H) ppm. |
| | 1,1-Difluoro-N-((5a,8- | | | |
| | trans)-(10aR,11S)-16-oxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)ethane-1-sulfonamide | | | |
| 38 | 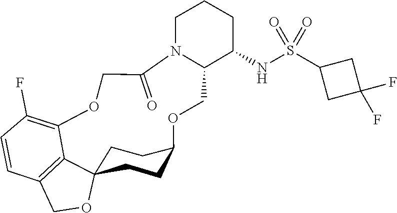 | Int-6/Int-2/ 3,3-difluoro- cyclobutane- 1-sulfonyl chloride | ESI-MS m/z calc. 544.185, found 545.5 (M + 1)⁺. | ¹H NMR (300 MHz, Methanol-d₄) δ 7.08 (dd, J = 11.3, 8.2 Hz, 1H), 6.94 (dd, J = 8.2, 4.0 Hz, 1H), 5.47 (dd, J = 12.8, 2.2 Hz, 1H), 5.16 (dd, J = 10.2, 4.9 Hz, 1H), 5.03- 4.94 (m, 2H), 4.21 (dd, J = 12.8, 2.0 Hz, 1H), 4.04-3.69 (m, 4H), 3.65-3.55 (m, 2H), 3.46 (t, J = 11.6 Hz, 1H), 3.11-2.69 (m, 5H), 2.05 (s, 3H), 1.90 (s, 1H), 1.63 (td, J = 37.9, 12.2 Hz, 7H) ppm; exchangeable H not observed. |
| | 3,3-Difluoro-N-((5a,8- | | | |
| | trans)-(10aR,11S)-1- | | | |
| | fluoro-16-oxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)cyclobutane-1-sulfonamide | | | |
| 39 | 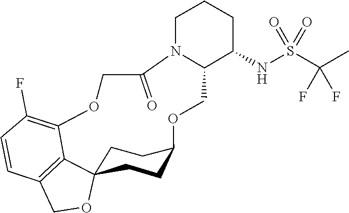 | Int-6/Int-2/ Int-38 | ESI-MS m/z calc. 518.170, found 519.5 (M + 1)⁺. | ¹H NMR (300 MHz,Methanol-d₄) δ 7.08 (dd, J = 11.3, 8.2 Hz, 1H), 6.94 (dd, J = 8.2, 4.0 Hz, 1H), 5.47 (dd, J = 12.8, 2.2 Hz, 1H), 5.21 (d, J = 5.3 Hz, 1H), 4.99 (s, 2H), 4.20 (dd, J = 12.8, 2.0 Hz, 1H), 4.01-3.85 (m, 2H), 3.80 (s, 1H), 3.72 (s, 1H), 3.64-3.51 (m, 1H), 3.46 (t, J = 12.8 Hz, 1H), 2.92-2.76 (m, 1H), 2.07 (d, J = 13.1 Hz, 3H), 1.94 (d, J = 5.9 Hz, 4H), 1.81-1.59 (m, 6H), 1.50 (d, J = 13.8 Hz, 1H) ppm; exchangeable H not observed. |
| | 1,1-Difluoro-N-((5a,8-trans)- | | | |
| | (10aR,11S)-1- | | | |
| | fluoro-16-oxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)ethane-1-sulfonamide | | | |
| 40 | 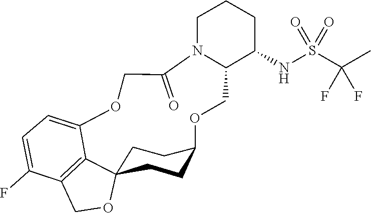 | Int-7/Int-2/ Int-38 | ESI-MS m/z calc. 518.170, found 519.5 (M + 1)⁺. | ¹H NMR (400 MHz, Methanol-d₄) δ 6.97-6.89 (m, 1H), 6.89-6.77 (m, 1H), 5.34 (dd, J = 11.6, 1.9 Hz, 1H), 5.17-5.00 (m, 3H), 4.14 (dt, J = 11.7, 1.4 Hz, 1H), 3.93-3.82 (m, 2H), 3.78 (dh, J = 5.6, 1.6 Hz, 1H), 3.72-3.62 (m, 1H), 3.58-3.50 (m, 1H), 3.52-3.37 (m, 1H), 2.94-2.76 (m, 1H), 2.17-2.03 (m, 2H), 2.03-1.55 (m, 11H), 1.48-1.38 (m, 1H) ppm; exchangeable H not observed. |
| | 1,1-Difluoro-N-((5a,8-trans)- | | | |
| | (10aR,11S)-3- | | | |
| | fluoro-16-oxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)ethane-1-sulfonamide | | | |
| 41 | 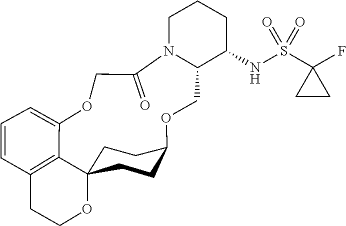 | Int-23/Int-2/ Int-4 | ESI-MS m/z calc. 508.204, found 509.6 (M + 1)⁺. | ¹H NMR (300 MHz, Chloroform- d) δ 7.11 (t, J = 7.8 Hz, 1H), 6.76 (d, J = 7.5 Hz, 1H), 6.59 (d, J = 8.2 Hz, 1H), 5.30 (dt, J = 10.0, 5.0 Hz, 1H), 5.05 (d, J = 10.1 Hz, 1H), 4.83 (d, J = 8.3 Hz, 1H), 4.24 (d, J = 10.1 Hz, 1H), 3.88-3.78 (m, 4H), 3.72 (s, 1H), 3.67-3.56 (m, 2H), 3.49-3.34 (m, 1H), 2.82-2.75 (m, 2H), 2.48 (ddd, J = 14.0, 11.5, 6.5 Hz, 1H), 1.92-1.80 (m, 2H), 1.79-1.68 (m, 4H), 1.66-1.47 (m, 9H) ppm. |
| | 1-Fluoro-N-((6a,9-trans)- | | | |
| | (11aR,12S)-17-oxo- | | | |
| | 4,5,8,9,11,11a,12,13,14,15, | | | |
| | 17,18-dodecahydro-7H- | | | |
| | 6a,9- | | | |
| | ethanoisochromeno[8,1- | | | |
| | hi]pyrido[2,1- | | | |
| | c][1,7]dioxa[4]azacyclotridecin- | | | |
| | 12-yl)cyclopropane-1-sulfonamide | | | |
| 42 | 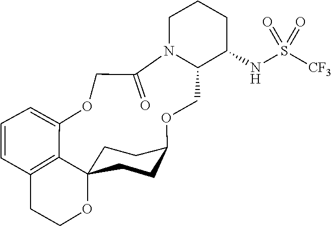 | Int-23/Int-2/ Tf₂O | ESI-MS m/z calc. 518.170, found 519.5 (M + 1)⁺. | ¹H NMR (300 MHz, Methanol- d₄) δ 7.11 (t, J = 7.8 Hz, 1H), 6.77 (d, J = 7.6 Hz, 1H), 6.60 (d, J = 8.0 Hz, 1H), 5.31 (d, J = 8.2 Hz, 1H), 5.25-5.15 (m, 1H), 5.07 (d, J = 10.2 Hz, 1H), 4.25 (d, J = 10.2 Hz, 1H), 3.96 (s, 1H), 3.84 (t, J = 5.4 Hz, 2H), 3.80-3.72 (m, 2H), 3.67 (dd, J = 8.7, 4.4 Hz, 2H), 3.54-3.40 (m, 1H), 2.78 (q, J = 5.4 Hz, 2H), 2.43 (dt, J = 13.9, 9.0 Hz, 1H), 2.02-1.85 (m, 2H), 1.85-1.60 (m, 7H), 1.49 (d, 2H) ppm. |
| | 1,1,1-Trifluoro-N-((6a,9- | | | |
| | trans)-(11aR,12S)-17-oxo- | | | |
| | 4,5,8,9,11,11a,12,13,14,15, | | | |
| | 17,18-dodecahydro-7H- | | | |
| | 6a,9- | | | |
| | ethanoisochromeno[8,1- | | | |
| | hi]pyrido[2,1- | | | |
| | c][1,7]dioxa[4]azacyclotridecin-12- | | | |
| | yl)methanesulfonamide | | | |
| 43 | 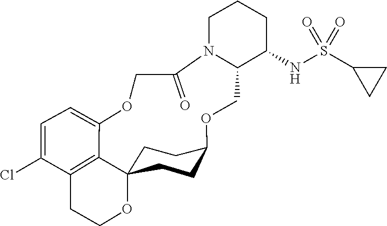 | Int-23/Int-2/ cyclopropane- sulfonyl chloride | ESI-MS m/z calc. 524.175, found 525.5 (M + 1)⁺. | ¹H NMR (300 MHz, Chloroform- d) δ 7.20 (d, J = 8.6 Hz, 1H), 6.56 (d, J = 8.7 Hz, 1H), 5.31 (dt, J = 10.3, 5.0 Hz, 1H), 5.04 (d, J = 10.0 Hz, 1H), 4.37-4.17 (m, 2H), 3.92- 3.78 (m, 3H), 3.71 (s, 2H), 3.64- 3.52 (m, 2H), 3.38 (t, J = 11.9 Hz, 1H), 2.80-2.73 (m, 2H), 2.56- 2.41 (m, 2H), 2.10-1.97 (m, 1H), 1.93-1.65 (m, 7H), 1.51 (dd, J = 28.8, 14.7 Hz, 3H), 1.32-0.95 (m, 4H) ppm. |
| | N-((6a,9-trans)- | | | |
| | (11aR,12S)-3-Chloro-17-oxo- | | | |
| | 4,5,8,9,11,11a,12,13,14,15, | | | |
| | 17,18-dodecahydro-7H- | | | |
| | 6a,9- | | | |
| | ethanoisochromeno[8,1- | | | |
| | hi]pyrido[2,1- | | | |
| | c][1,7]dioxa[4]azacyclotridecin-12- | | | |
| | yl)cyclopropanesulfonamide | | | |
| 44 | 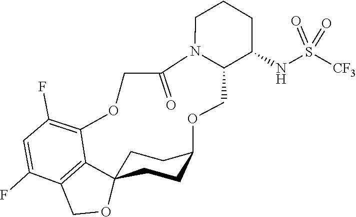 | Int-9/Int-2/ Tf₂O | ESI-MS m/z calc. 540.135, found 541.4 (M + 1)⁺. | ¹H NMR (300 MHz, Methanol-d₄) δ 6.93 (dd, J = 11.0, 8.4 Hz, 1H), 5.43 (dd, J = 12.9, 2.0 Hz, 1H), 5.23- 5.10 (m, 1H), 5.10-4.94 (m, 2H), 4.17 (dd, J = 12.9, 1.9 Hz, 1H), 4.04-3.41 (m, 6H), 2.88-2.69 (m, 1H), 2.27- 1.41 (m, 11H) ppm; exchangeable H not observed. |
| | N-((5a,8-trans)- | | | |
| | (10aR,11S)-1,3-Difluoro- | | | |
| | 16-oxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)-1,1,1-trifluoromethanesulfonamide | | | |
| 45 | 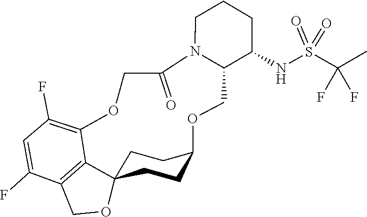 | Int-9/Int-2/ Int-38 | ESI-MS m/z calc. 536.160, found 537.4 (M + 1)⁺. | ¹H NMR (400 MHz, DMSO- d₆) δ 8.70 (d, J = 8.1 Hz, 1H), 7.26 (dd, J = 11.0, 8.5 Hz, 1H), 5.39 (dd, J = 12.8, 1.6 Hz, 1H), 4.97 (q, J = 12.4 Hz, 3H), 4.04 (dd, J = 12.7, 1.9 Hz, 1H), 3.89-3.66 (m, 3H), 3.45 (dd, J = 9.1, 3.2 Hz, 2H), 3.31-3.20 (m, 1H), 2.68- 2.59 (m, 1H), 2.22-1.88 (m, 5H), 1.82-1.34 (m, 9H) ppm. |
| | N-((5a,8-trans)- | | | |
| | (10aR,11S)-1,3-Difluoro- | | | |
| | 16-oxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)-1,1- | | | |
| | difluoroethane-1-sulfonamide | | | |
| 46 | 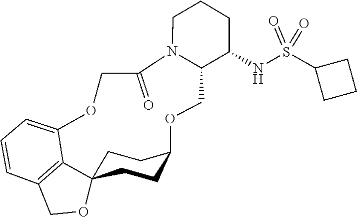 | Int-1/Int-2/ cyclo- butanesulfonyl chloride | ESI-MS m/z calc. 490.214, found 491.5 (M + 1)⁺. | ¹H NMR (400 MHz, DMSO-d₆) δ 7.31 (d, J = 7.7 Hz, 1H), 7.22 (t, J = 7.7 Hz, 1H), 6.89 (d, J = 7.4 Hz, 1H), 6.80 (d, J = 7.9 Hz, 1H), 5.34 (d, J = 11.4 Hz, 1H), 5.02-4.84 (m, 3H), 4.01 (d, J = 11.4 Hz, 1H), 3.97-3.89 (m, 1H), 3.82-3.74 (m, 1H), 3.70 (s, 1H), 3.43 (dd, J = 9.1, 3.3 Hz, 1H), 3.27 (t, J = 13.8 Hz, 2H), 2.82-2.71 (m, 1H), 2.34-2.18 (m, 4H), 2.07-1.84 (m, 4H), 1.79- 1.41 (m, 9H), 1.25 (d, J = 13.6 Hz, 1H) ppm. |
| | N-((5a,8-trans)- | | | |
| | (10aR,11S)-16-Oxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)cyclobutanesulfonamide | | | |
| 47 | 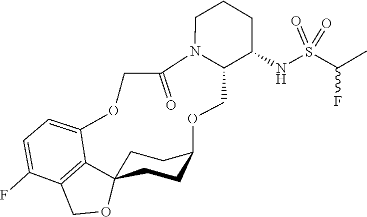 | Int-7/Int-2/ 1-fluoroethane-1- sulfonyl chloride | ESI-MS m/z calc. 500.179, found 501.5 (M + 1)⁺. | ¹H NMR (300 MHz, Methanol- d₄) δ 6.77-6.59 (m, 2H), 5.34- 5.05 (m, 2H), 4.94-4.76 (m, 3H), 3.92 (d, J = 11.8 Hz, 1H), 3.78- 3.50 (m, 3H), 3.45-3.12 (m, 3H), 2.72-2.53 (m, 1H), 2.01-1.74 (m, 2H), 1.70-1.35 (m, 11H), 1.20 (d, J = 13.7 Hz, 1H) ppm; exchangeable H not observed. |
| | dia-(R)-1-Fluoro-N-((5a,8- | | | |
| | trans)-(10aR*,11S*)-3- | | | |
| | fluoro-16-oxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)ethane-1-sulfonamide | | | |
| 48 | 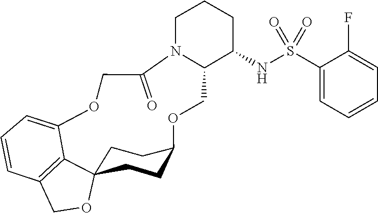 | Int-1/Int-2/2- fluoro- benzenesulfonyl chloride | ESI-MS m/z calc. 530.189, found 531.5 (M + 1)⁺. | |
| | 2-Fluoro-N-((5a,8-trans)- | | | |
| | (10aR,11S)-16-oxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)benzenesulfonamide | | | |
| 49 | 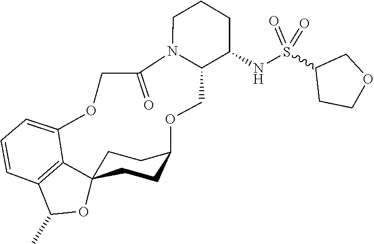 | Int-13/Int-2/ rac- tetrahydrofuran- 3-sulfonyl chloride | ESI-MS m/z calc. 520.224, found 521.6 (M + 1)⁺. | ¹H NMR (300 MHz, Chloroform-d) δ 7.27-7.19 (m, 1H), 6.85-6.69 (m, 2H), 5.41-4.51 (m, 4H), 4.41- 4.31 (m, 1H), 4.21-4.09 (m, 2H), 4.09-3.94 (m, 2H), 3.93- 3.69 (m, 5H), 3.68-3.44 (m, 2H), 2.96-2.80 (m, 1H), 2.44- 2.24 (m, 5H), 2.09-1.60 (m, 8H), 1.56-1.39 (m, 3H) ppm. |
| | dia-(S)-N-((5a,8-trans)- | | | |
| | (4R^{†},10aR*,11S*)-4- | | | |
| | Methyl-16-oxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)tetrahydrofuran- | | | |
| | 3-sulfonamide | | | |
| | (First eluting peak by SFC | | | |
| | carried out at amide | | | |
| | macrocyclisation step) | | | |
| 50 | 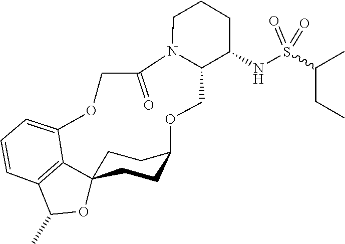 | Int-13/Int-2/ rac-butane-2- sulfonyl chloride | ESI-MS m/z calc. 506.245, found 507.6 (M + 1)⁺. | |
| | dia-(S)-N-((5a,8-trans)- | | | |
| | (4R^{†},10aR*,11S*)-4- | | | |
| | Methyl-16-oxo- | | | |
| | 7,8,10,10a,11,12,13,14,16, | | | |
| | 17-decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)butane-2-sulfonamide | | | |
| | (First eluting peak by SFC | | | |
| | carried out at amide | | | |
| | macrocyclisation step) | | | |
| 51 | 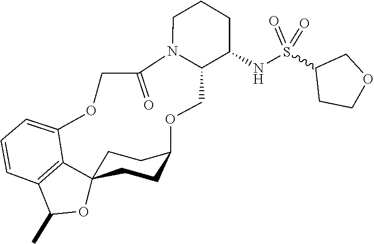 | Int-13/Int-2/ rac- tetrahydrofuran- 3-sulfonyl chloride | ESI-MS m/z calc. 520.224, found 521.6 (M + 1)⁺. | |
| | dia-(S)-N-((5a,8-trans)- | | | |
| | 4S^{†},10aR*,11S*)-4-Methyl- | | | |
| | 16-oxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)tetrahydrofuran- | | | |
| | 3-sulfonamide | | | |
| | (Second eluting peak by | | | |
| | SFC carried out at amide | | | |
| | macrocyclisation step) | | | |
| 52 | 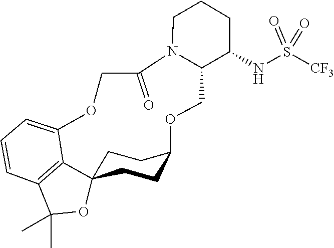 | Int-14/Int-2/ Tf₂O | ESI-MS m/z calc. 532.185, found 533.5 (M + 1)⁺. | ¹H NMR (300 MHz, DMSO- d₆) δ 7.22 (t, J = 7.8 Hz, 1H), 7.02-6.69 (m, 2H), 5.31 (d, J = 11.0 Hz, 1H), 4.98-4.48 (m, 1H), 4.03 (d, J = 11.0 Hz, 1H), 3.91-3.59 (m, 3H), 3.59- 3.39 (m, 2H), 2.85-2.53 (m, 2H), 2.27 (s, 1H), 2.12-1.87 (m, 1H), 1.78-1.29 (m, 15H), 1.23-1.13 (m, 1H) ppm. |
| | N-((5a,8-trans)- | | | |
| | (10aR,11S)-4,4-Dimethyl- | | | |
| | 16-oxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)-1,1,1- | | | |
| | trifluoromethanesulfonamide | | | |
| 53 | 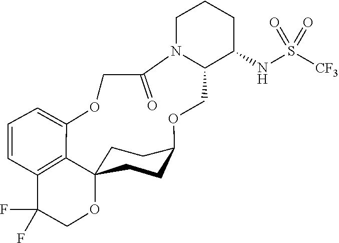 | Int-25/Int-2/ Tf₂O | ESI-MS m/z calc. 554.151, found 555.5 (M + 1)⁺. | ¹H NMR (300 MHz, Methanol-d₄) δ 7.46-7.22 (m, 2H), 7.05-6.96 (m, 1H), 5.33 (d, J = 10.2 Hz, ,1H), 5.21 (dt, J = 10.5, 4.8 Hz, 1H), 4.12 (d, J = 10.2 Hz, 1H), 4.04-3.87 (m, 3H), 3.75 (d, J = 12.7 Hz, 3H), 3.57-3.35 (m, 2H), 2.88 (td, J = 13.1, 4.8 Hz, 1H), 2.68- 2.42 (m, 1H), 2.10-1.42 (m, 11H) ppm. |
| | N-((6a,9-trans)- | | | |
| | (11aR,12S)-4,4-Difluoro- | | | |
| | 17-oxo- | | | |
| | 4,5,8,9,11,11a,12,13,14,15, | | | |
| | 17,18-dodecahydro-7H- | | | |
| | 6a,9- | | | |
| | ethanoisochromeno[8,1- | | | |
| | hi]pyrido[2,1- | | | |
| | c][1,7]dioxa[4]azacyclotridecin-12-yl)-1,1,1- | | | |
| | trifluoromethanesulfonamide | | | |
| 54 | 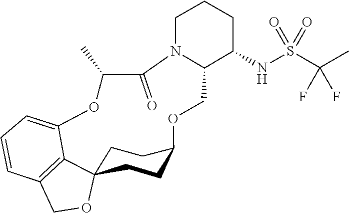 | Int-18/Int-2/ Int-38 | ESI-MS m/z calc. 514.195, found 515.4 (M + 1)⁺. | ¹H NMR (400 MHz, Methanol- d₄) δ 7.23 (t, J = 7.7 Hz, 1H), 7.00 (t, J = 7.5 Hz, 2H), 5.23-5.12 (m, 1H), 4.98 (s, 2H), 4.47 (q, J = 7.1 Hz, 1H), 4.37-4.26 (m, 1H), 3.89 (dd, J = 11.0, 8.9 Hz, 1H), 3.80- 3.76 (m, 1H), 3.66 (dt, J = 13.0, 4.9 Hz, 1H), 3.55 (dd, J = 8.9, 3.7 Hz, 1H), 3.43-3.34 (m, 1H), 2.91 (td, J = 13.8, 4.8 Hz, 1H), 2.15- 2.09 (m, 1H), 2.04-1.41 (m, 16H) ppm; exchangeable H not observed. |
| | 1,1-Difluoro-N-((5a,8- | | | |
| | trans)-(10aR,11S,17S)-17- | | | |
| | methyl-16-oxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)ethane-1-sulfonamide | | | |
| 55 | 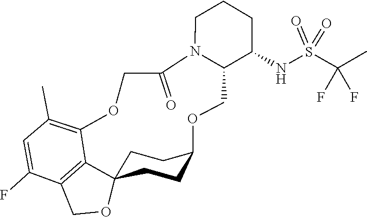 | Int-10/Int-2/ Int-38 | ESI-MS m/z calc. 532.185, found 533.5 (M + 1)⁺. | ¹H NMR (400 MHz, Methanol-d₄) δ 6.83 (d, J = 9.1 Hz, 1H), 5.30 (d, J = 13.0 Hz, 1H), 5.20 (ddd, J = 10.0, 5.8, 3.3 Hz, 1H), 4.98 (s, 2H), 4.09 (d, J = 13.0 Hz, 1H), 3.95 (d, J = 13.8 Hz, 1H), 3.85 (dd, J = 11.0, 8.9 Hz, 1H), 3.77 (s, 1H), 3.66 (dd, J = 12.1, 6.1 Hz, 1H), 3.56 (dd, J = 8.8, 3.3 Hz, 1H), 3.38 (t, J = 13.1 Hz, 1H), 2.71 (dd, J = 14.0, 4.7 Hz, 1H), 2.30 (s, 3H), 2.17-2.05 (m, 2H), 2.04-1.43 (m, 12H) ppm; exchangeable H not observed. |
| | 1,1-Difluoro-N-((5a,8- | | | |
| | trans)-(10aR,11S)-3- | | | |
| | fluoro-1-methyl-16-oxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)ethane-1-sulfonamide | | | |
| 56 | 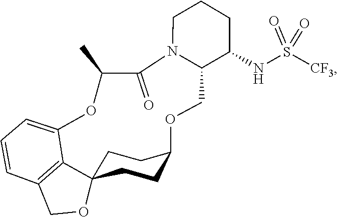 | Int-21/Int-2/ Tf₂O | ESI-MS m/z calc. 518.170, found 519.5 (M + 1)⁺. | ¹H NMR (400 MHz, Methanol-d₄) δ 7.22 (t, J = 7.7 Hz, 1H), 6.92 (dd, J = 7.7, 2.1 Hz, 2H), 5.34-5.25 (m, 1H), 5.10-5.04 (m, 1H), 5.02 (s, 2H), 3.85 (dd, J = 11.5, 9.3 Hz, 1H), 3.77-3.63 (m, 3H), 3.54-3.46 (m, 1H), 3.22-3.12 (m, 1H), 2.80 (ddd, J = 13.9, 11.6, 7.4 Hz, 1H), 2.09-2.01 (m, 2H), 1.86-1.43 (m, 12H) ppm; exchangeable H not observed. |
| | 1,1,1-Trifluoro-N-((5a,8- | | | |
| | trans)-(10aR,11S,17S)-17- | | | |
| | methyl-16-oxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)methanesulfonamide | | | |
| 57 | 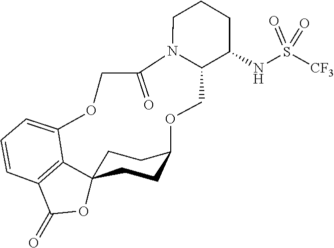 | Int-15/Int-2/ Tf₂O | ESI-MS m/z calc. 518.133, found 519.2 (M + 1)⁺. | ¹H NMR (300 MHz, Methanol-d₄) δ 7.58-7.40 (m, 2H), 7.23-7.12 (m, 1H), 5.41- 4.68 (m, 2H), 4.48-4.30 (m, 1H), 4.11-3.55 (m, 5H), 3.55-3.38 (m, 1H), 3.03-2.76 (m, 1H), 2.62- 2.41 (m, 1H), 2.30-2.01 (m, 1H), 1.97-1.90 (m, 2H), 1.83-1.61 (m, 4H), 1.50-1.34 (m, 2H), 1.34-1.15 (m, 1H) ppm; exchangeable H not observed. |
| | N-((5a,8-trans)- | | | |
| | (10aR,11S)-4,16-Dioxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)-1,1,1- | | | |
| | trifluoromethanesulfonamide | | | |
| 58 | 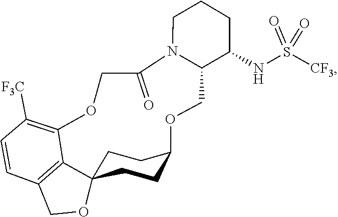 | Int-11/Int-2/ Tf₂O | ESI-MS m/z calc. 504.154, found 505.2 (M + 1)⁺. | ¹H NMR (300 MHz, Methanol-d₄) δ 7.23 (t, J = 7.8 Hz, 1H), 6.94-6.84 (m, 1H), 6.81 (dd, J = 8.0, 0.8 Hz, 1H), 5.35 (d, J = 11.6 Hz, 1H), 5.14 (dt, J = 9.9, 4.2 Hz, 1H), 5.05 (dd, J = 17.3, 4.7 Hz, 2H), 4.17 (d, J = 11.6 Hz, 1H), 4.09-3.82 (m, 2H), 3.81-3.64 (m, 2H), 3.57- 3.38 (m, 2H), 3.34 (s, 1H), 2.87 (td, J = 14.4, 5.2 Hz, 1H), 2.23-2.01 (m, 1H), 2.06 (s, 1H), 1.96-1.55 (m, 7H), 1.39 (d, J = 13.9 Hz, 1H) ppm; exchangeable H not observed. |
| | 1,1,1-Trifluoro-N-((5a,8- | | | |
| | trans)-(10aR,11S)-16-oxo- | | | |
| | 1-(trifluoromethyl)- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)methanesulfonamide | | | |
| 59 | 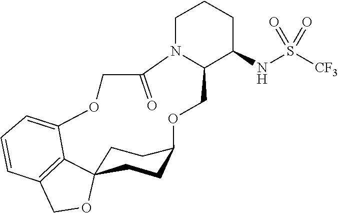 | Int-1/Int-27 | ESI-MS m/z calc. 518.145, found 519.5 (M + 1)⁺. | |
| | 1,1,1-Trifluoro-N-((5a,8- | | | |
| | trans)-(10aS,11R)-16-oxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)methanesulfonamide | | | |
| 60 | 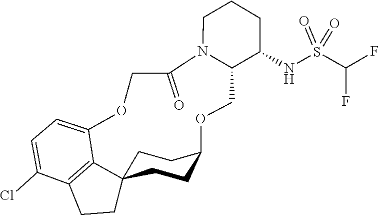 | Int-24/Int-2/ Int-5 | ESI-MS m/z calc. 518.145, found 519.5 (M + 1)⁺. | |
| | N-((5a,8-cis)-(10aR,11S)-3- | | | |
| | Chloro-16-oxo- | | | |
| | 4,5,7,8,10,10a,11,12,13,14, | | | |
| | 16,17-dodecahydro-6H- | | | |
| | 5a,8-ethanoindeno[7,1- | | | |
| | hi]pyrido[2,1- | | | |
| | c][1,7]dioxa[4]azacyclotridecin-11-yl)-1,1- | | | |
| | difluoromethanesulfonamide | | | |
| 61 | 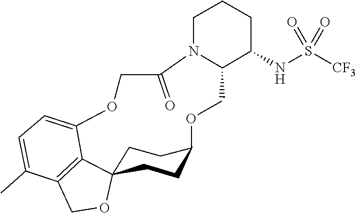 | Int-12/Int-2/ Tf₂O | ESI-MS m/z calc. 518.170, found 519.5 (M + 1)⁺. | |
| | 1,1,1-Trifluoro-N-((5a,8- | | | |
| | trans)-(10aR,11S)-3- | | | |
| | methyl-16-oxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)methanesulfonamide | | | |
| 62 | 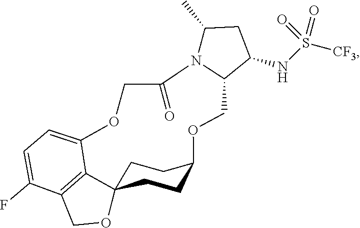 | Int-7/Int-29/ Tf₂O | ESI-MS m/z calc. 522.145, found 523.3 (M + 1)⁺. | |
| | 1,1,1-Trifluoro-N-((5a,8- | | | |
| | trans)-(10aR,11S,13R)-3- | | | |
| | fluoro-13-methyl-15-oxo- | | | |
| | 7,8,10a,11,12,13,15,16- | | | |
| | octahydro-4H,6H,10H- | | | |
| | 5a,8-ethanoisobenzofuro[7,1- | | | |
| | hi]pyrrolo[2,1- | | | |
| | c][1,7]dioxa[4]azacyclotridecin- | | | |
| | 11-yl)methanesulfonamide | | | |
| 63 | 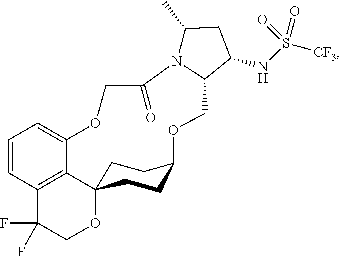 | Int-25/Int-29/ Tf₂O | ESI-MS m/z calc. 554.151 found 555.3 (M + 1)⁺. | ¹H NMR (300 MHz, Methanol-d₄) δ 7.53-7.15 (m, 3H), 4.89-4.86 (m, 1H, overlapped with solvent), 4.75-4.44 (m, 1H), 4.25 (s, 1H), 4.00 (t, J = 10.3 Hz, 2H), 3.78 (d, J = 14.0 Hz, 2H), 3.64- 3.42 (m, 1H), 2.84 (d, J = 12.1 Hz, 1H), 2.70-2.45 (m, 2H), 1.97- 1.31 (m, 12H) ppm; exchangeable H not observed. |
| | N-((6a,9-trans)- | | | |
| | (11aR,12S,14R)-4,4- | | | |
| | Difluoro-14-methyl-16- | | | |
| | oxo- | | | |
| | 4,5,8,9,11a,12,13,14,16,17- | | | |
| | decahydro-7H,11H-6a,9- | | | |
| | ethanoisochromeno[8,1- | | | |
| | hi]pyrrolo[2,1- | | | |
| | c][1,7]dioxa[4]azacyclotridecin- | | | |
| | 12-yl)-1,1,1- | | | |
| | trifluoromethanesulfonamide | | | |
| 64 | 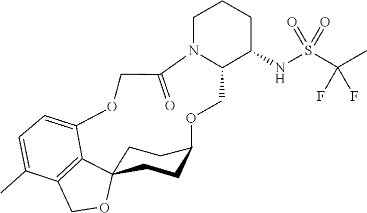 | Int-12/Int-2 Int-38 | ESI-MS m/z calc. 514.195, found 515.3 (M + 1)⁺. | ¹H NMR (300 MHz, Chloroform-d) δ 6.97 (dd, J = 8.0, 0.9 Hz, 1H), 6.63 (d, J = 8.1 Hz, 1H), 5.24 (d, J = 7.9 Hz, 1H), 5.15 (d, J = 11.8 Hz, 1H), 5.10-5.03 (m, 1H), 5.03-4.91 (m, 2H), 4.29 (d, J = 11.8 Hz, 1H), 3.99-3.85 (m, 1H), 3.81 (s, 2H), 3.75 (d, J = 5.6 Hz, 2H), 3.60-3.52 (m, 1H), 2.79 (td, J = 14.5, 5.3 Hz, 1H), 2.15 (s, 3H), 2.10-1.56 (m, 13H), 1.45 (d, J = 13.9 Hz, 1H) ppm. |
| | 1,1-Difluoro-N-((5a,8- | | | |
| | trans)-(10aR,11S)-3- | | | |
| | methyl-16-oxo- | | | |
| | 7,8,10,10a,11,12,13,14,16,17- | | | |
| | decahydro-4H,6H-5a,8- | | | |
| | ethanoisobenzofuro[7,1- | | | |
| | hi]pyrido[2,1- | | | |
| | c][1,7]dioxa[4]azacyclotridecin- | | | |
| | 11-yl)ethane-1-sulfonamide | | | |
| 65 | 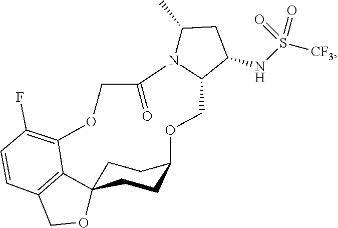 | Int-6/Int-29/ Tf₂O | ESI-MS m/z calc. 522.145, found 533.1 (M + 1)⁺. | |
| | 1,1,1-Trifluoro-N-((5a,8- | | | |
| | trans)-(10aR,11S,13R)-1- | | | |
| | fluoro-13-methyl-15-oxo- | | | |
| | 7,8,10a,11,13,15,16- | | | |
| | octahydro-4H,6H,10H- | | | |
| | 5a,8- | | | |
| | ethanoisobenzofuro[7,1- | | | |
| | c][1,7]dioxa[4]azacyclotridecin- | | | |
| | 11-yl)methanesulfonamide | | | |
| 66 | 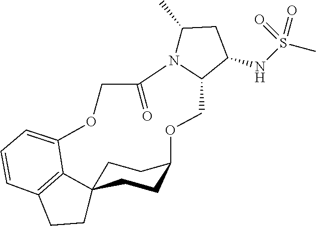 | Int-24/Int-29/ methane- sulfonyl chloride | ESI-MS m/z calc. 448.203, found 449.0 (M + 1)⁺. | ¹H NMR (300 MHz, Chloroform-d) δ 7.04 (t, J = 7.7 Hz, 1H), 6.79 (d, J = 7.4 Hz, 1H), 6.56 (d, J = 7.9 Hz, 1H), 4.96 (d, J = 10.6 Hz, 1H), 4.57- 4.36 (m, 3H), 4.36-4.17 (m, 2H), 3.90-3.79 (m, 1H), 3.32 (d, J = 9.2 Hz, 1H), 3.04 (td, 1H), 2.94 (s, 3H), 2.89-2.78 (m, 2H), 2.33-2.17 (m, 1H), 2.14-1.81 (m, 4H), 1.74 (q, J = 9.2 Hz, 2H), 1.65-1.46 (m, 4H), 1.39 (td, 1H), 1.18 (s, 1H), 1.07 (d, J = 13.6 Hz, 1H) ppm; exchangeable H not observed. |
| | N-((5a,8-cis)- | | | |
| | (10aR,11S,13R)-13- | | | |
| | Methyl-15-oxo- | | | |
| | 4,5,7,8,10a,11,12,13,15,16- | | | |
| | decahydro-6H,10H-5a,8- | | | |
| | ethanoindeno[7,1- | | | |
| | hi]pyrrolo[2,1- | | | |
| | c][1,7]dioxa[4]azacyclotridecin- | | | |
| | 11-yl)methanesulfonamide | | | |
| 67 | 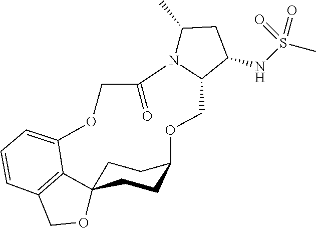 | Int-1/Int-29/ methane- sulfonyl chloride | ESI-MS m/z calc. 450.182, found 451.4 (M + 1)⁺. | ¹H NMR (300 MHz, Chloroform-d) δ 7.25-7.14 (m, 1H), 6.85 (d, J = 7.5 Hz, 1H), 6.74 (d, J = 8.0 Hz, 1H), 6.63 (d, J = 6.7 Hz, 1H), 5.18-4.95 (m, 3H), 4.56-4.40 (m, 2H), 4.40- 4.19 (m, 3H), 3.98 (s, 1H), 3.41 (d, J = 9.3 Hz, 1H), 3.00 (s, 3H), 2.28 (s, 1H), 2.11-1.76 (m, 5H), 1.76- 1.36 (m, 7H) ppm. |
| | N-((5a,8-trans)- | | | |
| | (10aR,11S,13R)-13- | | | |
| | Methyl-15-oxo- | | | |
| | 7,8,10a,11,12,13,15,16- | | | |
| | octahydro-4H,6H,10H- | | | |
| | 5a,8- | | | |
| | ethanoindeno[7,1- | | | |
| | hi]pyrrolo[2,1- | | | |
| | c][1,7]dioxa[4]azacyclotridecin- | | | |
| | 11-yl)methanesulfonamide | | | |
| 68 | 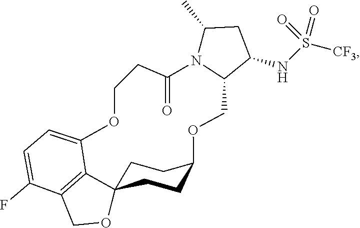 | Int-39/Int-29/ Tf₂O | ESI-MS m/z calc. 536.160, found 537.3 (M + 1)⁺. | ¹H NMR (300 MHz, Chloroform-d) δ 7.68 (s, 1H), 6.93-6.76 (m, 2H), 5.16-5.00 (m, 2H), 4.71 (d, J = 9.3 Hz, 1H), 4.44 (s, 3H), 4.29-4.16 (m, 2H), 3.91 (s, 1H), 3.32 (d, J = 9.3 Hz, 1H), 3.07 (ddd, J = 14.6, 12.1, 4.4 Hz, 1H), 2.65-2.39 (m, 2H), 2.35-2.23 (m, 1H), 2.23-1.87 (m, 5H), 1.76 (t, J = 15.5 Hz, 2H), 1.54 (d, J = 6.8 Hz, 3H), 1.38 (d, J = 13.6 Hz, 1H) ppm. |
| | 1,1,1-Trifluoro-N-((5a,8- | | | |
| | trans)-(10aR,11S,13R)-3- | | | |
| | fluoro-13-methyl-15-oxo- | | | |
| | 7,8,10a,11,12,13,16,17- | | | |
| | octahydro- | | | |
| | 4H,6H,10H,15H-5a,8- | | | |
| | ethanoisobenzofuro[7,1- | | | |
| | ij]pyrrolo[2,1- | | | |
| | c][1,8]dioxa[4]azacyclotetradecin- | | | |
| | 11-yl)methanesulfonamide | | | |
| 69 | 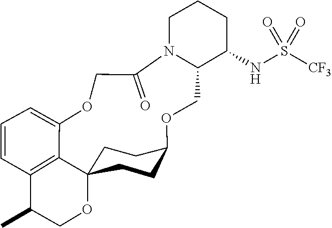 | Int-40/Int-2/ Tf₂O | ESI-MS m/z calc., 532.185 found 533.4 (M + 1)⁺. | ¹H NMR (300 MHz, Chloroform-d) δ 7.15 (t, J = 7.9 Hz, 1H), 6.85 (d, J = 7.7 Hz, 1H), 6.60 (d, J = 8.0 Hz, 1H), 5.34-5.13 (m, 2H), 5.06 (d, J = 10.2 Hz, 1H), 4.25 (d, J = 10.2 Hz, 1H), 4.03-3.37 (m, 8H), 2.87-2.41 (m, 3H), 1.99-1.60 (m, 9H), 1.44 (d, J = 14.2 Hz, 1H), 1.30 (d, J = 7.0 Hz, 3H) ppm. |
| | rel-1,1,1-Trifluoro-N- | | | |
| | ((6a,9-trans)- | | | |
| | (4S,11aR*,12S*)-4-methyl- | | | |
| | 17-oxo- | | | |
| | 4,5,8,9,11,11a,12,13,15, | | | |
| | 17,18-dodecahydro-7H- | | | |
| | 6a,9- | | | |
| | ethanoisochromeno[8,1- | | | |
| | hi]pyrido[2,1- | | | |
| | c][1,7]dioxa[4]azacyclotridecin- | | | |
| | 12-yl)methanesulfonamide | | | |
| | (Second eluting peak by | | | |
| | SFC* carried out at amide | | | |
| | macrocyclisation step) | | | |
| 70 | 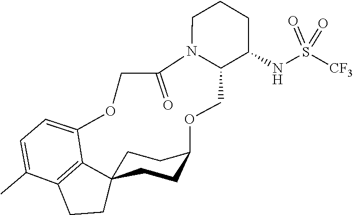 | Int-24/Int-2/ Tf₂O | ESI-MS m/z calc., 516.191 found 517.3 (M + 1)⁺. | ¹H NMR (300 MHz, DMSO-d₆) δ 9.71 (d, J = 8.0 Hz, 1H), 6.88 (d, J = 8.1 Hz, 1H), 6.58 (d, J = 8.0 Hz, 1H), 5.27 (d, J = 11.5 Hz, 1H), 4.95 (q, J = 5.0 Hz, 1H), 3.93 (d, J = 11.5 Hz, 1H), 3.82 (t, J = 10.0 Hz, 1H), 3.78-3.62 (m, 2H), 3.56 (d, J = 8.3 Hz, 1H), 3.42-3.20 (m, 2H), 3.08- 2.88 (m, 1H), 2.82-2.64 (m, 2H), 2.24-2.13 (m, 1H), 2.11 (s, 3H), 2.08-1.88 (m, 2H), 1.85-1.65 (m, 4H), 1.64-1.42 (m, 3H), 1.40-1.22 (m, 1H), 1.13 (d, J = 13.1 Hz, 1H), 0.98 (d, J = 13.0 Hz, 1H) ppm. |
| | 1,1,1-Trifluoro-N-((5a,8- | | | |
| | cis)-(10aR,11S)-3-methyl- | | | |
| | 16-oxo- | | | |
| | 4,5,7,8,10,10a,11,12,13,14, | | | |
| | 16,17-dodecahydro-6H- | | | |
| | 5a,8-ethanoindeno[7,1- | | | |
| | hi]pyrido[2,1- | | | |
| | c][1,7]dioxa[4]azacyclotridecin- | | | |
| | 11-yl)methanesulfonamide | | | |
| 71 | 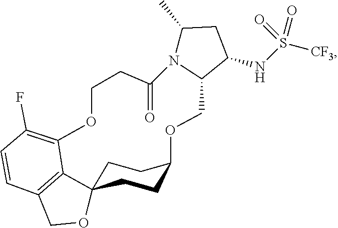 | Int-41/Int-43/ Tf₂O | ESI-MS m/z calc., 536.160 found 536.8 (M + 1)⁺. | ¹H NMR (300 MHz, DMSO- d₆) δ 9.78 (d, J = 76.9 Hz, 1H), 7.24-7.09 (m, 1H), 7.01-6.85 (m, 1H), 5.02-4.56 (m, 3H), 4.25-4.00 (m, 3H), 3.90 (d, J = 9.0 Hz, 1H), 3.79-3.67 (m, 2H), 3.53-2.99 (m, 2H), 2.74-2.57 (m, 1H), 2.52-2.23 (m, 2H), 2.22-2.06 (m, 1H), 1.96- 1.60 (m, 4H), 1.60-1.32 (m, 3H), 1.32-1.22 (m, 3H) ppm. |
| | 1,1,1-Trifluoro-N-((5a,8- | | | |
| | trans)-(10aR,11S,13R)-1- | | | |
| | fluoro-13-methyl-15-oxo- | | | |
| | 7,8,10a,11,12,13,16,17- | | | |
| | octahydro- | | | |
| | 4H,6H,10H,15H-5a,8- | | | |
| | ethanoisobenzofuro[7,1- | | | |
| | ij]pyrrolo[2,1- | | | |
| | c][1,8]dioxa[4]azacyclotetradecin- | | | |
| | 11-yl)methanesulfonamide | | | |
| 72 | 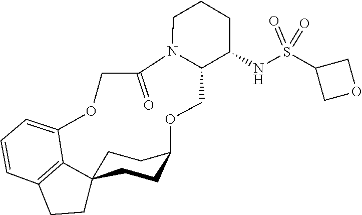 | Int-24/Int-2/ oxetane-3- sulfonyl chloride | ESI-MS m/z calc., 490.214 found 491.4 (M + 1)⁺. | ¹H NMR (300 MHz, Chloroform-d) δ 7.10 (t, J = 7.7 Hz, 1H), 6.86 (d, J = 7.5 Hz, 1H), 6.61 (d, J = 8.0 Hz, 1H), 5.14 (d, J = 11.4 Hz, 1H), 5.11-5.01 (m, 1H), 4.96-4.87 (m, 4H), 4.50 (dt, J = 14.5, 7.3 Hz, 1H), 4.38-4.27 (m, 2H), 3.86-3.69 (m, 4H), 3.56-3.41 (m, 2H), 3.01-2.87 (m, 3H), 2.19 (dt, J = 11.8, 6.2 Hz, 1H), 2.09-1.96 (m, 3H), 1.94-1.74 (m, 4H), 1.71-1.60 (m, 2H), 1.50-1.43 (m, 1H), 1.28 (d, J = 14.1 Hz, 1H), 1.17 (d, J = 14.1 Hz, 1H) ppm. |
| | N-((5a,8-cis)-(10aR,11S)- | | | |
| | 16-Oxo- | | | |
| | 4,5,7,8,10,10a,11,12,13,14, | | | |
| | 16,17-dodecahydro-6H- | | | |
| | 5a,8-ethanoindeno[7,1- | | | |
| | hi]pyrido[2,1- | | | |
| | c][1,7]diox[4]azacyclotridecin- | | | |
| | 11-yl)oxetane-3-sulfonamide | | | |
| 73 | 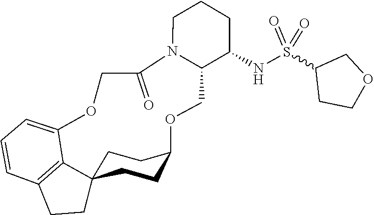 | Int-24/Int-2/ tetrahydro- furan 3-sulfonyl chloride | ESI-MS m/z calc., 504.229 found 505.4 (M + 1)⁺. | ¹H NMR (300 MHz, Chloroform-d) δ 7.10 (t, J = 7.7 Hz, 1H), 6.86 (d, J = 7.5 Hz, 1H), 6.61 (d, J = 7.9 Hz, 1H), 5.15 (d, J = 11.4 Hz, 2H), 4.32 (d, J = 11.4 Hz, 1H), 4.23 (d, J = 8.5 Hz, 1H), 4.19-4.06 (m, 2H), 4.07- 3.93 (m, 1H), 3.81 (td, J = 16.0, 7.3 Hz, 6H), 3.57-3.44 (m, 2H), 2.96- 2.85 (m, 2H), 2.32 (q, J = 6.2 Hz, 2H), 2.21 (dd, J = 12.2, 6.3 Hz, 1H), 2.02 (d, J = 8.3 Hz, 3H), 1.90-1.78 (m, 3H), 1.65 (d, J = 8.6 Hz, 2H), 1.51 (s, 2H), 1.28 (d, J = 14.3 Hz, 2H), 1.16 (d, J = 13.7 Hz, 1H) ppm. |
| | dia-(R)-N-((5a,8-cis)- | | | |
| | (10aR*,11S*)-16-Oxo- | | | |
| | 4,5,7,8,10,10a,11,12,13,14, | | | |
| | 16,17-dodecahydro-6H- | | | |
| | 5a,8-ethanoindeno[7,1- | | | |
| | c][1,7]dioxa[4]azacyclotridecin- | | | |
| | 11-yl)tetrahydrofuran- | | | |
| | 3-sulfonamide | | | |
| 74 | 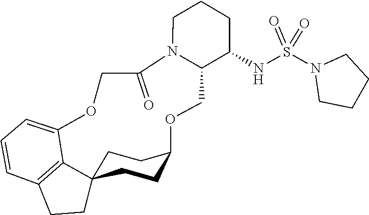 | Int-24/Int-2/ pyrrolidine-1- sulfonyl chloride | ESI-MS m/z calc., 503.245 found 504.5 (M + 1)⁺. | ¹H NMR (300 MHz, Chloroform-d) δ 7.10 (t, J = 7.7 Hz, 1H), 6.86 (dd, J = 7.5, 1.0 Hz, 1H), 6.61 (d, J = 8.0 Hz, 1H), 5.14 (d, J = 11.4 Hz, 2H), 4.31 (d, J = 11.4 Hz, 1H), 4.11 (d, J = 8.3 Hz, 1H), 3.89-3.69 (m, 3H), 3.66- 3.41 (m, 3H), 3.41-3.30 (m, 4H), 3.01 (td, J = 13.7, 4.1 Hz, 1H), 2.96-2.85 (m, 2H), 2.20 (dt, J = 12.1, 5.8 Hz, 1H), 2.14-1.97 (m, 2H), 2.01-1.90 (m, 3H), 1.90-1.59 (m, 5H), 1.44 (dd, J = 25.8, 11.7 Hz, 2H), 1.27 (d, J = 12.3 Hz, 2H), 1.15 (d, J = 13.4 Hz, 1H) ppm; exchangeable H not observed. |
| | N-((5a,8-cis)-(10aR,11S)- | | | |
| | 16-Oxo- | | | |
| | 4,5,7,8,10,10a,11,12,13,14, | | | |
| | 16,17-dodecahydro-6H- | | | |
| | 5a,8-ethanoindeno[7,1- | | | |
| | hi]pyrido[2,1- | | | |
| | c][1,7]dioxa[4]azacyclotridecin- | | | |
| | 11-yl)pyrrolidine-1-sulfonamide | | | |

*In this case only, the diastereomers were separated by Chiralpak ® AD-H column, 5 μm particle size, 15 cm × 30 mm from Daicel Corporation (Mobile
phase: 15% ⁱPrOH (supplement with 5 mM ammonia), 85% CO₂; Flow rate 80 mL/min).

### Example 31: Intermediate 1

### Benzyl 2-((4-oxo-3′H-spiro[cyclohexane-1,1′-isobenzofuran]-7′-yl)oxy)acetate (Int-1)

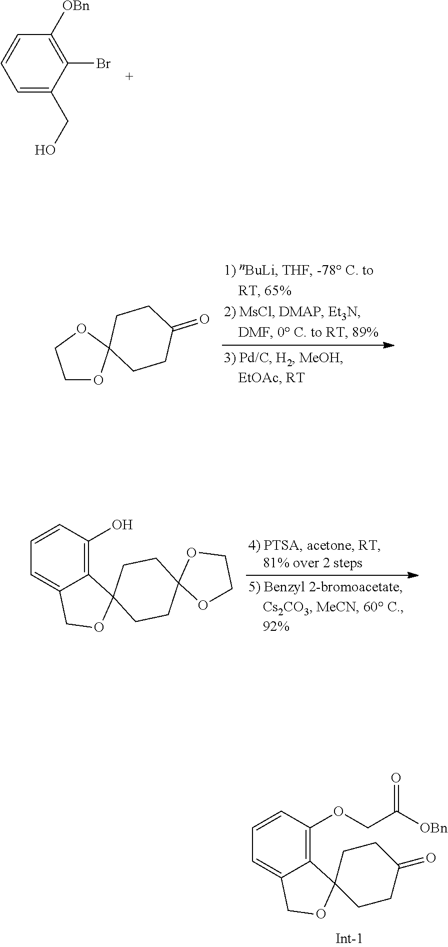 [SMILES: OCc1cccc(OCc2ccccc2)c1Br ; O=C1CCC2(CC1)OCCO2 ; Oc1cccc2c1C1(CCC3(CC1)OCCO3)OC2 ; O=C1CCC2(CC1)OCc1cccc(OCC(=O)OCc3ccccc3)c12]

### Step 1:

Under a nitrogen atmosphere, ⁿBuLi (23 mL, 1.6 M solution in hexanes, 36.80 mmol) was added to a stirred solution of (3-(benzyloxy)-2-bromophenyl)methanol (4.8 g, 16.37 mmol) in THF (80 mL) at −78° C. and the reaction mixture was stirred at −78° C. for 1 h. A solution of 1,4-dioxaspiro[4.5]decan-8-one (2.9 g, 18.57 mmol) in THF (20 mL) was added dropwise and the mixture was slowly warmed to ambient temperature and stirred overnight. The reaction was quenched by addition of a saturated NH₄Cl solution (250 mL). The aqueous layer was separated and extracted with EtOAc (2×). The combined organic layers were washed with brine, dried (Na₂SO₄), passed through a pad of silica and concentrated in vacuo. Purification by flash chromatography (80 g SiO₂, 0 to 80% EtOAc in heptane) gave 8-(2-(benzyloxy)-6-(hydroxymethyl)phenyl)-1,4-dioxaspiro[4.5]decan-8-ol (3.95 g, 65%) as a white solid. ¹H NMR (400 MHz, Chloroform-d) δ 7.53 (d, J=7.5 Hz, 2H), 7.44 (t, J=7.4 Hz, 2H), 7.41-7.34 (m, 1H), 7.22-7.12 (m, 1H), 7.03 (d, J=8.3 Hz, 1H), 6.91 (d, J=7.5 Hz, 1H), 5.17 (s, 2H), 5.00 (d, J=1.6 Hz, 2H), 4.02-3.90 (m, 2H), 3.81 (t, J=6.4 Hz, 2H), 3.09 (td, J=13.8, 4.4 Hz, 2H), 2.47 (s, 2H), 2.13 (td, J=13.1, 4.4 Hz, 2H), 1.81-1.68 (m, 2H), 1.68-1.52 (m, 2H) ppm.

### Step 2:

MsCl (720 μL, 9.303 mmol) was added dropwise to a stirred solution of 8-(2-(benzyloxy)-6-(hydroxymethyl)phenyl)-1,4-dioxaspiro[4.5]decan-8-ol (2.86 g, 7.720 mmol), DMAP (1.9 g, 15.55 mmol) and Et₃N (2.4 mL, 17.22 mmol) in DMF (30 mL) at 0° C. and the reaction mixture was stirred at 0° C. for 2 h and at ambient temperature for 30 min. The reaction was quenched by addition of a saturated aqueous NH₄Cl solution (250 mL). The aqueous layer was separated and extracted with EtOAc (2×). The combined organic layers were washed with brine and passed through a pad of Na₂SO₄ sitting on top of a layer of silica. The filtrates were collected and concentrated in vacuo. Purification by flash chromatography (80 g SiO₂, 0 to 40% EtOAc in heptane) gave 7-(benzyloxy)-3H-dispiro[isobenzofuran-1,1′-cyclohexane-4′,2″-[1,3]dioxolane](2.42 g, 89%) as a white solid. ¹H NMR (400 MHz, Chloroform-d) δ 7.59 (d, J=7.5 Hz, 2H), 7.44 (t, J=7.5 Hz, 2H), 7.40-7.33 (m, 1H), 7.26 (t, J=7.7 Hz, 1H), 6.85 (d, J=7.8 Hz, 2H), 5.21 (s, 2H), 5.12 (s, 2H), 4.06 (d, J=1.9 Hz, 4H), 2.80-2.59 (m, 2H), 2.20-2.02 (m, 2H), 1.88-1.66 (m, 4H) ppm.

### Step 3:

A suspension of 7-(benzyloxy)-3H-dispiro[isobenzofuran-1,1′-cyclohexane-4′,2″-[1,3]dioxolane](2.6 g, 7.377 mmol) and Pd/C (3 g, 5 wt. % loading (dry basis) Degussa, 1.410 mmol) in a mixture of MeOH (30 mL) and EtOAc (30 mL) was placed under a hydrogen atmosphere and the reaction mixture was stirred at ambient temperature for 2 h. The mixture was passed through a pad of silica, washing the filtered cake with EtOAc. The filtrates were concentrated in vacuo to give 3H-dispiro[isobenzofuran-1,1′-cyclohexane-4′,2″-[1,3]dioxolan]-7-ol, which was used without further purification in the next step.

### Step 4:

PTSA monohydrate (3 g, 15.77 mmol) was added to a stirred solution of 3H-dispiro[isobenzofuran-1,1′-cyclohexane-4′,2″-[1,3]dioxolan]-7-ol in acetone (30 mL) and the reaction mixture was stirred at ambient temperature for 2 h. The reaction was quenched by addition of a saturated NH₄Cl solution. The aqueous layer was separated and extracted with EtOAc (2×). The combined organic layers were washed with brine, dried (Na₂SO₄), filtered and passed through a pad of silica, washing the filtered cake with EtOAc. The filtrates were concentrated in vacuo. Purification by flash chromatography (40 g SiO₂, 0 to 80% EtOAc in heptane) gave 7′-hydroxy-3′H-spiro[cyclohexane-1,1′-isobenzofuran]-4-one (1.3 g, 81% over 2 steps) as a white solid. ¹H NMR (400 MHz, Chloroform-d) δ 7.20 (t, J=7.2 Hz, 1H), 6.84 (d, J=7.4 Hz, 1H), 6.67 (d, J=7.8 Hz, 1H), 5.19 (s, 2H), 2.93 (td, J=14.2, 6.1 Hz, 2H), 2.68 (td, J=13.8, 4.7 Hz, 2H), 2.46-2.36 (m, 2H), 2.17-2.10 (m, 2H) ppm; exchangeable H not observed.

### Step 5:

Benzyl 2-bromoacetate (1.1 mL, 6.944 mmol) was added to a suspension of 7′-hydroxy-3′H-spiro[cyclohexane-1,1′-isobenzofuran]-4-one (1.3 g, 5.956 mmol) and Cs₂CO₃ (3.8 g, 11.66 mmol) in MeCN (30 mL) and the reaction mixture was stirred at 60° C. for 2 h. Purification by flash chromatography (40 g SiO₂, 0 to 50% EtOAc in heptane) gave benzyl 2-((4-oxo-3′H-spiro[cyclohexane-1,1′-isobenzofuran]-7′-yl)oxy)acetate (Int-1, 2.23 g, 92%) as a colourless oil. ¹H NMR (400 MHz, Chloroform-d) δ 7.42-7.38 (m, 3H), 7.34-7.29 (m, 2H), 7.26 (t, J=7.8 Hz, 1H), 6.91 (d, J=7.5 Hz, 1H), 6.64 (d, J=8.1 Hz, 1H), 5.24 (s, 2H), 5.18 (s, 2H), 4.74 (s, 2H), 2.88 (td, J=14.2, 6.0 Hz, 2H), 2.69 (td, J=13.8, 4.7 Hz, 2H), 2.47-2.33 (m, 2H), 2.05 (dd, J=12.9, 5.3 Hz, 2H) ppm.

The following Intermediates were made using the method described in Intermediate 1 except that, in Step 5, different bromides were used in place of benzyl 2-bromoacetate and the conditions were those of Intermediate 6 Step 5:

| Intermediate | | | | |
|---|---|---|---|---|
| Number | Structure | Name | LC/MS | NMR (shifts in ppm) |
| Int-17 | 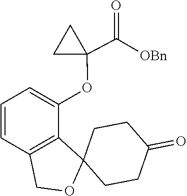 [SMILES: O=C1CCC2(CC1)OCc1cccc(OC3(C(=O)OCc4ccccc4)CC3)c12] | Benzyl 1-((4- oxo-3′H- spiro[cyclo- hexane-1,1′- isobenzofuran]- 7′- yl)oxy)cyclo- propane-1- carboxylate | ESI-MS m/z calc. 392.162, found 393.3 (M + 1)⁺. | |
| Int-19 | 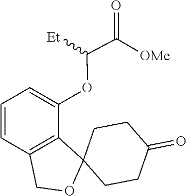 [SMILES: CCC(Oc1cccc2c1C1(CCC(=O)CC1)OC2)C(=O)OC] | Methyl rac-2- ((4-oxo-3′H- spiro[cyclo- hexane-1,1′- isobenzofuran]- 7′- yl)oxy)butanoate | ESI-MS m/z calc. 318.147, found 319.3 (M + 1)⁺. | ¹H NMR (300 MHz, Chloroform-d) δ 7.13 (dd, J = 8.1, 7.5 Hz, 1H), 6.80-6.72 (m, 1H), 6.54-6.46 (m, 1H), 5.07 (s, 2H), 4.64 (dd, J = 6.5, 5.5 Hz, 1H), 3.64 (s, 3H), 2.93-2.73 (m, 2H), 2.67 (td, J = 13.4, 4.2 Hz, 1H), 2.55 (td, J = 13.7, 4.7 Hz, 1H), 2.39-2.22 (m, 2H), 2.10- 1.97 (m, 2H), 1.97-1.84 (m, 2H), 1.01-0.90 (m, 3H) ppm. |

The following Intermediate was made using the method described in Intermediate 1 except that, in Step 5, ethyl 2-bromo-2,2-difluoroacetate was used in place of benzyl 2-bromoacetate and the conditions were those of Intermediate 8 Step 5:

| Intermediate | | | | |
|---|---|---|---|---|
| Number | Structure | Name | LC/MS | NMR (shifts in ppm) |
| Int-20 | 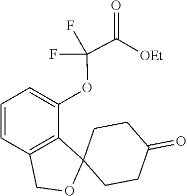 [SMILES: CCOC(=O)C(F)(F)Oc1cccc2c1C1(CCC(=O)CC1)OC2] | Ethyl 2,2- difluoro-2-((4- oxo-3′H- spiro[cyclo- hexane-1,1′- isobenzofuran]- 7′-yl)oxy)acetate | ESI-MS m/z calc. 340.112, found 341.2 (M + 1)⁺. | ¹H NMR (300 MHz, Chloroform-d) δ 7.34-7.27 (m, 1H), 7.20 (d, J = 0.9 Hz, 1H), 7.10 (d, J = 0.9 Hz, 1H), 5.16 (s, 2H), 4.38 (q, J = 7.1 Hz, 2H), 2.88 (td, J = 14.1, 6.2 Hz, 2H), 2.53 (td, J = 13.7, 4.8 Hz, 2H), 2.43- 2.29 (m, 2H), 2.17-1.97 (m, 2H), 1.33 (t, J = 7.2 Hz, 3H) ppm. |

The following Intermediate was made using the method described in Intermediate 1 except that, in Step 1, Int-35 was used as the starting material in place of (3-(benzyloxy)-2-bromophenyl)methanol and the conditions were those of Intermediate 9 Step 1. The conditions of Step 2 were those of Intermediate 23 Step 3. The conditions of Step 5 were those of Intermediate 7 Step 5:

| Intermediate | | | | |
|---|---|---|---|---|
| Number | Structure | Name | LC/MS | NMR (shifts in ppm) |
| Int-10 | 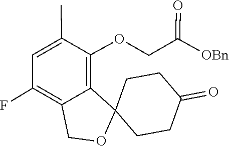 [SMILES: Cc1cc(F)c2c(c1OCC(=O)OCc1ccccc1)C1(CCC(=O)CC1)OC2] | Benzyl 2-((4′- fluoro-6′- methyl-4-oxo- 3′H- spiro[cyclo- hexane-1,1′- isobenzofuran]- 7′-y1)oxy)acetate | ESI-MS m/z calc. 398.153, found 399.4 (M + 1)⁺. | |

The following Intermediate was made using the method described in Intermediate 1 except that, in Step 1, Int-36 was used as the starting material in place of (3-(benzyloxy)-2-bromophenyl)methanol and the conditions were those of Intermediate 9 Step 1. The conditions of Step 2 were those of Intermediate 7 Step 2. The conditions of Step 5 were those of Intermediate 23 Step 5:

| Intermediate | | | | |
|---|---|---|---|---|
| Number | Structure | Name | LC/MS | NMR (shifts in ppm) |
| Int-11 | 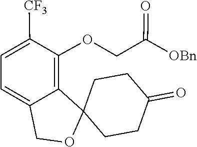 [SMILES: O=C1CCC2(CC1)OCc1ccc(C(F)(F)F)c(OCC(=O)OCc3ccccc3)c12] | Benzyl 2-((4- oxo-6′- (trifluoromethyl)- 3′H- spiro[cyclo- hexane-1,1′- isobenzofuran]- 7′-yl)oxy)acetate | | ¹H NMR (400 MHz, Chloroform-d) δ 7.58 (d, J = 7.9 Hz, 1H), 7.41-7.35 (m, 5H), 7.12 (dq, J = 7.9, 0.9 Hz, 1H), 5.25 (s, 2H), 5.18- 5.10 (m, 2H), 4.61 (s, 2H), 2.84 (td, J = 14.3, 6.2 Hz, 2H), 2.55 (td, J = 13.6, 4.9 Hz, 2H), 2.39-2.32 (m, 2H), 2.16-2.06 (m, 2H) ppm. |

### Example 32: Intermediate 2

### Benzyl (2R,3S)-2-(hydroxymethyl)-3-(2,2,2-trifluoroacetamido)piperidine-1-carboxylate (Int-2)

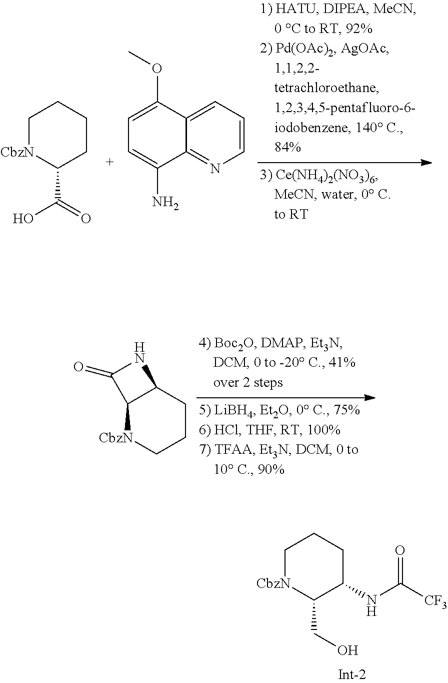

### Step 1:

HATU (61 g, 160.4 mmol) and DIPEA (70 mL, 401.9 mmol) were successively added to a solution of (R)-1-((benzyloxy)carbonyl)piperidine-2-carboxylic acid (35 g, 132.9 mmol) and 5-methoxyquinolin-8-amine (24 g, 137.8 mmol) in MeCN (1.3 L) at 0° C. and the reaction mixture was warmed to ambient temperature over 2 h and stirred at this temperature for an additional 4 h. The reaction was partitioned between DCM (500 mL) and water (1 L) and stirred for 10 min. The organic phase was separated, washed with water (2×400 mL), dried (MgSO₄), filtered and concentrated in vacuo. Purification by flash chromatography (800 g SiO₂, 0 to 100% EtOAc in heptane) gave benzyl (R)-2-((5-methoxyquinolin-8-yl)carbamoyl)piperidine-1-carboxylate (51.4 g, 92%) as a yellow foam. ¹H NMR (300 MHz, DMSO-d₆) δ 10.09 (s, 1H), 8.87 (dd, J=4.3, 1.7 Hz, 1H), 8.62-8.44 (m, 2H), 7.62 (dd, J=8.5, 4.2 Hz, 1H), 7.53-7.12 (m, 5H), 7.06 (d, J=8.6 Hz, 1H), 5.18 (s, 2H), 5.08 (d, J=5.5 Hz, 1H), 4.06 (d, J=12.7 Hz, 1H), 3.98 (s, 3H), 3.25-3.05 (m, 1H), 2.27 (d, J=13.6 Hz, 1H), 1.79-1.57 (m, 3H), 1.49-1.26 (m, 2H) ppm. ESI-MS m/z calc. 419.185, found 420.8 (M+1)⁺.

### Step 2:

A solution of benzyl (R)-2-((5-methoxyquinolin-8-yl)carbamoyl)piperidine-1-carboxylate (2.2 g, 4.877 mmol) in a mixture of 1,1,2,2-tetrachloroethane (18 mL) and 1,2,3,4,5-pentafluoro-6-iodobenzene (2.8 mL, 20.99 mmol) was added to a vial containing AgOAc (1.8 g, 10.78 mmol) and Pd(OAc)₂ (110 mg, 0.490 mmol) and the reaction mixture was heated at 140° C. for 3 h. The mixture was filtered and the filtrates were concentrated in vacuo. Purification by flash chromatography (110 g SiO₂, 20 to 100% EtOAc in heptane followed by 40 g SiO₂, 20 to 100% EtOAc in heptane) gave benzyl (1R,6S)-7-(5-methoxyquinolin-8-yl)-8-oxo-2,7-diazabicyclo[4.2.0]octane-2-carboxylate (8.7 g, 84%) as a brown solid. H NMR (300 MHz, DMSO-d₆) δ 8.94 (d, J=4.3 Hz, 1H), 8.56 (dd, J=8.5, 1.8 Hz, 1H), 8.17 (dd, J=8.5, 5.1 Hz, 1H), 7.59 (dd, J=8.5, 4.2 Hz, 1H), 7.51-7.24 (m, 5H), 7.11 (d, J=8.6 Hz, 1H), 5.51 (t, J=6.7 Hz, 1H), 5.41 (s, 1H), 5.17 (d, J=3.6 Hz, 2H), 3.99 (s, 3H), 3.69-3.37 (m, 2H), 1.89-1.53 (m, 4H) ppm. ESI-MS m/z calc. 417.169, found 418.4 (M+1)⁺.

### Step 3:

A solution of Ce(NH₄)₂(NO₃)₆ (4 g, 7.296 mmol) in water (10 mL) was added to a stirred suspension of benzyl (1R,6S)-7-(5-methoxyquinolin-8-yl)-8-oxo-2,7-diazabicyclo[4.2.0]octane-2-carboxylate (940 mg, 2.147 mmol) in MeCN (50 mL) at 0° C. and the reaction mixture was stirred at ambient temperature for 2 days. The reaction was extracted with MTBE (2×200 mL). The combined organic layers were washed with water (300 mL), dried (Na₂SO₄), filtered, and concentrated in vacuo to give benzyl (1R,6S)-8-oxo-2,7-diazabicyclo[4.2.0]octane-2-carboxylate, which was used without further purification in the next step.

### Step 4:

DMAP (30 mg, 0.246 mmol), Et₃N (300 μL, 2.152 mmol), and Boc₂O (1 mL, 4.353 mmol) were successively added to a solution of benzyl (1R,6S)-8-oxo-2,7-diazabicyclo[4.2.0]octane-2-carboxylate in DCM (30 mL) at 0° C. and the reaction mixture was kept at −20° C. for 3 weeks. The mixture was washed with water (2×10 mL) and the combined organic layers were concentrated in vacuo. Purification by flash chromatography (40 g SiO₂, 0 to 100% EtOAc in heptane) gave 2-benzyl 7-(tert-butyl) (1R,6S)-8-oxo-2,7-diazabicyclo[4.2.0]octane-2,7-dicarboxylate (330 mg, 41% over 2 steps) as a yellow viscous oil. ESI-MS m/z calc. 360.169, found 361.4 (M+1)⁺.

### Step 5:

LiBH₄ (800 mg, 36.73 mmol) was added to a suspension of 2-benzyl 7-(tert-butyl) (1R,6S)-8-oxo-2,7-diazabicyclo[4.2.0]octane-2,7-dicarboxylate (2.5 g, 6.590 mmol) in Et₂O (300 mL) at 0° C. and the reaction mixture was stirred at 0° C. for 1 h. The reaction was quenched by addition of a saturated NH₄Cl solution (200 mL) and stirred for 30 min. The organic layer was separated and concentrated in vacuo. Purification by flash chromatography (24 g SiO₂, 20 to 100% EtOAc in heptane) gave benzyl (2R,3S)-3-((tert-butoxycarbonyl)amino)-2-(hydroxymethyl)piperidine-1-carboxylate (1900 mg, 75%) as a pale yellow solid. ESI-MS m/z calc. 364.200, found 365.4 (M+1)⁺.

### Step 6:

HCl (100 mL, 4 M solution in 1,4-dioxane, 400 mmol) was added to a suspension of benzyl (2R,3S)-3-((tert-butoxycarbonyl)amino)-2-(hydroxymethyl)piperidine-1-carboxylate (15 g, 40.98 mmol) in THF (40 mL) at ambient temperature and the reaction mixture was stirred at ambient temperature for 2 h. The mixture was concentrated in vacuo to give benzyl (2R,3S)-3-amino-2-(hydroxymethyl)piperidine-1-carboxylate hydrochloride (13.4 g, 100%) as a yellow foam. ¹H NMR (300 MHz, DMSO-d₆) δ 8.31 (s, 3H), 7.47-7.22 (m, 5H), 5.09 (s, 2H), 4.51 (q, J=6.2 Hz, 1H), 3.87 (dd, J=13.8, 4.3 Hz, 1H), 3.82-3.60 (m, 2H), 3.22 (s, 1H), 2.89 (t, J=12.8 Hz, 1H), 1.92-1.61 (m, 3H), 1.46 (ddt, J=23.6, 11.8, 7.0 Hz, 1H) ppm. ESI-MS m/z calc. 264.147, found 265.6 (M+1)⁺.

### Step 7:

TFAA (7.5 mL, 53.96 mmol) was added over 15 min to a stirred solution of benzyl (2R,3S)-3-amino-2-(hydroxymethyl)piperidine-1-carboxylate hydrochloride (12.3 g, 40.89 mmol) and Et₃N (28 mL, 200.9 mmol) in DCM (175 mL) cooled in an ice-water bath, keeping the internal temperature below 10° C. and the reaction mixture was stirred at 0° C. for 40 min. The reaction was quenched by addition of water (100 mL). The organic phase was separated and washed with a saturated aqueous NaHCO₃ solution (150 mL) and brine (150 mL), dried (MgSO₄) filtered and concentrated in vacuo. Purification by flash chromatography (200 g SiO₂, 0 to 100% EtOAc in heptane) gave benzyl (2R,3S)-2-(hydroxymethyl)-3-(2,2,2-trifluoroacetamido)piperidine-1-carboxylate (Int-2, 13.3 g, 90%) as a pale yellow viscous and glassy solid. ¹H NMR (300 MHz, Chloroform-d) δ 8.21 (s, 1H), 7.42-7.29 (m, 5H), 5.14 (d, J=3.2 Hz, 2H), 4.55 (dt, J=9.8, 4.8 Hz, 1H), 4.28-3.95 (m, 3H), 3.82 (dt, J=10.8, 4.3 Hz, 1H), 2.87-2.68 (m, 1H), 2.11 (d, J=9.7 Hz, 1H), 1.83-1.52 (m, 4H) ppm. ESI-MS m/z calc. 360.130, found 361.3 (M+1)⁺.

### Example 33: Intermediate 4

### 1-((1-Fluorocyclopropyl)sulfonyl)-3-methyl-1H-imidazol-3-ium triflate (Int-4)

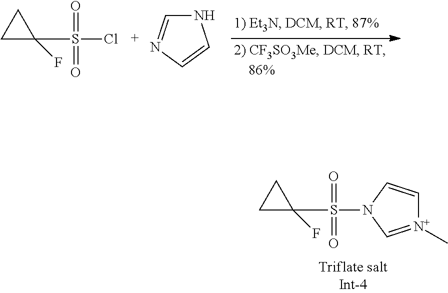

### Step 1:

1-Fluorocyclopropanesulfonyl chloride (500 mg, 3.153 mmol) was added dropwise to a mixture of imidazole (220 mg, 3.232 mmol) and Et₃N (500 μL, 3.587 mmol) in DCM (10 mL) at ambient temperature and the reaction mixture was stirred at ambient temperature for 1 h. The reaction was concentrated in vacuo. Purification by flash chromatography (40 g SiO₂, 0 to 100% EtOAc in hexanes) gave 1-((1-fluorocyclopropyl)sulfonyl)-1H-imidazole (533 mg, 87%) as a colourless oil. ¹H NMR (400 MHz, Chloroform-d) δ 7.99 (s, 1H), 7.33 (s, 1H), 7.22 (s, 1H), 1.84-1.72 (m, 2H), 1.66-1.55 (m, 2H) ppm.

### Step 2:

Methyl trifluoromethanesulfonate (350 μL, 3.191 mmol) was added dropwise to a stirred mixture of 1-((1-fluorocyclopropyl)sulfonyl)-1H-imidazole (533 mg, 87%) in DCM (10 mL) at 0° C. and the reaction mixture was stirred at ambient temperature for 1 h. The reaction was concentrated in vacuo to give 1-((1-fluorocyclopropyl)sulfonyl)-3-methyl-1H-imidazol-3-ium triflate (Int-4, 984 mg, 86%) as a white solid, which was used without further purification in the next step.

The following Intermediates were made using the method described in Intermediate 4 except that, in Step 1, different sulfonyl chlorides were used in place of 1-fluorocyclopropanesulfonyl chloride:

| Intermediate | | | | |
|---|---|---|---|---|
| Number | Structure | Name | LC/MS | NMR (shifts in ppm) |
| Int-5 | 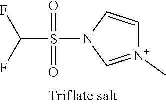 | 1- ((Difluoromethyl) sulfonyl)-3- methyl-1H- imidazol-3-ium triflate | No ionisation | |
| Int-38 | 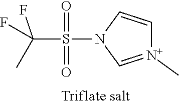 | 1-((1,1- Difluoroethyl) sulfonyl)-3- methyl-1H- imidazol-3-ium triflate | No ionisation | |

### Example 34: Intermediate 6

### Benzyl 2-((6′-fluoro-4-oxo-3′H-spiro[cyclohexane-1,1′-isobenzofuran]-7′-yl)oxy)acetate (Int-6)

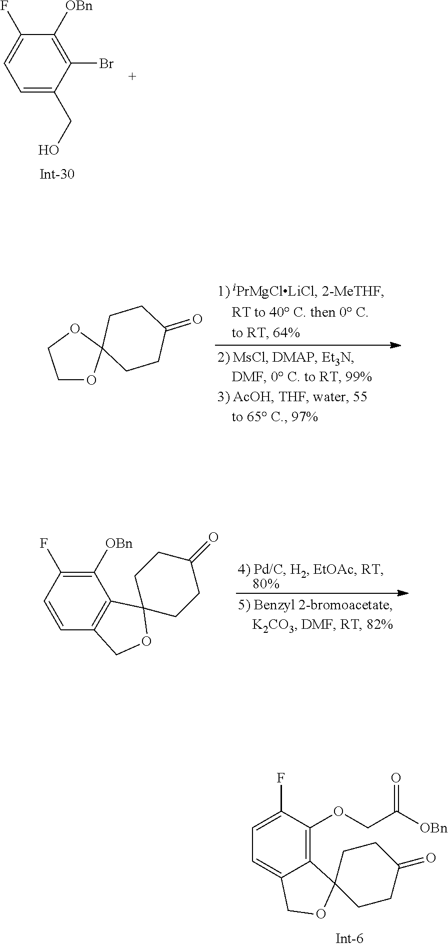

### Step 1:

A solution of ⁱPrMgCl·LiCl (3.6 mL, 1.3 M solution in THF, 4.680 mmol) was added over 1 min to a solution of (3-(benzyloxy)-2-bromo-4-fluorophenyl)methanol (500 mg, 1.591 mmol) in 2-MeTHF (5 mL) at ambient temperature and the reaction mixture was stirred at 40° C. for 2 h. The reaction was cooled with an ice bath. A solution of 1,4-dioxaspiro[4.5]decan-8-one (400 mg, 2.561 mmol) in 2-MeTHF (1 mL) was added and the reaction was stirred at ambient temperature for 15 min. The reaction was quenched by addition of a saturated aqueous NH₄Cl solution (5 mL) and water (1 mL) and the mixture was extracted with EtOAc (2×10 mL). The combined organic layers were washed with brine (20 mL), dried (Na₂SO₄), filtered, and concentrated in vacuo. Purification by flash chromatography (SiO₂, 0 to 30% EtOAc in heptane) gave 8-(2-(benzyloxy)-3-fluoro-6-(hydroxymethyl)phenyl)-1,4-dioxaspiro[4.5]decan-8-ol (400 mg, 64%). ¹H NMR (300 MHz, Chloroform-d) δ 7.79-7.54 (m, 2H), 7.50-7.32 (m, 3H), 7.11-6.80 (m, 2H), 5.11 (s, 2H), 4.93 (d, J=6.0 Hz, 2H), 4.15-3.90 (m, 3H), 3.85 (td, J=6.1, 1.1 Hz, 2H), 3.00-2.59 (m, 3H), 2.11 (td, J=13.4, 4.6 Hz, 2H), 1.78 (dt, J=13.7, 2.5 Hz, 2H), 1.70-1.60 (m, 2H) ppm. ESI-MS m/z calc. 388.168, found 371.3 (M-17)⁺.

### Step 2:

MsCl (500 μL, 6.461 mmol) was added dropwise to a stirred solution of 8-(2-(benzyloxy)-3-fluoro-6-(hydroxymethyl)phenyl)-1,4-dioxaspiro[4.5]decan-8-ol (1.8 g, 4.402 mmol), DMAP (1.2 g, 9.822 mmol) and Et₃N (1.8 mL, 12.91 mmol) in DMF (100 mL) at 0° C. and the reaction mixture was warmed and stirred at ambient temperature for 3 h. The reaction was quenched by addition of a saturated aqueous NH₄Cl solution (250 mL) and extracted with EtOAc (2×300 mL). The combined organic layers were washed with water (2×500 mL) and brine (500 mL), dried (Na₂SO₄), filtered, and concentrated in vacuo. Purification by flash chromatography (120 g SiO₂, 20 to 100% EtOAc in heptane) gave 7-(benzyloxy)-6-fluoro-3H-dispiro[isobenzofuran-1,1′-cyclohexane-4′,2″-[1,3]dioxolane](1.8 g, 99%) as a colourless solid. ¹H NMR (300 MHz, Chloroform-d) δ 7.66-7.56 (m, 2H), 7.49-7.29 (m, 3H), 7.04 (dd, J=12.2, 8.1 Hz, 1H), 6.83 (dd, J=8.1, 3.8 Hz, 1H), 5.23 (d, J=1.3 Hz, 2H), 5.02 (t, J=1.3 Hz, 2H), 4.01 (d, J=1.6 Hz, 4H), 2.47 (td, J=13.8, 4.3 Hz, 2H), 2.12-1.96 (m, 2H), 1.85-1.57 (m, 4H) ppm. ESI-MS m/z calc. 370.158, found 371.4 (M+1)⁺.

### Step 3:

Water (18 mL) and acetic acid (18 mL, 316.5 mmol) were successively added to a stirred solution of 7-(benzyloxy)-6-fluoro-3H-dispiro[isobenzofuran-1,1′-cyclohexane-4′,2″-[1,3]dioxolane](3.4 g, 9.179 mmol) in THF (18 mL) and the solution was stirred at 55° C. for 4 h, then at 65° C. for 2 h. The reaction was quenched by addition of an ice-water mixture and a saturated aqueous NaHCO₃ solution (500 mL, 1:1 ratio) and the pH of the solution was adjusted to pH 7-8. The mixture was extracted with DCM (3×300 mL). The combined organic layers were washed with NaHCO₃ (200 mL) and water (200 mL), passed through a pad containing a mixture of silica and Celite®. The mother liquors were concentrated in vacuo to give 7′-(benzyloxy)-6′-fluoro-3′H-spiro[cyclohexane-1,1′-isobenzofuran]-4-one (2.94 g, 97%) as an oil.

### Step 4:

Pd/C (2.1 g, 1.973 mmol) was added to a degassed solution of 7′-(benzyloxy)-6′-fluoro-3′H-spiro[cyclohexane-1,1′-isobenzofuran]-4-one (2.94 g, 97%) in EtOAc (36 mL) and the reaction mixture was stirred at ambient temperature under an atmospheric pressure of hydrogen for 16.5 h. The reaction mixture was filtered through a pad of silica, washing the filtered cake with EtOAc. Additional Pd/C (2.1 g, 1.973 mmol) was added to the solution and the reaction was stirred at ambient temperature under an atmospheric pressure of hydrogen. The reaction mixture was filtered through a pad of Celite®, washing the filtered cake with EtOAc (4×50 mL). The filtrates were concentrated in vacuo. Purification by trituration from 25% MTBE in heptane gave 6′-fluoro-7′-hydroxy-3′H-spiro[cyclohexane-1,1′-isobenzofuran]-4-one (1.87 g, 80%) as a white solid. ¹H NMR (300 MHz, Chloroform-d) δ 7.02 (dd, J=10.3, 8.2 Hz, 1H), 6.68 (ddt, J=8.2, 4.1, 1.0 Hz, 1H), 5.46 (s, 1H), 5.10 (t, J=1.3 Hz, 2H), 2.87 (td, J=14.1, 6.2 Hz, 2H), 2.72-2.52 (m, 2H), 2.38 (ddt, J=14.3, 4.2, 2.0 Hz, 2H), 2.17-1.94 (m, 2H) ppm. ESI-MS m/z calc. 236.085, found 237.2 (M+1)⁺.

### Step 5:

K₂CO₃ (1.28 g, 9.262 mmol) was added to a stirred solution of 6′-fluoro-7′-hydroxy-3′H-spiro[cyclohexane-1,1′-isobenzofuran]-4-one (1.12 g, 4.693 mmol) and benzyl 2-bromoacetate (870 μL, 5.492 mmol) in DMF (8.30 mL) and the reaction mixture was stirred at ambient temperature for 16 h. The mixture was diluted with EtOAc and filtered. The organic layer was separated, washed with brine (2×30 mL) and concentrated in vacuo. Purification by flash chromatography (80 g SiO₂, 0 to 60% EtOAC in heptane) gave benzyl 2-((6′-fluoro-4-oxo-3′H-spiro[cyclohexane-1,1′-isobenzofuran]-7′-yl)oxy)acetate (Int-6, 1.480 g, 82%). ¹H NMR (300 MHz, Chloroform-d) δ 7.41-7.30 (m, 5H), 7.00 (dd, J=12.8, 8.2 Hz, 1H), 6.81 (ddt, J=8.1, 3.8, 1.0 Hz, 1H), 5.21 (s, 2H), 5.07 (t, J=1.3 Hz, 2H), 4.83 (d, J=2.6 Hz, 2H), 2.83 (td, J=14.2, 6.2 Hz, 2H), 2.59 (td, J=13.6, 4.7 Hz, 2H), 2.34 (ddt, J=14.3, 4.5, 1.9 Hz, 2H), 2.10-2.00 (m, 2H) ppm. ESI-MS m/z calc. 384.137, found 385.2 (M+1)⁺.

### Example 35: Intermediate 7

### Benzyl 2-((4′-fluoro-4-oxo-3′H-spiro[cyclohexane-1,1′-isobenzofuran]-7′-yl)oxy)acetate (Int-7)

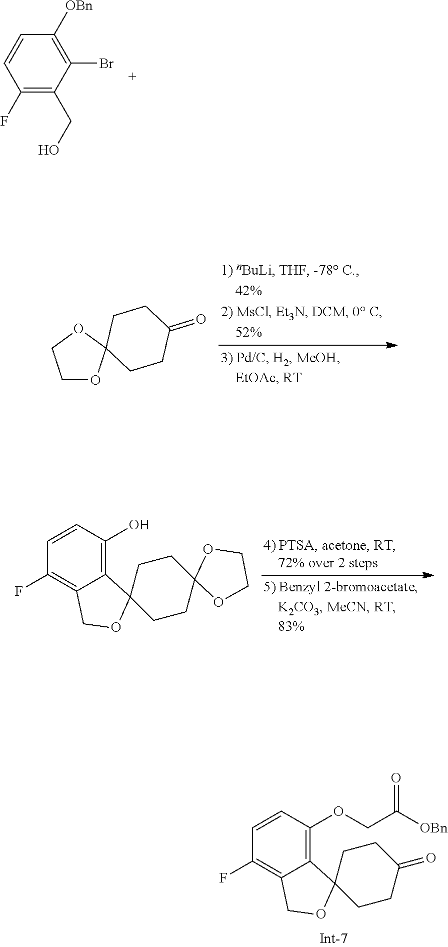 [SMILES: OCc1c(F)ccc(OCc2ccccc2)c1Br ; O=C1CCC2(CC1)OCCO2 ; Oc1ccc(F)c2c1C1(CCC3(CC1)OCCO3)OC2 ; O=C1CCC2(CC1)OCc1c(F)ccc(OCC(=O)OCc3ccccc3)c12]

### Step 1:

ⁿBuLi (31 mL, 1.6 M solution in hexanes, 49.60 mmol) was added at −78° C. to a solution of (3-(benzyloxy)-2-bromo-6-fluorophenyl)methanol (7 g, 22.50 mmol) in THF (140 mL) and the reaction mixture was stirred at −78° C. for 1 h. A solution of 1,4-dioxaspiro[4.5]decan-8-one (4.9 g, 31.37 mmol) in THF (35 mL) was added and the mixture was stirred at −78° C. for 1 h. The reaction was concentrated in vacuo. Purification by flash chromatography (SiO₂, 0 to 35% EtOAc in heptane) gave 8-(6-(benzyloxy)-3-fluoro-2-(hydroxymethyl)phenyl)-1,4-dioxaspiro[4.5]decan-8-ol (3.7 g, 42%) as a white solid. ¹H NMR (400 MHz, Chloroform-d) δ 7.58-7.31 (m, 5H), 7.03-6.90 (m, 2H), 5.12 (d, J=2.2 Hz, 4H), 3.96 (td, J=6.4, 1.6 Hz, 2H), 3.82 (dt, J=7.3, 3.7 Hz, 2H), 3.13-2.98 (m, 2H), 2.18-2.03 (m, 2H), 1.81-1.65 (m, 4H) ppm; exchangeable H not observed.

### Step 2:

MsCl (6.23 mL, 80.50 mmol) was added dropwise to a stirred solution of 8-(6-(benzyloxy)-3-fluoro-2-(hydroxymethyl)phenyl)-1,4-dioxaspiro[4.5]decan-8-ol (17.24 g, 43.99 mmol) and Et₃N (22 mL, 157.8 mmol) in DCM (515 mL) at 0° C., whilst maintaining the temperature below 5° C. The reaction mixture was stirred for 90 min, then quenched by addition of water (100 mL), and warmed to ambient temperature. The reaction was diluted with a saturated aqueous NaHCO₃ solution (200 mL). The aqueous phase was separated and extracted with DCM (200 mL). The combined organic layers were washed with brine, dried (Na₂SO₄), filtered, and concentrated in vacuo. Purification by flash chromatography (330 g SiO₂, 0 to 20% EtOAc in heptane followed by 330 g SiO₂, 0 to 5% MeCN in DCM) gave 7-(benzyloxy)-4-fluoro-3H-dispiro[isobenzofuran-1,1′-cyclohexane-4′,2″-[1,3]dioxolane](8.57 g, 52%) as a white solid. H NMR (300 MHz, Chloroform-d) δ 7.57-7.47 (m, 2H), 7.45-7.27 (m, 3H), 6.86 (dd, J=8.8, 8.2 Hz, 1H), 6.73 (dd, J=8.8, 3.5 Hz, 1H), 5.15-5.08 (m, 4H), 4.01 (s, 4H), 2.61 (td, J=14.2, 4.8 Hz, 2H), 2.12-1.95 (m, 2H), 1.84-1.66 (m, 4H) ppm. ESI-MS m/z calc. 370.158, found 371.3 (M+1)⁺.

### Step 3:

Pd (2.2 g, 2.5% w/w on carbon, 0.517 mmol) was added to a stirred solution of 7-(benzyloxy)-4-fluoro-3H-dispiro[isobenzofuran-1,1′-cyclohexane-4′,2″-[1,3]dioxolane](2.1 g, 5.669 mmol) in a mixture of MeOH (20 mL) and EtOAc (20 mL) and the reaction mixture was stirred under an atmospheric pressure of hydrogen at ambient temperature overnight. The mixture was filtered through a pad of Celite®, washing the filtered cake with EtOAc and the liquors were concentrated in vacuo to give 4-fluoro-3H-dispiro[isobenzofuran-1,1′-cyclohexane-4′,2″-[1,3]dioxolan]-7-ol, which was used without further purification in the next step.

### Step 4:

PTSA (1.8 mL, 11.18 mmol) was added to a stirred solution of 4-fluoro-3H-dispiro[isobenzofuran-1,1′-cyclohexane-4′,2″-[1,3]dioxolan]-7-ol in acetone (25 mL) and the reaction mixture was stirred at ambient temperature for 2 h. The reaction was partitioned between a saturated aqueous NH₄Cl solution and EtOAc. The organic layer was separated, filtered through a pad of silica and concentrated in vacuo. Purification by flash chromatography (SiO₂, 0 to 60% EtOAc in heptane) gave 4′-fluoro-7′-hydroxy-3′H-spiro[cyclohexane-1,1′-isobenzofuran]-4-one (1.1 g, 72% over 2 steps) as a white solid. ¹H NMR (400 MHz, Chloroform-d) δ 6.91-6.82 (m, 1H), 6.66-6.58 (m, 1H), 5.40 (s, 1H), 5.22 (s, 2H), 2.91 (td, J=14.2, 6.0 Hz, 2H), 2.66 (td, J=13.9, 4.7 Hz, 2H), 2.42 (ddd, J=14.7, 5.0, 2.3 Hz, 2H), 2.18-2.09 (m, 2H) ppm. ESI-MS m/z calc. 236.085, found 236.3 (M+1)⁺.

### Step 5:

K₂CO₃ (1.3 g, 9.406 mmol) was added to a stirred solution of 4′-fluoro-7′-hydroxy-3′H-spiro[cyclohexane-1,1′-isobenzofuran]-4-one (1.1 g, 4.237 mmol) and benzyl 2-bromoacetate (807 μL, 5.094 mmol) in MeCN (25 mL) and the reaction mixture was stirred at ambient temperature for 18 h. The mixture was filtered through a pad of silica, washing the filtered cake with EtOAc, and the liquors were concentrated in vacuo. Purification by flash chromatography (40 g SiO₂, 0 to 40% EtOAc in heptane) gave benzyl 2-((4′-fluoro-4-oxo-3′H-spiro[cyclohexane-1,1′-isobenzofuran]-7′-yl)oxy)acetate (Int-7, 1.35 g, 83%) as a white solid. ¹H NMR (400 MHz, Chloroform-d) δ 7.44-7.37 (m, 3H), 7.37-7.31 (m, 2H), 6.97-6.87 (m, 1H), 6.62-6.53 (m, 1H), 5.25-5.19 (m, 4H), 4.73-4.66 (m, 2H), 2.87 (tdd, J=13.5, 5.8, 3.0 Hz, 2H), 2.67 (td, J=14.3, 5.2 Hz, 2H), 2.43-2.33 (m, 2H), 2.13-1.99 (m, 2H) ppm.

The following Intermediate was made using the method described in Intermediate 7 except that the conditions used in Steps 1 and 2 were those of Intermediate Int-1 Steps 1 and 2 using rac-1-(3-(benzyloxy)-2-bromophenyl)ethan-1-ol as the starting material in place of (3-(benzyloxy)-2-bromophenyl)methanol. The conditions used in Steps 3 and 4 were those of Intermediate Int-6 Steps 4 and 3:

| Intermediate | | | | |
|---|---|---|---|---|
| Number | Structure | Name | LC/MS | NMR (shifts in ppm) |
| Int-13 | 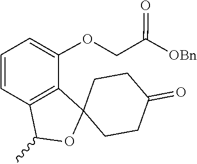 [SMILES: CC1OC2(CCC(=O)CC2)c2c(OCC(=O)OCc3ccccc3)cccc21] | Benzyl rac-2- ((3′-methyl-4- oxo-3′H- spiro[cyclo- hexane-1,1′- isobenzofuran]- 7′-yl)oxy)acetate | ESI-MS m/z calc. 380.162, found 381.3 (M + 1)⁺. | ¹H NMR (300 MHz, Chloroform-d) δ 7.38-7.26 (m, 5H), 7.25-7.17 (m, 1H), 6.79 (d, J = 7.5 Hz, 1H), 6.60 (d, J = 8.1 Hz, 1H), 5.35 (q, J = 6.1 Hz, 1H), 5.20 (d, J = 1.9 Hz, 2H), 4.70 (d, J = 4.2 Hz, 2H), 2.99-2.73 (m, 3H), 2.62- 2.45 (m, 1H), 2.41-2.23 (m, |
| | | | | 2H), 2.03-1.90 (m, 2H), |
| | | | | 1.52 (d, J = 6.4 Hz, 3H) |
| | | | | ppm. |

### Example 36: Intermediate 8

### Benzyl 2-((4′,5′-difluoro-4-oxo-3′H-spiro[cyclohexane-1,1′-isobenzofuran]-7′-yl)oxy)acetate (Int-8)

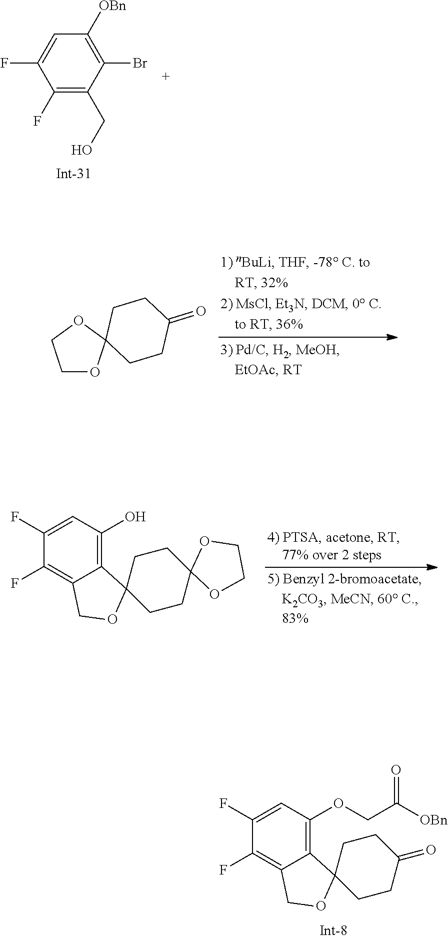 [SMILES: OCc1c(F)c(F)cc(OCc2ccccc2)c1Br ; O=C1CCC2(CC1)OCCO2 ; Oc1cc(F)c(F)c2c1C1(CCC3(CC1)OCCO3)OC2 ; O=C1CCC2(CC1)OCc1c(F)c(F)cc(OCC(=O)OCc3ccccc3)c12]

### Step 1:

(3-(Benzyloxy)-2-bromo-5,6-difluorophenyl)methanol (Int-31, 1.1 g, 3.342 mmol) and 1,4-dioxaspiro[4.5]decan-8-one (783 mg, 5.013 mmol) were treated via the method described in Intermediate 1 Step 1 to give, after purification by flash chromatography (SiO₂, 10 to 100% EtOAc in heptane), 8-(6-(benzyloxy)-3,4-difluoro-2-(hydroxymethyl)phenyl)-1,4-dioxaspiro[4.5]decan-8-ol (436 mg, 32%). ¹H NMR (400 MHz, Chloroform-d) δ 7.54-7.35 (m, 5H), 6.89-6.79 (m, 1H), 5.10 (d, J=11.5 Hz, 4H), 3.95 (t, J=6.1 Hz, 2H), 3.80 (t, J=6.1 Hz, 2H), 3.09-2.96 (m, 2H), 2.16-2.01 (m, 4H), 1.71 (d, J=13.8 Hz, 2H), 1.61 (d, J=13.4 Hz, 2H) ppm.

### Step 2:

MsCl (1.0 mL, 12.92 mmol) was added to a stirred solution of 8-(6-(benzyloxy)-3,4-difluoro-2-(hydroxymethyl)phenyl)-1,4-dioxaspiro[4.5]decan-8-ol (4.4 g, 10.83 mmol) and Et₃N (5.34 mL, 38.31 mmol) in DCM (75 mL) at 0° C. and the reaction mixture was stirred at 0° C. for 30 min and at ambient temperature for 1 h. The reaction was quenched by addition of a saturated aqueous NH₄Cl solution (100 mL). The aqueous phase was separated and extracted with DCM (2×20 mL). The combined organic layers were washed with brine (20 mL), dried (Na₂SO₄), filtered, and concentrated in vacuo. Purification by flash chromatography (24 g SiO₂, 0 to 40% EtOAc in heptane) gave 7-(benzyloxy)-4,5-difluoro-3H-dispiro[isobenzofuran-1,1′-cyclohexane-4′,2″-[1,3]dioxolane](1.5 g, 36%) as a white solid. ¹H NMR (300 MHz, Chloroform-d) δ 7.73-7.33 (m, 5H), 6.66 (dd, J=11.9, 5.5 Hz, 1H), 5.14 (d, J=0.9 Hz, 2H), 5.11 (s, 2H), 4.03 (s, 4H), 2.59 (td, J=14.1, 4.8 Hz, 2H), 2.05 (td, J=13.7, 4.5 Hz, 2H), 1.91-1.67 (m, 4H) ppm.

### Step 3:

7-(Benzyloxy)-4,5-difluoro-3H-dispiro[isobenzofuran-1,1′-cyclohexane-4′,2″-[1,3]dioxolane](1.48 g, 3.810 mmol) was treated via the method described in Intermediate 1 Step 3 to give 4,5-difluoro-3H-dispiro[isobenzofuran-1,1′-cyclohexane-4′,2″-[1,3]dioxolan]-7-ol, which was used without further purification in the next step.

### Step 4:

4,5-Difluoro-3H-dispiro[isobenzofuran-1,1′-cyclohexane-4′,2″-[1,3]dioxolan]-7-ol was treated via the method described in Intermediate 1 Step 4 to give 4′,5′-difluoro-7′-hydroxy-3′H-spiro[cyclohexane-1,1′-isobenzofuran]-4-one (930 mg, 77% over 2 steps) as a white solid. ¹H NMR (300 MHz, Chloroform-d) δ 6.52 (dd, J=10.7, 5.4 Hz, 1H), 5.22 (q, J=1.0 Hz, 2H), 2.87 (td, J=14.1, 6.2 Hz, 2H), 2.59 (td, J=13.7, 4.7 Hz, 2H), 2.45-2.32 (m, 2H), 2.15-1.98 (m, 2H) ppm; exchangeable H not observed.

### Step 5:

Benzyl 2-bromoacetate (421 μL, 2.658 mmol) was added to a stirred suspension of 4′,5′-difluoro-7′-hydroxy-3′H-spiro[cyclohexane-1,1′-isobenzofuran]-4-one (520 mg, 2.045 mmol) and K₂CO₃ (642 mg, 4.645 mmol) in MeCN (50 mL) and the reaction mixture was stirred at 60° C. for 2 h. The mixture was filtered through a pad of Celite® and the liquors were concentrated in vacuo. Purification by flash chromatography (24 g SiO₂, 0 to 50% EtOAc in heptane) gave benzyl 2-((4′,5′-difluoro-4-oxo-3′H-spiro[cyclohexane-1,1′-isobenzofuran]-7′-yl)oxy)acetate (Int-8, 760 mg, 83%) as a colourless oil. ¹H NMR (400 MHz, Chloroform-d) δ 7.49-7.33 (m, 5H), 6.50 (dd, J=11.5, 5.3 Hz, 1H), 5.24 (s, 4H), 4.67 (d, J=2.0 Hz, 2H), 2.85 (td, J=14.3, 6.0 Hz, 2H), 2.62 (td, J=13.8, 4.7 Hz, 2H), 2.42-2.33 (m, 2H), 2.08-1.94 (m, 2H) ppm.

### Example 37: Intermediate 9

### Benzyl 2-((4′,6′-difluoro-4-oxo-3′H-spiro[cyclohexane-1,1′-isobenzofuran]-7′-yl)oxy)acetate (Int-9)

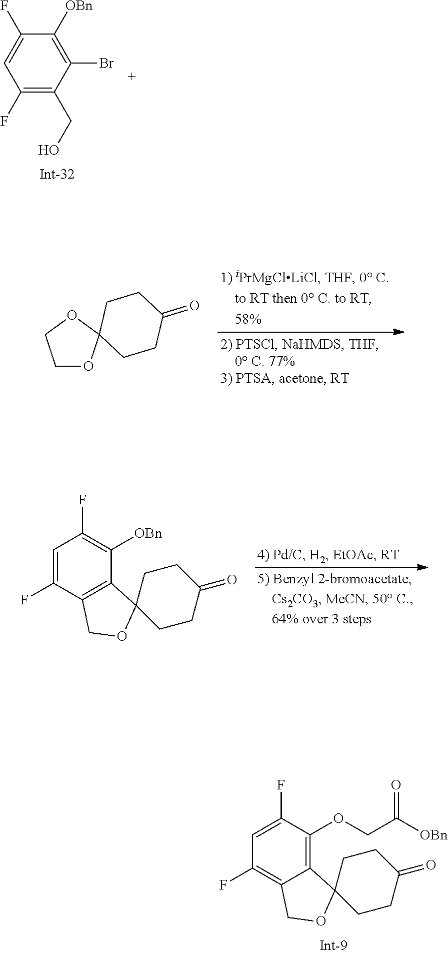 [SMILES: OCc1c(F)cc(F)c(OCc2ccccc2)c1Br ; O=C1CCC2(CC1)OCCO2 ; O=C1CCC2(CC1)OCc1c(F)cc(F)c(OCc3ccccc3)c12 ; O=C1CCC2(CC1)OCc1c(F)cc(F)c(OCC(=O)OCc3ccccc3)c12]

### Step 1:

ⁱPrMgCl·LiCl (54.7 mL, 1.3 M solution in THF, 71.11 mmol) was added to a stirred solution of (3-(benzyloxy)-2-bromo-4,6-difluorophenyl)methanol (Int-32, 7.8 g, 23.70 mmol) in THF (78 mL) at 0° C. and the reaction mixture was stirred at ambient temperature for 30 min. The mixture was cooled back to 0° C. A solution of 1,4-dioxaspiro[4.5]decan-8-one (5.55 g, 35.54 mmol) in THF (67.2 mL) was added and the reaction mixture was warmed and stirred at ambient temperature for 15 min. The reaction was quenched by addition of a saturated aqueous NH₄Cl solution (100 mL) and water (100 mL) and the mixture was extracted with EtOAc (2×10 mL). The combined organic layers were washed with brine (50 mL), dried (Na₂SO₄), filtered, and concentrated in vacuo. Purification by flash chromatography (80 g SiO₂, 10 to 60% EtOAc in heptane) gave 8-(2-(benzyloxy)-3,5-difluoro-6-(hydroxymethyl)phenyl)-1,4-dioxaspiro[4.5]decan-8-ol (5.64 g, 58%) as a white solid. ¹H NMR (400 MHz, Chloroform-d) δ 7.64-7.57 (m, 2H), 7.54-7.30 (m, 3H), 6.88 (dd, J=10.9, 9.7 Hz, 1H), 5.05 (d, J=2.5 Hz, 4H), 4.15-3.75 (m, 4H), 2.83 (td, J=13.7, 4.4 Hz, 2H), 2.11 (td, J=13.4, 4.6 Hz, 2H), 1.81 (ddt, J=14.0, 4.9, 2.5 Hz, 2H), 1.68-1.62 (m, 2H) ppm; exchangeable H not observed. ESI-MS m/z calc. 406.159, found 407.2 (M+1)⁺.

### Step 2:

NaHMDS (41.3 mL, 1 M solution in THF, 41.30 mmol) was added to a solution of 8-(2-(benzyloxy)-3,5-difluoro-6-(hydroxymethyl)phenyl)-1,4-dioxaspiro[4.5]decan-8-ol (5.6 g, 13.74 mmol) in THF (110 mL) at 0° C. and the reaction mixture was stirred at 0° C. for 10 min. A solution of PTSCl (3.9 g, 20.46 mmol) in THF (56 mL) was added and the reaction mixture was stirred at 0° C. for 10 min. The reaction was quenched by addition of a saturated aqueous NH₄Cl solution (100 mL), diluted with water (100 mL) and extracted with EtOAc (2×100 mL). The combined organic layers were washed with brine (100 mL), dried (Na₂SO₄), filtered, and concentrated in vacuo. Purification by flash chromatography (24 g SiO₂, 0 to 10% EtOAc in DCM) gave 7-(benzyloxy)-4,6-difluoro-3H-dispiro[isobenzofuran-1,1′-cyclohexane-4′,2″-[1,3]dioxolane](4.1 g, 77%) as a white solid. ¹H NMR (400 MHz, Chloroform-d) δ 7.49-7.41 (m, 2H), 7.35-7.22 (m, 3H), 6.67 (dd, J=11.5, 8.0 Hz, 1H), 5.01 (s, 2H), 4.92 (t, J=1.2 Hz, 2H), 3.99-3.57 (m, 4H), 2.30 (td, J=13.8, 4.2 Hz, 2H), 1.95-1.82 (m, 2H), 1.72-1.50 (m, 4H) ppm.

### Step 3:

7-(Benzyloxy)-4,6-difluoro-3H-dispiro[isobenzofuran-1,1′-cyclohexane-4′,2″-[1,3]dioxolane](5.1 g, 13.13 mmol) was treated via the method described in Intermediate 1 Step 4 to give 7′-(benzyloxy)-4′,6′-difluoro-3′H-spiro[cyclohexane-1,1′-isobenzofuran]-4-one as a white solid, which was used without further purification in the next step.

### Step 4:

A suspension of 7′-(benzyloxy)-4′,6′-difluoro-3′H-spiro[cyclohexane-1,1′-isobenzofuran]-4-one and Pd/C (2.61 g, 2.453 mmol) in EtOAc (51 mL) was stirred under a hydrogen atmosphere at ambient temperature for 45 min. The reaction was filtered through a pad of Celite®, washing the filtered cake with EtOAc (200 mL). The filtrates were concentrated in vacuo to give 4′,6′-difluoro-7′-hydroxy-3′H-spiro[cyclohexane-1,1′-isobenzofuran]-4-one, which was used without further purification in the next step.

### Step 5:

4′,6′-Difluoro-7′-hydroxy-3′H-spiro[cyclohexane-1,1′-isobenzofuran]-4-one and benzyl 2-bromoacetate (3.6 g, 15.72 mmol) were treated via the method described in Intermediate 1 Step 5 to give, after purification by flash chromatography (SiO₂, 2 to 30% EtOAc in heptane), benzyl 2-((4′,6′-difluoro-4-oxo-3′H-spiro[cyclohexane-1,1′-isobenzofuran]-7′-yl)oxy)acetate (Int-9, 3.4 g, 64% over 3 steps) as a colourless oil, which crystallised into a white solid upon standing. ¹H NMR (400 MHz, Chloroform-d) δ 7.45-7.34 (m, 5H), 6.79 (dd, J=12.1, 7.9 Hz, 1H), 5.23 (s, 2H), 5.17-5.12 (m, 2H), 4.79 (d, J=2.2 Hz, 2H), 2.91-2.77 (m, 2H), 2.65-2.53 (m, 2H), 2.37 (ddt, J=14.6, 4.6, 2.0 Hz, 2H), 2.18-2.01 (m, 2H) ppm.

### Example 38: Intermediate 12

### Benzyl 2-((4′-methyl-4-oxo-3′H-spiro[cyclohexane-1,1′-isobenzofuran]-7′-yl)oxy)acetate (Int-12)

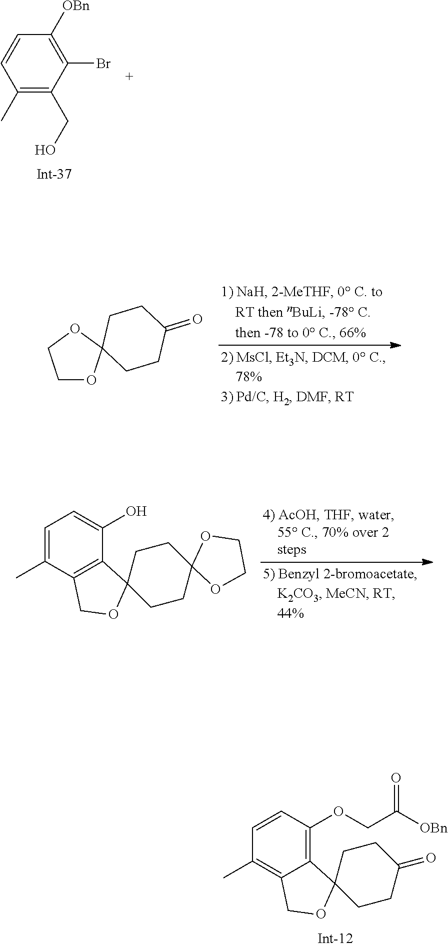 [SMILES: Cc1ccc(OCc2ccccc2)c(Br)c1CO ; O=C1CCC2(CC1)OCCO2 ; Cc1ccc(O)c2c1COC21CCC2(CC1)OCCO2 ; Cc1ccc(OCC(=O)OCc2ccccc2)c2c1COC21CCC(=O)CC1]

### Step 1:

NaH (218.9 mg, 60% dispersion in mineral oil, 5.473 mmol) was added in a single portion to a stirred solution of (3-(benzyloxy)-2-bromo-6-methylphenyl)methanol (950 mg, 3.069 mmol) in 2-MeTHF (14 mL) at 0° C. and the reaction mixture was warmed to ambient temperature and stirred for 1 h. The reaction was cooled to −78° C. ⁿBuLi (3.9 mL, 1.6 M in hexanes, 6.240 mmol) was added dropwise over 5 min and the reaction was stirred at −78° C. for 35 min. A solution of 1,4-dioxaspiro[4.5]decan-8-one (840.6 mg, 5.382 mmol) in 2-MeTHF (3 mL) was added and the reaction mixture was stirred at −78° C. for 2 h. The reaction was warmed to 0° C., quenched by addition of water (25 mL) and a saturated aqueous NH₄Cl solution (25 mL) and warmed to ambient temperature. The mixture was extracted with EtOAc (2×50 mL). The combined organic layers were washed with brine (50 mL), dried (Na₂SO₄), filtered, and concentrated in vacuo. Purification by flash chromatography (24 g SiO₂, 0 to 85% EtOAc in heptane) gave 8-(6-(benzyloxy)-2-(hydroxymethyl)-3-methylphenyl)-1,4-dioxaspiro[4.5]decan-8-ol (780 mg, 66%) as a clear waxy solid. ¹H NMR (300 MHz, Chloroform-d) δ 7.52-7.45 (m, 2H), 7.43-7.28 (m, 3H), 7.03 (dd, J=8.4, 0.7 Hz, 1H), 6.85 (d, J=8.4 Hz, 1H), 5.08 (s, 2H), 4.95 (d, J=5.8 Hz, 2H), 3.96-3.87 (m, 2H), 3.85-3.73 (m, 3H), 3.02 (td, J=13.7, 4.5 Hz, 2H), 2.90-2.78 (m, 1H), 2.38 (d, J=0.6 Hz, 3H), 2.15-1.96 (m, 2H), 1.81-1.69 (m, 2H), 1.64-1.52 (m, 2H) ppm. ESI-MS m/z calc. 384.194, found 384.3 (M+1)⁺.

### Step 2:

8-(6-(Benzyloxy)-2-(hydroxymethyl)-3-methylphenyl)-1,4-dioxaspiro[4.5]decan-8-ol (6.2 g, 16.08 mmol) was treated via the method described in Intermediate 7 Step 2 to give, after purification by flash chromatography (120 g SiO₂, 0 to 80% EtOAc in heptane), 7-(benzyloxy)-4-methyl-3H-dispiro[isobenzofuran-1,1′-cyclohexane-4′,2″-[1,3]dioxolane](4.86 g, 78%) as a white solid. ¹H NMR (300 MHz, Chloroform-d) δ 7.57-7.48 (m, 2H), 7.43-7.28 (m, 3H), 6.98 (d, J=8.1, 0.9 Hz, 1H), 6.73 (d, J=8.1 Hz, 1H), 5.13 (s, 2H), 5.02 (s, 2H), 4.01 (s, 4H), 2.75-2.51 (m, 2H), 2.16 (s, 3H), 2.13-1.97 (m, 2H), 1.86-1.65 (m, 4H) ppm. ESI-MS m/z calc. 366.183, found 367.4 (M+1)⁺.

### Step 3:

Pd/C (2.731 g, 2.566 mmol) was added to a stirred solution of 7-(benzyloxy)-4-methyl-3H-dispiro[isobenzofuran-1,1′-cyclohexane-4′,2″-[1,3]dioxolane](4.86 g, 12.61 mmol) in DMF (60 mL) and the reaction mixture was stirred under a hydrogen atmosphere at ambient temperature overnight. The reaction was filtered through a pad of Celite®, washing the filtered cake with EtOAc. The filtrates were concentrated in vacuo to give 4-methyl-3H-dispiro[isobenzofuran-1,1′-cyclohexane-4′,2″-[1,3]dioxolan]-7-ol (4.6 g, 115%) as an off-white solid, which was used without further purification in the next step.

### Step 4:

4-Methyl-3H-dispiro[isobenzofuran-1,1′-cyclohexane-4′,2″-[1,3]dioxolan]-7-ol (4.6 g, 115%) was treated via the method described in Example 6 Step 3 to give, after purification by flash chromatography (80 g SiO₂, 0 to 100% EtOAc in heptane), 7′-hydroxy-4′-methyl-3′H-spiro[cyclohexane-1,1′-isobenzofuran]-4-one (2.38 g, 70% over 2 steps) as a white solid. ¹H NMR (300 MHz, Chloroform-d) δ 6.92 (d, J=7.9 Hz, 1H), 6.55 (d, J=7.9 Hz, 1H), 5.12 (s, 1H), 5.09 (s, 2H), 2.88 (td, J=14.1, 6.1 Hz, 2H), 2.63 (td, J=13.7, 4.7 Hz, 2H), 2.37 (ddt, J=14.2, 4.3, 2.0 Hz, 2H), 2.16 (s, 3H), 2.07 (ddt, J=14.2, 6.0, 3.2 Hz, 2H) ppm. ESI-MS m/z calc. 232.110, found 233.1 (M+1)⁺.

### Step 5:

7′-Hydroxy-4′-methyl-3′H-spiro[cyclohexane-1,1′-isobenzofuran]-4-one (2.38 g, 8.835 mmol) was treated via the method described in Example 7 Step 5 to give, after purification by reverse phase chromatography (275 g C₁₈ column, 0 to 100% MeCN in water with 0.2% formic acid), benzyl 2-((4′-methyl-4-oxo-3′H-spiro[cyclohexane-1,1′-isobenzofuran]-7′-yl)oxy)acetate (Int-12, 1.5 g, 44%) as a clear oil. ¹H NMR (300 MHz, Chloroform-d) δ 7.34-7.18 (m, 5H), 6.89 (dd, J=8.2, 0.8 Hz, 1H), 6.44 (d, J=8.2 Hz, 1H), 5.11 (s, 2H), 5.00 (s, 2H), 4.58 (s, 2H), 2.76 (td, J=14.1, 6.0 Hz, 2H), 2.57 (td, J=13.6, 4.6 Hz, 2H), 2.25 (dq, J=12.3, 2.4 Hz, 2H), 2.10 (s, 3H), 2.00-1.82 (m, 2H) ppm. ESI-MS m/z calc.
380.162, found 381.3 (M+1)⁺.

### Example 39: Intermediate 14

### Benzyl 2-((3′,3′-dimethyl-4-oxo-3′H-spiro[cyclohexane-1,1′-isobenzofuran]-7′-yl)oxy)acetate (Int-14)

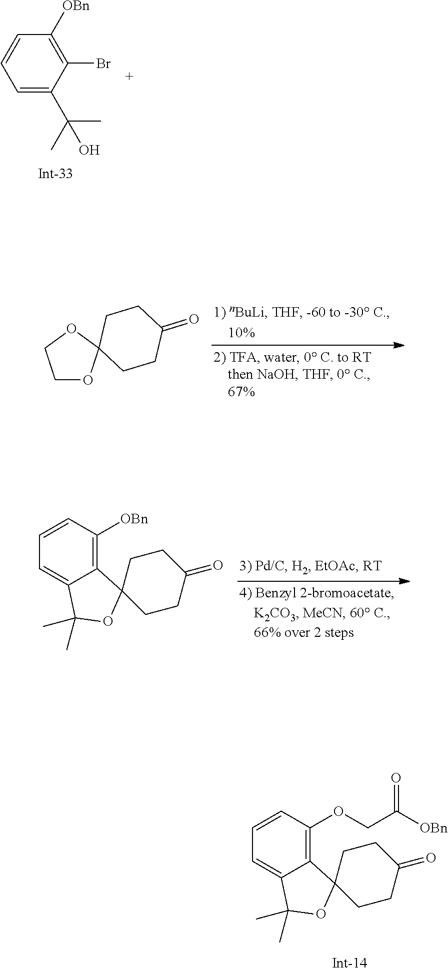 [SMILES: CC(C)(O)c1cccc(OCc2ccccc2)c1Br ; O=C1CCC2(CC1)OCCO2 ; CC1(C)OC2(CCC(=O)CC2)c2c(OCc3ccccc3)cccc21 ; CC1(C)OC2(CCC(=O)CC2)c2c(OCC(=O)OCc3ccccc3)cccc21]

### Step 1:

ⁿBuLi (300 μL, 2.5 M solution in hexanes, 0.750 mmol) was added dropwise to a stirred solution of 2-(3-(benzyloxy)-2-bromophenyl)propan-2-ol (Int-33, 100 mg, 0.287 mmol) in THF (2 mL) at −60° C. and the reaction mixture was stirred at −60° C. for 1 h. A solution of 1,4-dioxaspiro[4.5]decan-8-one (90 mg, 0.576 mmol) in THF (0.5 mL) was added dropwise and the reaction mixture was warmed to −30° C. The reaction was quenched by addition of a saturated aqueous NH₄Cl solution (300 mL). The mixture was warmed to ambient temperature and diluted with MTBE (10 mL) and water (5 mL). The organic layer was separated, washed with brine (5 mL), dried (MgSO₄), filtered and concentrated in vacuo. Purification by flash chromatography gave 8-(2-(benzyloxy)-6-(2-hydroxypropan-2-yl)phenyl)-1,4-dioxaspiro[4.5]decan-8-ol (12 mg, 10%) as a clear oil. ¹H NMR (300 MHz, Chloroform-d) δ 7.54-7.46 (m, 2H), 7.43-7.32 (m, 3H), 7.11-7.03 (m, 1H), 7.03-6.95 (m, 1H), 6.88-6.82 (m, 1H), 5.10 (s, 2H), 3.95-3.87 (m, 2H), 3.78-3.72 (m, 2H), 3.13 (td, J=13.6, 4.6 Hz, 2H), 2.16-2.03 (m, 3H), 1.80-1.69 (m, 8H), 1.59-1.49 (m, 2H) ppm; exchangeable H not observed. ESI-MS m/z calc. 398.209, found 402.4 (M+1)⁺.

### Step 2:

TFA (4 mL, 51.92 mmol) cooled to 0° C. was added to a stirred solution of 8-(2-(benzyloxy)-6-(2-hydroxypropan-2-yl)phenyl)-1,4-dioxaspiro[4.5]decan-8-ol (2 g, 4.567 mmol) in water (1 mL, 55.51 mmol) at 0° C. and the reaction mixture was stirred at 0° C. for 10 min, then at ambient temperature for 10 min. The reaction was cooled to 0° C. and diluted with THF (20 mL). NaOH (8.5 mL, 6 M aqueous solution, 51 mmol) was added dropwise over 5 min and the resulting yellow suspension was stirred at 0° C. for 1 h. The solid was filtered, rinsed with water (20 mL) and heptane (20 mL) to give 7′-(benzyloxy)-3′,3′-dimethyl-3′H-spiro[cyclohexane-1,1′-isobenzofuran]-4-one (1.1411 g, 67%) as a white solid. ¹H NMR (300 MHz, DMSO-d₆) δ 7.45-7.23 (m, 6H), 6.96 (dd, J=8.2, 0.7 Hz, 1H), 6.87 (dd, J=7.5, 0.7 Hz, 1H), 5.16 (s, 2H), 2.77 (td, J=14.0, 5.9 Hz, 2H), 2.58 (dd, J=13.4, 4.5 Hz, 2H), 2.24-2.09 (m, 2H), 1.98-1.83 (m, 2H), 1.49 (s, 6H) ppm. ESI-MS m/z calc. 336.173, found 337.3 (M+1)⁺. The mother liquors were filtered, rinsed with heptane to give a second crop of 7′-(benzyloxy)-3′,3′-dimethyl-3′H-spiro[cyclohexane-1,1′-isobenzofuran]-4-one (80.9 mg, 5%) as a white solid. ESI-MS m/z calc. 336.173, found 337.3 (M+1)⁺.

### Step 3:

7′-(Benzyloxy)-3′,3′-dimethyl-3′H-spiro[cyclohexane-1,1′-isobenzofuran]-4-one (1.140 g, 3.389 mmol) was treated via the method described in Intermediate 6 Step 4 to give to give 7′-hydroxy-3′,3′-dimethyl-3′H-spiro[cyclohexane-1,1′-isobenzofuran]-4-one as an off-white solid, which was used without further purification in the next step.

### Step 4:

Benzyl 2-bromoacetate (700 μL, 4.419 mmol) and 7′-hydroxy-3′,3′-dimethyl-3′H-spiro[cyclohexane-1,1′-isobenzofuran]-4-one were treated via the method described in Intermediate 8 Step 5 to give, after purification by flash chromatography (SiO₂, 0 to 100% EtOAc in heptane), benzyl 2-((3′,3′-dimethyl-4-oxo-3′H-spiro[cyclohexane-1,1′-isobenzofuran]-7′-yl)oxy)acetate (Int-14, 1.170 g, 66%) as a white solid. ¹H NMR (300 MHz, DMSO-d₆) δ 7.44-7.26 (m, 5H), 7.26-7.20 (m, 1H), 6.90 (dd, J=7.5, 0.7 Hz, 1H), 6.83 (dd, J=8.2, 0.7 Hz, 1H), 5.18 (s, 2H), 4.92 (s, 2H), 2.81-2.54 (m, 4H), 2.17 (d, J=13.2 Hz, 2H), 1.89-1.79 (m, 2H), 1.49 (s, 6H) ppm. ESI-MS m/z calc. 394.178, found 395.3 (M+1)⁺.

### Example 40: Intermediate 15

### Benzyl 2-((3′,4-dioxo-3′H-spiro[cyclohexane-1,1′-isobenzofuran]-7′-yl)oxy)acetate (Int-15)

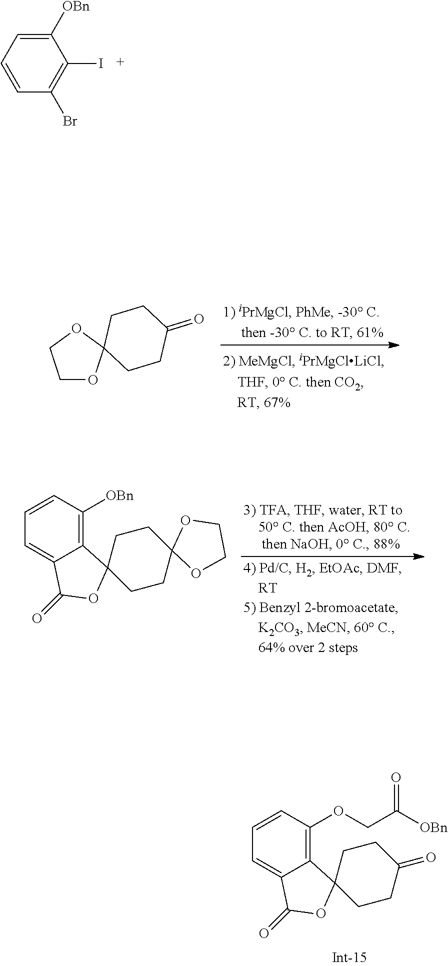 [SMILES: Brc1cccc(OCc2ccccc2)c1I ; O=C1CCC2(CC1)OCCO2 ; O=C1OC2(CCC3(CC2)OCCO3)c2c(OCc3ccccc3)cccc21 ; O=C1CCC2(CC1)OC(=O)c1cccc(OCC(=O)OCc3ccccc3)c12]

### Step 1:

ⁱPrMgCl (70 mL, 2 M solution in THF, 140 mmol) was added dropwise to a stirred solution of 1-(benzyloxy)-3-bromo-2-iodobenzene (50 g, 128.5 mmol) in toluene (250 mL) at −30° C., whilst maintaining the internal temperature below −25° C., and the reaction mixture was stirred at −30° C. for 20 min. A solution of 1,4-dioxaspiro[4.5]decan-8-one (26 g, 166.5 mmol) in toluene (90 mL) was added over 40 min and the suspension was warmed to ambient temperature and stirred for 2.5 h. The thick yellow suspension was filtered, washing the filtered cake with MTBE (˜150 mL). The solid was partitioned between water (600 mL), MTBE (200 mL) and EtOAc (600 mL). The organic layer was separated and washed with water (2×500 mL) and brine (500 mL), dried (MgSO₄), filtered and concentrated in vacuo to give 8-(2-(benzyloxy)-6-bromophenyl)-1,4-dioxaspiro[4.5]decan-8-ol (34.029 g, 61%) as a white solid. ¹H NMR (300 MHz, DMSO-d₆) δ 7.56-7.47 (m, 2H), 7.47-7.29 (m, 3H), 7.23 (dd, J=7.7, 1.5 Hz, 1H), 7.17-7.00 (m, 2H), 5.14 (s, 2H), 4.72 (s, 1H), 3.84-3.78 (m, 4H), 2.74-2.55 (m, 2H), 2.02-1.89 (m, 2H), 1.89-1.77 (m, 2H), 1.48-1.38 (m, 2H) ppm. ESI-MS m/z calc. 418.078, found 441.2 (M+Na)⁺.

### Step 2:

Under a nitrogen atmosphere, MeMgCl (4.1 mL, 3 M solution in THF, 12.30 mmol) and ⁱPrMgCl·LiCl (18 mL, 1.3 M solution in THF, 23.40 mmol) were successively added dropwise to a stirred solution of 8-(2-(benzyloxy)-6-bromophenyl)-1,4-dioxaspiro[4.5]decan-8-ol (5 g, 11.68 mmol) in THF (25 mL) at 0° C. and the reaction mixture was stirred at 0° C. for 5 h. The nitrogen atmosphere was replaced by a carbon dioxide atmosphere and the reaction mixture was stirred for 1 h. The reaction was quenched by addition of a saturated aqueous NH₄Cl solution (20 mL) and the mixture was stirred at ambient temperature overnight. The suspension was diluted with water (50 mL). The solid was filtered, washed with water (50 mL) and heptane (20 mL) to give 7-(benzyloxy)-3H-dispiro[isobenzofuran-1,1′-cyclohexane-4′,2″-[1,3]dioxolan]-3-one (767 mg, 18%) as a white solid. A second crop was isolated from the filtrates, partitioned between DCM (50 mL), water (50 mL) and 1 N aqueous HCl (5 mL). The organic layer was separated, washed with water (50 mL) and brine (50 mL) and passed through a phase separation cartridge. The filtrates were concentrated in vacuo to give 7-(benzyloxy)-3H-dispiro[isobenzofuran-1,1′-cyclohexane-4′,2″-[1,3]dioxolan]-3-one (2.260 g, 49%) as a white solid. ¹H NMR (300 MHz, DMSO-d₆) δ 7.61-7.51 (m, 3H), 7.50-7.38 (m, 4H), 7.38-7.25 (m, 1H), 5.32 (s, 2H), 4.05-3.90 (m, 4H), 2.72 (td, J=13.8, 4.9 Hz, 2H), 1.96-1.71 (m, 4H), 1.71-1.54 (m, 2H) ppm. ESI-MS m/z calc. 366.147, found 367.2 (M+1)⁺.

### Step 3:

TFA (500 μL, 6.490 mmol) was added to a stirred mixture of 7-(benzyloxy)-3H-dispiro[isobenzofuran-1,1′-cyclohexane-4′,2″-[1,3]dioxolan]-3-one (750 mg, 2.047 mmol) in a mixture of THF (12 mL) and water (250 μL, 13.88 mmol) and the reaction mixture was stirred at ambient temperature for 10 min. Additional amounts of water (1 mL, 55.51 mmol) and TFA (4 mL, 51.92 mmol) were added and the mixture was heated and stirred at 50° C. overnight. Additional water (10 mL, 555.1 mmol) and AcOH (10 mL) were added and the reaction was stirred and heated at 80° C. for 3.5 h. The reaction was cooled to ambient temperature and stirred overnight. The mixture was cooled to 0° C. and NaOH (40 mL, 6 M aqueous solution, 240 mmol) was added. The suspension was filtered and the solid was washer with water (˜20 mL) and heptane (˜20 mL) and dried to give 7′-(benzyloxy)-3′H-spiro[cyclohexane-1,1′-isobenzofuran]-3′,4-dione (582 mg, 88%) as a pale yellow solid.

### Step 4:

A suspension of 7′-(benzyloxy)-3′H-spiro[cyclohexane-1,1′-isobenzofuran]-3′,4-dione (582 mg) and Pd (250 mg, 5% w/w on carbon, 0.118 mmol) in EtOAc (5 mL) and DMF (5 mL) was stirred under a hydrogen atmosphere for 2 h. The reaction mixture was filtered through a pad of Celite®, washing the filtered cake with methanol (5 mL) and water (5 mL). The filtrates were concentrated in vacuo, and co-evaporated with MeCN (2×20 mL) to give 7′-hydroxy-3′H-spiro[cyclohexane-1,1′-isobenzofuran]-3′,4-dione, which was used without further purification in the next step.

### Step 5:

7′-Hydroxy-3′H-spiro[cyclohexane-1,1′-isobenzofuran]-3′,4-dione was treated via the method described in Intermediate 8 Step 5 to give, after purification by flash chromatography (SiO₂, 0 to 100% EtOAc in heptane), benzyl 2-((3′,4-dioxo-3′H-spiro[cyclohexane-1,1′-isobenzofuran]-7′-yl)oxy)acetate (Int-15, 536 mg, 64% over 2 steps) as a white solid. ¹H NMR (300 MHz, DMSO-d₆) δ 7.62-7.50 (m, 1H), 7.51-7.44 (m, 1H), 7.44-7.28 (m, 6H), 5.18 (s, 2H), 5.08 (s, 2H), 2.84-2.68 (m, 4H), 2.31 (d, J=9.7 Hz, 2H), 1.94 (d, J=8.2 Hz, 2H) ppm. ESI-MS m/z calc. 380.126, found 381.2 (M+1)⁺.

### Example 41: Intermediate 16

### Benzyl 2-((4-oxo-2′,3′-dihydrospiro[cyclohexane-1,1′-inden]-7′-yl-3′,3′-d₂)oxy)acetate (Int-16)

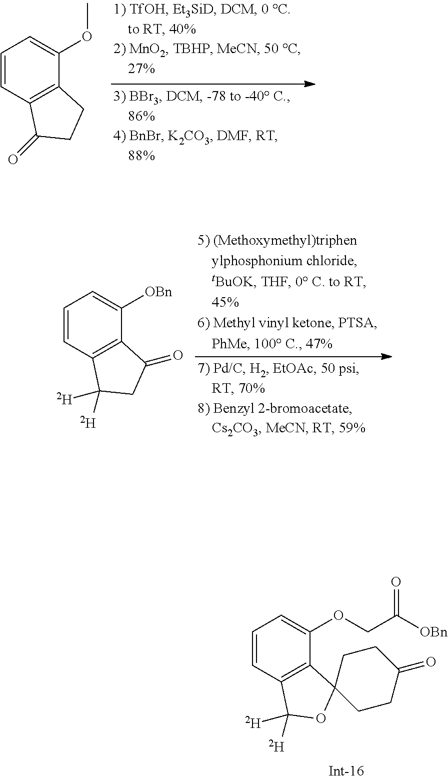 [SMILES: COc1cccc2c1CCC2=O ; [2H]C1([2H])CC(=O)c2c(OCc3ccccc3)cccc21 ; [2H]C1([2H])OC2(CCC(=O)CC2)c2c(OCC(=O)OCc3ccccc3)cccc21]

### Step 1:

Et₃SiD (500 mg, 4.263 mmol) was added dropwise over 2 min to a stirred suspension of 4-methoxy-2,3-dihydro-1H-inden-1-one (230 mg, 1.418 mmol) in DCM (4 mL) and TfOH (400 μL, 4.521 mmol) at 0° C. and the reaction mixture was warmed and stirred at ambient temperature overnight. The mixture was diluted with a saturated aqueous NaHCO₃ solution (10 mL). The organic layer was separated and concentrated in vacuo. Purification by flash chromatography (80 g SiO₂, 0 to 50% DCM in heptane) gave 4-methoxy-2,3-dihydro-1H-indene-1,1-d₂ (90 mg, 40%) as a colourless oil. ¹H NMR (300 MHz, Chloroform-d) δ 7.15 (ddt, J=8.2, 7.4, 0.8 Hz, 1H), 6.88 (dd, J=7.4, 0.8 Hz, 1H), 6.69 (dd, J=8.1, 0.8 Hz, 1H), 3.86 (s, 3H), 2.97-2.84 (m, 2H), 2.08 (t, J=7.4 Hz, 2H) ppm. ESI-MS m/z calc. 150.101, found 150.5 (M+1)⁺.

### Step 2:

TBHP (200 μL, 5 M solution in decane, 1 mmol) was added to a stirred suspension of 4-methoxy-2,3-dihydro-1H-indene-1,1-d₂ (30 mg, 0.190 mmol) and MnO₂ (200 mg, 2.301 mmol) in MeCN (1 mL) and the suspension was stirred at 50° C. for 1 h. The mixture was filtered through a pad of Celite® and the filtrates were concentrated in vacuo. Purification by flash chromatography (24 g SiO₂, 20 to 100% MTBE in heptane) gave 7-methoxy-2,3-dihydro-1H-inden-1-one-3,3-d₂ (9 mg, 27%) as a colourless solid. H NMR (300 MHz, Chloroform-d) δ 7.54 (dd, J=8.2, 7.5 Hz, 1H), 7.04 (dd, J=7.6, 0.7 Hz, 1H), 6.81 (d, J=8.2 Hz, 1H), 3.98 (s, 3H), 2.69 (s, 2H) ppm. ESI-MS m/z calc. 164.081, found 165.0 (M+1)⁺.

### Step 3:

Under a nitrogen atmosphere, BBr₃ (70 mL, 1 M solution in DCM, 70 mmol) was added to a stirred solution of 7-methoxy-2,3-dihydro-1H-inden-1-one-3,3-d₂ (2.4 g, 13.89 mmol) in DCM (300 mL) at −78° C. and the reaction mixture was stirred at −78° C. for 1.5 h. Additional BBr₃ (20 mL, 1 M solution in DCM, 20 mmol) was added at −78° C. and the mixture was stirred for an additional 30 min at −40° C. The reaction was quenched by slow addition of MeOH (100 mL) and the mixture was concentrated in vacuo. The residue was partitioned between DCM (50 mL) and water (50 mL). The aqueous layer was separated and extracted with DCM (50 mL). The combined organic phases were concentrated in vacuo to give 7-hydroxy-2,3-dihydro-1H-inden-1-one-3,3-d₂ (2 g, 86%) as a yellow solid, which was used without further purification in the next step. ¹H NMR (300 MHz, Chloroform-d) δ 7.23 (dd, J=8.2, 7.5 Hz, 1H), 6.71 (dd, J=7.5, 0.8 Hz, 1H), 6.49 (d, J=8.2 Hz, 1H), 2.44 (d, J=4.8 Hz, 2H) ppm; exchangeable H not observed. ESI-MS m/z calc. 150.065, found 150.9 (M+1)⁺.

### Step 4:

BnBr (3 mL, 25.22 mmol) was added to a stirred suspension of 7-hydroxy-2,3-dihydro-1H-inden-1-one-3,3-d₂ (2 g, 11.99 mmol) and K₂CO₃ (11 g, 79.59 mmol) in DMF (50 mL) and the reaction mixture was stirred at ambient temperature for 2 days. The reaction was diluted with water (300 mL) and EtOAc (300 mL). The organic layer was separated, washed with water (3×500 mL) and brine (500 mL), dried (Na₂SO₄), filtered and concentrated in vacuo. Purification by flash chromatography (220 g SiO₂, 0 to 100% EtOAc in heptane) gave 7-(benzyloxy)-2,3-dihydro-1H-inden-1-one-3,3-d₂ (2.7 g, 88%) as a yellow oil. ¹H NMR (300 MHz, Chloroform-d) δ 7.67-7.31 (m, 6H), 7.02 (dd, J=7.5, 0.7 Hz, 1H), 6.80 (d, J=8.2 Hz, 1H), 5.31 (s, 2H), 2.71 (d, J=5.0 Hz, 2H) ppm. ESI-MS m/z calc. 240.112, found 240.8 (M+1)⁺.

### Step 5:

^{t}BuOK (24 mL, 1 M solution in THF, 24 mmol) was added to a stirred suspension of (methoxymethyl)triphenylphosphonium chloride (9 g, 26.25 mmol) in THF (40 mL) at 0° C. and the reaction mixture was stirred at ambient temperature for 1 h. A solution of 7-(benzyloxy)-2,3-dihydro-1H-inden-1-one-3,3-d₂ (2.7 g, 10.56 mmol) in THF (40 mL) was added and the reaction was stirred at ambient temperature for 1 h then at reflux overnight. The mixture was cooled to ambient temperature and filtered. The filtrates were added at ambient temperature to an additional suspension of the ylid prepared by adding ^{t}BuOK (24 mL, 1 M solution in THF, 24 mmol) to a suspension of (methoxymethyl)triphenylphosphonium chloride (9 g, 26.25 mmol) in THF (40 mL) at 0° C. The mixture was stirred for 30 min and heated to reflux for 30 min. The mixture was cooled to ambient temperature, poured onto heptane (1 L) at 0° C. and stirred for 30 min. The solid was filtered. The filtrates were collected and diluted with MTBE (500 mL) and water (500 mL). The organic layer was separated, washed with water (2×0.5 L) and brine (0.5 L), dried (Na₂SO₄), filtered, and concentrated in vacuo. Purification by flash chromatography (220 g SiO₂, 0 to 30% EtOAc in heptane) gave (E)-7-(benzyloxy)-1-(methoxymethylene)-2,3-dihydro-1H-indene-3,3-d₂ (1.4 mg, 45%) as a yellow oil. ¹H NMR (300 MHz, Chloroform-d) δ 7.60-7.30 (m, 5H), 7.11 (dd, J=8.2, 7.4 Hz, 1H), 6.88 (dd, J=7.3, 0.9 Hz, 1H), 6.80 (dd, J=8.1, 0.9 Hz, 1H), 6.13 (t, J=1.6 Hz, 1H), 5.15 (s, 2H), 3.52 (s, 3H), 2.64 (d, J=1.6 Hz, 2H) ppm. ESI-MS m/z calc. 268.143, found 269.2 (M+1)⁺.

### Step 6:

Methyl vinyl ketone (800 μL, 9.610 mmol) was added to a stirred mixture of (E)-7-(benzyloxy)-1-(methoxymethylene)-2,3-dihydro-1H-indene-3,3-d₂ (1.4 g, 4.956 mmol) and PTSA (900 mg, 4.731 mmol) in toluene (30 mL) and the reaction mixture was stirred at 100° C. for 2 h. The mixture was concentrated in vacuo and diluted with a saturated aqueous NaHCO₃ solution (50 mL) and EtOAc (50 mL). The organic layer was separated, washed with a saturated aqueous NaHCO₃ solution (3×200 mL) and brine (200 mL), dried (Na₂SO₄), filtered, and concentrated in vacuo. Purification by flash chromatography (120 g SiO₂, 0 to 50% EtOAc in heptane) gave 7′-(benzyloxy)-2′,3′-dihydrospiro[cyclohexane-1,1′-inden]-2-en-4-one-3′,3′-d₂ (800 mg, 47%) as a brown oil. ¹H NMR (300 MHz, Chloroform-d) δ 7.36 (d, J=3.2 Hz, 5H), 7.21 (dd, J=8.1, 7.5 Hz, 1H), 7.00 (dd, J=10.1, 1.9 Hz, 1H), 6.90 (dd, J=7.5, 0.8 Hz, 1H), 6.80 (dd, J=8.2, 0.9 Hz, 1H), 5.98 (dd, J=10.1, 0.7 Hz, 1H), 5.09 (s, 2H), 2.71-2.46 (m, 3H), 2.42-2.21 (m, 1H), 2.05-1.90 (m, 2H) ppm. ESI-MS m/z calc. 306.159, found 307.1 (M+1)⁺.

### Step 7:

Under a nitrogen atmosphere, Pd (350 mg, 10% w/w on carbon, 0.329 mmol) was added to a stirred solution of 7′-(benzyloxy)-2′,3′-dihydrospiro[cyclohexane-1,1′-inden]-2-en-4-one-3′,3′-d₂ (0.8 g, 2.611 mmol) in EtOAc (10 mL) and the reaction mixture was stirred under 50 psi of hydrogen at ambient temperature for 1 h. The suspension was filtered through a pad of Celite® and concentrated in vacuo to give 7′-hydroxy-2′,3′-dihydrospiro[cyclohexane-1,1′-inden]-4-one-3′,3′-d₂ (420 mg, 70%) as a light yellow solid. ¹H NMR (300 MHz, Chloroform-d) δ 7.07 (t, J=7.7 Hz, 1H), 6.89-6.78 (m, 1H), 6.57 (dd, J=7.9, 0.9 Hz, 1H), 5.55 (s, 1H), 2.74 (td, J=13.0, 5.2 Hz, 2H), 2.67-2.41 (m, 4H), 2.25 (d, J=1.6 Hz, 2H), 1.89 (ddt, J=13.4, 5.7, 3.0 Hz, 2H) ppm. ESI-MS m/z calc. 218.128, found 219.1 (M+1)⁺.

### Step 8:

7′-Hydroxy-2′,3′-dihydrospiro[cyclohexane-1,1′-inden]-4-one-3′,3′-d₂ (550 mg, 2.520 mmol) was treated via the method described in Intermediate 23 Step 6 to give, after purification by flash chromatography (80 g SiO₂, 20 to 100% EtOAc in heptane), benzyl 2-((4-oxo-2′,3′-dihydrospiro[cyclohexane-1,1′-inden]-7′-yl-3′,3′-d2)oxy)acetate (Int-16, 570 mg, 59%) as a colourless oil. ¹H NMR (300 MHz, Chloroform-d) δ 7.60-7.30 (m, 5H), 7.13 (dd, J=8.1, 7.5 Hz, 1H), 6.89 (dd, J=7.5, 0.8 Hz, 1H), 6.55 (d, J=8.1 Hz, 1H), 5.22 (s, 2H), 4.70 (s, 2H), 2.76 (td, J=13.1, 5.4 Hz, 2H), 2.66-2.31 (m, 4H), 2.25 (d, J=6.0 Hz, 2H), 1.88-1.76 (m, 2H) ppm. ESI-MS m/z calc. 366.180, found 367.3 (M+1)⁺.

### Example 42: Intermediate 18

### Benzyl (R)-2-((4-oxo-3′H-spiro[cyclohexane-1,1′-isobenzofuran]-7′-yl)oxy)propanoate (Int-18)

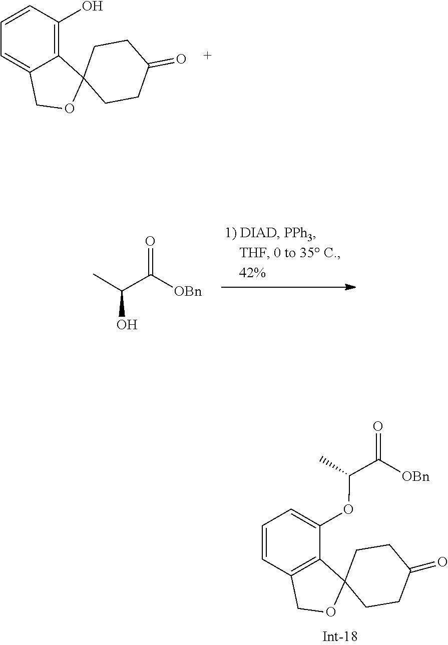 [SMILES: O=C1CCC2(CC1)OCc1cccc(O)c12 ; C[C@H](O)C(=O)OCc1ccccc1 ; C[C@@H](Oc1cccc2c1C1(CCC(=O)CC1)OC2)C(=O)OCc1ccccc1]

### Step 1:

DIAD (4.3 mL, 21.90 mmol) was added to a stirred mixture of 7′-hydroxy-3′H-spiro[cyclohexane-1,1′-isobenzofuran]-4-one (1.86 g, prepared using the method described in Intermediate 1 Steps 1 to 4, 8.437 mmol), benzyl (S)-2-hydroxypropanoate (2.7 g, 14.98 mmol) and PPh₃ (5.6 g, 21.35 mmol) in THF (100 mL) at 0° C. and the reaction mixture was heated and stirred at 35° C. for 2 h. The reaction was concentrated in vacuo. Purification by flash chromatography (SiO₂ 0 to 30% EtOAc in heptane) gave benzyl (R)-2-((4-oxo-3′H-spiro[cyclohexane-1,1′-isobenzofuran]-7′-yl)oxy)propanoate (Int-18, 1.5 g, 42%) as a colourless oil. ¹H NMR (300 MHz, Chloroform-d) δ 7.36-7.28 (m, 4H), 7.21-7.13 (m, 2H), 6.83 (dt, J=7.6, 0.8 Hz, 1H), 6.56 (d, J=8.1 Hz, 1H), 5.15-5.09 (m, 4H), 4.89 (q, J=6.8 Hz, 1H), 2.95-2.75 (m, 2H), 2.72-2.52 (m, 2H), 2.42-2.25 (m, 2H), 2.09-2.00 (m, 1H), 1.95-1.86 (m, 1H), 1.61 (d, J=6.7 Hz, 3H) ppm.

The following Intermediate was made using the method described in Intermediate 18 except that, in Step 1, benzyl (R)-2-hydroxypropanoate was used in place of benzyl (S)-2-hydroxypropanoate:

| Intermediate | | | | |
|---|---|---|---|---|
| Number | Structure | Name | LC/MS | NMR (shifts in ppm) |
| Int-21 | 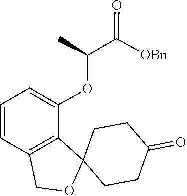 [SMILES: C[C@H](Oc1cccc2c1C1(CCC(=O)CC1)OC2)C(=O)OCc1ccccc1] | Benzyl (S)-2- ((4-oxo-3′H- spiro[cyclo- hexane-1,1′- isobenzofuran]- 7′- yl)oxy)propanoate | ESI-MS m/z calc. 380.162, found 381.4 (M + 1)⁺. | |

### Example 43: Intermediate 22

### Benzyl (E)-3-((4-oxo-3′H-spiro[cyclohexane-1,1′-isobenzofuran]-7′-yl)oxy)acrylate (Int-22)

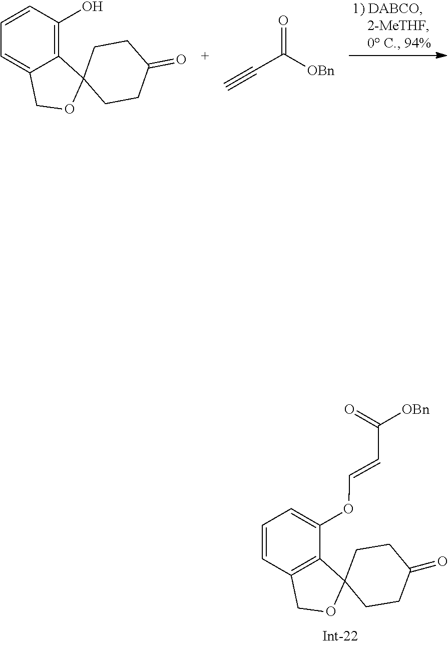 [SMILES: O=C1CCC2(CC1)OCc1cccc(O)c12 ; C#CC(=O)OCc1ccccc1 ; O=C1CCC2(CC1)OCc1cccc(O/C=C/C(=O)OCc3ccccc3)c12]

### Step 1:

A solution of benzyl propiolate (11.35 g, 70.86 mmol) in 2-MeTHF (90 mL) was added to a stirred suspension of 7′-hydroxy-3′H-spiro[cyclohexane-1,1′-isobenzofuran]-4-one (15 g, 68.73 mmol) and DABCO (750 mg, 6.686 mmol) in 2-MeTHF (150 mL) at 0° C. and the reaction mixture was stirred for 18 h. The reaction was diluted with HCl (200 mL, 0.1 N aqueous solution). The organic layer was separated, washed with HCl (200 mL, 0.1 N aqueous solution), NaOH (200 mL, 0.1 N aqueous solution), and brine (200 mL), dried (MgSO₄), filtered and concentrated in vacuo. Purification by flash chromatography (120 g SiO₂, 50% EtOAc in heptane) gave benzyl (E)-3-((4-oxo-3′H-spiro[cyclohexane-1,1′-isobenzofuran]-7′-yl)oxy)acrylate (Int-22, 24.8 g, 94%) as a white solid. ¹H NMR (300 MHz, Chloroform-d) δ 7.76 (d, J=12.2 Hz, 1H), 7.38-7.24 (m, 6H), 7.09-6.97 (m, 1H), 6.93-6.84 (m, 1H), 5.61 (d, J=12.2 Hz, 1H), 5.16 (s, 2H), 5.13 (s, 2H), 2.94-2.76 (m, 2H), 2.46-2.25 (m, 4H), 2.14-1.98 (m, 2H) ppm. ESI-MS m/z calc. 378.147, found 379.7 (M+1)⁺.

The following Intermediate was made using the method described in Intermediate 22 except that, in Step 1, different alcohols were used as the starting material in place of 7′-hydroxy-3′H-spiro[cyclohexane-1,1′-isobenzofuran]-4-one. In the case of Int-39, starting material 4′-fluoro-7′-hydroxy-3′H-spiro[cyclohexane-1,1′-isobenzofuran]-4-one was prepared using the method described in Intermediate 7 Steps 1 to 4. In the case of Int-41, 6′-fluoro-7′-hydroxy-3′H-spiro[cyclohexane-1,1′-isobenzofuran]-4-one, product of Intermediate 6 Step 4, was used as the starting material:

| Intermediate | | | | |
|---|---|---|---|---|
| Number | Structure | Name | LC/MS | NMR (shifts in ppm) |
| Int-39 | 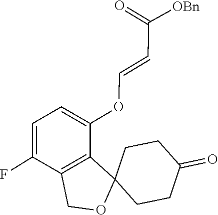 [SMILES: O=C1CCC2(CC1)OCc1c(F)ccc(O/C=C/C(=O)OCc3ccccc3)c12] | Benzyl (E)-3- ((4′-fluoro-4- oxo-3′H- spiro[cyclo- hexane-1,1′- isobenzofuran]- 7′- yl)oxy)acrylate | | ¹H NMR (300 MHz, DMSO- d₆) δ 7.88 (d, J = 12.2 Hz, 1H), 7.45-7.30 (m, 5H), 7.30-7.16 (m, 2H), 5.69 (d, J = 12.2 Hz, 1H), 5.21 (s, 2H), 5.16 (s, 2H), 2.71 (td, J = 13.7, 6.2 Hz, 2H), 2.42- 2.17 (m, 4H), 2.14-1.96 (m, 2H) ppm. |
| Int-41 | 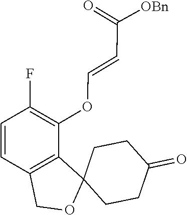 [SMILES: O=C1CCC2(CC1)OCc1ccc(F)c(O/C=C/C(=O)OCc3ccccc3)c12] | Benzyl (E)-3- ((6′-fluoro-4- oxo-3′H- spiro[cyclo- hexane-1,1′- isobenzofuran]- 7′- yl)oxy)acrylate | ESI-MS m/z calc. 396.137, found 397.8 (M + 1)⁺. | |

### Example 44: Intermediate 23

### Benzyl 2-((4-oxospiro[cyclohexane-1,1′-isochroman]-8′-yl)oxy)acetate (Int-23)

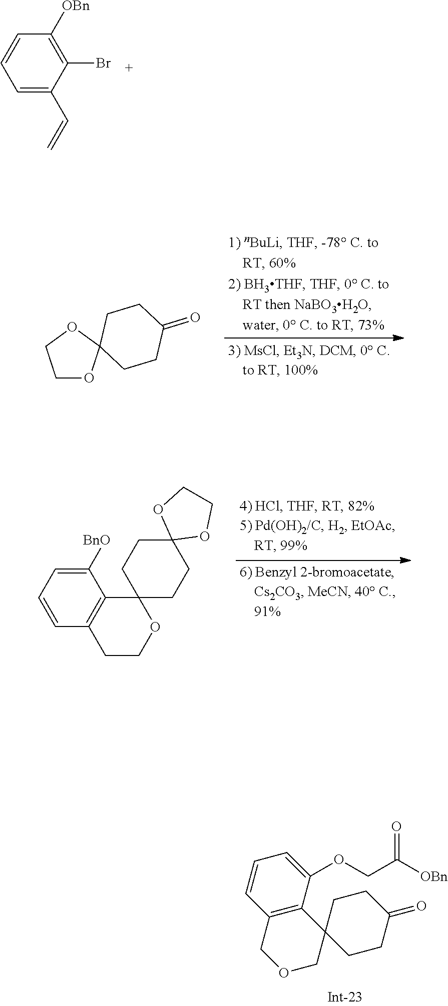 [SMILES: C=Cc1cccc(OCc2ccccc2)c1Br ; O=C1CCC2(CC1)OCCO2 ; c1ccc(COc2cccc3c2C2(CCC4(CC2)OCCO4)OCC3)cc1 ; O=C1CCC2(CC1)COCc1cccc(OCC(=O)OCc3ccccc3)c12]

### Step 1:

1-Benzyloxy-2-bromo-3-vinyl-benzene (6.24 g, 21.58 mmol) and 1,4-dioxaspiro[4.5]decan-8-one (4.05 g, 25.93 mmol) were treated via the method described in Intermediate 1 Step 1 to give, after purification by flash chromatography (SiO₂, 0 to 30% EtOAc in heptane), 8-(2-(benzyloxy)-6-vinylphenyl)-1,4-dioxaspiro[4.5]decan-8-ol (5.12 g, 60%) as a white solid. ¹H NMR (300 MHz, Methanol-d₄) δ 7.54-7.41 (m, 3H), 7.46-7.29 (m, 3H), 7.13 (ddd, J=8.1, 7.5, 0.5 Hz, 1H), 7.03 (dd, J=8.3, 1.5 Hz, 1H), 6.89 (ddd, J=7.5, 1.5, 0.6 Hz, 1H), 5.21-5.11 (m, 3H), 5.07 (dd, J=10.8, 2.0 Hz, 1H), 4.02 (s, 1H), 3.93-3.80 (m, 4H), 2.80 (td, J=13.7, 4.3 Hz, 2H), 2.03-1.97 (m, 2H), 1.83-1.70 (m, 2H), 1.58-1.45 (m, 2H) ppm. ESI-MS m/z calc. 366.183, found 389.3 (M+Na)⁺.

### Step 2:

Under an argon atmosphere, BH₃·THF (45 mL, 1 M solution in THF, 45 mmol) was added dropwise over 5 min to a stirred solution of 8-(2-(benzyloxy)-6-vinylphenyl)-1,4-dioxaspiro[4.5]decan-8-ol (5.12 g, 12.96 mmol) and THF (76 mL) at 0° C. and the reaction mixture was warmed up and stirred at ambient temperature for 70 min. The reaction was cooled to 0° C. Water (76 mL) was added dropwise over 2 min causing a gas evolution. NaBO₃·H₂O (5.17 g, 51.8 mmol) was added to the mixture and the cooling bath was replaced with a water bath. The reaction was stirred vigorously for 1 h and partitioned between water and Et₂O. The aqueous phase was separated and extracted with Et₂O (2×). The combined organic layers were washed with brine, dried (Na₂SO₄), filtered and concentrated in vacuo. Purification by flash chromatography (SiO₂, 0 to 70% EtOAc in DCM) gave 8-(2-(benzyloxy)-6-(2-hydroxyethyl)phenyl)-1,4-dioxaspiro[4.5]decan-8-ol (4.34 g, 73%) as a white solid. ¹H NMR (300 MHz, Methanol-d₄) δ 7.53-7.46 (m, 2H), 7.42-7.28 (m, 3H), 7.08 (dd, J=8.2, 7.6 Hz, 1H), 6.92 (dd, J=8.2, 1.4 Hz, 1H), 6.78 (dd, J=7.6, 1.4 Hz, 1H), 5.09 (s, 2H), 3.93-3.73 (m, 7H), 3.00 (td, J=13.7, 4.4 Hz, 2H), 2.13-1.97 (m, 2H), 1.71-1.59 (m, 2H), 1.56-1.42 (m, 3H) ppm; exchangeable H not observed. ESI-MS m/z calc. 384.194, found 367.4 (M-OH)⁺.

### Step 3:

Under a nitrogen atmosphere, MsCl (884 μL, 11.42 mmol) was added to a stirred solution of 8-(2-(benzyloxy)-6-(2-hydroxyethyl)phenyl)-1,4-dioxaspiro[4.5]decan-8-ol (4.34 g, 9.514 mmol) and Et₃N (4.6 mL, 33.0 mmol) in DCM (100 mL) at 0° C. and the reaction mixture was warmed and stirred at ambient temperature for 10 min. The reaction was quenched by addition of a half-saturated aqueous NH₄Cl solution (50 mL). The aqueous phase was separated and extracted with DCM (2×). The combined organic layers were dried by passing the solution through a phase separation cartridge and the mother liquors were concentrated in vacuo. Purification by flash chromatography (SiO₂, 0 to 50% EtOAc in DCM) gave 8-(benzyloxy)dispiro[isochromane-1,1′-cyclohexane-4′,2″-[1,3]dioxolane](3.52 g, 100%) as a white solid. ¹H NMR (300 MHz, Methanol-d₄) δ 7.61-7.54 (m, 2H), 7.43-7.35 (m, 2H), 7.35-7.27 (m, 1H), 7.09 (dd, J=8.2, 7.5 Hz, 1H), 6.89 (dd, J=7.9, 1.0 Hz, 1H), 6.73 (dq, J=7.5, 0.9 Hz, 1H), 5.09 (s, 2H), 3.90-3.81 (m, 6H), 2.90 (td, J=13.8, 4.4 Hz, 2H), 2.79 (t, J=5.5 Hz, 2H), 2.05-1.88 (m, 2H), 1.71 (ddt, J=14.5, 5.1, 2.7 Hz, 2H), 1.51 (ddt, J=13.5, 5.2, 2.7 Hz, 2H) ppm. ESI-MS m/z calc. 366.183, found 367.3 (M+1)⁺.

### Step 4:

HCl (12.8 mL, 6 M aqueous solution, 76.80 mmol) was added to a stirred solution of 8-(benzyloxy)dispiro[isochromane-1,1′-cyclohexane-4′,2″-[1,3]dioxolane](2.78 g, 7.586 mmol) in THF (28 mL) and the reaction mixture was stirred at ambient temperature for 1.5 h. The reaction was diluted with water and extracted with DCM (3×). The combined organic layers were washed with a saturated aqueous NaHCO₃ solution, dried by passing the solution through a phase separation cartridge and concentrated in vacuo. Purification by flash chromatography (SiO₂, 0 to 50% EtOAc in heptane) gave 8′-(benzyloxy)spiro[cyclohexane-1,1′-isochroman]-4-one (2.03 g, 82%) as a white solid. ¹H NMR (400 MHz, Chloroform-d) δ 7.43-7.36 (m, 2H), 7.36-7.29 (m, 3H), 7.13 (t, J=7.6 Hz, 1H), 6.78 (d, J=7.9 Hz, 2H), 5.09 (s, 2H), 3.95 (t, J=5.5 Hz, 2H), 2.98 (td, J=13.6, 5.0 Hz, 2H), 2.88 (t, J=5.7 Hz, 2H), 2.77 (td, J=14.0, 6.2 Hz, 2H), 2.25-2.16 (m, 2H), 2.16-2.06 (m, 2H) ppm. ESI-MS m/z calc. 322.157, found 323.2 (M+1)⁺.

### Step 5:

Under a nitrogen atmosphere, a solution of 8′-(benzyloxy)spiro[cyclohexane-1,1′-isochroman]-4-one (2.03 g, 6.296 mmol) in EtOAc (37 mL) was added to a suspension of Pd(OH)₂/C (884 mg, 0.630 mmol) in EtOAc (37 mL) and the reaction mixture was stirred under a hydrogen atmosphere at ambient temperature for 45 min. The reaction was filtered through a pad of Celite®, washing the filtered cake with EtOAc. The filtrates were concentrated in vacuo to give 8′-hydroxyspiro[cyclohexane-1,1′-isochroman]-4-one (1.50 g, 99%) as a white solid. ¹H NMR (400 MHz, Chloroform-d) δ 7.03 (t, J=7.8 Hz, 1H), 6.73 (d, J=7.6 Hz, 1H), 6.58 (d, J=7.9 Hz, 1H), 5.61 (s, 1H), 3.95 (t, J=5.5 Hz, 2H), 3.00 (td, J=13.7, 4.8 Hz, 2H), 2.90-2.76 (m, 4H), 2.32-2.22 (m, 2H), 2.16-2.05 (m, 2H) ppm. ESI-MS m/z calc. 232.110, found 233.2 (M+1)⁺.

### Step 6:

Benzyl 2-bromoacetate (1.24 mL, 7.827 mmol) was added to a suspension of 8′-hydroxyspiro[cyclohexane-1,1′-isochroman]-4-one (1.50 g, 6.242 mmol) and Cs₂CO₃ (4.08 g, 12.52 mmol) in MeCN (31 mL) and the reaction mixture was stirred at 40° C. for 40 min. The reaction was filtered through a pad of Celite®, washing the filtered cake with EtOAc. The filtrates were concentrated in vacuo. Purification by flash chromatography (SiO₂, 0 to 60% EtOAc in heptane) gave benzyl 2-((4-oxospiro[cyclohexane-1,1′-isochroman]-8′-yl)oxy)acetate (Int-23, 2.33 g, 91%) as a thick colourless oi. ¹H NMR (400 MHz, Chloroform-d) δ 7.40-7.27 (m, 5H), 7.10 (t, J=7.8 Hz, 1H), 6.80 (d, J=7.6 Hz, 1H), 6.55 (d, J=8.2 Hz, 1H), 5.21 (s, 2H), 4.66 (s, 2H), 3.93 (t, J=5.3 Hz, 2H), 3.01 (td, J=13.6, 5.1 Hz, 2H), 2.87 (t, J=5.7 Hz, 2H), 2.78 (td, J=13.9, 6.2 Hz, 2H), 2.31-2.19 (m, 2H), 2.10-1.99 (m, 2H) ppm. ESI-MS m/z calc. 380.162, found 381.3 (M+1)⁺.

### Example 45: Intermediate 24

### Benzyl 2-((4-oxo-2′,3′-dihydrospiro[cyclohexane-1,1′-inden]-7′-yl)oxy)acetate (Int-24)

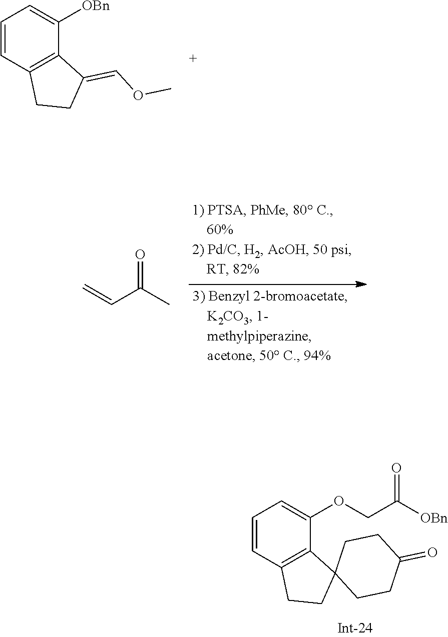

### Step 1:

But-3-en-2-one (22.9 g, 277.7 mmol) was added to a stirred solution of (E)-7-(benzyloxy)-1-(methoxymethylene)-2,3-dihydro-1H-indene (38 g, 138.8 mmol) and PTSA monohydrate (10.6 g, 55.73 mmol) in toluene (380 mL) and the reaction mixture was stirred at 80° C. for 2 h. The mixture was partitioned with a saturated aqueous NaHCO₃ solution (300 mL) and extracted with EtOAc (3×100 mL). The combined organic extracts were dried (Na₂SO₄), filtered and concentrated in vacuo. Purification by flash chromatography (330 g SiO₂, 0 to 40% EtOAc in heptane) gave 7′-(benzyloxy)-2′,3′-dihydrospiro[cyclohexane-1,1′-inden]-2-en-4-one (28.5 g, 60%) as an orange oil. ¹H NMR (300 MHz, Chloroform-d) δ 7.46-7.30 (m, 5H), 7.21 (t, J=7.4 Hz, 1H), 7.00 (dd, J=10.1, 1.8 Hz, 1H), 6.90 (d, J=0.9 Hz, 1H), 6.80 (d, J=8.1 Hz, 1H), 5.98 (dd, J=10.1, 0.7 Hz, 1H), 5.09 (s, 2H), 3.14-2.92 (m, 2H), 2.67-2.47 (m, 3H), 2.46-2.33 (m, 1H), 2.16-1.92 (m, 2H) ppm. ESI-MS m/z calc. 304.146, found 305.1 (M+1)⁺.

### Step 2:

Pd/C (2.4 g, 10% w/w, 2.255 mmol) was added to a stirred solution of 7′-(benzyloxy)-2′,3′-dihydrospiro[cyclohexane-1,1′-inden]-2-en-4-one (7 g, 23 mmol) in AcOH (50 mL) under a nitrogen atmosphere and the reaction mixture was stirred under a pressure of 50 psi of hydrogen at ambient temperature overnight. The suspension was filtered over Celite® and the liquors were concentrated in vacuo. Purification by trituration from water (300 mL) gave 7′-hydroxy-2′,3′-dihydrospiro[cyclohexane-1,1′-inden]-4-one (4.3 g, 82%) as a light yellow solid. ¹H NMR (300 MHz, Chloroform-d) δ 7.07 (t, J=7.7 Hz, 1H), 6.89-6.80 (m, 1H), 6.53 (d, J=7.8 Hz, 1H), 4.68 (s, 1H), 3.01 (t, J=7.6 Hz, 2H), 2.73 (dt, J=13.1, 6.5 Hz, 2H), 2.65-2.38 (m, 4H), 2.27 (t, J=7.4 Hz, 2H), 1.95-1.83 (m, 2H) ppm. ESI-MS m/z calc. 216.115, found 217.2 (M+1)⁺.

### Step 3:

Benzyl 2-bromoacetate (27.50 mL) was added to a stirred mixture of 7′-hydroxy-2′,3′-dihydrospiro[cyclohexane-1,1′-inden]-4-one (34.97 g, 160 mmol) and K₂CO₃ (66.44 g, 480.7 mmol) in acetone (865 mL) and the reaction mixture was heated at 50° C. for 6 h. Additional benzyl 2-bromoacetate (3.665 mL, 24 mmol) was added and the reaction was stirred at 50° C. overnight. 1-Methylpiperazine (5.289 g, 52.80 mmol) was added and the mixture was stirred at 50° C. for an additional 2 h. The reaction was cooled to ambient temperature, filtered, washing the resulting solid with acetone (200 ml). The mother liquors were concentrated in vacuo. The residue was dissolved in EtOAc (1 L), washed with a 10% aqueous citric acid solution (2×100 mL), water (100 mL), and brine (100 mL), dried (Na₂SO₄), filtered, and concentrated in vacuo to give benzyl 2-((4-oxo-2′,3′-dihydrospiro[cyclohexane-1,1′-inden]-7′-yl)oxy)acetate (Int-24, 61.80 g, 94%) as a light orange oil. ¹H NMR (300 MHz, Chloroform-d) δ 7.42-7.27 (m, 5H), 7.11 (dd, J=8.1, 7.5 Hz, 1H), 6.90-6.84 (m, 1H), 6.53 (d, J=8.2 Hz, 1H), 5.20 (s, 2H), 4.67 (s, 2H), 3.03-2.92 (m, 2H), 2.74 (td, J=13.1, 5.3 Hz, 2H), 2.59-2.30 (m, 4H), 2.29-2.18 (m, 2H), 1.87-1.74 (m, 2H) ppm. ESI-MS m/z calc. 364.167, found 365.2 (M+1)⁺.

### Example 46: Intermediate 25

### Benzyl 2-((4′,4′-difluoro-4-oxospiro[cyclohexane-1,1′-isochroman]-8′-yl)oxy)acetate (Int-25)

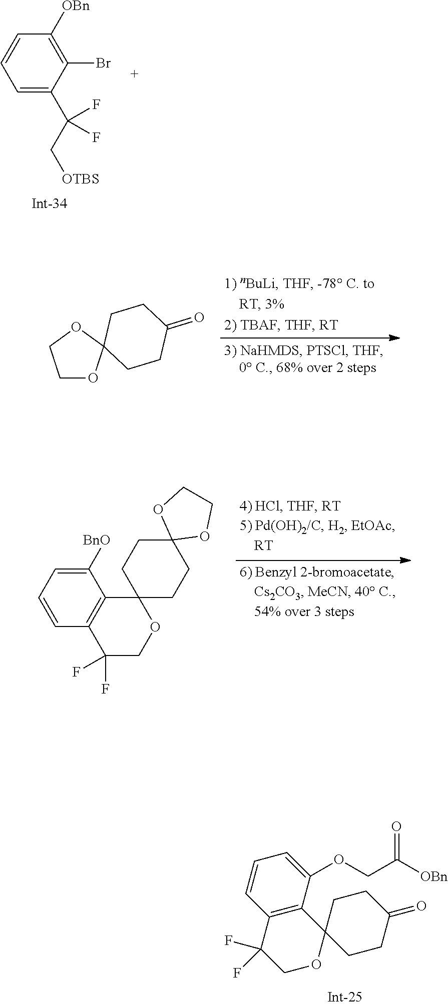 [SMILES: CC(C)(C)[Si](C)(C)OCC(F)(F)c1cccc(OCc2ccccc2)c1Br ; O=C1CCC2(CC1)OCCO2 ; FC1(F)COC2(CCC3(CC2)OCCO3)c2c(OCc3ccccc3)cccc21 ; O=C1CCC2(CC1)OCC(F)(F)c1cccc(OCC(=O)OCc3ccccc3)c12]

### Step 1:

(2-(3-(Benzyloxy)-2-bromophenyl)-2,2-difluoroethoxy)(tert-butyl)dimethylsilane (7.9 g, 17.27 mmol) and 1,4-dioxaspiro[4.5]decan-8-one (4.0 g, 25.61 mmol) were treated via the method described in Intermediate 1 Step 1 to give, after purification by flash chromatography (SiO₂, 0 to 40% EtOAc in heptane), 8-(2-(benzyloxy)-6-(2-((tert-butyldimethylsilyl)oxy)-1,1-difluoroethyl)phenyl)-1,4-dioxaspiro[4.5]decan-8-ol (310 mg, 3%) as a colourless oil. ¹H NMR (400 MHz, Chloroform-d) δ 7.77-7.09 (m, 7H), 7.00 (dd, J=8.1, 1.4 Hz, 1H), 5.08 (s, 2H), 4.39 (t, J=13.9 Hz, 2H), 3.77 (dt, J=63.8, 6.3 Hz, 4H), 3.04 (td, J=13.7, 4.4 Hz, 2H), 2.06-1.93 (m, 2H), 1.64-1.40 (m, 4H), 0.82 (s, 9H), 0.01 (d, J=7.1 Hz, 6H) ppm; exchangeable H not observed. ESI-MS m/z calc. 534.261, found 535.4 (M+1)⁺.

### Step 2:

TBAF (2.9 mL, 1 M solution in THF, 2.9 mmol) was added to a stirred solution of 8-(2-(benzyloxy)-6-(2-((tert-butyldimethylsilyl)oxy)-1,1-difluoroethyl)phenyl)-1,4-dioxaspiro[4.5]decan-8-ol (540 mg, 0.981 mmol) in THF (12.1 mL) and the reaction mixture was stirred at ambient temperature for 20 min. The reaction was quenched by addition of a saturated aqueous NH₄Cl solution (1 mL), diluted with water (5 mL) and extracted with DCM (2×10 mL). The combined organic layers were dried (Na₂SO₄), filtered, and concentrated in vacuo to give 8-(2-(benzyloxy)-6-(1,1-difluoro-2-hydroxyethyl)phenyl)-1,4-dioxaspiro[4.5]decan-8-ol which was used without further purification in the next step.

### Step 3:

NaHMDS (4.9 mL, 0.6 M solution in toluene, 2.94 mmol) was added to a stirred solution of 8-(2-(benzyloxy)-6-(1,1-difluoro-2-hydroxyethyl)phenyl)-1,4-dioxaspiro[4.5]decan-8-ol in THF (24.2 mL) at 0° C. and the reaction mixture was stirred at 0° C. for 10 min. A solution of PTSCl (224 mg, 1.175 mmol) in THF (6.1 mL) was added and the reaction was stirred at 0° C. for 1 h. Additional NaHMDS (4.9, 0.6 M solution in toluene, 2.94 mmol) was added and the reaction was stirred at 0° C. for a further 30 min. The reaction was quenched by addition of a saturated aqueous NH₄Cl solution (5 mL), diluted with water (5 mL) and extracted with DCM (2×20 mL). The combined organic layers were dried (Na₂SO₄), filtered, and concentrated in vacuo. Purification by flash chromatography (SiO₂, 0 to 30% EtOAc in heptane) gave 8-(benzyloxy)-4,4-difluorodispiro[isochromane-1,1′-cyclohexane-4′,2″-[1,3]dioxolane](270 mg, 68% over 2 steps) as a white solid. ¹H NMR (400 MHz, Chloroform-d) δ 7.50 (d, J=7.5 Hz, 2H), 7.41-7.26 (m, 5H), 7.03-6.97 (m, 1H), 5.09 (s, 2H), 3.99 (t, J=10.5 Hz, 2H), 3.94-3.78 (m, 4H), 2.84 (td, J=14.0, 4.2 Hz, 2H), 1.97 (td, J=13.5, 4.2 Hz, 2H), 1.90-1.71 (m, 2H), 1.66-1.54 (m, 2H) ppm. ESI-MS m/z calc. 402.164, found 403.4 (M+1)⁺.

### Step 4 to 6:

8-(Benzyloxy)-4,4-difluorodispiro[isochromane-1,1′-cyclohexane-4′,2″-[1,3]dioxolane](270 mg, 0.671 mmol) was treated via the method described in Intermediate 23 Steps 4 to 6 to give, after purification by flash chromatography (SiO₂, 0 to 50% EtOAc in heptane), benzyl 2-((4′,4′-difluoro-4-oxospiro[cyclohexane-1,1′-isochroman]-8′-yl)oxy)acetate (Int-25, 150 mg, 54% over 3 steps) as a colourless oil. ¹H NMR (400 MHz, Chloroform-d) δ 7.63-7.16 (m, 7H), 6.80 (d, J=8.1 Hz, 1H), 5.20 (s, 2H), 4.71 (s, 2H), 4.11 (t, J=10.3 Hz, 2H), 3.00 (td, J=13.8, 5.0 Hz, 2H), 2.76 (td, J=14.1, 6.2 Hz, 2H), 2.29 (ddd, J=14.8, 5.0, 2.4 Hz, 2H), 2.19-2.05 (m, 2H) ppm.

### Example 47: Intermediate 26

### Benzyl rac-(2R,3R)-4,4-difluoro-2-(hydroxymethyl)-3-(2,2,2-trifluoroacetamido)piperidine-1-carboxylate (Int-26)

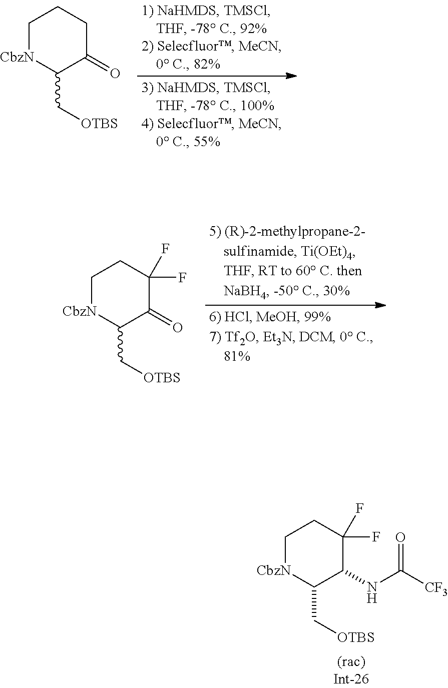 [SMILES: CC(C)(C)[Si](C)(C)OCC1C(=O)CCCN1C(=O)OCc1ccccc1 ; CC(C)(C)[Si](C)(C)OCC1C(=O)C(F)(F)CCN1C(=O)OCc1ccccc1 ; CC(C)(C)[Si](C)(C)OC[C@H]1[C@@H](NC(=O)C(F)(F)F)C(F)(F)CCN1C(=O)OCc1ccccc1]

### Step 1:

Under a nitrogen atmosphere, a solution of benzyl rac-2-(((tert-butyldimethylsilyl)oxy)methyl)-3-oxopiperidine-1-carboxylate (12.3 g, 32.546 mmol) in THF (120 mL) was added dropwise over 1.5 h to a solution of NaHMDS (39 mL, 1 M solution in THF, 39.0 mmol) in THF (370 mL) at −78° C. and the reaction mixture was stirred at −78° C. for 30 min. TMSCl (5.650 g, 6.6 mL, 52.002 mmol) was added dropwise at −78° C. and the reaction mixture was stirred at −78° C. for 30 min. The reaction was diluted with MTBE (100 mL) and quenched by addition of a saturated aqueous NaHCO₃ solution (50 mL) and water (150 mL). The aqueous layer was separated and extracted with MTBE (2×100 mL). The combined organic layers were washed with water (75 mL), brine (75 mL), dried (Na₂SO₄), filtered, and concentrated in vacuo to give benzyl rac-6-(((tert-butyldimethylsilyl)oxy)methyl)-5-((trimethylsilyl)oxy)-3,6-dihydropyridine-1(2H)-carboxylate (18.9 g, 92%) as a yellow oil, which was used without further purification in the next step. ¹H NMR (400 MHz, DMSO-d₆) δ 7.46-7.22 (m, 5H), 5.29-5.01 (m, 3H), 4.38-3.68 (m, 4H), 3.10 (m, 1H, overlapped with solvent), 2.23 (m, 1H), 1.95 (m, 1H), 0.82 (m, 9H), 0.23-−0.08 (m, 15H, overlapped with impurities) ppm. ESI-MS m/z calc. 449.242, found 450.2 (M+1)⁺.

### Step 2:

Under a nitrogen atmosphere, Selectfluor™ (2 g, 5.646 mmol) was added in one portion to a stirred solution of benzyl rac-6-(((tert-butyldimethylsilyl)oxy)methyl)-5-((trimethylsilyl)oxy)-3,6-dihydropyridine-1(2H)-carboxylate (2.54 g, 4.4053 mmol) in MeCN (25 mL) at 0° C. and the reaction mixture was stirred at 0° C. for 2 h. The reaction was quenched by addition of a saturated aqueous NaHCO₃ solution (50 mL). The aqueous layer was separated and extracted with DCM (3×75 mL). The combined organic layers were washed with brine (50 mL), dried (Na₂SO₄), filtered, and concentrated in vacuo. Purification by flash chromatography (40 g SiO₂, 0 to 20% MeOH in DCM) gave benzyl rac-2-(((tert-butyldimethylsilyl)oxy)methyl)-4-fluoro-3-oxopiperidine-1-carboxylate (1.5 g, 82%) as a light yellow oil. ¹H NMR (400 MHz, DMSO-d₆) δ 7.38-7.31 (m, 5H), 5.27 (m, 1H), 5.16-5.00 (m, 2H), 4.56 (m, 1H), 4.24 (m, 1H), 3.96-3.81 (m, 2H), 3.47 (m, 1H), 2.41 (m, 1H), 2.07 (m, 1H, overlapped with MeCN), 0.81 (m, 9H), −0.01 (m, 6H) ppm. ESI-MS m/z calc. 395.193, found 396.2 (M+1)⁺.

### Step 3:

Under a nitrogen atmosphere, a solution of benzyl rac-2-(((tert-butyldimethylsilyl)oxy)methyl)-4-fluoro-3-oxopiperidine-1-carboxylate (11.2 g, 25.966 mmol) in THF (112 mL) was added dropwise over 1.5 h to NaHMDS (33 mL, 1 M solution in THF, 33 mmol) in THF (336 mL) at −78° C. and the reaction mixture was stirred at −78° C. for 30 min. TMSCl (4.537 g, 5.3 mL, 41.759 mmol) was added dropwise at −78° C. and the reaction mixture was stirred at −78° C. for 30 min. The reaction was diluted with MTBE (100 mL) and quenched by addition of a saturated aqueous NaHCO₃ solution (50 mL) and water (150 mL). The aqueous layer was separated and extracted with MTBE (2×100 mL). The combined organic layers were washed with water (75 mL) and brine (75 mL), dried (Na₂SO₄), filtered, and concentrated in vacuo to give benzyl rac-6-(((tert-butyldimethylsilyl)oxy)methyl)-4-fluoro-5-((trimethylsilyl)oxy)-3,6-dihydropyridine-1(2H)-carboxylate (14.6 g, 100%) as a yellow oil, which was used without further purification in the next step. ¹H NMR (400 MHz, DMSO-d₆) δ 7.41-7.27 (m, 5H), 5.37-4.99 (m, 3H), 4.28 (m, 1H), 4.15-3.67 (m, 3H), 2.31-1.50 (m, 2H, overlapped with THF), 0.82 (m, 9H), 0.22-−0.07 (m, 15H, overlapped with impurities) ppm.

### Step 4:

Under a nitrogen atmosphere, Selectfluor™ (1.54 g, 4.347 mmol) was added in one portion to a stirred solution of benzyl rac-6-(((tert-butyldimethylsilyl)oxy)methyl)-4-fluoro-5-((trimethylsilyl)oxy)-3,6-dihydropyridine-1(2H)-carboxylate (1.88 g, 3.577 mmol) in MeCN (20 mL) at 0° C. and the reaction mixture was stirred at 0° C. for 1.5 h. The reaction was quenched by addition of a saturated aqueous NaHCO₃ solution (50 mL). The aqueous layer was separated and extracted with DCM (3×50 mL). The combined organic layers were washed with brine (50 mL), dried (Na₂SO₄), filtered, and concentrated in vacuo. Purification by flash chromatography (40 g SiO₂, 0 to 10% MeOH in DCM) gave benzyl rac-2-(((tert-butyldimethylsilyl)oxy)methyl)-4,4-difluoro-3-oxopiperidine-1-carboxylate (1.23 g, 55%) as a yellow oil. ¹H NMR (400 MHz, DMSO-d₆, T°=80° C.) δ 7.43-7.21 (m, 5H), 5.22-4.93 (m, 2H), 4.84 (m, 1H), 4.25-3.65 (m, 3H), 3.07 (m, 1H, overlapped with water), 2.35-1.79 (m, 2H), 0.90-0.80 (m, 9H), 0.05-−0.04 (m, 6H) ppm. ESI-MS m/z calc. 413.183, found 432.2 (M+19)⁺.

### Step 5:

Under a nitrogen atmosphere, Ti(OEt)₄ (2.720 g, 2.5 mL, 11.924 mmol) was added dropwise to a stirred solution of benzyl rac-2-(((tert-butyldimethylsilyl)oxy)methyl)-4,4-difluoro-3-oxopiperidine-1-carboxylate (1.75 g, 4.228 mmol) and (R)-2-methylpropane-2-sulfinamide (900 mg, 7.426 mmol) in THF (50 mL) at ambient temperature and the reaction mixture was warmed to 60° C. and was stirred for 18 h. The reaction was cooled to −50° C. NaBH₄ (480 mg, 12.688 mmol) was added in one portion and the reaction mixture was stirred at −50° C. for 2.5 h. Additional NaBH₄ (160 mg, 4.229 mmol) was added and the mixture was stirred at −50° C. for an additional 30 min. The reaction was quenched by addition of a saturated aqueous NaHCO₃ (75 mL) solution. The aqueous layer was separated and extracted with DCM (3×150 mL). The combined organic layers were dried (Na₂SO₄), filtered, and concentrated in vacuo. Purification by flash chromatography (120 g SiO₂, 1 to 10% MeOH in DCM) followed by reverse phase chromatography (150 g C₁₈ column, 5 to 95% MeCN in water with 0.1% formic acid) gave, after lyophilisation, benzyl dia-(2R,3R)-2-(((tert-butyldimethylsilyl)oxy)methyl)-3-(((R*)-tert-butylsulfinyl)amino)-4,4-difluoropiperidine-1-carboxylate (712 mg, 30%) as a thick yellow oil. ¹H NMR (400 MHz, DMSO-d₆, T°=80° C.) δ 7.42-7.20 (m, 5H), 5.32-4.94 (m, 3H), 4.68-4.58 (m, 0.5H), 4.56-4.49 (m, 0.5H), 4.13-3.99 (m, 1.5H), 3.98-3.90 (m, 0.5H), 3.88-3.67 (m, 2H), 3.13-3.08 (m, 1H), 2.19-1.90 (m, 2H), 1.16 (s, 9H), 0.85 (s, 4.5H), 0.83 (s, 4.5H), 0.04 (s, 3H), 0.01 (s, 3H) ppm. ESI-MS m/z calc. 518.245, found 519.4 (M+1)⁺.

### Step 6:

HCl (1.5 mL, 4 M solution in 1,4-dioxane, 6.0 mmol) was added dropwise to a stirred solution of benzyl dia-(2R,3R)-2-(((tert-butyldimethylsilyl)oxy)methyl)-3-(((R)-tert-butylsulfinyl)amino)-4,4-difluoropiperidine-1-carboxylate (662 mg, 1.183 mmol) in MeOH (30 mL) at ambient temperature and the reaction mixture was stirred at ambient temperature for 1 h. The reaction was concentrated in vacuo. The residue was subsequently co-evaporated with MeOH (20 mL) and MTBE (2×20 mL) to give benzyl rac-(2R,3R)-3-amino-4,4-difluoro-2-(hydroxymethyl)piperidine-1-carboxylate hydrochloride (549 mg, 99%) as an orange semi-solid, which was used without further purification in the next step. ¹H NMR (400 MHz, DMSO-d₆) δ 8.80 (br s, 3H), 7.45-7.27 (m, 5H), 5.16-5.05 (m, 2H), 4.78 (m, 1H), 4.17-3.94 (m, 2H), 3.71-3.51 (m, 3H), 3.07 (m, 1H, overlapped with MTBE), 2.29-2.02 (m, 2H) ppm. ESI-MS m/z calc. 300.129, found 301.2 (M+1)⁺.

### Step 7:

Tf₂O (2.267 g, 1.50 mL, 10.791 mmol) was added to a stirred solution of benzyl rac-(2R,3R)-3-amino-4,4-difluoro-2-(hydroxymethyl)piperidine-1-carboxylate hydrochloride (2.025 g, 5.123 mmol) and Et₃N (2.686 g, 3.70 mL, 26.546 mmol) in DCM (100 mL) at 0° C. and the reaction mixture was stirred at 0° C. for 2 h. The reaction was quenched at 0° C. by addition of a saturated aqueous NH₄Cl solution (50 mL) and diluted with water (100 mL). The aqueous layer was separated and extracted with DCM (3×150 mL). The combined organic layers were dried (Na₂SO₄), filtered, and concentrated in vacuo. Purification by reverse phase chromatography (100 g C₁₈ column, 5 to 70% MeCN in water with NH₄HCO₃ at pH 10) gave, after lyophilisation, benzyl rac-(2R,3R)-4,4-difluoro-2-(hydroxymethyl)-3-(2,2,2-trifluoroacetamido)piperidine-1-carboxylate (Int-26, 1.68 g, 81%) as an off-white solid. ¹H NMR (400 MHz, DMSO-d₆) δ 9.73 (br d, J=8.6 Hz, 1H), 7.46-7.23 (m, 5H), 5.18-5.05 (m, 2H), 5.02-4.91 (m, 1H), 4.64-4.47 (m, 2H), 4.13-4.02 (m, 1H), 3.72-3.53 (m, 2H), 3.18-3.05 (m, 1H), 2.31-2.10 (m, 2H) ppm. ESI-MS m/z calc. 396.111, found 397.1 (M+1)⁺.

### Example 48: Intermediate 27

### Benzyl (2S,3R)-2-(hydroxymethyl)-3-((trifluoromethyl)sulfonamido)piperidine-1-carboxylate (Int-27)

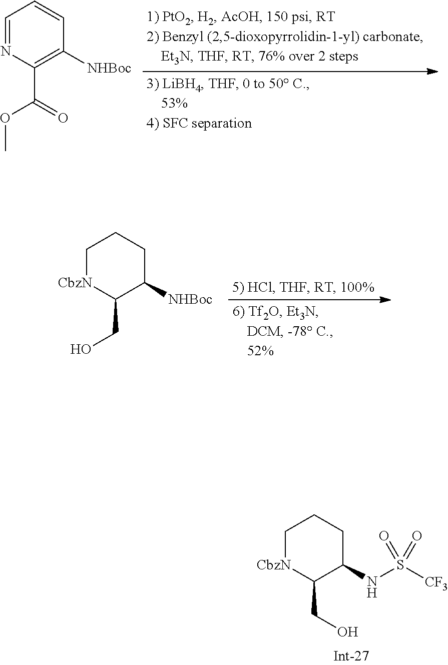 [SMILES: COC(=O)c1ncccc1NC(=O)OC(C)(C)C ; CC(C)(C)OC(=O)N[C@@H]1CCCN(C(=O)OCc2ccccc2)[C@@H]1CO ; O=C(OCc1ccccc1)N1CCC[C@@H](NS(=O)(=O)C(F)(F)F)[C@H]1CO]

### Step 1:

A mixture of methyl 3-((tert-butoxycarbonyl)amino)picolinate (20 g, 79.281 mmol) and PtO₂ (3.50 g, 15.413 mmol) in AcOH (60 mL) was stirred under a hydrogen atmosphere at a pressure of 150 psi over 48 h. The reaction mixture was filtered on a pad of Celite® and the filtrates were concentrated in vacuo to give methyl rac-(2S,3R)-3-((tert-butoxycarbonyl)amino)piperidine-2-carboxylate, which was used without further purification in the next step.

### Step 2:

Et₃N (9.583 g, 13.2 mL, 94.705 mmol) was added to a stirred mixture of methyl rac-(2S,3R)-3-((tert-butoxycarbonyl)amino)piperidine-2-carboxylate and benzyl (2,5-dioxopyrrolidin-1-yl) carbonate (27.8 g, 111.55 mmol) in THF (100 mL) and the reaction mixture was stirred at ambient temperature overnight. Purification by flash chromatography (330 g SiO₂, 0 to 40% EtOAc in hexanes) gave 1-benzyl 2-methyl rac-(2S,3R)-3-((tert-butoxycarbonyl)amino)piperidine-1,2-dicarboxylate (26.3 g, 76% over 2 steps). ESI-MS m/z calc. 392.195, found 393.2 (M+1)⁺.

### Step 3:

Under a nitrogen atmosphere, LiBH₄ (1.121 mL, 2 M solution in THF, 2.242 mmol) was slowly added to a stirred solution of 1-benzyl 2-methyl rac-(2S,3R)-3-((tert-butoxycarbonyl)amino)piperidine-1,2-dicarboxylate (400 mg, 1.0192 mmol) in THF (5 mL) at 0° C. and the reaction mixture was heated and stirred at 50° C. for 2 h. The reaction was quenched by slow addition of water (25 mL) and extracted with DCM (2×20 mL). The combined organic layers were washed with brine, dried (Na₂SO₄), filtered and concentrated in vacuo. Purification by flash chromatography (12 g C₁₈ column, 10 to 65% MeCN in water with 10 mM NH₄HCO₃) gave benzyl rac-(2S,3R)-3-((tert-butoxycarbonyl)amino)-2-(hydroxymethyl)piperidine-1-carboxylate (218.5 mg, 53%). ¹H NMR (400 MHz, DMSO-d₆) δ 7.33 (m, 4H), 7.27 (m, 1H), 6.85 (s, 1H), 5.01-5.08 (m, 2H), 4.56 (br s, 1H), 4.36 (s, 1H), 3.82 (d, J=13.29 Hz, 1H), 3.60 (br s, 1H), 3.48 (br s, 2H), 2.81 (br s, 1H), 1.61 (d, J=12.69 Hz, 1H), 1.50 (m, 2H), 1.36 (s, 9H) ppm; exchangeable H not observed. ESI-MS m/z calc. 364.200, found 265.3 (M-99)-.

### Step 4:

The enantiomers of benzyl rac-(2S,3R)-3-((tert-butoxycarbonyl)amino)-2-(hydroxymethyl)piperidine-1-carboxylate were separated by chiral SFC using a Chiralpak® IC column, 5 mm particle size, 25 cm×21 mm from Daicel Corporation (Mobile phase: 20% ⁱPrOH (supplemented with 5 mM ammonia), 80% CO₂; Flow rate 100 mL/min) to give:

First Eluting Isomer: benzyl (2S,3R)-3-((tert-butoxycarbonyl)amino)-2-(hydroxymethyl)piperidine-1-carboxylate as a white solid. ¹H NMR (400 MHz, Chloroform-d) δ 7.37-7.28 (m, 5H), 5.20-5.10 (m, 3H), 4.58 (br s, 1H), 3.96 (br d, 1H), 3.95-3.87 (m, 1H), 3.95-3.70 (m, 2H), 2.92-2.80 (m, 1H), 1.93-1.45 (m, 5H), 1.44 (s, 9H) ppm. ESI-MS m/z calc. 364.200, found 365.3 (M+1)⁺.

Second Eluting Isomer: benzyl (2R,3S)-3-((tert-butoxycarbonyl)amino)-2-(hydroxymethyl)piperidine-1-carboxylate as a white solid. ¹H NMR (400 MHz, Chloroform-d) δ 7.40-7.28 (m, 5H), 5.15 (dd, 3H), 4.58 (br s, 1H), 4.12-3.97 (m, 1H), 3.97-3.87 (m, 1H), 3.87-3.70 (m, 2H), 2.87 (t, 1H), 1.90-1.45 (m, 5H), 1.44 (s, 9H) ppm. ESI-MS m/z calc. 364.200, found 365.2 (M+1)⁺.

### Step 5:

Benzyl (2S,3R)-3-((tert-butoxycarbonyl)amino)-2-(hydroxymethyl)piperidine-1-carboxylate (5.9 g, 16.19 mmol) was treated via the method described in Intermediate 2 Step 6 to give benzyl (2S,3R)-3-amino-2-(hydroxymethyl)piperidine-1-carboxylate hydrochloride (4.9 g, 101%) as a white foam. ¹H NMR (300 MHz, DMSO-d₆) δ 8.27 (s, 3H), 7.44-7.25 (m, 5H), 5.18-5.01 (m, 3H), 4.50 (q, J=6.3 Hz, 1H), 3.87 (dd, J=13.7, 4.3 Hz, 1H), 3.81-3.59 (m, 1H), 3.22 (s, 1H), 2.89 (t, J=13.2 Hz, 1H), 1.89-1.64 (m, 3H), 1.44 (quint, J=12.6, 6.7 Hz, 1H) ppm. ESI-MS m/z calc. 264.147, found 265.5 (M+1)⁺.

### Step 6:

Benzyl (2S,3R)-3-amino-2-(hydroxymethyl)piperidine-1-carboxylate hydrochloride was treated via the method described in Example 24 Step 6 to give, after purification by flash chromatography (80 g SiO₂, 0 to 100% EtOAc in heptane), benzyl (2S,3R)-2-(hydroxymethyl)-3-((trifluoromethyl)sulfonamido)piperidine-1-carboxylate (Int-27, 1.72 g, 52%) as a pale yellow viscous oil. ¹H NMR (300 MHz, DMSO-d₆) δ 9.60 (s, 1H), 7.39-7.27 (m, 5H), 5.07 (d, J=1.6 Hz, 2H), 4.80-4.50 (s, 1H), 4.39 (dt, J=9.8, 4.9 Hz, 1H), 3.89-3.56 (m, 3H), 3.50 (s, 1H), 2.88 (s, 1H), 1.70 (d, J=17.4 Hz, 3H), 1.55-1.30 (m, 1H) ppm. ESI-MS m/z calc. 396.097, found 397.4 (M+1)⁺.

### Example 49: Intermediate 28

### Benzyl (2R,3S)-2-(hydroxymethyl)-3-((trifluoromethyl)sulfonamido)piperidine-1-carboxylate (Int-28)

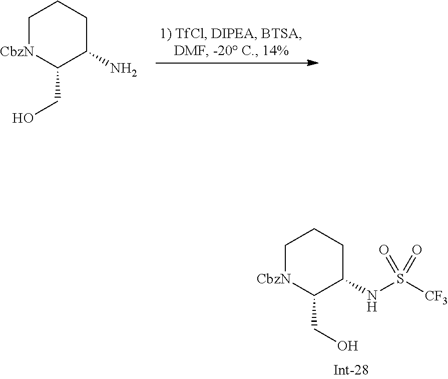 [SMILES: N[C@H]1CCCN(C(=O)OCc2ccccc2)[C@H]1CO ; O=C(OCc1ccccc1)N1CCC[C@H](NS(=O)(=O)C(F)(F)F)[C@@H]1CO]

### Step 1:

A solution of TfCl (5 g, 29.67 mmol) in DMF (20 mL) was added dropwise over 30 min to a stirred mixture of benzyl (2R,3S)-3-amino-2-(hydroxymethyl)piperidine-1-carboxylate hydrochloride (3.66 g, 12.17 mmol), DIPEA (11 mL, 63.15 mmol) and BTSA (4 mL, 16.32 mmol) in DMF (200 mL) at −20° C. and the reaction mixture was stirred overnight at −20° C. The mixture was diluted with a saturated aqueous NaHCO₃ solution (100 mL) and DCM (100 mL) and stirred for 1 h. The aqueous layer was separated and extracted with DCM (3×100 mL). The combined organic layers were concentrated in vacuo. Purification by flash chromatography (120 g SiO₂, 20 to 100% EtOAc in heptane) gave benzyl (2R,3S)-2-(hydroxymethyl)-3-(trifluoromethylsulfonylamino)piperidine-1-carboxylate (Int-28, 730 mg, 14%) as a colourless oil. ¹H NMR (300 MHz, DMSO-d₆) δ 9.59 (s, 1H), 7.39-7.27 (m, 5H), 5.07 (d, J=1.5 Hz, 2H), 4.75 (s, 1H), 4.38 (dd, J=9.7, 4.8 Hz, 1H), 3.84 (d, J=10.7 Hz, 1H), 3.72 (d, J=10.4 Hz, 1H), 3.63 (s, 1H), 3.49 (s, 1H), 2.88 (s, 1H), 1.67 (s, 3H), 1.45 (d, J=13.4 Hz, 1H) ppm. ESI-MS m/z calc. 396.097, found 397.2 (M+1)⁺.

### Example 50: Intermediate 33

### 2-(3-(Benzyloxy)-2-bromophenyl)propan-2-ol (Int-33)

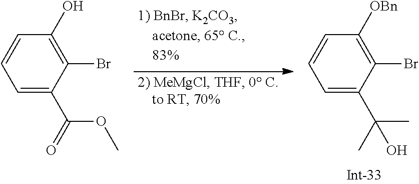 [SMILES: COC(=O)c1cccc(O)c1Br ; CC(C)(O)c1cccc(OCc2ccccc2)c1Br]

### Step 1:

BnBr (6.4 mL, 53.81 mmol) was added to a suspension of methyl 2-bromo-3-hydroxybenzoate (10 g, 43.28 mmol) and K₂CO₃ (18 g, 130.2 mmol) in acetone (200 mL) and the reaction mixture was stirred at 65° C. for 15 h. The reaction was cooled to ambient temperature and filtered through a pad of silica, washing the filtered cake with EtOAc. The filtrates were concentrated in vacuo. Purification by flash chromatography (330 g SiO₂, 0 to 30% EtOAc in hexane) gave methyl 3-(benzyloxy)-2-bromobenzoate (11.5 g, 83%). ¹H NMR (400 MHz, Chloroform-d) δ 7.52 (d, J=7.5 Hz, 2H), 7.44 (t, J=7.4 Hz, 2H), 7.42-7.36 (m, 1H), 7.32 (d, J=4.9 Hz, 2H), 7.08 (t, J=4.7 Hz, 1H), 5.23 (s, 2H), 3.98 (s, 3H) ppm.

### Step 2:

MeMgCl (800 μL, 3 M solution in THF, 2.4 mmol) was added to a stirred mixture of methyl 3-(benzyloxy)-2-bromobenzoate (326.7 mg, 1 mmol) in THF (10 mL) at 0° C. and the reaction mixture was warmed and stirred at ambient temperature for 1 h. Additional amounts of MeMgCl (200 μL, 3 M solution in THF, 0.6 mmol then 400 μL, 1.2 mmol) were added and the reaction was stirred for additional times (1 h and 3 h respectively). The reaction was quenched by addition of a saturated aqueous NH₄Cl solution (20 mL) and the mixture was diluted with MTBE (20 mL). The organic layer was separated, washed with brine (10 mL), dried (MgSO₄), filtered and concentrated in vacuo. Purification by flash chromatography (SiO₂, 0 to 40% EtOAc in heptane) gave 2-(3-(benzyloxy)-2-bromophenyl)propan-2-ol (Int-33, 244 mg, 70%) as a clear oil. ¹H NMR (300 MHz, DMSO-d₆) δ 7.53-7.25 (m, 7H), 7.07 (dd, J=8.1, 1.5 Hz, 1H), 5.22 (s, 1H), 5.17 (s, 2H), 1.64 (s, 6H) ppm. ESI-MS m/z calc. 320.041, found 319.3 (M+1)⁺.

### Example 51: Intermediate 34

### (2-(3-(Benzyloxy)-2-bromophenyl)-2,2-difluoroethoxy)(tert-butyl)dimethylsilane (Int-34)

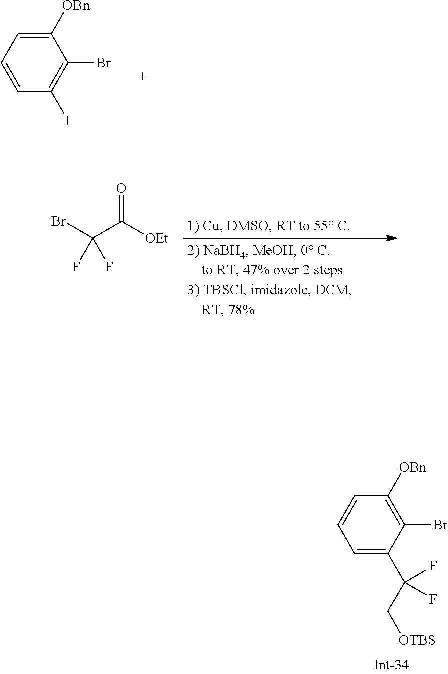 [SMILES: Brc1c(I)cccc1OCc1ccccc1 ; CCOC(=O)C(F)(F)Br ; CC(C)(C)[Si](C)(C)OCC(F)(F)c1cccc(OCc2ccccc2)c1Br]

### Step 1:

Cu (13.4 g, 210.9 mmol) was added to a stirred solution of ethyl 2-bromo-2,2-difluoroacetate (10.3 mL, 79.67 mmol) in DMSO (175 mL) and the reaction mixture was stirred at ambient temperature for 30 min. 1-(Benzyloxy)-2-bromo-3-iodobenzene (20.6 g, 52.95 mmol) was added and the reaction was stirred at 55° C. for 16 h. The mixture was cooled to ambient temperature, filtered through a pad of Celite®, washing the filtered cake with EtOAc (100 mL). The filtrates were collected, washed with water (100 mL) and brine (100 mL), dried (Na₂SO₄), filtered, and concentrated in vacuo. Purification by flash chromatography (SiO₂, 0 to 40% EtOAc in heptane) gave ethyl 2-(3-(benzyloxy)-2-bromophenyl)-2,2-difluoroacetate as a white solid.

### Step 2:

NaBH₄ (3.0 g, 79.30 mmol) was added to a stirred solution of ethyl 2-(3-(benzyloxy)-2-bromophenyl)-2,2-difluoroacetate in MeOH (175 mL) at 0° C. and the reaction mixture was stirred at 0° C. for 5 min, then at ambient temperature for 1 h. The mixture was cooled to 0° C. and carefully quenched by addition of a saturated aqueous NH₄Cl solution. The mixture was partitioned between water (100 mL) and DCM (100 mL). The aqueous layer was separated and extracted with DCM (100 mL). The combined organic layers were washed with brine (100 mL), dried (Na₂SO₄), filtered, and concentrated in vacuo. Purification by flash chromatography (40 g SiO₂, 0 to 50% EtOAc in heptane) gave 2-(3-(benzyloxy)-2-bromophenyl)-2,2-difluoroethan-1-ol (8.9 g, 47% over 2 steps) as a colourless gum. ¹H NMR (400 MHz, Chloroform-d) δ 7.47-7.21 (m, 7H), 6.98 (dd, J=8.1, 1.6 Hz, 1H), 5.11 (s, 2H), 4.32-4.05 (m, 2H), 1.88 (s, 1H) ppm.

### Step 3:

TBSCl (6.82 g, 45.25 mmol) was added to a solution of 2-(3-(benzyloxy)-2-bromophenyl)-2,2-difluoroethan-1-ol (12.1 g, 35.26 mmol) and imidazole (3.61 g, 53.03 mmol) in DCM (120 mL) and the reaction mixture was stirred at ambient temperature for 30 min. The reaction was partitioned with water (100 mL). The organic layer was separated, washed with brine (100 mL), dried (Na₂SO₄), filtered, and concentrated in vacuo. Purification by flash chromatography (SiO₂, 0 to 20% EtOAc in heptane) gave (2-(3-(Benzyloxy)-2-bromophenyl)-2,2-difluoroethoxy)(tert-butyl)dimethylsilane (Int-34, 12.5 g, 78%) as a colourless oil. ¹H NMR (400 MHz, Chloroform-d) δ 7.69-7.18 (m, 7H), 7.03 (dd, J=7.9, 1.8 Hz, 1H), 5.21 (s, 2H), 4.27 (t, J=13.1 Hz, 2H), 0.82 (d, J=0.6 Hz, 9H), 0.00 (s, 6H) ppm.

### Example 52: Intermediate 35

### (3-(Benzyloxy)-2-bromo-6-fluoro-4-methylphenyl)methanol (Int-35)

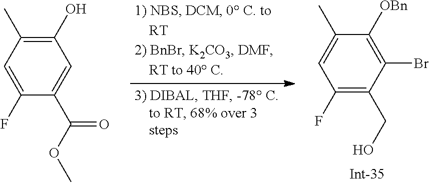 [SMILES: COC(=O)c1cc(O)c(C)cc1F ; Cc1cc(F)c(CO)c(Br)c1OCc1ccccc1]

### Step 1:

NBS (4.9 g, 27.53 mmol) was added to a stirred solution of methyl 2-fluoro-5-hydroxy-4-methylbenzoate (5 g, 27.15 mmol) in DCM (100 mL) at 0° C. and the reaction mixture was stirred at ambient temperature for 16 h. The reaction was diluted with a saturated aqueous Na₂S₂O₃ solution (10 mL) and stirred vigorously for 5 min. The mixture was extracted with DCM (2×10 mL). The combined organic layers were dried (Na₂SO₄), filtered through a short plug of silica, and concentrated in vacuo to give methyl 2-bromo-6-fluoro-3-hydroxy-4-methylbenzoate, which was used without further purification in the next step. ESI-MS m/z calc. 261.964, found 263.2 (M+1)⁺.

### Step 2:

BnBr (4.2 mL, 35.31 mmol) was added to a stirred mixture of methyl 2-bromo-6-fluoro-3-hydroxy-4-methylbenzoate and K₂CO₃ (7.5 g, 54.27 mmol) in DMF (100 mL) at ambient temperature and the reaction mixture was stirred at 40° C. for 1 h. The mixture was partitioned between water and EtOAc. The aqueous phase was separated and extracted with EtOAc. The combined organic phases were washed with brine, dried (Na₂SO₄), filtered and concentrated in vacuo. Purification by flash chromatography (SiO₂, 0 to 30% EtOAc in heptane) gave methyl 3-(benzyloxy)-2-bromo-6-fluoro-4-methylbenzoate as a colourless oil, which was used without further purification in the next step.

### Step 3:

DIBAL (61 mL, 1 M solution in THF, 61 mmol) was added dropwise to a stirred solution of methyl 3-(benzyloxy)-2-bromo-6-fluoro-4-methylbenzoate in THF (200 mL) at −78° C. and the reaction mixture was warmed and stirred at ambient temperature overnight. The reaction was quenched by addition of MeOH and the formed emulsion was filtered through a pad of silica. The filtrates were concentrated in vacuo. Purification by flash chromatography (SiO₂, 0 to 30% EtOAc in heptane) gave (3-(benzyloxy)-2-bromo-6-fluoro-4-methylphenyl)methanol (Int-35, 6.0 g, 68% over 3 steps) as a white solid. ¹H NMR (300 MHz, Chloroform-d) δ 7.61-7.53 (m, 2H), 7.47-7.37 (m, 3H), 6.95 (dd, J=9.8, 0.8 Hz, 1H), 4.93 (s, 2H), 4.90-4.84 (m, 2H), 2.33 (s, 3H) ppm; exchangeable H not observed.

The following Intermediate was made using the method described in Intermediate 35 except that, in Step 1, methyl 2,4-difluoro-5-hydroxybenzoate was used as the starting material in place of methyl 2-fluoro-5-hydroxy-4-methylbenzoate. Step 3 was carried out in DCM as the solvent in place of THF:

| Intermediate | | | | |
|---|---|---|---|---|
| Number | Structure | Name | LC/MS | NMR (shifts in ppm) |
| Int-32 | 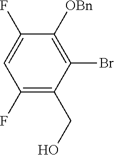 [SMILES: OCc1c(F)cc(F)c(OCc2ccccc2)c1Br] | (3-(Benzyloxy)- 2-bromo-4,6- difluorophenyl) methanol | | ¹H NMR (300 MHz, Chloroform-d) δ 7.67-7.35 (m, 5H), 6.95 (dd, J = 10.4, 9.2 Hz, 1H), 5.10 (s, 2H), 4.85 (s, 2H), 2.07 (s, 1H) ppm. |

The following Intermediates were made using the method described in Intermediate 35 except that Step 1 was omitted. In Step 2, different starting materials were used in place of methyl 2-bromo-6-fluoro-3-hydroxy-4-methylbenzoate and the reaction was carried out in acetone as the solvent. In the case of Int-30, the reaction was carried out at 50° C. In the case of Int-36 and Int-37, the reaction was carried out at 65° C.:

| Intermediate | | | | |
|---|---|---|---|---|
| Number | Structure | Name | LC/MS | NMR (shifts in ppm) |
| Int-30 | 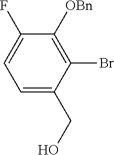 [SMILES: OCc1ccc(F)c(OCc2ccccc2)c1Br] | (3-(Benzyloxy)- 2-bromo-4- fluorophenyl) methanol | ESI-MS m/z calc. 310.000, found 334.1 (M + Na)⁺. | ¹H NMR (300 MHz, Chloroform-d) δ 7.60-7.51 (m, 2H), 7.41 (dddd, J = 7.7, 6.9, 5.7, 1.9 Hz, 3H), 7.23 (ddt, J = 8.5, 5.3, 0.7 Hz, 1H), 7.12 (dd, J = 10.3, 8.6 Hz, 1H), 5.18-5.09 (m, 2H), 4.75 (dt, J = 6.4, 0.9 Hz, 2H), 2.01 (t, J = 6.4 Hz, 1H) ppm. |
| Int-36 | 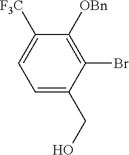 [SMILES: OCc1ccc(C(F)(F)F)c(OCc2ccccc2)c1Br] | (3-(Benzyloxy)- 2-bromo-4- (trifluoromethyl) phenyl)methanol | | ¹H NMR (300 MHz, Chloroform-d) δ 7.67 (d, J = 8.1 Hz, 1H), 7.64-7.56 (m, 2H), 7.52-7.33 (m, 4H), 5.11 (s, 2H), 4.86 (d, J = 6.1 Hz, 2H), 2.14-2.08 (m, 1H) ppm. |
| Int-37 | 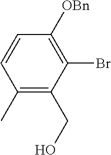 [SMILES: Cc1ccc(OCc2ccccc2)c(Br)c1CO] | (3-(Benzyloxy)- 2-bromo-6- methylphenyl) methanol | | ¹H NMR (300 MHz, Chloroform-d) δ 7.53-7.28 (m, 5H), 7.07 (dd, J = 8.4, 0.8 Hz, 1H), 6.81 (d, J = 8.4 Hz, 1H), 5.14 (s, 2H), 4.88 (d, J = 6.7 Hz, 2H), 2.42 (d, J = 0.7 Hz, 3H), 1.93 (t, J = 6.7 Hz, 1H) ppm. |

The following Intermediate was made using the method described in Intermediate 35 except that, in Step 1, 2,3-difluoro-5-hydroxybenzaldehyde was used in place of methyl 2-fluoro-5-hydroxy-4-methylbenzoate. The conditions of Step 3 were those of Intermediate 34 Int-34 Step 2:

| Intermediate | | | | |
|---|---|---|---|---|
| Number | Structure | Name | LC/MS | NMR (shifts in ppm) |
| Int-31 | 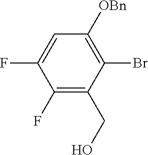 [SMILES: OCc1c(F)c(F)cc(OCc2ccccc2)c1Br] | (3-(Benzyloxy)- 2-bromo-5,6- difluorophenyl) methanol | ESI-MS m/z calc. 327.991, found 328.3 (M + 1)⁺. | ¹H NMR (300 MHz, Chloroform-d) δ 7.48-7.31 (m, 5H), 6.85-6.75 (m, 1H), 5.11 (d, J = 1.9 Hz, 2H), 4.89 (s, 2H), 2.21 (s, 1H) ppm. |

### Example 53: Intermediate 40

### Benzyl rac-2-((4′-methyl-4-oxospiro[cyclohexane-1,1′-isochroman]-8′-yl)oxy)acetate (Int-40)

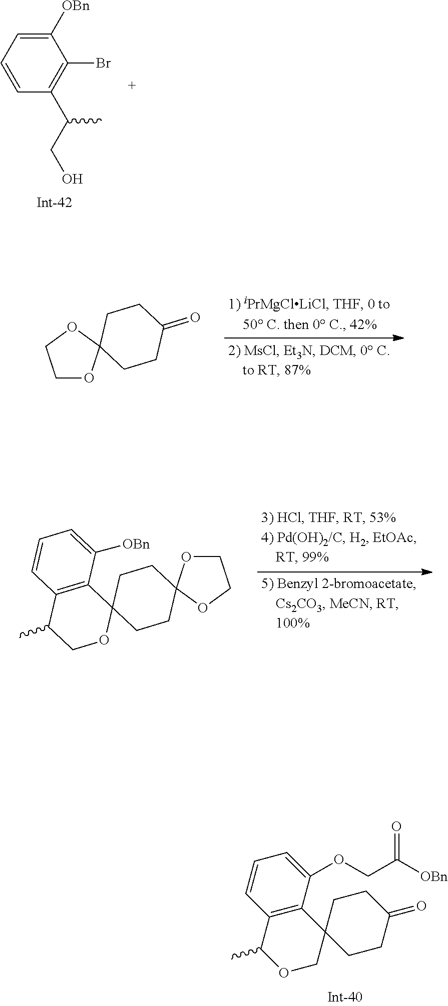

### Step 1:

Under an argon atmosphere, ⁱPrMgCl·LiCl (28.6 mL, 1.3 M solution in THF, 37.18 mmol) was added dropwise over 2 min to a stirred solution of rac-2-(3-(benzyloxy)-2-bromophenyl)propan-1-ol (3.99 g, 12.42 mmol) in THF (64 mL) at 0° C. and the reaction mixture was stirred at 50° C. for 2.5 h. The reaction was cooled to 0° C. A solution of 1,4-dioxaspiro[4.5]decan-8-one (3.88 g, 24.84 mmol) in THF (17 mL) was added dropwise over 2 min and the mixture was stirred at 0° C. for 60 min. The reaction was quenched by addition of a saturated aqueous NH₄Cl solution (50 mL) and extracted with DCM (2×). The combined organic layers were dried by passing the solution through a phase separator cartridge and concentrated in vacuo. Purification by flash chromatography (SiO₂, 0 to 60% EtOAc in heptane) gave rac-8-(2-(benzyloxy)-6-(1-hydroxypropan-2-yl)phenyl)-1,4-dioxaspiro[4.5]decan-8-ol (2.600 g, 42%) as a thick colourless oil. ESI-MS m/z calc. 398.209, found 381.2 (M-OH)⁺.

### Step 2:

Under a nitrogen atmosphere, MsCl (63 μL, 0.814 mmol) was added to a solution of rac-8-(2-(benzyloxy)-6-(1-hydroxypropan-2-yl)phenyl)-1,4-dioxaspiro[4.5]decan-8-ol (320 mg, 0.683 mmol) and Et₃N (334 μL, 2.396 mmol) in DCM (7.7 mL) at 0° C. The cooling bath was removed and the reaction was stirred at ambient temperature for 2 h. The reaction was quenched by addition of a half saturated aqueous NH₄Cl solution (5 mL). The aqueous phase was separated and extracted with DCM (2×). The combined organic layers were dried, by passing the solution through a phase separator cartridge, and concentrated in vacuo. Purification by flash chromatography (SiO₂, 0 to 20% EtOAc in DCM) gave rac-8-(benzyloxy)-4-methyldispiro[isochromane-1,1′-cyclohexane-4′,2″-[1,3]dioxolane](244 mg, 87%) as a white solid. ESI-MS m/z calc. 380.199, found 381.2 (M+1)⁺.

### Step 3:

HCl (1.15 mL, 6 M aqueous solution, 6.9 mmol) was added to a stirred solution of rac-8-(benzyloxy)-4-methyldispiro[isochromane-1,1′-cyclohexane-4′,2″-[1,3]dioxolane] (244 mg) in THF (2.7 mL) and the reaction mixture was stirred at ambient temperature for 30 min. The reaction was diluted with water and extracted with DCM (3×). The combined organic extracts were washed with a saturated aqueous NaHCO₃ solution, dried by passing the solution through a phase separator cartridge, and concentrated in vacuo. Purification by flash chromatography (SiO₂, 0 to 40% EtOAc in heptane) gave rac-8′-(benzyloxy)-4′-methylspiro[cyclohexane-1,1′-isochroman]-4-one (124 mg, 53%) as a white solid. ¹H NMR (400 MHz, Chloroform-d) δ 7.43-7.29 (m, 5H), 7.20-7.12 (m, 1H), 6.85 (d, J=7.7 Hz, 1H), 6.78 (d, J=8.2 Hz, 1H), 5.09 (s, 2H), 4.02-3.91 (m, 1H), 3.73-3.63 (m, 1H), 3.07 (td, J=13.4, 5.1 Hz, 1H), 2.95-2.67 (m, 4H), 2.27-2.12 (m, 3H), 2.10-1.98 (m, 1H), 1.36 (d, J=6.8 Hz, 3H) ppm. ESI-MS m/z calc. 336.173, found 337.1 (M+1)⁺.

### Step 4:

Under a nitrogen atmosphere, a solution of rac-8′-(benzyloxy)-4′-methylspiro[cyclohexane-1,1′-isochroman]-4-one (135 mg, 0.401 mmol) in EtOAc (2.2 mL) was added to a slurry of Pd(OH)₂ (56 mg, on carbon, 0.040 mmol) in EtOAc (2.2 mL) and the reaction mixture was stirred under a hydrogen atmosphere at ambient temperature for 90 min. The reaction was filtered through a pad of Celite®, washing the filtered cake with EtOAc. The filtrates were concentrated in vacuo to give rac-8′-hydroxy-4′-methylspiro[cyclohexane-1,1′-isochroman]-4-one (109 mg, 99%) as a white solid. ¹H NMR (400 MHz, Chloroform-d) δ 7.07 (t, J=7.7 Hz, 1H), 6.82 (d, J=7.6 Hz, 1H), 6.55 (d, J=7.9 Hz, 1H), 5.12 (s, 1H), 3.96 (dd, J=11.3, 3.6 Hz, 1H), 3.69 (dd, J=11.3, 3.9 Hz, 1H), 3.07 (td, J=13.5, 5.0 Hz, 1H), 2.95-2.74 (m, 4H), 2.33-2.21 (m, 2H), 2.21-2.12 (m, 1H), 2.09-2.00 (m, 1H), 1.35 (d, J=7.0 Hz, 3H) ppm. ESI-MS m/z calc. 246.126, found 247.1 (M+1)⁺.

### Step 5:

Benzyl 2-bromoacetate (318 μL, 2.007 mmol) was added to a suspension of rac-8′-hydroxy-4′-methylspiro[cyclohexane-1,1′-isochroman]-4-one (405 mg, 1.604 mmol) and Cs₂CO₃ (1.0 g, 3.069 mmol) in MeCN (8.4 mL) and the reaction mixture was stirred at ambient temperature for 4.5 h. The reaction was filtered through pad of Celite®, washing the filtered cake with EtOAc. The filtrates were concentrated in vacuo. Purification by flash chromatography (SiO₂, 0 to 60% EtOAc in heptane) gave benzyl rac-2-((4′-methyl-4-oxospiro[cyclohexane-1,1′-isochroman]-8′-yl)oxy)acetate (Int-40, 698 mg, 100%) as athick colourless oil. ¹H NMR (300 MHz, Chloroform-d) δ 7.39-7.27 (m, 5H), 7.13 (t, J=7.9 Hz, 1H), 6.87 (dt, J=7.7, 0.9 Hz, 1H), 6.55 (dd, J=8.3, 1.1 Hz, 1H), 5.21 (s, 2H), 4.65 (s, 2H), 3.94 (dd, J=11.3, 3.7 Hz, 1H), 3.67 (dd, J=11.3, 3.8 Hz, 1H), 3.09 (td, J=13.3, 5.2 Hz, 1H), 2.99-2.67 (m, 4H), 2.32-2.18 (m, 2H), 2.17-2.06 (m, 1H), 2.04-1.93 (m, 1H), 1.35 (d, J=7.0 Hz, 3H) ppm. ESI-MS m/z calc. 394.178, found 395.1 (M+1)⁺.

### Example 54: Intermediate 42

### rac-2-(3-(Benzyloxy)-2-bromophenyl)propan-1-ol (Int-42)

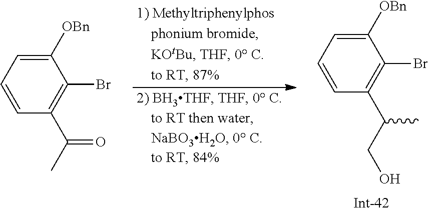 [SMILES: CC(=O)c1cccc(OCc2ccccc2)c1Br ; CC(CO)c1cccc(OCc2ccccc2)c1Br]

### Step 1:

Under a nitrogen atmosphere, KO^{t}Bu (21.5 mL, 1 M solution in THF, 21.5 mmol) was added to a stirred solution of methyltriphenylphosphonium bromide (7.70 g, 21.55 mmol) in THF (55 mL) at 0° C. and the reaction mixture was stirred at 0° C. for 30 min. A solution of 1-(3-(benzyloxy)-2-bromophenyl)ethan-1-one (5.48 g, 17.96 mmol) in THF (55 mL) was added dropwise over 5 min to the reaction mixture. The cooling bath was removed and the reaction was stirred at ambient temperature for 5.5 h. The mixture was filtered, washing the filtered cake with Et₂O. The filtrates were concentrated in vacuo. Purification by flash chromatography (SiO₂, 0 to 20% EtOAc in heptane) gave 1-(benzyloxy)-2-bromo-3-(prop-1-en-2-yl)benzene (4.715 g, 87%) as a white solid. ¹H NMR (400 MHz, Chloroform-d) δ 7.50 (d, J=7.5 Hz, 2H), 7.41 (t, J=7.7 Hz, 2H), 7.36-7.29 (m, 1H), 7.23-7.14 (m, 1H), 6.88-6.78 (m, 2H), 5.22 (s, 1H), 5.17 (s, 2H), 4.95-4.91 (m, 1H), 2.11 (s, 3H) ppm.

### Step 2:

Under an argon atmosphere, BH₃·THF (44 mL, 1 M solution in THF, 44 mmol) was added over 5 min to a stirred solution of 1-(benzyloxy)-2-bromo-3-(prop-1-en-2-yl)benzene (3.935 g, 12.98 mmol) in THF (64 mL) at 0° C. The cooling bath was removed and the reaction was stirred at ambient temperature for 70 min. The reaction was cooled to 0° C., quenched by dropwise addition of water (64 mL) and NaBO₃·H₂O (5.17 g, 51.8 mmol), both causing gas evolution. The cooling bath was removed and the reaction was vigorously stirred for 1 h. The mixture was filtered, washing the filtered cake with Et₂O and the filtrates were partitioned between water and Et₂O. The aqueous phase was extracted with Et₂O (2×). The combined organic layers were washed with brine, dried (Na₂SO₄), filtered, and concentrated in vacuo. Purification by flash chromatography (SiO₂, 0 to 100% EtOAc in heptane) gave rac-2-(3-(benzyloxy)-2-bromophenyl)propan-1-ol (Int-42, 3.71 g, 84%) as a white solid. ¹H NMR (400 MHz, Chloroform-d) δ 7.53-7.47 (m, 2H), 7.41 (t, J=7.7 Hz, 2H), 7.33 (t, J=7.3 Hz, 1H), 7.26-7.21 (m, 1H), 6.94-6.89 (m, 1H), 6.87-6.80 (m, 1H), 5.16 (s, 2H), 3.81 (ddt, J=10.7, 6.5, 1.0 Hz, 1H), 3.77-3.59 (m, 2H), 1.29 (d, J=6.8 Hz, 3H) ppm; exchangeable H not observed. ESI-MS m/z calc. 320.041, found 303.1 (M-OH)⁺.

### Example 55: Intermediate 43

### Benzyl (2R,3S,5R)-2-(hydroxymethyl)-5-methyl-3-(2,2,2-trifluoro-N-(4-methoxybenzyl)acetamido)pyrrolidine-1-carboxylate (Int-43)

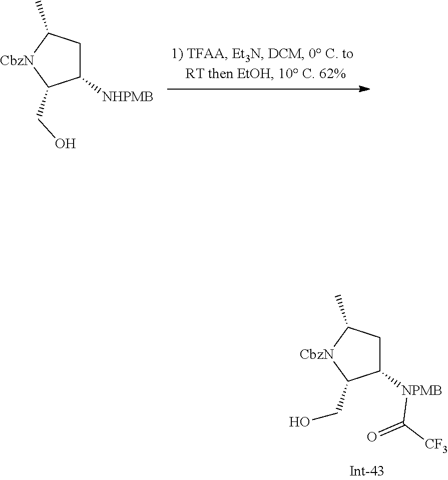

### Step 1:

TFAA (162.33 g, 772.89 mmol, 107.43 mL) was added dropwise to a solution of benzyl (2R,3S,5R)-2-(hydroxymethyl)-3-((4-methoxybenzyl)amino)-5-methylpyrrolidine-1-carboxylate (141.5 g, 368.04 mmol) and Et₃N (148.97 g, 1.47 mol, 204.91 mL) in DCM (1.4 L) at 0° C. and the reaction mixture was stirred at 20° C. for 1 h. EtOH (16.96 g, 368.04 mmol, 21.49 mL) was added at 10° C. and the mixture was stirred for 30 min at 10° C. The reaction was quenched by addition of HCl (1 M aqueous solution, 1 L) and extracted with DCM (2×700 mL). The combined organic layers were concentrated in vacuo. Purification by flash chromatography (SiO₂, 0 to 12% EtOAc in hexanes) gave benzyl (2R,3S,5R)-2-(hydroxymethyl)-5-methyl-3-(2,2,2-trifluoro-N-(4-methoxybenzyl)acetamido)pyrrolidine-1-carboxylate (Int-43, 110 g, 62%) as a light-yellow solid. ¹H NMR (400 MHz, Chloroform-d) δ 7.44-7.30 (m, 5H), 7.07 (d, J=8.4 Hz, 2H), 6.90 (d, J=8.4 Hz, 2H), 5.13 (br s, 2H), 4.98-4.72 (m, 2H), 4.60-4.31 (m, 2H), 3.89-3.78 (m, 4H), 3.73 (br d, J=4.4 Hz, 1H), 3.59-3.43 (m, 1H), 2.08-1.97 (m, 1H), 1.83-1.73 (m, 1H), 1.31 (br d, J=3.6 Hz, 3H) ppm; exchangeable H not observed. ESI-MS m/z calc. 480.187, found 481.3 (M+H)⁺.

### Example 56: Human OX2R IP1 Assay

The G protein-coupled protein receptor (GPCR) OX2R signals through the Gq/11 signaling pathway. Activation of this pathway can be readily detected by measuring the accumulation of inositol monophosphate (IP-one or IP1) in the presence of excess LiCl. This can be quantified using the commercially available cell-based IP-One Gq kit (Revvity cat #: 62IPAPEB) coupled with a cell line expressing OX2R. IP1 generated within the cell by OX2R agonism competes with the IP1 analog coupled to a d2 fluorophore (FRET acceptor) for binding to an anti-IP1 monoclonal antibody labeled with Eu cryptated (FRET donor). The measured HTRF-FRET based signal is inversely proportional to the IP1 concentration produced.

Tetracycline-inducible suspension human embryonic kidney cells stably expressing tet-responsive human OX2R (2F7-hOX2R) were generated by first transfecting Expi293F Inducible cells (ThermoFisher #A39241) with pcZeo TetO DNA containing hOX2R cDNA. In this plasmid, the TET operator sequences have been inserted between the TATA box of the CMV promoter and the transcriptional start site for OX2R. These cells were induced with 4 μg/mL doxycycline at 32° C. After 24 hours of induction, cells were harvested, aliquoted, and cryopreserved in Expi293 medium containing 10% or 7.5% DMSO. On the day of the assay, cells are thawed, washed with PBS, and resuspended in 1× Cisbio stimulation buffer. Cells were then plated at a concentration of 6,000 or 5,000 cells/well into a 384-well assay plate containing test compounds to give a final concentration of 0.5% or 0.55% DMSO. Following a one-hour incubation at 37° C., the reaction was terminated by addition of the Cisbio detection mix containing IP1-d2 and anti-IP1-cryptate in lysis buffer. Following a one-hour incubation at room temperature, HTRF-FRET values were measured using a Envision Multilabel Plate Reader (Perkin Elmer) and the data were expressed as the standard 665 nm/620 nm ratio.

Compounds 1-28, 30-57 and 60-74 were found to have EC₅₀ values of less than 25 nM. Compounds 29, 58 and 59 were found to have EC₅₀ values of greater than or equal to 25 nM to less than 200 nM.

### INCORPORATION BY REFERENCE

All of the U.S. patents and U.S. and PCT patent application publications cited herein are hereby incorporated by reference.

### EQUIVALENTS

Those skilled in the art will recognize or be able to ascertain using no more than routine experimentation, many equivalents to the specific embodiments of the invention described herein. Such equivalents are intended to be encompassed by the following claims.

While there have been shown and described what are at present considered the preferred embodiments of the invention, those skilled in the art may make various changes and modifications which remain within the scope of the invention defined by the appended claims.

1. A compound of formula (I): 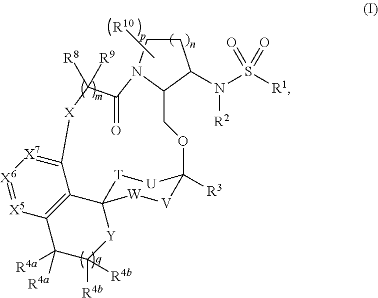
or a pharmaceutically acceptable salt thereof, wherein:
T is O, CR¹¹R¹², or absent;
U is CR¹³R¹⁴;
V is CR¹⁵R¹⁶;
W is O, CR¹⁷R¹⁸, or absent;
X is O or absent;
X⁵ is CR⁵ or N;
X⁶ is CR⁶ or N;
X⁷ is CR⁷ or N;
Y is O or C(R¹⁹)₂;
R¹ is selected from —NR^{a}R^{b}, —C₁-C₃ alkylene-NR^{a}R^{b}, —C₁-C₃ alkylene-OH, —C₁-C₆ alkyl, —C₁-C₆ haloalkyl, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, and —C₁-C₃ alkylene-(5- to 10-membered heteroaryl), wherein said —C₁-C₃ alkylene-NR^{a}R^{b}, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, or —C₁-C₃ alkylene-(5- to 10-membered heteroaryl) is optionally substituted with 1-3 substituents independently selected from halo, —OH, —C₁-C₃ alkyl, and —C₁-C₃ alkoxy;
R² is —H or —C₁-C₃ alkyl;
R³ is —H, halo, —C₁-C₆ alkyl, —C₁-C₆haloalkyl, or —C₁-C₆ alkoxy;
each R^{4a} is independently selected from —H, halo, —OH, —CN, —C₁-C₆ alkyl, —C₁-C₆ haloalkyl, and —C₁-C₆ alkoxy; or
two R^{4a} together form an oxo;
each R^{4b} is independently selected from —H, halo, —OH, —CN, —C₁-C₆ alkyl, —C₁-C₆haloalkyl, and —C₁-C₆ alkoxy;
R⁵, R⁶, and R are each independently selected from —H, halo, —OH, —CN, —C₁-C₆ alkyl, —C₁-C₆ haloalkyl, and —C₁-C₆ alkoxy;
each R⁸ and each R⁹ is independently selected from —H, halo, —C₁-C₆ alkyl, —C₁-C₆ haloalkyl, and —C₁-C₆ alkoxy; or
R⁸ and R⁹, together with together with the carbon atom to which they are attached, form a C₃-C₆ cycloalkyl optionally substituted with halo, —C₁-C₃ alkyl, —C₁-C₃ haloalkyl, or —C₁-C₃ alkoxy;
each R¹⁰ is independently selected from halo, —C₁-C₆ alkyl, —C₁-C₆haloalkyl, and —C₁-C₆ alkoxy;
R¹¹, R¹², R¹³, R¹⁴, R, R¹, R¹⁷, and R¹⁸ are each independently selected from —H, halo, —C₁-C₆ alkyl, —C₁-C₆ haloalkyl, and —C₁-C₆ alkoxy;
each R¹⁹ is independently selected from —H, halo, and —C₁-C₃ alkyl;
R^{a} and R^{b} are each independently selected from —H and —C₁-C₃ alkyl;
m is 1, 2, 3, or 4;
n is 0, 1, 2, or 3;
p is 0, 1, 2, 3, or 4; and
q is 0 or 1;
provided that:
no more than one of T and W is absent; and
no more than one of X⁵, X⁶, and X⁷ is N.

2. The compound of claim 1, or a pharmaceutically acceptable salt thereof, wherein:
X⁵ is CRS;
X⁶ is CR⁶;
X⁷ is CR⁷; and
R¹ is selected from —NR^{a}R^{b}, —C₁-C₃ alkylene-NR^{a}R^{b}, —C₁-C₆ alkyl, —C₁-C₆ haloalkyl, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, and —C₁-C₃ alkylene-(5- to 10-membered heteroaryl), wherein said —C₁-C₃ alkylene-NR^{a}R^{b}, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, or —C₁-C₃ alkylene-(5- to 10-membered heteroaryl) is optionally substituted with 1-3 substituents independently selected from halo, —OH, —C₁-C₃ alkyl, and —C₁-C₃ alkoxy.

3. The compound of claim 1, or a pharmaceutically acceptable salt thereof, wherein:
X⁵ is CR⁵;
X⁶ is CR⁶;
X⁷ is CR⁷;
q is 0;
R¹ is selected from —NR^{a}R^{b}, —C₁-C₃ alkylene-NR^{a}R^{b}, —C₁-C₆ alkyl, —C₁-C₆ haloalkyl, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, and —C₁-C₃ alkylene-(5- to 10-membered heteroaryl), wherein said —C₁-C₃ alkylene-NR^{a}R^{b}, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, or —C₁-C₃ alkylene-(5- to 10-membered heteroaryl) is optionally substituted with 1-3 substituents independently selected from halo, —OH, —C₁-C₃ alkyl, and —C₁-C₃ alkoxy; and
each R^{4a} is independently selected from —H, halo, —OH, —CN, —C₁-C₆ alkyl, —C₁-C₆haloalkyl, and —C₁-C₆ alkoxy.

4-33. (canceled)

34. A compound of formula (I-A): 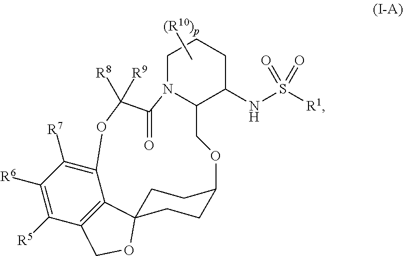
or a pharmaceutically acceptable salt thereof, wherein:
R¹ is selected from —NR^{a}R^{b}, —C₁-C₃ alkylene-NR^{a}R^{b}, —C₁-C₃ alkylene-OH, —C₁-C₆ alkyl, —C₁-C₆ haloalkyl, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, and —C₁-C₃ alkylene-(5- to 10-membered heteroaryl), wherein said —C₁-C₃ alkylene-NR^{a}R^{b}, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, or —C₁-C₃ alkylene-(5- to 10-membered heteroaryl) is optionally substituted with 1-3 substituents independently selected from halo, —OH, —C₁-C₃ alkyl, and —C₁-C₃ alkoxy;
R⁵, R⁶, and R⁷ are each independently selected from —H, halo, —OH, —CN, —C₁-C₆ alkyl, —C₁-C₆haloalkyl, and —C₁-C₆ alkoxy;
each R⁸ and each R⁹ is independently selected from —H, halo, —C₁-C₆ alkyl, —C₁-C₆ haloalkyl, and —C₁-C₆ alkoxy; or
R⁸ and R⁹, together with together with the carbon atom to which they are attached, form a C₃-C₆ cycloalkyl optionally substituted with halo, —C₁-C₃ alkyl, —C₁-C₃ haloalkyl, or —C₁-C₃ alkoxy;
each R¹⁰ is independently selected from halo, —C₁-C₆ alkyl, —C₁-C₆haloalkyl, and —C₁-C₆ alkoxy;
R^{a} and R^{b} are each independently selected from —H and —C₁-C₃ alkyl; and
p is 0, 1, 2, 3, or 4.

35. A compound of formula (I-B): 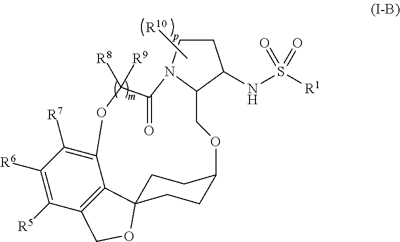
or a pharmaceutically acceptable salt thereof, wherein:
R¹ is selected from —NR^{a}R^{b}, —C₁-C₃ alkylene-NR^{a}R^{b}, —C₁-C₃ alkylene-OH, —C₁-C₆ alkyl, —C₁-C₆ haloalkyl, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, and —C₁-C₃ alkylene-(5- to 10-membered heteroaryl), wherein said —C₁-C₃ alkylene-NR^{a}R^{b}, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, or —C₁-C₃ alkylene-(5- to 10-membered heteroaryl) is optionally substituted with 1-3 substituents independently selected from halo, —OH, —C₁-C₃ alkyl, and —C₁-C₃ alkoxy;
R⁵, R⁶, and R⁷ are each independently selected from —H, halo, —OH, —CN, —C₁-C₆ alkyl, —C₁-C₆haloalkyl, and —C₁-C₆ alkoxy;
each R⁸ and each R⁹ is independently selected from —H, halo, —C₁-C₆ alkyl, —C₁-C₆ haloalkyl, and —C₁-C₆ alkoxy; or
R⁸ and R⁹, together with together with the carbon atom to which they are attached, form a C₃-C₆ cycloalkyl optionally substituted with halo, —C₁-C₃ alkyl, —C₁-C₃ haloalkyl, or —C₁-C₃ alkoxy;
each R¹⁰ is independently selected from halo, —C₁-C₆ alkyl, —C₁-C₆haloalkyl, and —C₁-C₆ alkoxy;
R^{a} and R^{b} are each independently selected from —H and —C₁-C₃ alkyl;
m is 1, 2, 3, or 4;
n is 0, 1, 2, or 3; and
p is 0, 1, 2, 3, or 4.

36. A compound of formula (I-C): 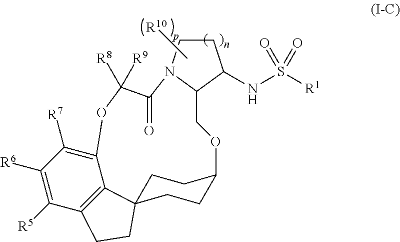
or a pharmaceutically acceptable salt thereof, wherein:
R¹ is selected from —NR^{a}R^{b}, —C₁-C₃ alkylene-NR^{a}R^{b}, —C₁-C₃ alkylene-OH, —C₁-C₆ alkyl, —C₁-C₆ haloalkyl, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, and —C₁-C₃ alkylene-(5- to 10-membered heteroaryl), wherein said —C₁-C₃ alkylene-NR^{a}R^{b}, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, or —C₁-C₃ alkylene-(5- to 10-membered heteroaryl) is optionally substituted with 1-3 substituents independently selected from halo, —OH, —C₁-C₃ alkyl, and —C₁-C₃ alkoxy;
R⁵, R⁶, and R⁷ are each independently selected from —H, halo, —OH, —CN, —C₁-C₆ alkyl, —C₁-C₆haloalkyl, and —C₁-C₆ alkoxy;
each R⁸ and each R⁹ is independently selected from —H, halo, —C₁-C₆ alkyl, —C₁-C₆ haloalkyl, and —C₁-C₆ alkoxy; or
R⁸ and R⁹, together with together with the carbon atom to which they are attached, form a C₃-C₆ cycloalkyl optionally substituted with halo, —C₁-C₃ alkyl, —C₁-C₃ haloalkyl, or —C₁-C₃ alkoxy;
each R¹⁰ is independently selected from halo, —C₁-C₆ alkyl, —C₁-C₆haloalkyl, and —C₁-C₆ alkoxy;
R^{a} and R^{b} are each independently selected from —H and —C₁-C₃ alkyl;
n is 0, 1, 2, or 3; and
p is 0, 1, 2, 3, or 4.

37. A compound of formula (I-D): 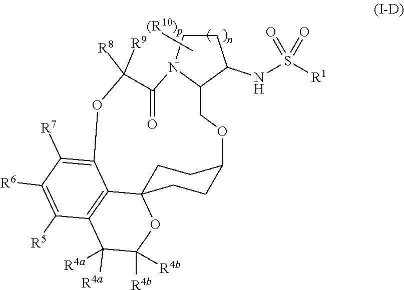
or a pharmaceutically acceptable salt thereof, wherein:
R¹ is selected from —NR^{a}R^{b}, —C₁-C₃ alkylene-NR^{a}R^{b}, —C₁-C₃ alkylene-OH, —C₁-C₆ alkyl, —C₁-C₆ haloalkyl, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, and —C₁-C₃ alkylene-(5- to 10-membered heteroaryl), wherein said —C₁-C₃ alkylene-NR^{a}R^{b}, —C₂-C₆ alkenyl, —C₂-C₆ alkynyl, —C₃-C₈ cycloalkyl, —C₁-C₃ alkylene-(C₃-C₈ cycloalkyl), 4- to 10-membered heterocyclyl, —C₁-C₃ alkylene-(4- to 10-membered heterocyclyl), —C₆-C₁₀ aryl, —C₁-C₃ alkylene-(C₆-C₁₀ aryl), 5- to 10-membered heteroaryl, or —C₁-C₃ alkylene-(5- to 10-membered heteroaryl) is optionally substituted with 1-3 substituents independently selected from halo, —OH, —C₁-C₃ alkyl, and —C₁-C₃ alkoxy;
each R^{4a} is independently selected from —H, halo, —OH, —CN, —C₁-C₆ alkyl, —C₁-C₆haloalkyl, and —C₁-C₆ alkoxy; or
two R^{4a} together form an oxo;
each R^{4b} is independently selected from —H, halo, —OH, —CN, —C₁-C₆ alkyl, —C₁-C₆haloalkyl, and —C₁-C₆ alkoxy;
R⁵, R⁶, and R⁷ are each independently selected from —H, halo, —OH, —CN, —C₁-C₆ alkyl, —C₁-C₆ haloalkyl, and —C₁-C₆ alkoxy;
each R⁸ and each R⁹ is independently selected from —H, halo, —C₁-C₆ alkyl, —C₁-C₆ haloalkyl, and —C₁-C₆ alkoxy; or
R⁸ and R⁹, together with together with the carbon atom to which they are attached, form a C₃-C₆ cycloalkyl optionally substituted with halo, —C₁-C₃ alkyl, —C₁-C₃ haloalkyl, or —C₁-C₃ alkoxy;
each R¹⁰ is independently selected from halo, —C₁-C₆ alkyl, —C₁-C₆haloalkyl, and —C₁-C₆ alkoxy;
R^{a} and R^{b} are each independently selected from —H and —C₁-C₃ alkyl;
n is 0, 1, 2, or 3; and
p is 0, 1, 2, 3, or 4.

38. The compound of claim 1, or a pharmaceutically acceptable salt thereof, wherein R¹ is selected from —CH₃, —CH₂CH₃, —CH₂CH₂CH₃, —CH(CH₃)₂, —CH(CH₃)CH₂CH₃, —CH₂F, —CHF₂, —CF₃, —CHFCH₃—CH₂CF₃—CF₂CH₃, —CH₂CH₂OH, 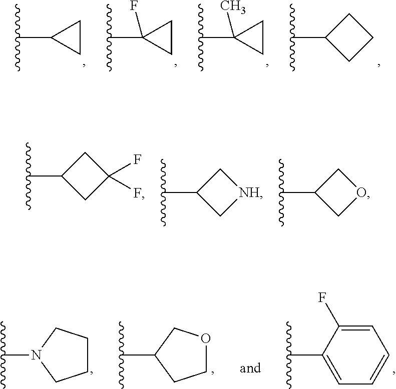
optionally wherein R¹ is selected from —CHF₂, —CF₃, and —CF₂CH₃.

39. The compound of claim 1, or a pharmaceutically acceptable salt thereof, wherein:
R5 is —H, —F, —Cl, —Br, or —CH3;
R6 is —H —F, or —Br; and
R7 is —H, —F, —Br, —CH3, or —CF3.

40. The compound of claim 1, or a pharmaceutically acceptable salt thereof, wherein each R⁸ and each R⁹ is independently selected from —H, —F, —CH₃, and —CH₂CH₃.

41. The compound of claim 1, or a pharmaceutically acceptable salt thereof, wherein p is 0, 1, or 2.

42. The compound of claim 1, or a pharmaceutically acceptable salt thereof, wherein each R¹⁰ is independently selected from —F and —CH₃.

43. The compound of claim 1, or a pharmaceutically acceptable salt thereof, wherein:
X is O; m is 1; and nis 1;
X is O; m is 2; and n is 1; or
X is O; m is 1; and nis 2.

44. The compound of claim 1, or a pharmaceutically acceptable salt thereof, wherein:
X is O; R¹ is —CF₃; m is 1; and n is 1;
X is O; R¹ is —CF₃; m is 2; and n is 1;
X is O; R¹ is —CHF₂, —CF₃, or —CF₂CH₃; m is 1; and n is 2;
X is O; Y is O; m is 1; and nis 1;
X is O; Y is O; mis 2; and n is 1;
X is O; Y is O; m is 1; and n is 2;
X is O; Y is CH₂; m is 1; and n is 1;
X is O; Y is CH₂; m is 2; and n is 1; or
X is O; Y is CH₂; m is 1; and n is 2.

45. The compound of claim 1, or a pharmaceutically acceptable salt thereof, wherein:
X is O; Y is O; R¹ is —CF₃; m is 1; and n is 1;
X is O; Y is O; R¹ is —CF₃; m is 2; and n is 1;
X is O; Y is O; R¹ is —CHF₂, —CF₃, or —CF₂CH₃; m is 1; and n is 2;
X is O; Y is CH₂; R¹ is —CF₃; m is 1; and n is 1;
X is O; Y is CH₂; R¹ is —CF₃; m is 2; and n is 1;
X is O; Y is CH₂; R¹ is —CF₃; m is 1; and n is 2; or
X is O; Y is O; R¹ is —CF₃; m is 1; n is 1; and q is 0.

46. The compound of claim 1, or a pharmaceutically acceptable salt thereof, wherein:
X is O; Y is O; R¹ is —CF₃; m is 2; n is 1; and q is 0;
X is O; Y is O; R¹ is —CHF₂, —CF₃, or —CF₂CH₃; m is 1; n is 2; and q is 0;
X is O; Y is CH₂; R¹ is —CF₃; m is 1; n is 1; and q is 0;
X is O; Y is CH₂; R¹ is —CF₃; m is 2; n is 1; and q is 0; or
X is O; Y is CH₂; R¹ is —CF₃; m is 1; n is 2; and q is 0.

47. The compound of claim 1, wherein the compound is selected from: 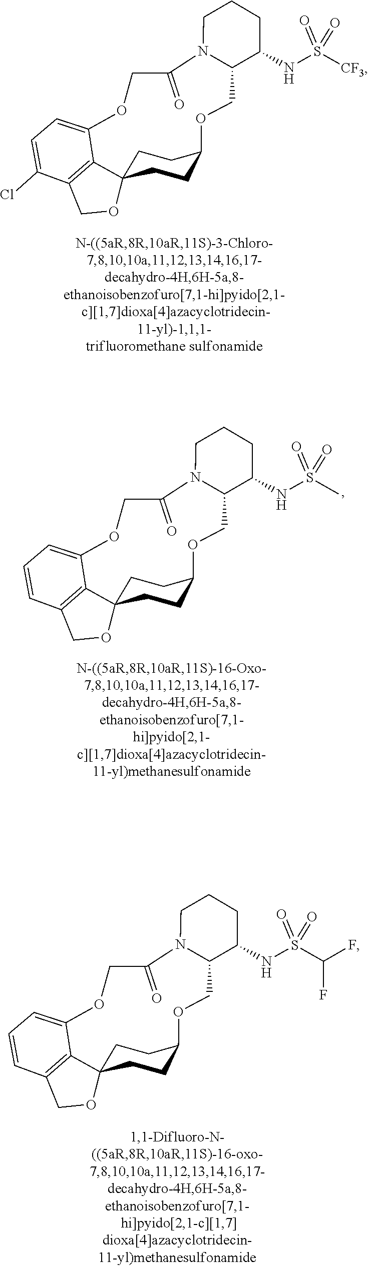 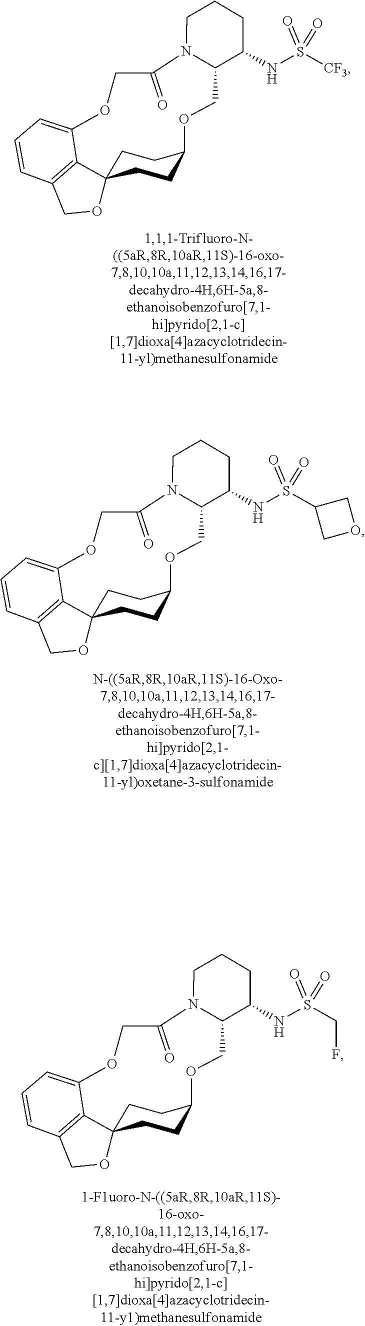 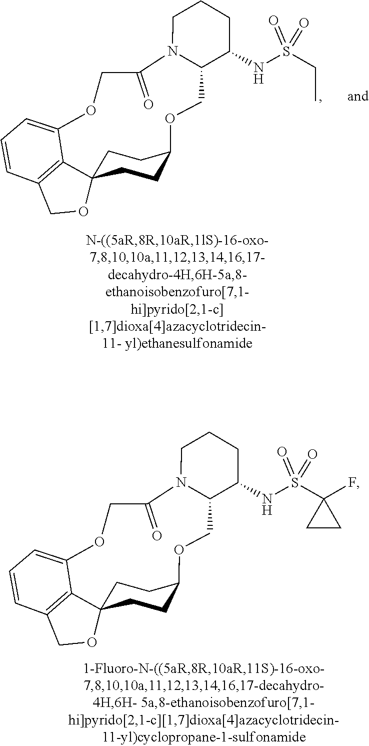
or a pharmaceutically acceptable salt thereof.

48. The compound of claim 1, wherein the compound is selected from: 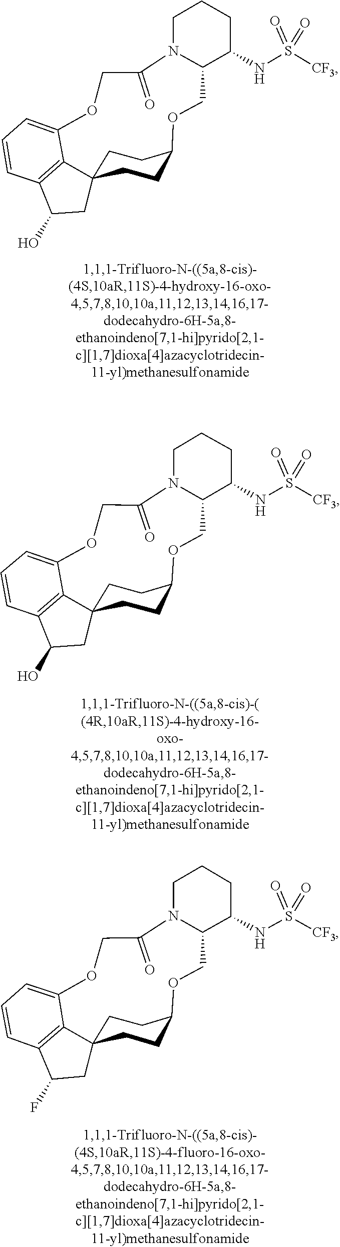 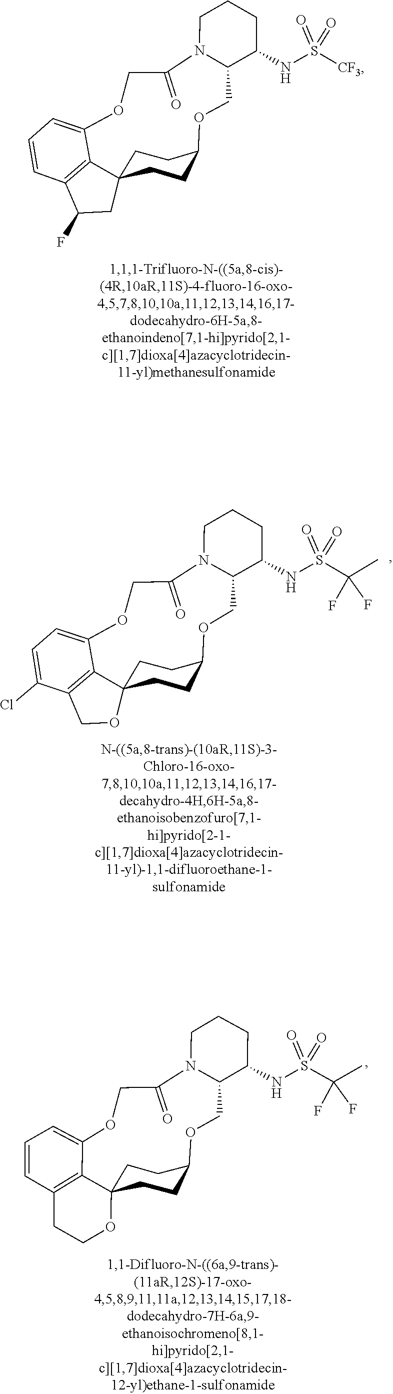 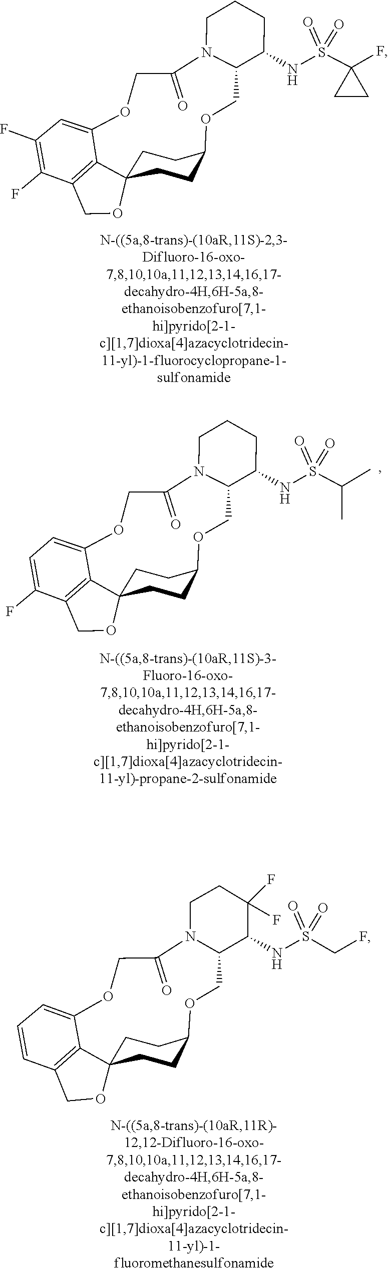 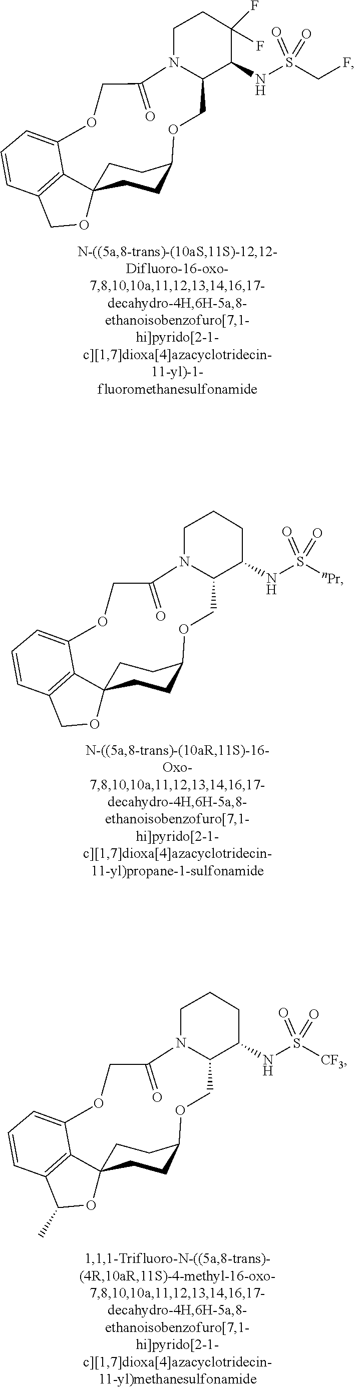 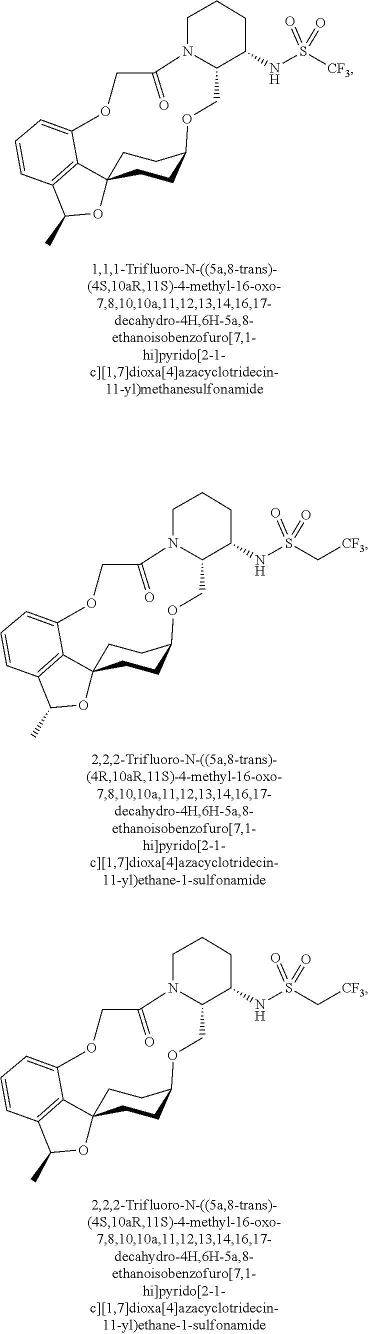 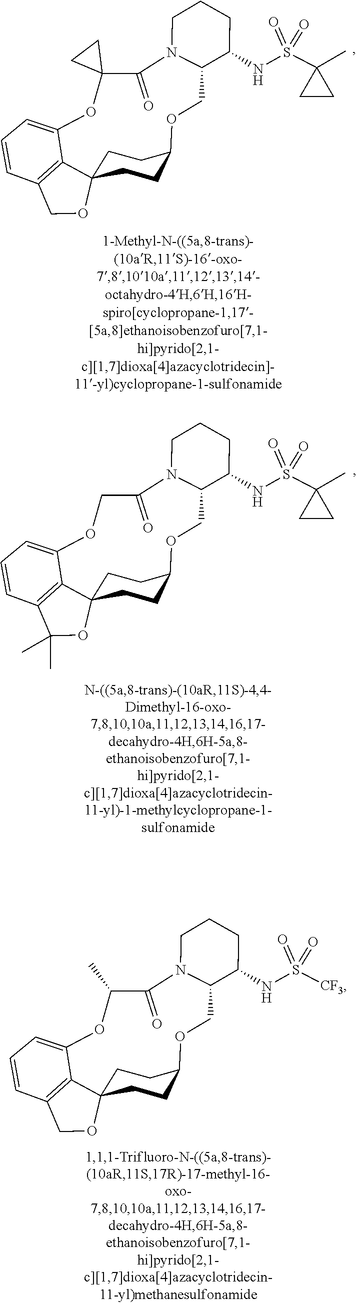 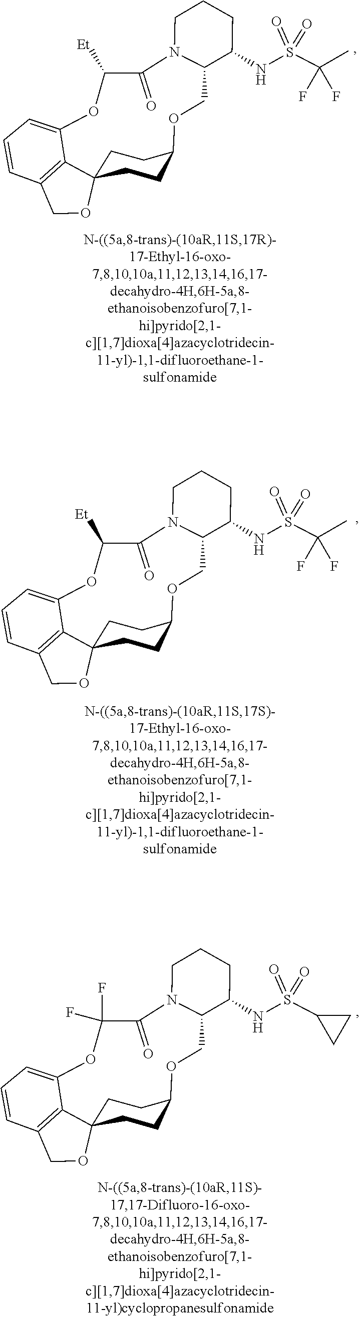 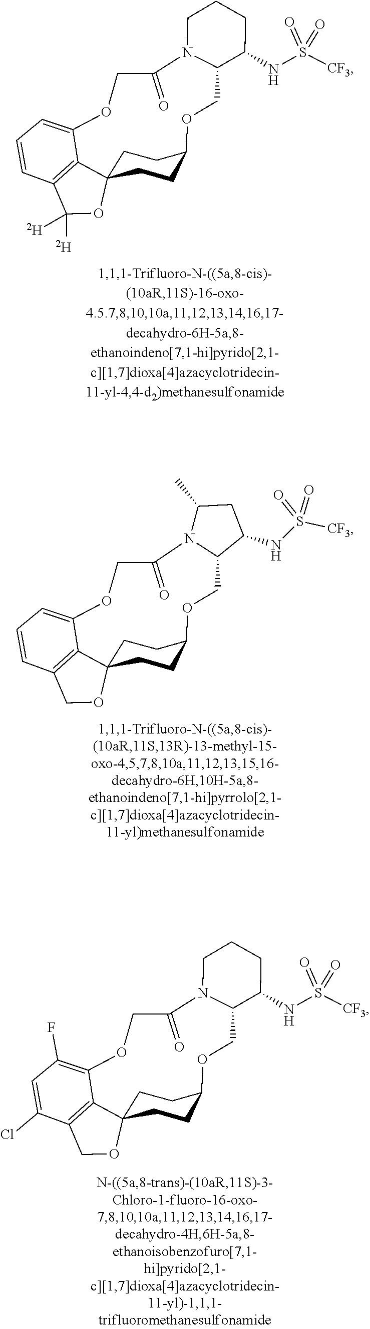 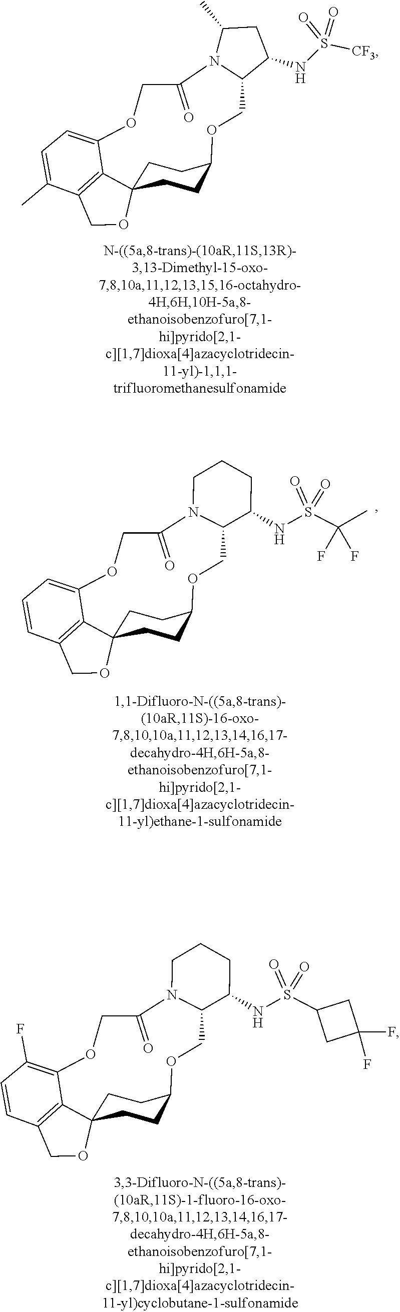 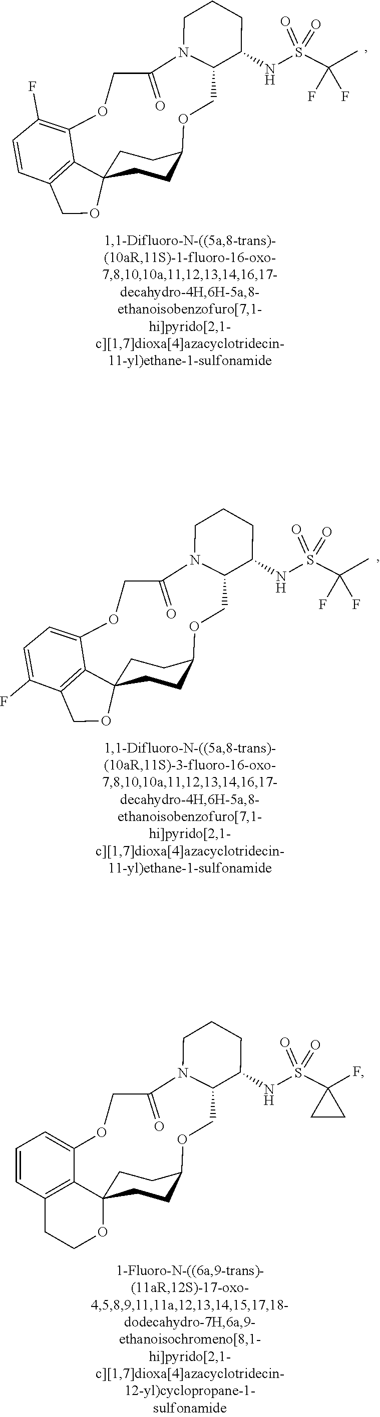 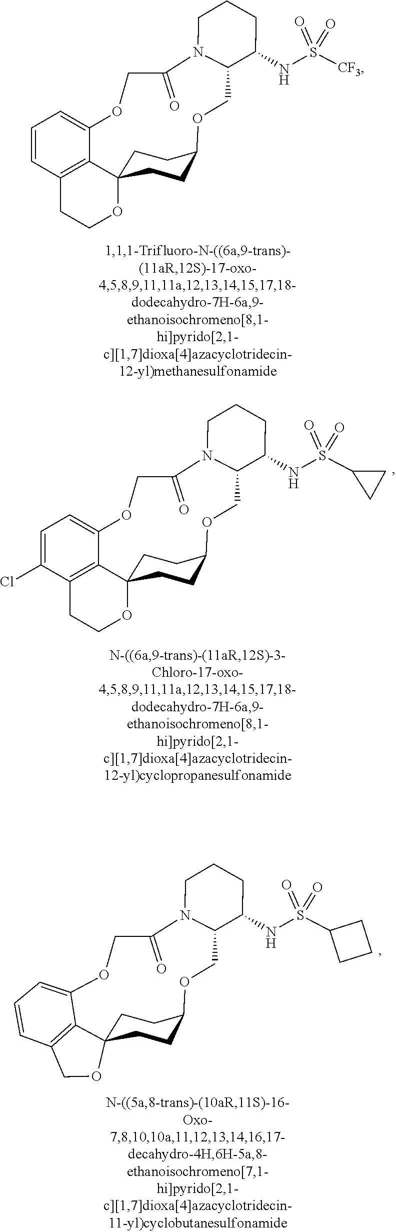 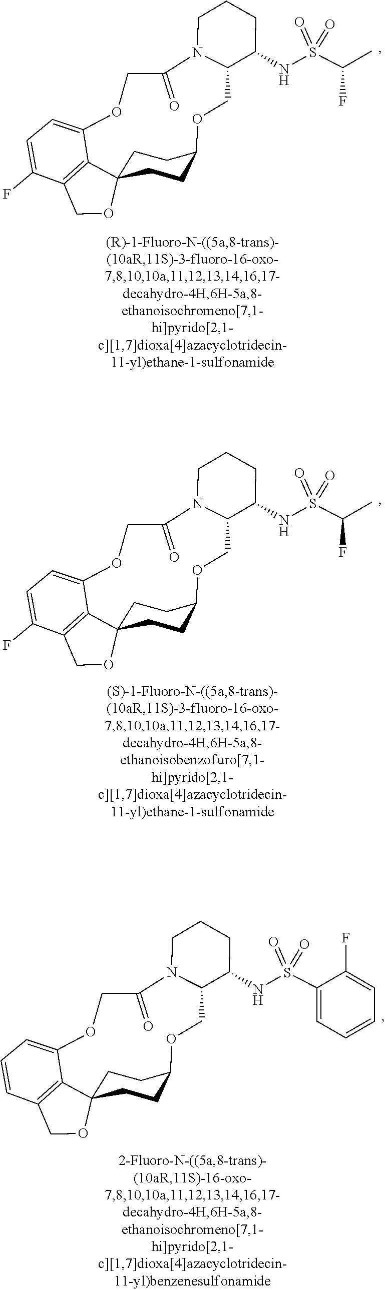 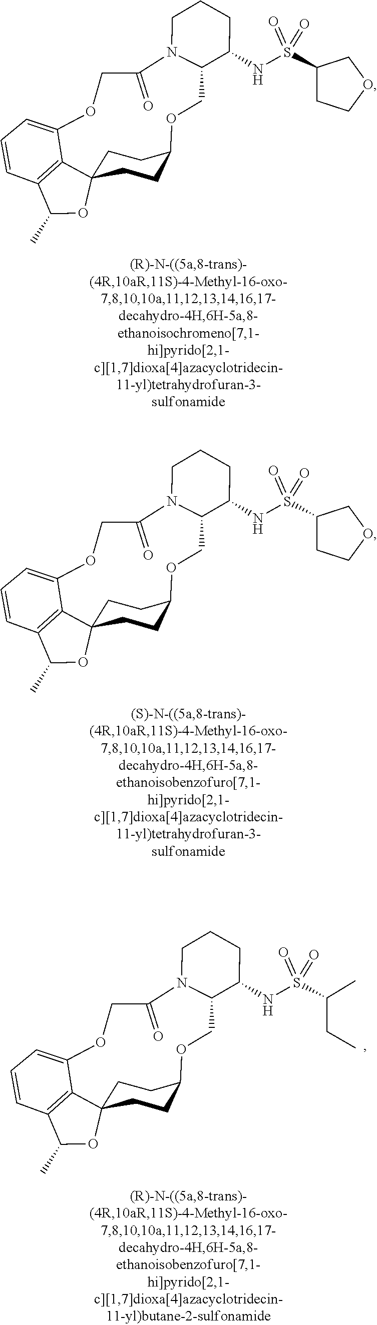 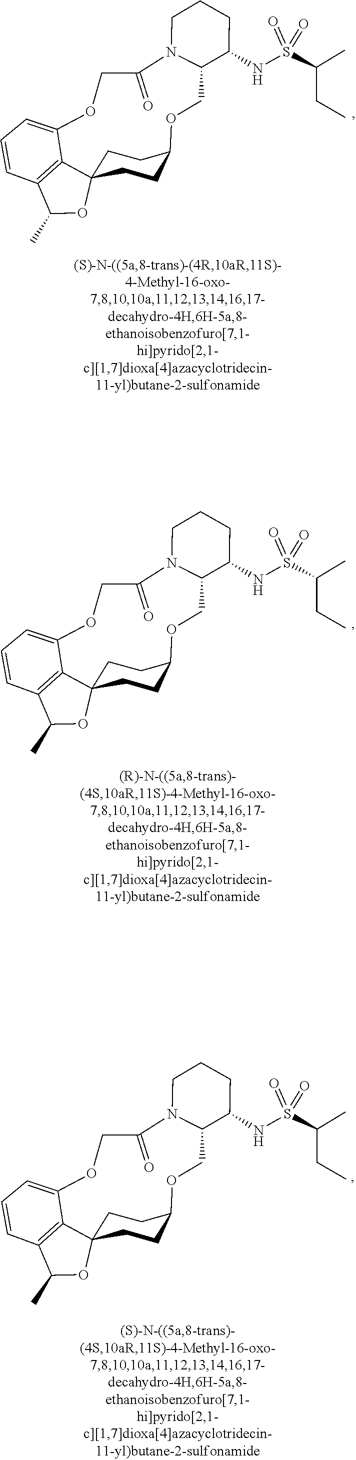 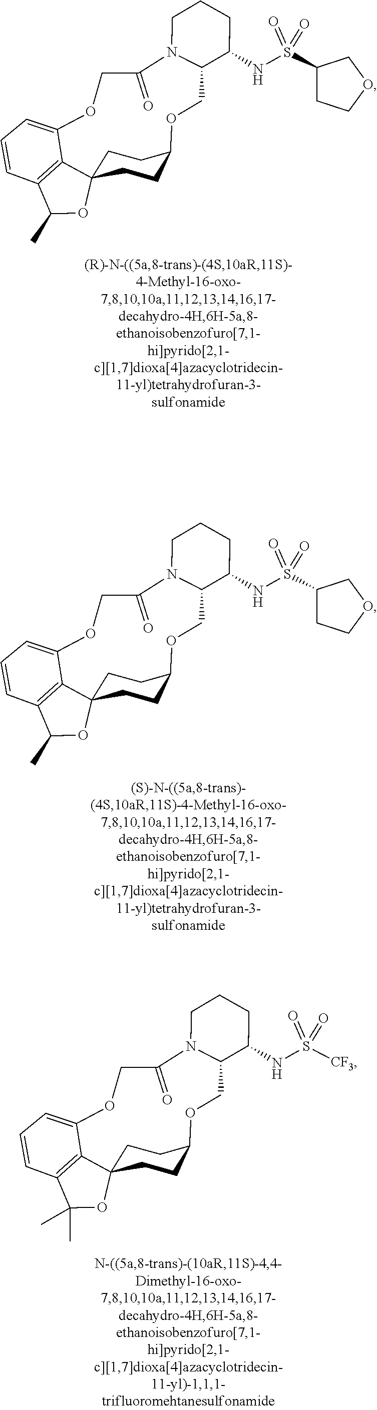 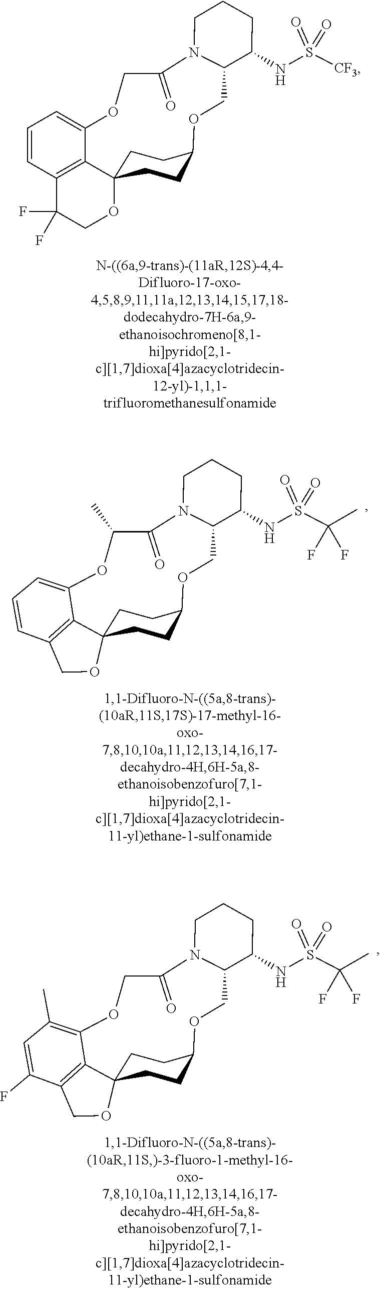 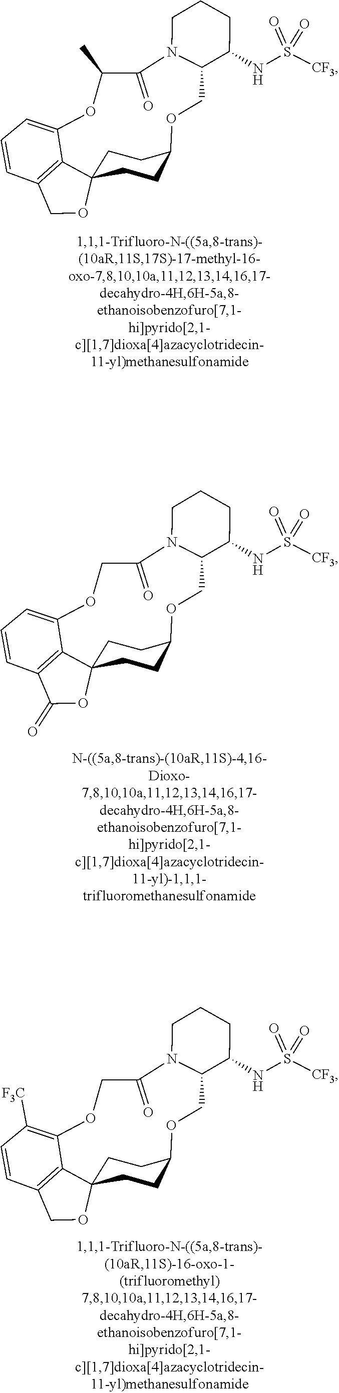 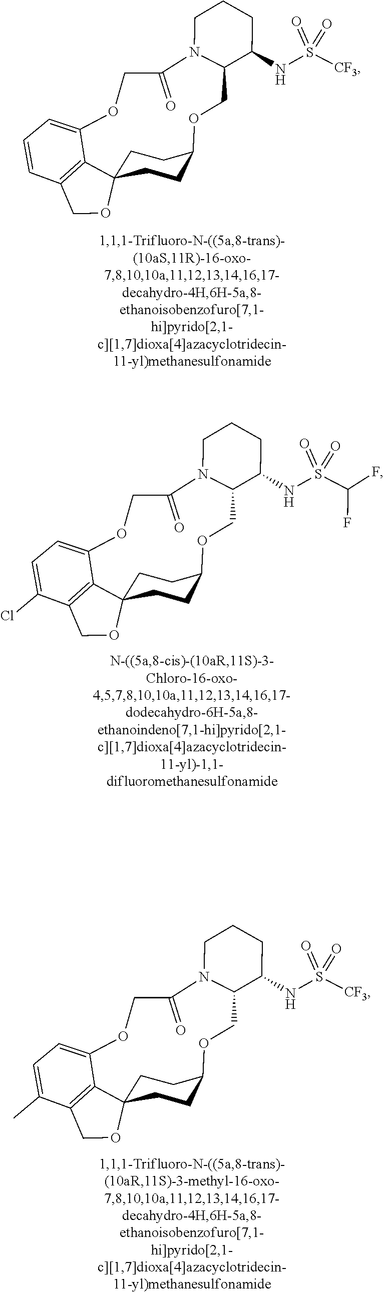 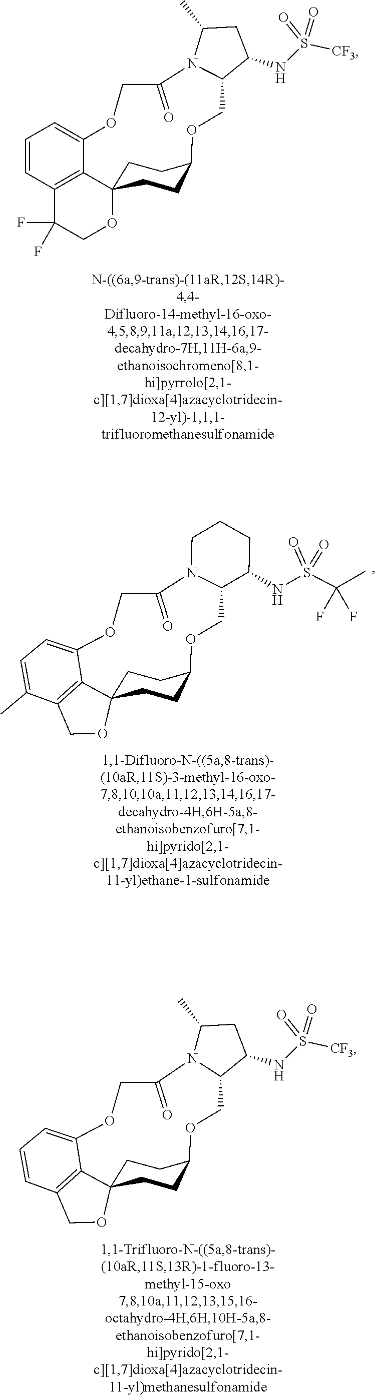 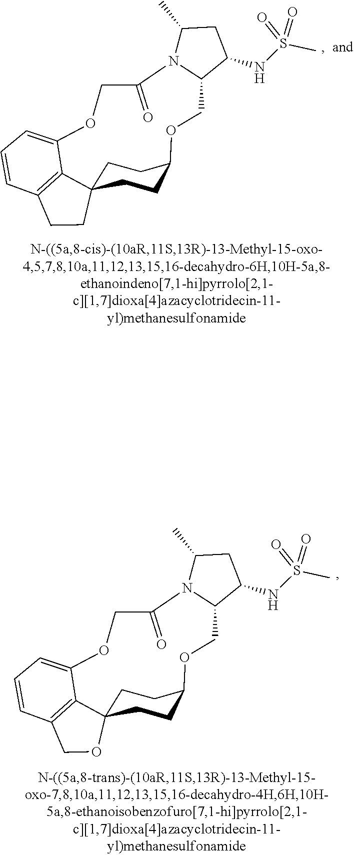
or a pharmaceutically acceptable salt thereof.

49. The compound of claim 1, wherein the compound is selected from: 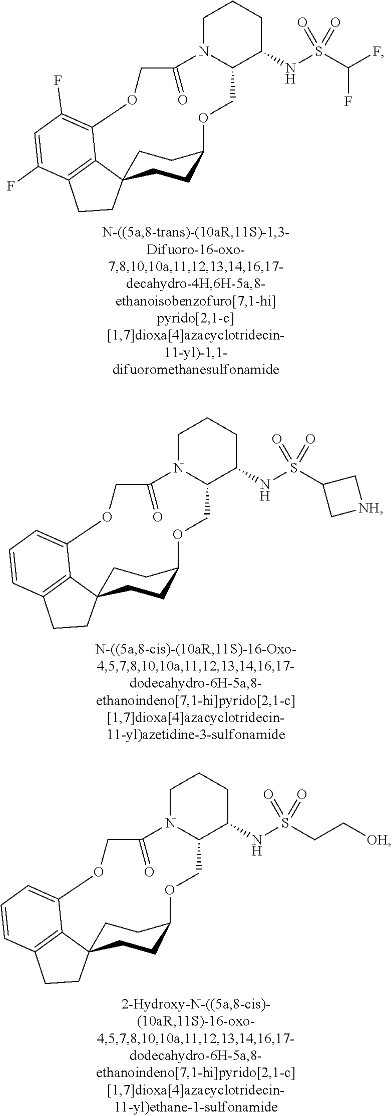 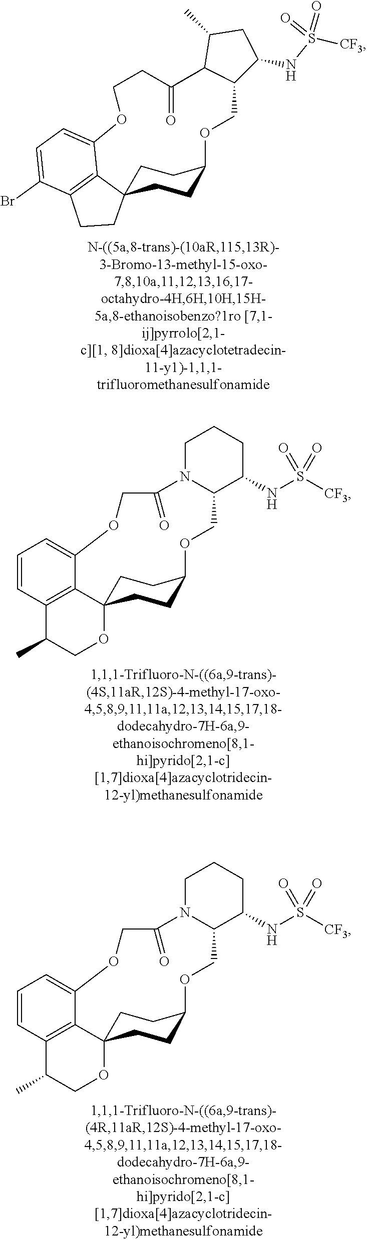 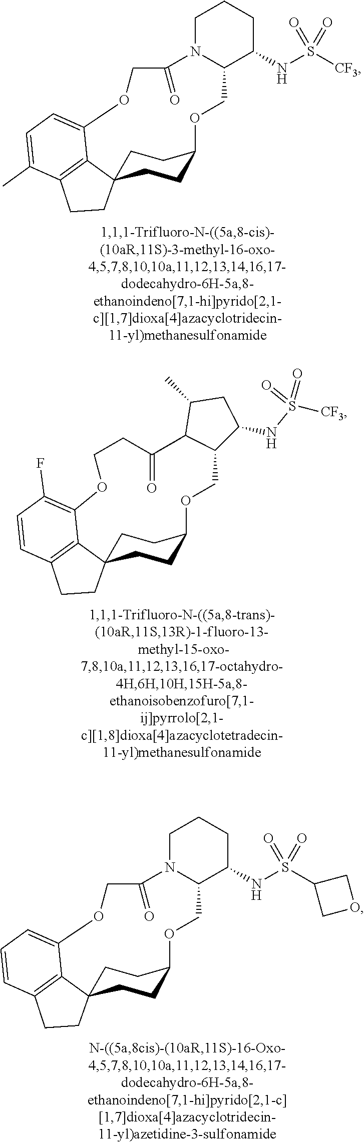 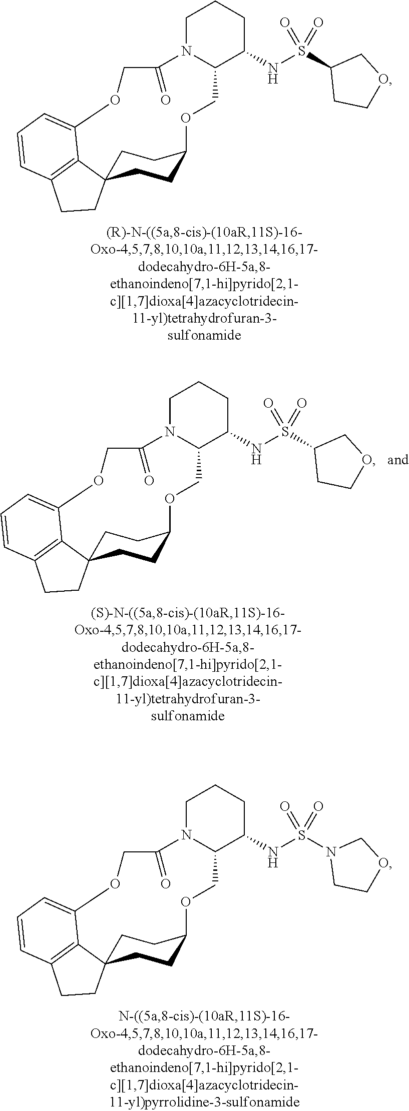
or a pharmaceutically acceptable salt thereof.

50. The compound of claim 1 in non-salt form.

51. A pharmaceutical composition comprising a compound of claim 1, or a pharmaceutically acceptable salt thereof, and at least one pharmaceutically acceptable excipient.

52. A method of preventing or treating a disease associated with a deficiency in orexin 2 receptor signaling, comprising administering to a subject in need thereof a therapeutically effective amount of a compound of claim 1, or a pharmaceutically acceptable salt thereof, optionally wherein the disease is selected from narcolepsy, idiopathic hypersomnia, hypersomnia, sleep apnea syndrome, narcolepsy syndrome involving narcolepsy-like symptom, hypersomnia associated with Parkinson's disease, hypersomnia associated with dementia with Lewy body, hypersomnia syndrome involving daytime hypersomnia (e.g., Kleine-Levin syndrome, major depression accompanied by hypersomnia, dementia with Lewy body, Parkinson's disease, progressive supranuclear palsy, Prader-Willi syndrome, Moebius syndrome, hypoventilation syndrome, Niemann-Pick disease type C, brain contusion, cerebral infarction, brain tumor, muscular dystrophy, multiple sclerosis, acute disseminated encephalomyelitis, Guillain-Barre syndrome, Rasmussen's encephalitis, Wernicke's encephalopathy, limbic encephalitis, Hashimoto encephalopathy), coma, loss of consciousness, obesity (e.g., malignant mast cell, extrinsic obesity, hyperinsulinar obesity, hyperplasmic obesity, hypophysial obesity, hypoplasmic obesity, hypothyroid obesity, hypothalamic obesity, symptomatic obesity, childhood obesity, upper body obesity, alimentary obesity, gonadal obesity, systemic mastocytosis, primary obesity, central obesity), insulin resistance syndrome, Alzheimer, impaired consciousness such as coma, side effect or complication caused by anesthesia, sleep disturbance, sleep problem, insomnia, intermittent sleep, night myoclonus, REM sleep interruption, jet lag, jet lag syndrome, sleep disorder of shift workers, dyssomnia, sleep terror, depression, major depression, sleepwalking, enuresis, sleep disorder, Alzheimer's sundown syndrome, disease associated with circadian rhythm, fibromyalgia, condition resulting from decrease in sleeping quality, bulimia, obsessive eating disorder, obesity-related diseases, hypertension, diabetes, elevated plasma insulin level/insulin resistance, hyperlipemia, hyperlipidaemia, endometrial cancer, breast cancer, prostate cancer, colon cancer, cancer, osteoarthritis, obstructive sleep apnea, cholelithiasis, gallstone, heart disease, abnormal heartbeat, arrhythmia, myocardial infarction, congestive heart failure, heart failure, coronary heart disease, cardiovascular disease, sudden death, polycystic ovary, craniopharyngioma, Prader Willi syndrome, Froehlich syndrome, growth hormone deficiency, normal variant short stature, Turner syndrome, children suffering from acute lymphoblastic leukemia, syndrome X, reproductive hormone abnormality, decrease of fecundability, infertility, hypogonadism in men, sexual/reproductive-function dysfunction such as hirsutism in women, fetal defect associated with maternity obesity, gastrointestinal motility disorder such as obesity-related gastroesophageal reflux, obesity hypoventilation syndrome (Pickwickian syndrome), respiratory disease such as respiratory distress, inflammation such as vascular systemic inflammation, arteriosclerosis, hypercholesterolemia, hyperuricemia, low back pain, gallbladder disease, gout, renal cancer, secondary risk of obesity such as risk of left ventricle hypertrophy, migraine, headache, neuropathic pain, Parkinson's disease, psychosis, schizophrenia, facial flushing, night sweat, disease in genitalium/urinary system, disease associated with sexual function or fecundability, dysthymic disorder, bipolar disorder, bipolar I disorder, bipolar II disorder, cyclothymic disorder, acute stress disorder, agoraphobia, generalized anxiety disorder, obsessive-compulsive disorder, panic attack, panic disorder, posttraumatic stress disorder, separation anxiety disorder, social phobia, anxiety disorder, acute neurological and psychiatric disorder such as cerebral deficiency developed after heart bypass surgery or heart transplant, stroke, ischemic stroke, cerebral ischemia, spinal cord trauma, head injury, periparturient hypoxia, cardiac arrest, hypoglycemic nerve injury, Huntington's disease, amyotrophic lateral sclerosis, multiple sclerosis, eye damage, retinopathy, cognitive impairment, muscle spasm, tremor, epilepsy, disorder associated with muscle spasm, delirium, amnestic disorder, age-associated cognitive decline, schizoaffective disorder, paranoia, drug addiction, movement disorder, chronic fatigue syndrome, fatigue, medication-induced parkinsonian syndrome, Gilles de la Tourette syndrome, chorea, myoclonus, tic, restless legs syndrome, dystonia, dyskinesia, attention deficit hyperactivity disorder (ADHD), conduct disorder, urinary incontinence, withdrawal symptom, trigeminal neuralgia, hearing loss, tinnitus, nerve injury, retinopathy, macular degeneration, vomiting, cerebral edema, pain, bone pain, arthralgia, toothache, cataplexy, and traumatic brain injury.

53. A method of preventing or treating a disease selected from narcolepsy, idiopathic hypersomnia, hypersomnia, sleep apnea syndrome, narcolepsy syndrome involving narcolepsy-like symptoms, hypersomnia associated with Parkinson's disease, and hypersomnia associated with dementia with Lewy body, comprising administering to a subject in need thereof a therapeutically effective amount of a compound of claim 1, or a pharmaceutically acceptable salt thereof.
